(19) United States
(12) Patent Application Publication
(10) Pub. No.: US 2020/0077658 A1
(43) Pub. Date: Mar. 12, 2020

(54) Pesticidal Compounds

(71) Applicant: BASF SE, Ludwigshafen am Rhein (DE)

(72) Inventors: Sunderraman SAMBASIVAN, Navi Mumbai (IN); Arun NARINE, Ludwigshafen (DE); Rupsha CHAUDHURI, Navi Mumbai (IN); Ramakrishnan VALLINAYAGAM, Navi Mumbai (IN); Devendra VYAS, Navi Mumbai (IN); Ashokkumar ADISECHAN, Navi Mumbai (IN); Gopal Krishna DATTA, Goettingen (DE)

(21) Appl. No.: 16/469,876

(22) PCT Filed: Dec. 6, 2017
(86) PCT No.: PCT/EP2017/081700

§ 371 Date: Jun. 14, 2019

(30) Foreign Application Priority Data
Dec. 16, 2016 (EP) 16204569.4

(51) Int. Cl. A01N 43/54; C07D 239/42; C07D 403/12; C07D 417/12; C07H 15/26; A01N 43/78; A01N 43/50

(52) U.S. Cl. CPC A01N 43/54; C07D 239/42; C07D 403/12; A01N 43/50; C07H 15/26; A01N 43/78; C07D 417/12

(57) ABSTRACT

The present invention relates to the compounds of formula (I), and the N-oxides, stereoisomers, tautomers and agriculturally or veterinarily acceptable salts thereof wherein the variables are defined according to the description,

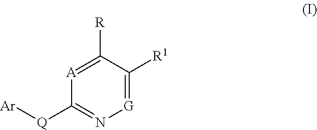

The compounds of formula (I), as well as the N-oxides, stereoisomers, tautomers and agriculturally or veterinarily acceptable salts thereof, are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

Invertebrate pests and in particular insects, arachnids and nematodes destroy growing and harvested crops and attack wooden dwelling and commercial structures, thereby causing large economic loss to the food supply and to property. Accordingly, there is an ongoing need for new agents for combating invertebrate pests.

Carbamoylated and thiocarbamoylated oxime derivatives are known for pesticidal use, for example, in patent publications WO 2016/156076, semi-carbazones and thiosemicarbazones derivatives are known for pesticidal use in patent publication WO 2016/116445.

Due to the ability of target pests to develop resistance to pesticidally-active agents, there is an ongoing need to identify further compounds, which are suitable for combating invertebrate pests such as insects, arachnids and nematodes. Furthermore, there is a need for new compounds having a high pesticidal activity and showing a broad activity spectrum against a large number of different invertebrate pests, especially against difficult to control insects, arachnids and nematodes.

It is therefore an object of the present invention to identify and provide compounds, which exhibit a high pesticidal activity and have a broad activity spectrum against invertebrate pests.

It has been found that these objects can be achieved by substituted bicyclic compounds of formula I, as depicted and defined below, including their stereoisomers, their salts, in particular their agriculturally or veterinarily acceptable salts, their tautomers and their N-oxides.

In a first aspect, the present invention relates to the compounds of formula I,

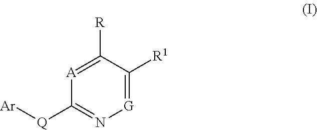

Wherein
A is N or CR^{A};
G is N or CR^{B};
R, R^{A}, and R^{B} are H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₁-C₄-alkyl-C₃-C₆-cycloalkoxy, which are unsubstituted or substituted with halogen, C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘR^{e}, one radical may also be phenyl, phenoxy, phenylcarbonyl, phenylthio or benzyl, wherein the rings are unsubstituted or substituted with R^{f};
Q is NR², O, or S(═O)ₘ, wherein
   R² is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen,
   C(O)—OR^{a}, C₁-C₆-alkylen-NR^{b}R^{c}, C₁-C₆-alkylen-CN,
   C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
Ar is phenyl or 5- or 6-membered hetaryl, which are unsubstituted or substituted with R^{Ar}, wherein
   R^{Ar} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which are unsubstituted or substituted with halogen,
   C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘR^{e}, one radical may also be phenyl, phenoxy, phenylcarbonyl, phenylthio, or benzyl, where the rings are unsubstituted or substituted with R^{f};
R¹ is a moiety of formula X—Y—Z-T-R¹¹ or X—Y—Z-T-R¹²; wherein
   X is —CR^{xa}R^{xb}—, —O—, —S—, —NR^{xc}—, —CR^{xa}═CR^{xb}—, —CR^{xa}R^{xb}—CR^{xa}R^{xb}—, —O—CR^{xa}R^{xb}—, —S—CR^{xa}R^{xb}—, —N═CR^{xa}—, —NR^{xc}—CR^{xa}R^{xb}—, —NR^{xc}—C(═S)—, —N═C(S—R^{e})—, or —NR^{xc}—C(═O)—;
   Y is —CR^{ya}═N—, wherein the N is bound to Z;
      —NR^{yc}—C(═O)—, wherein C(═O) is bound to Z; and
      —NR^{yc}—C(═S)—, wherein C(═S) is bound to Z;
   Z is a single bond;
      —NR^{zc}—C(═S)—, wherein C(═S) is bound to T;
      —NR^{zc}—C(═O)—, wherein C(═O) is bound to T;
      —N═C(S—R^{za})—, wherein T is bound to the carbon atom;
      —O—C(═O)—, wherein T is bound to the carbon atom;
      —O—C(═S)—, wherein T is bound to the carbon atom; and
      —NR^{zc}—C(S—R^{za})═, wherein T is bound to the carbon atom;
   T is O, N or N—R^{T};
   R¹¹ is C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₁-C₄-alkyl-C₃-C₆-cycloalkoxy, which are unsubstituted or substituted with halogen,
      C₁-C₆-alkylen-NR^{b}R^{c}, C₁-C₆-alkylen-CN, C(O)—NR^{b}R^{c}, C(O)—R^{d}, aryl, arylcarbonyl, aryl-C₁-C₄-alkyl, aryloxy-C₁-C₄-alkyl, hetaryl, carbonylhetaryl, hetaryl-C₁-C₄-alkyl, or hetaryloxy-C₁-C₄-alkyl, where the rings are unsubstituted or substituted with R^{g} and wherein the hetaryl is a 5- or 6-membered monocyclic hetaryl or a 8-, 9- or 10-membered bicyclic hetaryl;
   R¹² is a radical of the formula A¹;

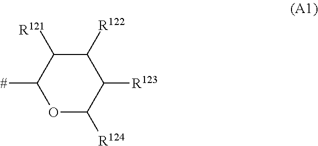

wherein # indicates the point of attachment to T;
   R¹²¹, R¹²², R¹²³ are H, halogen, C₁-C₆-alkyl, C₁-C₆-haloalkyl, C₂-C₆-alkenyl, C₂-C₆-alkynyl, C₁-C₆-alkoxy-C₁-C₄-alkyl, C₁-C₆-alkoxy, C₁-C₆-haloalkoxy, C₂-C₆-alkenyloxy, C₂-C₆-alkynyloxy, C₁-C₆-alkoxy-C₁-C₄-alkoxy, C₁-C₆-alkylcarbonlyoxy, C₁-C₆-haloalkylcarbonlyoxy, C₁-C₆-alkenylcarbonlyoxy, C₃-C₆-cycloalkylcarbonlyoxy, or NR^{b}R^{c}, or one of R¹²¹, R¹²², R¹²³ may also be oxo;
   R¹²⁴ is H, C₁-C₆-alkyl, C₁-C₆-haloalkyl, C₁-C₆-alkoxy-C₁-C₄-alkyl, C₁-C₆-alkoxy, C₁-C₆-haloalkoxy, or C₂-C₆-alkenyloxy;
and where
R^{xa}, R^{xb}, R^{ya} are H, halogen, C₁-C₆-alkyl, C₁-C₆-alkoxy, C₁-C₆-haloalkyl, C₁-C₆-haloalkoxy, C₂-C₆-alkenyl, C₂-C₆-alkynyl, C₁-C₆-alkoxy-C₁-C₄-alkyl, C₃-C₆-cycloalkyl, C₁-C₄-alkyl-C₃-C₆-cycloalkyl, C₁-C₄-alkyl-C₃-C₆-cycloalkoxy, which are unsubstituted or substituted with halogen,
   C(O)—OR^{a}, C₁-C₆-alkylen-NR^{b}R^{c}, C₁-C₆-alkylen-CN, C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
R^{xc}, R^{yc}, R^{zc} are H, C₁-C₆-alkyl, C₁-C₆-haloalkyl, C₂-C₆-alkenyl, C₂-C₆-alkynyl, C₁-C₄-alkyl-C₁-C₆-alkoxy, C₃-C₆-cycloalkyl, C₁-C₄-alkyl-C₃-C₆-cycloalkyl, or C₁-C₄-alkyl-C₃-C₆-cycloalkoxy, which are unsubstituted or substituted with halogen;
R^{T} is H, C₁-C₆-alkyl, C₁-C₆-haloalkyl, C₂-C₆-alkenyl, C₂-C₆-alkynyl, C₁-C₄-alkyl-C₁-C₆-alkoxy, C₃-C₆-cycloalkyl, C₃-C₆-cycloalkyl-C₁-C₄-alkyl, C₃-C₆-cycloalkoxy-C₁-C₄-alkyl, where the alkyl, which are unsubstituted or substituted with halogen, C(O)—OR^{a}, C₁-C₆-alkylen-NR^{b}R^{c}, C₁-C₆-alkylen-CN, C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
R^{zc} together with R^{T} if present, may form C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene are unsubstituted or substituted with R^{h};
R^{za} is H, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₄-alkyl-C₁-C₆-alkoxy, C₃-C₆-cycloalkyl, C₁-C₄-alkyl-C₃-C₆-cycloalkoxy, or C₁-C₄-alkyl-C₃-C₆-cycloalkyl, which are unsubstituted or substituted with halogen,
   C₁-C₆-alkylen-NR^{b}R^{c}, C₁-C₆-alkylen-CN, C(O)—NR^{b}R^{c}, C(O)—R^{d}, phenyl, phenylcarbonyl and benzyl, wherein the rings are unsubstituted or substituted with R^{f};
R^{za} together with R^{T} if present, may form C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene may be unsubstituted or substituted with R^{h};
R^{a}, R^{b} and R^{c} are H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen,
   C₁-C₆-alkylen-CN, phenyl, or benzyl, wherein the rings are unsubstituted or substituted with R^{f};
R^{d} is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen,
   phenyl, or benzyl, wherein the rings are unsubstituted or substituted with R^{f};
R^{e} is C₁-C₆-alkyl, C₁-C₆-haloalkyl, C₃-C₆-cycloalkyl, C₃-C₆-cycloalkyl-C₁-C₄-alkyl, which are unsubstituted or substituted with halogen,
   phenyl, or benzyl, wherein the rings are unsubstituted or substituted with R^{f};
R^{f} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₀₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which are unsubstituted or substituted with halogen,
   C(O)—OR^{a}, NR^{b}R^{c}, C₁-C₆-alkylen-NR^{b}R^{c}, O—C₁-C₆-alkylen-NR^{b}R^{c},
   C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘRe;
R^{g} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₀₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which are unsubstituted or substituted with halogen,
   C(O)—OR^{a}, NR^{b}R^{c}, C₁-C₆-alkylen-NR^{b}R^{c}, O—C₁-C₆-alkylen-NR^{b}R^{c},
   C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘRe;
R^{h} halogen, OH, C₁-C₆-alkyl, C₃-C₆-cycloalkyl, or CN;
m is 0, 1, or 2;

and the N-oxides, stereoisomers, tautomers and agriculturally or veterinarily acceptable salts thereof.

Moreover, the present invention also relates to processes and intermediates for preparing compounds of formula I and to active compound combinations comprising them. Moreover, the present invention relates to agricultural or veterinary compositions comprising the compounds of formula I, and to the use of the compounds of formula I or compositions comprising them for combating or controlling invertebrate pests and/or for protecting crops, plants, plant propagation material and/or growing plants from attack and/or infestation by invertebrate pests. The present invention also relates to methods of applying the compounds of formula I. Furthermore, the present invention relates to seed comprising compounds of formula I. Wherein the compounds of formula I includes N-oxides, stereoisomers, tautomers and agriculturally or veterinarily acceptable salts thereof.

With due modification of the starting compounds, the compounds of formula I can be prepared by procedures as given in below schemes.

The compounds of the formula (I) can be prepared by the methods described herein after in below reactions and in the synthesis description of the preparation examples. In the reactions below, the radicals Ar, Q, G, R and R¹, R², R^{xa}, R^{ya}, R^{yz}, R^{yc}, R^{xc}, R¹¹, R¹² are as defined above for formula (I), unless otherwise specified.

Compounds of formula (I) in which Z is a single bond or —NR^{zc}—C(═S)— or —NR^{zc}—C(═O)— or O—C(═O)— or —O—C(═S)— and T is O, N or N—R^{T} denotes compounds of formula Ia and can be prepared in accordance with the methods described in the examples and by analogy to the methods described in WO 2011/017504 and as depicted in below reaction.

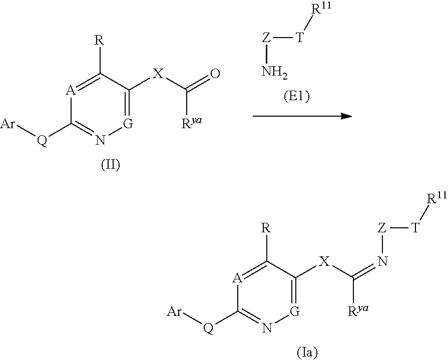

In one embodiment of the above reaction, an aldehyde or ketone of the formula (II) is reacted with a compound of formula (E1) wherein Z is —NR^{zc}—C(═S)— or —NR^{zc}—C(═O)— and T is N, in the presence or in the absence of a solvent. Suitable solvents are polar protic solvents, preferably Ethanol. If the reaction is performed in the absence of a solvent, the compound of the formula (E1) usually also act as solvent. Compounds of the formula (E1) are commercially available or can be prepared according to Journal of Medicinal Chemistry, 2010, 53(8), 3048 or Bioorganic & Medicinal Chemistry Letters, 2009, 19(4), 1152-1154 or WO2007003944.

According to another embodiment of the above reaction, an aldehyde or ketone compound of the formula (II) is first reacted with a hydrazine of the formula R^{zc}NHNH₂ followed by the reaction with an isocyanate of the formula R¹¹—NCO or with an isothiocyanate R¹¹—NCS to yield a compound of the formula (Ia), wherein Z is —N(R^{zc})—C(═O) or —N(R^{z}c)—C(═S) and T is N.

According to another embodiment of the above reaction, an aldehyde or ketone compound of the formula (II) is first reacted with a hydroxylamine followed by the reaction with a compounds R¹²-L, where L is a suitable leaving group, such as halogen or activated OH. Thereby, a compound of the formula (Ia) will result, wherein Z is a single bond and T is O.

According to another embodiment of the above reaction, an aldehyde or ketone compound of formula (II) is first reacted with a hydroxylamine followed by reaction with an isocyanate of the formula R¹¹—NCO or with an isothiocyanate R¹¹—NCS to yield a compound of the formula (Ia), wherein Z is —O—C(═O)— or —O—C(═S)— and T is N.

Compounds of formula (I) in which Z is —NR^{zc}—C(═S)— or —NR^{zc}—C(═O)—, wherein C(═S) or C(═O) is bound to T and T is O, N or N—R^{T}, denotes compounds of formula Ib and can be prepared as shown in reaction below by analogy to the method described in Synthesis, 2010, 2990-2966.

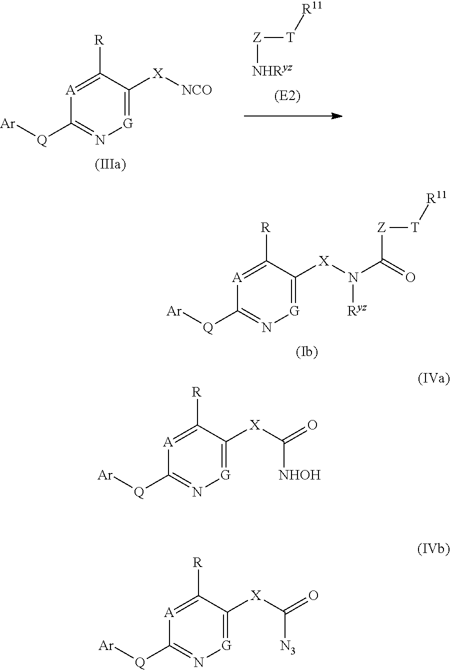

According to the method depicted in the above reaction, an isocyanate compound of the formula (IIIa) is reacted with the compound of formula (E2) by methods known to a person skilled in the art. The isocyanate of the formula (IIIa) may be obtained e.g. via Lossen rearrangement of the corresponding hydroxamic acid (IVa). The isocyanate of the formula (IIIa) may also be obtained via Curtius rearrangement of the corresponding azide of the formula (IVb), e.g. by analogy to the method described in WO 2014/204622. To this end, the hydroxamic acid is reacted with 1-propanephosphonic acid cyclic anhydride (T3P) in the presence of a base. The base is preferably N-methylmorpholine.

For converting compounds of formula (Ib) in which R^{yz} or R^{zc} is H into compounds (Ib) in which R^{yz} or R^{xz} is not H, compounds of formula (Ib) in which R^{yz} or R^{zc} is H can be reacted with compounds of formulae R^{yz}-Lg or R^{zc}-Lg wherein R^{yz} or R^{zc} is not H and Lg is a leaving group, such as a bromine, chlorine or iodine atom or a tosylate, mesylate or triflate, to yield compounds of formula (Ib), wherein R^{yz} or R^{zc} is different from H. The reaction is suitably carried out in the presence of a base such as sodium hydride or potassium hydride, suitably in a polar aprotic solvent such as N,N-dimethylformamide, tetrahydrofuran, dioxane, acetonitrile, dimethylsulfoxide or pyridine, or mixtures of these solvents, in a temperature range of from 0° C. and 100° C.

Compounds of formula (I) in which Z is a single bond and T is O, N or N—R^{T}, denotes compounds of formula Ic and can be prepared as shown in reaction below by analogy to the methods described in WO 2011/017513.

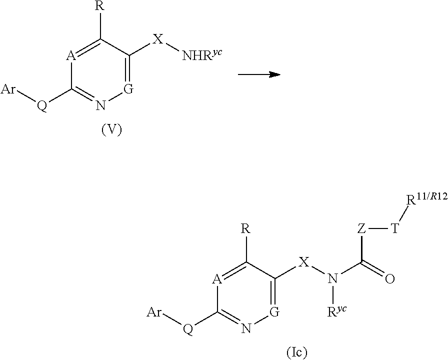

In the above reaction, R^{11/12} corresponds to radicals R¹¹ or R¹² respectively. The reaction shown above can be performed by analogy to conventional methods of preparing carbamates. According to a first embodiment, the amine of the formula (V) is converted into either an isocyanate or p-nitrophenyl carbamate followed by treatment with an alcohol of the formula R¹¹—OH or R¹²—OH, respectively, in the presence of an organic or inorganic base. According to another embodiment, the compound of the formula (V) is reacted with a chloroformate of the formula R^{11/112}—O—C(═O)—Cl. The chloroformate is prepared from the alcohols R^{11/12}OH by treatment with phosgene or triphosgene in the presence of a base, e.g. pyridine.

Compounds of formula (Ic), in which Z is —N(R^{zc})—C(═O)— or —N(R^{zc})—C(═S)— can be prepared by analogy to the methods described in WO 2013/009791, especially in reactions described therein or by the methods described in US 2012/0202687.

Compounds of the formula (II) where X═—CR^{xa}═CR^{xb}— or —CR^{xa}R^{xb}—CR^{xa}R^{xb}— can be prepared by analogy to the methods described in the examples or prepared by the reactions shown in the following reaction.

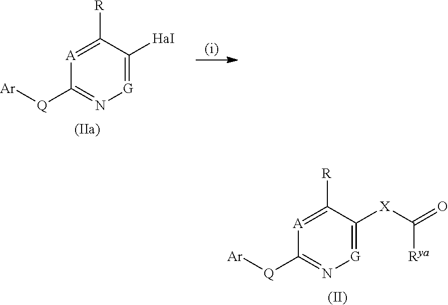

In the above reaction, Hal is halogen, preferably chlorine or bromine, in particular, bromine. Suitable reaction conditions for performing the above reaction (reaction step (i)) is by a Cu-catalyzed cross-coupling reaction of (IIa) with a alkenyl boronic acid or a alkenyl boronate ester using the methodology described in Journal of the American Chemical Society 2012, 134, 15165-15168. The alkenyl boronic acid or the alkenyl boronate ester can be prepared from the corresponding propargylic compounds.

Compounds of the formula (II) where X═—CR^{xa}═CR^{xb}— can also be prepared by reacting compounds of formula (IIb) with appropriate organophosphoranes (J. Heterocyclic Chem. 28: 1281 (1991)) or with appropriate organostannanes (Eur. Pat. Appl., 308736) (reaction step (ii), below reaction).

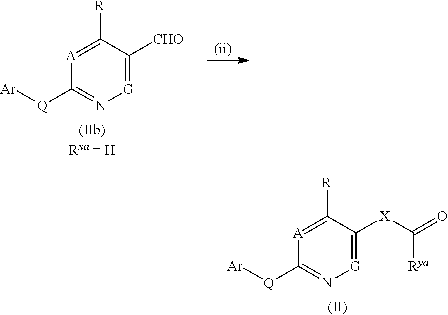

Compounds of the formula (II) where X═—CR^{xa}R^{xb}—CR^{xa}R^{xb}— can be prepared from compounds of formula (II) where X═—CR^{xa}═CR^{xb}— by standard hydrogenation protocols known in organic chemistry such as using hydrogen gas and a suitable metal catalyst as described in March's Advanced Organic Chemistry 6^{th} edition, Michael B. Smith and Jerry March.

Compounds of the formula (II) where X═—O—CR^{xa}R^{xb}— or —S—CR^{xa}R^{xb}— can be prepared by analogy to the methods described in below reaction and in accordance with the methods described in the examples

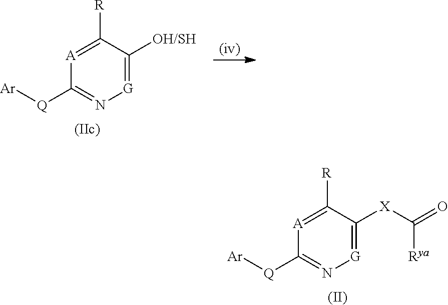

Compounds of the formula (II) where X═—O—CR^{xa}R^{xb}— or —S—CR^{xa}R^{xb}— can be prepared by first reacting compounds of formula (IIc) with compounds of the formula Lg-CR^{xa}R^{xb}—C(O)R^{ya} or Lg-CR^{xa}R^{xb}—C(O)OR″ or Lg-CR^{xa}R^{xb}—CN or Lg-CR^{xa}R^{xb}—C(OR″)₂ with appropriate protecting groups and where Lg is a leaving group such as a bromine, chlorine or iodine atom or a tosylate, mesylate or triflate, to yield compounds of formula (II) (step (iv)). R″ is alkyl, preferably methyl or ethyl. The resulting compounds can then be converted to compounds of the formula (II) by standard deprotection methods—acidic hydrolysis for acetals as described in Greene's Protecting Groups in Organic Synthesis, reduction with reducing agents such as Diisobutylaluminium hydride for nitriles and esters as described in March's Advanced Organic Chemistry 6^{th} edition, Michael B. Smith and Jerry March.

In another embodiment of the reaction, compounds of the formula (II) where X═—O—CR^{xa}R^{xb}— or —S—CR^{xa}R^{xb}— can be prepared by first reacting compounds of formula (IId) with compounds of the formula HO/HS—CR^{xa}R^{xb}—C(O)R^{ya} or HO/HS—CR^{xa}R^{xb}—C(O)OR″ or HO/HS—CR^{xa}R^{xb}—CN with appropriate protecting groups, by Cu or Pd catalysed reactions or uncatalysed reactions as described in WO2011159839 or WO2016027249 or US20070032485 and as depicted in below reaction. Wherein -Hal is bromine, chlorine or iodine atom or a tosylate, mesylate or triflate; R′″ is a boronic acid or an ester of a boronic acid.

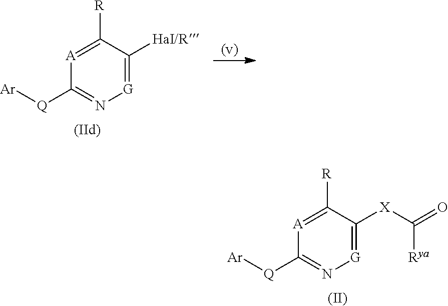

Compounds of the formula (II) where X═—N═CR^{xa}—, —NR^{xc}—CR^{xa}R^{xb}—, —NR^{xc}—C(═S)—, —N═C(S—R^{e})—, or —NR^{xc}—C(═O)— can be prepared in accordance with the methods described in the examples, from compounds of the formula (IIe) or can be prepared in accordance with below reaction.

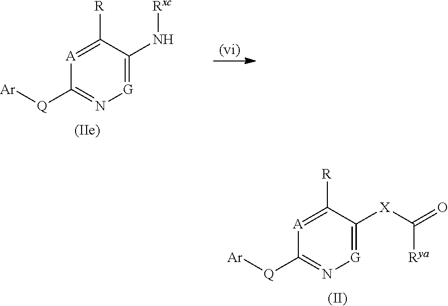

Compounds of the formula (II) where X═—N═CR^{xa}—, —NR^{xc}—CR^{xa}R^{xb}—, —NR^{xc}—C(═S)—, —N═C(S—R^{e})—, or —NR^{xc}—C(═O)— can be prepared by first reacting compounds of formula (IIe) with compounds of the formula Lg-CR^{xa}R^{xb}—C(O)R^{ya} or Lg-CR^{xa}R^{xb}—C(O)OR″ or Lg-CR^{xa}R^{xb}—CN or H(OC)R^{xa}—C(O)R^{ya} or Lg-(OC)R^{xa}—C(O)R^{ya} or Lg-(OC)R^{xa}—CN with appropriate protecting groups and where Lg is a leaving group such as a bromine, chlorine or iodine atom or a tosylate, mesylate or triflate, to yield compounds of formula (II) (step (vi)). R″ is alkyl, preferably methyl or ethyl as described in WO2006065703 or WO 2011079305. The resulting compounds can then be converted to compounds of the formula (II) by methods described in March's Advanced Organic Chemistry 6^{th} edition, Michael B. Smith and Jerry March.

Compounds of the formula (IIb), (IIc), (IId) and (IIe) can be prepared by analogy to compounds prepared in the literature and in accordance with the compounds prepared in the examples. Usually compounds of the formula (IIb), (IIc), (IId) and (IIe) are prepared by the reactions shown in the following reactions.

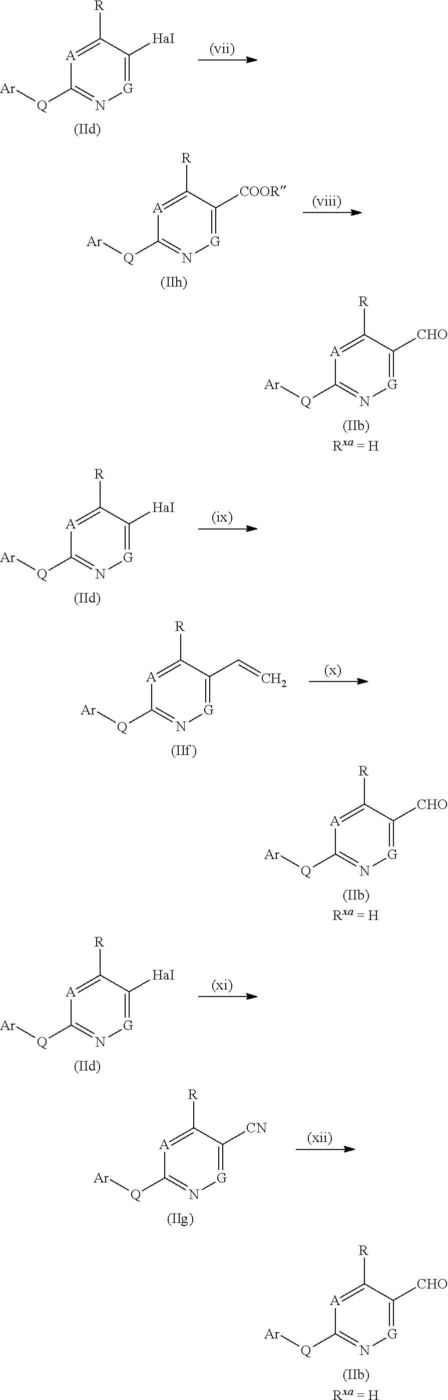

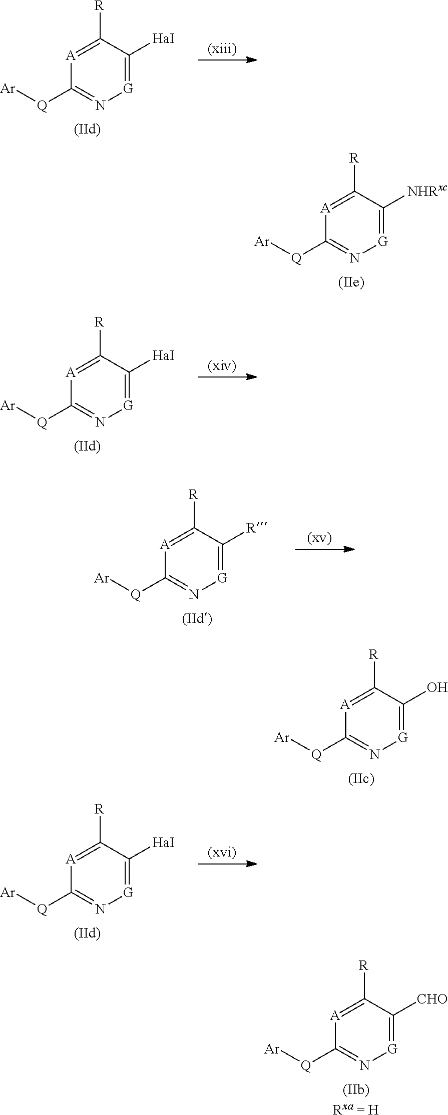

In the above reactions, -Hal is bromine, chlorine or iodine atom or a tosylate, mesylate or triflate; R′″ is a boronic acid of an ester of a boronic acid.

Suitable reaction conditions for performing the preparation of the cyanide compound of the formula (IIg) (reaction step (x)) by a Pd-catalyzed aromatic cyanation reaction of an aryl bromide of the formula (IId) with an alkalimetal cyanide, preferably NaCN, can be taken from Journal of the American Chemical Society, 133 (28), 10999-11005; 2011. The reduction of a cyanide compound (IIg) to an aldehyde compound (IIb) shown in step (xii) of the above reactions can be performed with a metal alkoxyaluminum hydride. Suitable alkoxyaluminum hydrides are lithium alkoxyaluminum hydrides and sodium alkoxyaluminum hydrides, e.g. Na[Al(OC₂H₅)₃H]. Suitable reaction conditions for step (viii) of the above reaction can be taken from Organic Reactions (Hooboken, N.J., United States), pp 36, 1988. The conversion of the aryl bromide (IId) into the ester compound (IIh) is shown in reaction step (vii) of the above reaction. Suitable reaction conditions for this palladium-catalysed reaction can be taken from Journal of Medicinal Chemistry, 52 (22), 7258-7272; 2009. Suitable reaction conditions for performing step (viii) of the above reaction can be taken from Synlett, (6), 869-872; 2006. Suitable reaction conditions for performing the reaction step (ix) of the above reaction can be taken from Journal of the American Chemical Society, 124(22), 6343-6348, 2002. Suitable reaction conditions for performing the reaction step (x) of the above reaction can be taken from European Journal of Medicinal Chemistry, 49, 310-323; 2012. Compounds of the formula (IIb) can also made from compounds of formula (IId) by reaction with as strong base like for example n-butyl lithium and with an electrophile, for example N,N-Dimethylformamide as shown in reaction step (xvi), of the above reaction.

Compounds of the formula (IIe) (reaction step (xiii) of the above reaction) can be prepared by reacting compounds of the formula (IId) with ammonia or amines of the formula R^{xc}NH₂ in the presence of a metal catalyst or its salts, preferably copper or its salts as described in Chem. Commun., 2009, 3035-3037. Compounds of formula (IIc) can be made from compounds of formula (IId) by oxidation with various oxidation reagents for example, hydrogen peroxide as described in Bioorganic and Medicinal chemistry letters, 2013, 23, 4705-4712. Compounds of formula (IId′) can be made from compounds of the formula (IId) by reacting with a Palladium (II) catalyzed reaction with pinacol boronates or by reaction with a base such as n-Butyl lithium and trialkylborates as described in Bioorganic and medicinal chemistry letters, 2013, 23, 4705-4712.

Compounds of the formula (IId) can be prepared from compounds of formula (IIh) as per below reaction.

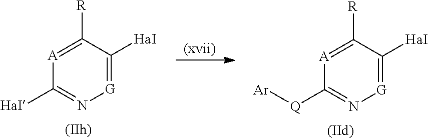

In the above reaction, Hal′ can be fluorine, chlorine, bromine or iodine, preferably chlorine or tosylate, mesylate or triflate. Hal can be chlorine, bromine or iodine, preferably bromine or tosylate, mesylate or triflate. Compounds of the formula (IId) can be prepared from compounds of formula (IIh) by reacting with compounds of the formula Ar—OH or Ar—NHR² by heating in a polar protic or aprotic solvents in an acidic, basic or neutral conditions as described in WO2010129053, WO2007146824 or Chemical Communications, 2014, 50, 1465.

Compounds of formula (IId) can also be prepared from compounds of formula (IIi) by reaction with aromatic halogen compounds or aromatic boronic acids or their esters under Cu(I), Cu(II) or Pd(II) catalysed conditions as described in WO 2007056075 or WO2002066480 or by using methods described in Organic Letters 2009, 11, 2514 as shown in below reaction.

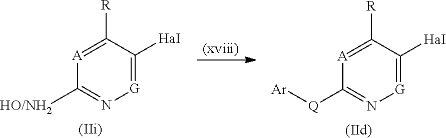

Compounds of the formula (IIh) and (IIi) can be obtained from commercial sources or alternatively be prepared by using methods given in US 20050222228 and Journal of Organic Chemistry, 2002, 77(16), 6908, respectively.

Individual compounds of formula I can also be prepared by derivatisation of other compounds of formula I or the intermediates thereof.

If the synthesis yields mixtures of isomers, a separation is generally not necessarily required since in some cases the individual isomers can be interconverted during work-up for use or during application (for example under the action of light, acids or bases). Such conversions may also take place after use, for example in the treatment of plants in the treated plant, or in the harmful fungus to be controlled.

A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

Unless otherwise indicated, the term “compound(s) according to the invention” or “compound(s) of the invention” or “compound(s) of formula (I)”, refers to the compounds of formula I.

The term “compound(s) according to the invention”, or “compounds of formula I” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The term “composition(s) according to the invention” or “composition(s) of the present invention” encompasses composition(s) comprising at least one compound of formula I according to the invention as defined above. The compositions of the invention are preferably agricultural or veterinary compositions.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The present invention relates to amorphous and crystalline compounds according to the invention, mixtures of different crystalline states of the respective compounds according to the invention, as well as amorphous or crystalline salts thereof.

The term “tautomers” encompasses isomers, which are derived from the compounds of formula I by the shift of an H-atom involving at least one H-atom located at a nitrogen, oxygen or sulphur atom. Examples of tautomeric forms are keto-enol forms, imine-enamine forms, urea-isourea forms, thiourea-isothiourea forms, (thio)amide-(thio)imidate forms etc.

The term “stereoisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon ring atom of the isothiazoline ring carrying radical R¹. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO). To be more precise, it relates to any compound of the present invention which has at least one tertiary nitrogen atom that is oxidized to an N-oxide moiety. N-oxides of compounds I can in particular be prepared by oxidizing e.g. the ring nitrogen atom of an N-heterocycle, e.g. a pyridine or pyrimidine ring present in Ar or R¹¹, or an imino-nitrogen present in central tricyclic core, with a suitable oxidizing agent, such as peroxo carboxylic acids or other peroxides. The person skilled in the art knows if and in which positions compounds of the present invention may form N-oxides.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally or veterinarily acceptable salts are especially the salts of those cations or the acid addition salts of those acids whose cations and anions, which are known and accepted in the art for the formation of salts for agricultural or veterinary use respectively, and do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium. Suitable acid addition veterinarily acceptable salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “modified plants” and in particular “cultivated plants”.

The term “modified plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinatetolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as 6-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual members. The prefix Cₙ-Cₘ indicates in each case the possible number of carbon atoms in the group.

The term halogen denotes in each case F, Br, Cl or I, in particular F, Cl or Br.

The term “alkyl” as used herein and in the alkyl moieties of alkoxy, alkylthio, and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”) or 1 to 6 (“C₁-C₆-alkyl”) carbon atoms. C₁-C₂-Alkyl is CH₃ or C₂H₅. C₁-C₃-Alkyl is additionally propyl and isopropyl. C₁-C₄-Alkyl is additionally butyl, 1-methylpropyl (secbutyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”) or 1 to 6 (“C₁-C₆-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like.

The term “alkylene” (or alkanediyl) as used herein in each case denotes an alkyl radical as defined above, wherein one hydrogen atom at any position of the carbon backbone is replaced by one further binding site, thus forming a bivalent moiety. Alkylene has preferably 1 to 6 carbon atoms (C₁-C₆-alkylene), 2 to 6 carbon atoms (C₂-C₆-alkylene), in particular 1 to 4 carbon atoms (C₁-C₄-alkylene) or 2 to 4 carbon atoms (C₂-C₄-alkylene). Examples of alkylene are methylene (CH2), 1,1-ethandiyl, 1,2-ethandiyl, 1,3-propandiyl, 1,2-propandiyl, 2,2-propandiyl, 1,4-butandiyl, 1,2-butandiyl, 1,3-butandiyl, 2,3-butandiyl, 2,2-butandiyl, 1,5-pentandiyl, 2,2-dimethylpropan-1,3-diyl, 1,3-dimethyl-1,3-propandiyl, 1,6-hexandiyl etc.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”) or 2 to 6 (“C₂-C₆-alkenyl) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like.

The term “alkynyl” as used herein refers to straight-chain or branched hydrocarbon groups having 2 to 3 (“C₂-C₃-alkynyl”), 2 to 4 (“C₂-C₄-alkynyl”) or 2 to 6 (“C₂-C₆-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “cycloalkyl” as used herein refers to mono- or bi- or polycyclic saturated hydrocarbon radicals having in particular 3 to 6 (“C₃-C₆-cycloalkyl”) or 3 to 5 (“C₃-C₅-cycloalkyl”) or 3 to 4 (“C₃-C₄-cycloalkyl”) carbon atoms. Examples of monocyclic radicals having 3 to 4 carbon atoms comprise cyclopropyl and cyclobutyl. Examples of monocyclic radicals having 3 to 5 carbon atoms comprise cyclopropyl, cyclobutyl and cyclopentyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “cycloalkoxy” as used herein refers to a cycloalkyl radical, in particular a monocyclic cycloalkyl radical, as defined above having in particular 3 to 6 (“C₃-C₆-cycloalkoxy”) or 3 to 5 (“C₃-C₅-cycloalkoxy”) or 3 to 4 (“C₃-C₄-cycloalksoxy”) carbon atoms, which is bound via an oxygen atom to the remainder of the molecule.

The term “cycloalkyl-C₁-C₄-alkyl” refers to a C₃-C₈-cycloalkyl (“C₃-C₈-cycloalkyl-C₁-C₄-alkyl”), preferably a C₃-C₆-cycloalkyl (“C₃-C₆-cycloalkyl-C₁-C₄-alkyl”), more preferably a C₃-C₄-cycloalkyl (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C—C₄-alkyl, are cyclopentylmethyl, cyclopentylethyl, cyclopentylpropyl, cyclohexylmethyl, cyclohexylethyl and cyclohexylpropyl.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is OCH₃ or OC₂H₅. C₁-C₃-Alkoxy is additionally, for example, n-propoxy and 1-methylethoxy (isopropoxy). C₁-C₄-Alkoxy is additionally, for example, butoxy, 1-methylpropoxy (secbutoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1—(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₆-alkoxy-C₁-C₄-alkyl” as used herein, refers to a straight-chain or branched alkyl having 1 to 4 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “alkoxyalkoxy” as used herein refers to an alkoxyalkyl radical, in particular a C₁-C₆-alkoxy-C₁-C₄-alkyl radical, as defined above, which is bound via an oxygen atom to the remainder of the molecule. Examples thereof are OCH₂—OCH₃, OCH₂—OC₂H₅, n-propoxymethoxy, OCH₂—OCH(CH₃)₂, n-butoxymethoxy, (1-methylpropoxy)methoxy, (2-methylpropoxy)methoxy, OCH₂—OC(CH₃)₃, 2-(methoxy)ethoxy, 2-(ethoxy)ethoxy, 2-(n-propoxy)ethoxy, 2-(1-methylethoxy)ethoxy, 2-(n-butoxy)ethoxy, 2-(1-methylpropoxy)ethoxy, 2-(2-methylpropoxy)ethoxy, 2-(1,1-dimethylethoxy)ethoxy, etc.

The substituent “oxo” replaces a CH₂ by a C(═O) group.

The term “aryl” relates to phenyl and bi- or polycyclic carbocycles having at least one fused phenylene ring, which is bound to the remainder of the molecule. Examples of bi- or polycyclic carbocycles having at least one phenylene ring include naphthyl, tetrahydronaphthyl, indanyl, indenyl, anthracenyl, fluorenyl etc.

The term “aryl-C₁-C₄-alkyl” relates to C₁-C₄-alkyl, as defined above, wherein one hydrogen atom has been replaced by an aryl radical, in particular a phenyl radical. Particular examples of aryl-C₁-C₄-alkyl include benzyl, 1-phenethyl, 2-phenetyl, 1-phenylpropyl, 2-phenylpropyl, 3-phenyl-1-propyl and 2-phenyl-2-propyl.

The term “aryloxy-C₁-C₄-alkyl” relates to C₁-C₄-alkyl, as defined above, wherein one hydrogen atom has been replaced by an aryloxy radical, in particular a phenoxy radical. Particular examples of aryloxy-C₁-C₄-alkyl include phenoxymethyl, 1-phenoxyethyl, 2-phenoxyetyl, 1-phenoxypropyl, 2-phenoxypropyl, 3-phenoxy-1-propyl and 2-phenoxy-2-propyl.

The term “aryl-C₁-C₄-carbonyl” relates to aryl as defined above, in particular a phenyl radical, which is bound by a carbonyl to the remainder of the molecule. Particular examples of arylcarbonyl include benzoyl, 1-naphthoyl and 2-naphthoyl.

The term hetaryl relates to aromatic heterocycles having either 5 or 6 ring atoms (5- or 6-membered hetaryl) and being monocyclic or 8, 9 or 10 ring atoms and bing bicyclic. Hetaryl will generally have at least one ring atom selected from O, S and N, which in case of N may be an imino-nitrogen or an amino-nitrogen, which carries hydrogen or a radical different from hydrogen. Hetaryl may have 1, 2, 3 or 4 further nitrogen atoms as ring members, which are imino nitrogens. Examples of 5- or 6-membered hetaryl include 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 1,3,4-oxadiazolyl-2-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl and 1,3,5-triazin-2-yl. Examples of 8-, 9- or 10-membered hetaryl include, for example, quinolinyl, isoquinolinyl, cinnolinyl, indolyl, indolizynyl, isoindolyl, indazolyl, benzofuryl, benzothienyl, benzo[b]thiazolyl, benzoxazolyl, benzthiazolyl, benzimidazolyl, imidazo[1,2-a]pyridine-2-yl, thieno[3,2-b]pyridine-5-yl, imidazo-[2,1-b]-thiazol-6-yl and 1,2,4-triazolo[1,5-a]pyridine-2-yl.

Examples of N-bound 5-, 6-, 7 or 8-membered saturated heterocycles include: pyrrolidin-1-yl, pyrazolidin-1-yl, imidazolidin-1-yl, oxazolidin-3-yl, isoxazolidin-2-yl, thiazolidin-3-yl, isothiazolidin-2-yl, piperidin-1-yl, piperazin-1-yl, morpholin-4-yl, thiomorpholin-4-yl, 1-oxothiomorpholin-4-yl, 1,1-dioxothiomorpholin-4-yl, azepan-1-yl and the like.

The term “hetaryl-C₁-C₄-alkyl” relates to C₁-C₄-alkyl, as defined above, wherein one hydrogen atom has been replaced by a hetaryl radical, in particular a pyridyl radical. Particular examples of hetaryl-C₁-C₄-alkyl include 2-pyridylmethyl, 3-pyridylmethyl, 4-pyridylmethyl, 1-(2-pyridyl)ethyl, 2-(2-pyridyl)ethyl, 1-(3-pyridyl)ethyl, 2-(3-pyridyl)ethyl, 1-(4-pyridyl)ethyl, 2-(4-pyridyl)ethyl etc.

The term “hetaryloxy-C—C₄-alkyl” relates to C₁-C₄-alkyl, as defined above, wherein one hydrogen atom has been replaced by an hetaryloxy radical, in particular a pyridyloxy radical. Particular examples of hetaryloxy-C₁-C₄-alkyl include 2-pyridyloxymethyl, 3-pyridyloxymethyl, 4-pyridyloxymethyl, 1-(2-pyridyloxy)ethyl, 2-(2-pyridyloxy)ethyl, 1-(3-pyridyloxy)ethyl, 2-(3-pyridyloxy)ethyl, 1-(4-pyridyloxy)ethyl, 2-(4-pyridyloxy)ethyl etc.

The term “hetaryl-C₁-C₄-carbonyl” relates to hetaryl as defined above, in particular a C-bound hetaryl radical, e.g. 2-, 3- or 4-pyridyl, 2- or 3-thienyl, 2- or 3-furyl, 1-, 2- or 3-pyrrolyl, 2- or 4-pyrimidinyl, pyridazinyl, 1-, 3- or 4-pyrazolyl, 1-, 2- or 4-imidazolyl radical, which is bound by a carbonyl to the remainder of the molecule.

The term “substituted” if not specified otherwise refers to substituted with 1, 2 or maximum possible number of substituents. If substituents as defined in compounds of formula I are more than one then they are independently from each other are same or different if not mentioned otherwise.

With respect to the variables, the embodiments of the compounds of the formula I are,

In one embodiment, A is CR^{A}

In another embodiment, A is N.

In one embodiment, G is CR^{B}.

In another embodiment, G is N.

In one embodiment, A is CR^{A} and G is N.

In another embodiment, A is N and G is CR^{B}.

In another embodiment, A is N and G is N.

In another embodiment, A is CR^{A} and G is CR^{B}.

In one embodiment, R is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In more preferred embodiment, R is H, halogen, OH, CN, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In most preferred embodiment, R is H, Cl, Br, F, OH, CN, CH₃, C₂H₅, n-C₃H₇, isopropyl, cyclopropyl, allyl and propargyl, CH₂F, CHF₂, CF₃, OCH₃, OC₂H₅, OCH₂F, OCHF₂, OCF₃, OCH₂CH₂CF₃, OCH₂CF₂CHF₂, or OCH₂CF₂CF₃.

In one embodiment, R^{A} is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In more preferred embodiment, R^{A} is H, halogen, OH, CN, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In most preferred embodiment, R^{A} is H, Cl, Br, F, OH, CN, CH₃, C₂H₅, n-C₃H₇, isopropyl, cyclopropyl, allyl and propargyl, CH₂F, CHF₂, CF₃, OCH₃, OC₂H₅, OCH₂F, OCHF₂, OCF₃, OCH₂CH₂CF₃, OCH₂CF₂CHF₂, or OCH₂CF₂CF₃.

In one embodiment, R^{B} is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In more preferred embodiment, R^{B} is H, halogen, OH, CN, C₁-C₆-alkyl, C₁-C₆-haloalkyl, C₁-C₆-alkoxy, C₁-C₆-haloalkoxy, C₂-C₆-alkenyl, or tri-C₁-C₆-alkylsilyl.

In most preferred embodiment, R^{B} is H, Cl, Br, F, OH, CN, CH₃, C₂H₅, n-C₃H₇, isopropyl, cyclopropyl, allyl and propargyl, CH₂F, CHF₂, CF₃, OCH₃, OC₂H₅, OCH₂F, OCHF₂, OCF₃, OCH₂CH₂CF₃, OCH₂CF₂CHF₂, or OCH₂CF₂CF₃.

In one embodiment, Q is NR².

In another embodiment, Q is O.

In another embodiment, Q is S.

In another embodiment, Q is S(═O).

In another embodiment, Q is S(═O)₂.

In more preferred embodiment compounds of formula I are selected from compounds of formula I.A to I.T wherein R¹, R^{A}, R^{B}, R² and Ar are as defined herein.

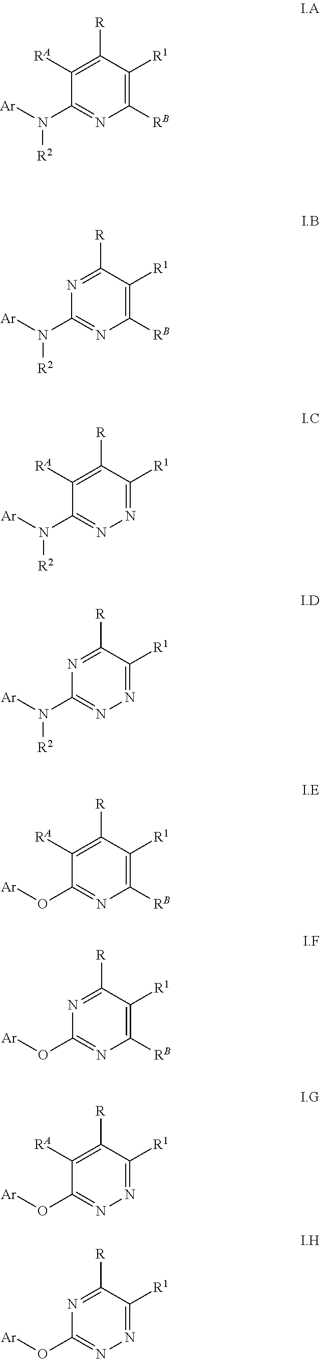

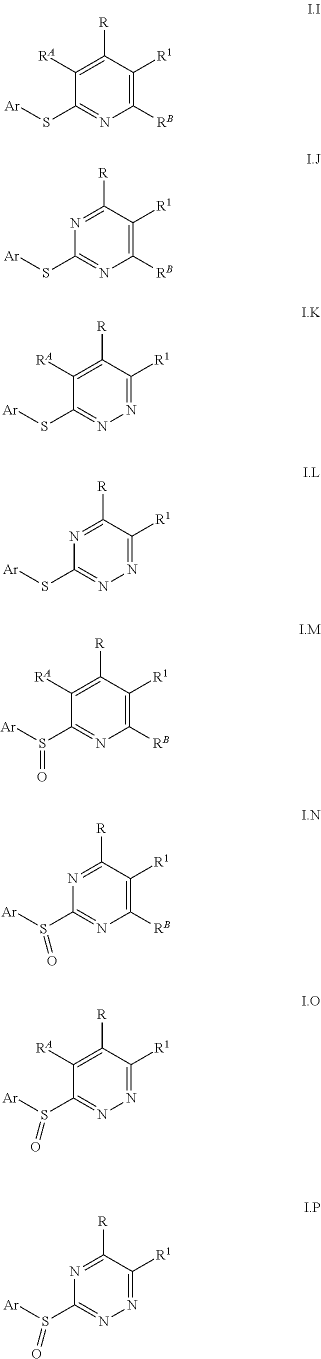

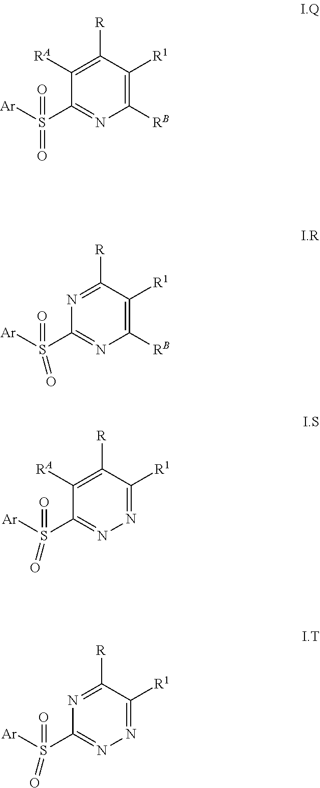

wherein, Ar is phenyl or 5- or 6-membered hetaryl ring which is substituted with R^{Ar};

R^{Ar} is halogen, OH, CN, NO₂, SCN, C₁-C₆-alkyl, C₁-C₆-haloalkyl, C₁-C₆-alkoxy, C₁-C₆-haloalkoxy, or S—R^{e}, which are unsubstituted or substituted with halogen;

R² is H, C₁-C₆-alkyl, C₁-C₆-haloalkyl, C₂-C₆-alkenyl, C₂-C₆-alkynyl, C₁-C₄-alkyl-C₁-C₆-alkoxy, or C₃-C₆-cycloalkyl, which are unsubstituted or substituted with halogen,

and phenyl which is unsubstituted or substituted with R^{f};

R is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, or C₂-C₆-alkenyl;

R^{A} is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, or C₂-C₆-alkenyl;

R^{B} is H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, or C₂-C₆-alkenyl;

and R¹ is X—Y—Z-T-R¹¹ and X—Y—Z-T-R¹², as defined in formula I.

In another more preferred embodiment compounds of formula I are selected from compounds of formula I.A.1, I.A.2, I.A.3, I.B.1, I.B.2, I.B.3, I.B.4, I.B.5, I.C.1, I.C.1, I.C.2, I.D.1, I.D.2, I.E.1, I.E.2, I.E.3, I.E.4, I.F.1, I.F.2, I.F.3, I.F.4, I.F.5, I.G.1, I.G.2, I.G.1, I.H.1, I.H.2, I.1.1, I.1.2, I.1.3, I.J.1, I.J.2, I.J.3, I.J.4, I.J.5, I.K.1, I.K.2, I.K.3, I.L.1, I.L.2, I.M.1, I.M.2, I.M.3, I.N.1, I.N.2, I.N.3, I.N.4, I.O.1, I.O.2, I.O.3, I.P.1, I.P.2, I.Q.1, I.Q.2, I.Q.3, I.R.1, I.R.2, I.R.3, I.R.4, I.S.1, I.S.2, I.S.3, I.T.1, I.T.2, and I.T.3, wherein R¹, R², and Ar are as defined herein.

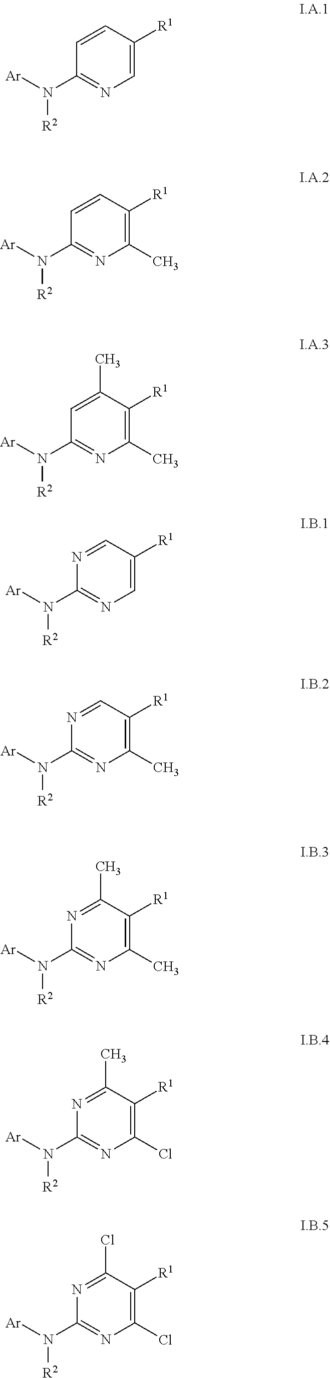

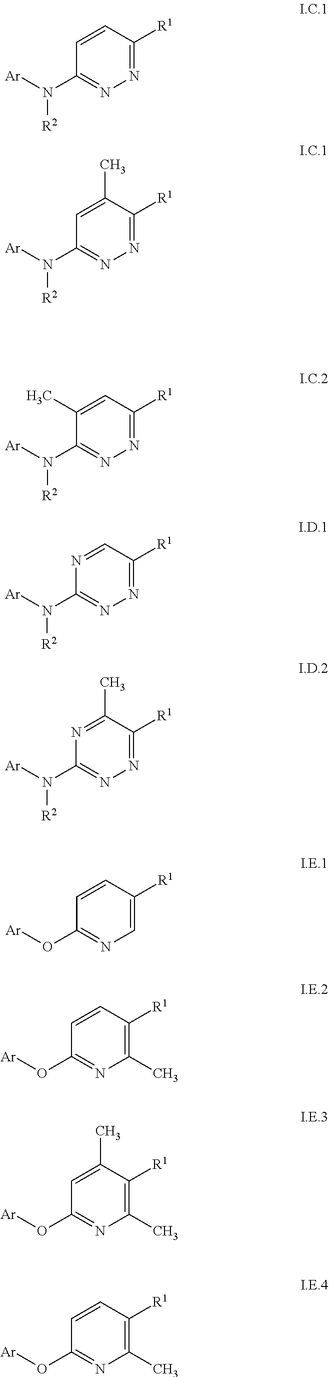

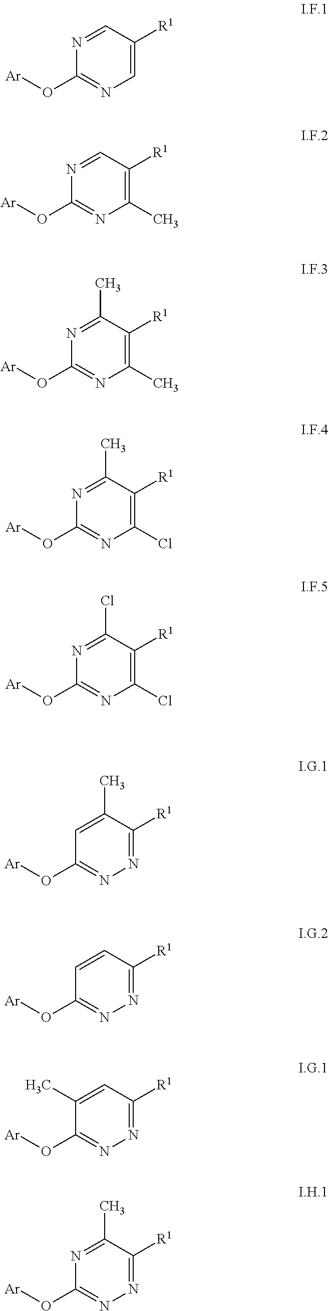

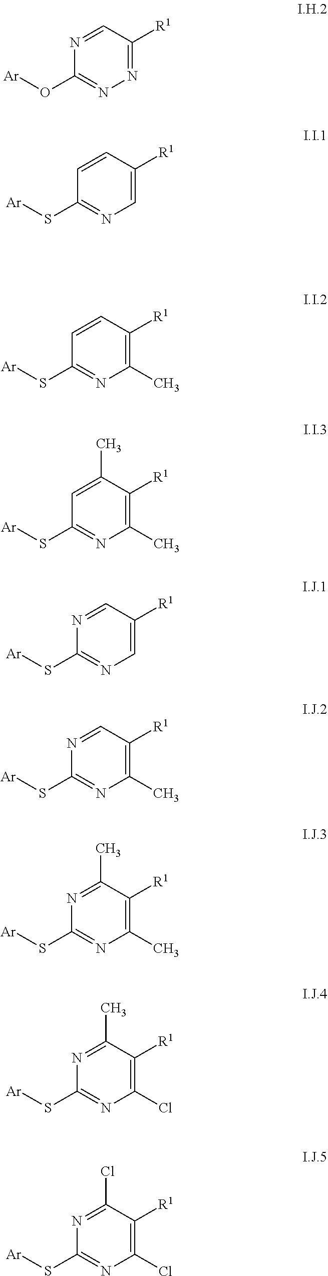

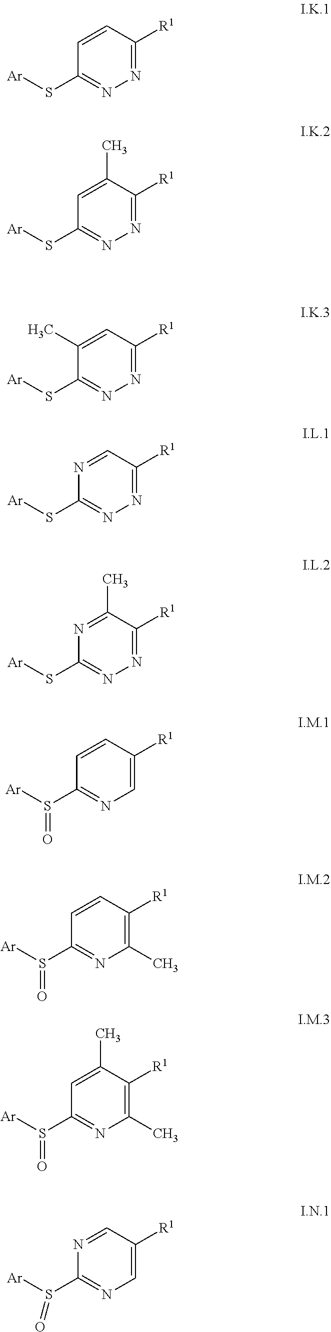

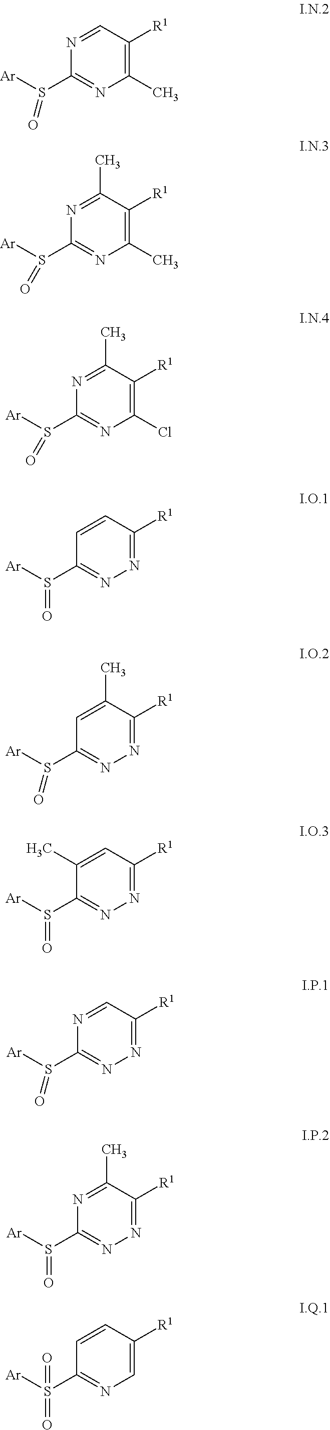

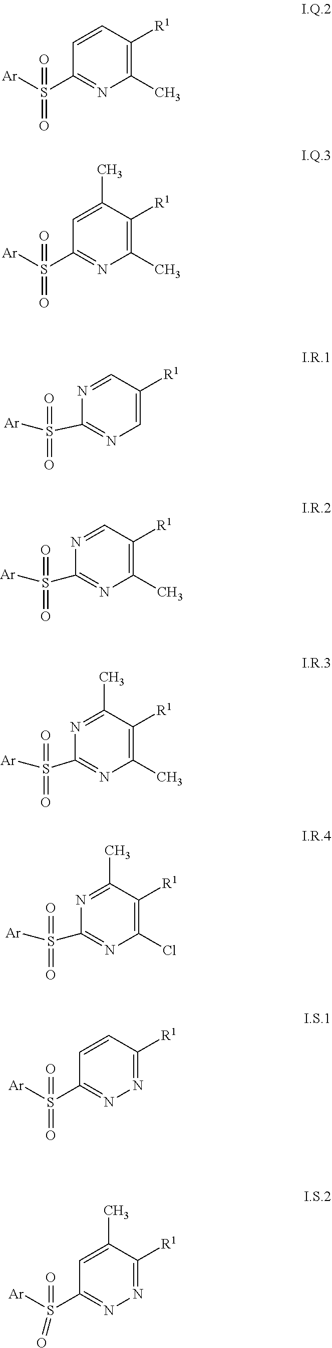

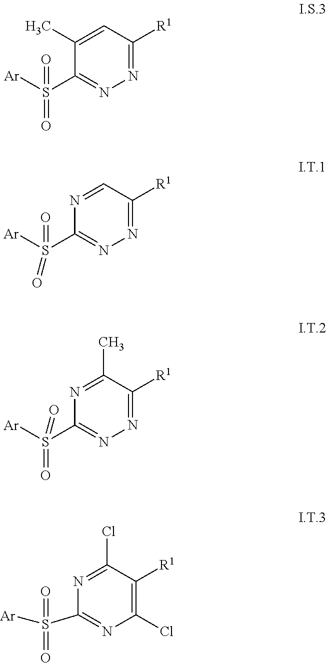

In one embodiment, R² is H, C₁-C₆-alkyl, C₁-C₆-haloalkyl, C₂-C₆-alkenyl, C₂-C₆-alkynyl, C₁-C₄-alkyl-C₁-C₆-alkoxy, C₃-C₆-cycloalkyl, which are unsubstituted or substituted with halogen,

phenyl one embodiment, which are unsubstituted or substituted with R^{f}.

In more preferred embodiment, R² is H, C₁-C₆-alkyl, C₁-C₆-haloalkyl, C₂-C₆-alkenyl, C₂-C₆-alkynyl, C₁-C₄-alkyl-C₁-C₆-alkoxy, or C₃-C₆-cycloalkyl.

In most preferred embodiment, R² is H, CH₃, C₂H₅, n-C₃H₇, isopropyl, cyclopropyl, allyl and propargyl, CH₂F, CHF₂, CF₃, CH₂F, CHF₂, CF₃, or phenyl which is unsubstituted or substituted with R^{f}.

In one embodiment, Ar is phenyl which is unsubstituted or substituted with R^{Ar}.

In another embodiment, Ar is 5- or 6-membered hetaryl, which is unsubstituted or substituted with R^{Ar}.

In another embodiment, Ar is phenyl, pyrimidinyl, pyridazinyl, or pyridyl, which are unsubstituted substituted with R^{Ar}.

In one embodiment, R^{Ar} is halogen, OH, CN, NO₂, SCN, C₁-C₆-alkyl, C₁-C₆-haloalkyl, C₁-C₆-alkoxy, C₁-C₆-haloalkoxy, or S—R^{e}, which are unsubstituted substituted with halogen.

In more preferred embodiment, R^{Ar} is F, Cl, Br, OH, CN, NO₂, SCN, CH₃, C₂H₅, n-C₃H₇, isopropyl, CH₂F, CHF₂, CF₃, CH₂CF₃, CF₂CHF₂, C₂F₅, CH₂CH₂CF₃, CH₂CF₂CHF₂, CH₂CF₂CF₃, OCH₃, OC₂H₅, n-propyloxy, isopropyloxy, OCH₂F, OCHF₂, OCF₃, OCH₂CF₃, OCF₂CHF₂, OC₂F₅, OCH₂CH₂CF₃, OCH₂CF₂CHF₂, OCH₂CF₂CF₃, or S—R^{e}, where R^{e} is C₁-C₆-alkyl, in particular C₁-C₃-alkyl such as CH₃, C₂H₅, n-C₃H₇ or isopropyl, or C₁-C₆-haloalkyl, in particular fluorinated C₁-C₃-alkyl such as CH₂F, CHF₂, CF₃, CH₂CF₃, CF₂CHF₂, C₂F₅, CH₂CH₂CF₃, CH₂CF₂CHF₂ or CH₂CF₂CF₃.

Preferred Ar are the radicals Ar-1 to Ar-12 summarized in Table A below.

TABLE A
Examples of radicals Ar

| | |
|---|---|
| Ar-1 | 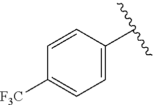 [SMILES: *c1ccc(C(F)(F)F)cc1] |
| Ar -2 | 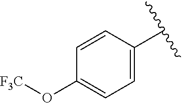 [SMILES: *c1ccc(OC(F)(F)F)cc1] |
| Ar-3 | 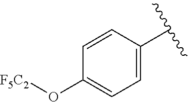 |
| Ar-4 | 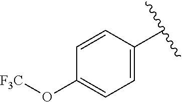 [SMILES: *c1ccc(OC(F)(F)F)cc1] |
| Ar-5 | 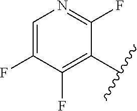 [SMILES: *c1c(F)ncc(F)c1F] |
| Ar-6 | 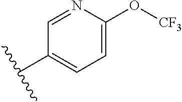 [SMILES: *c1ccc(OC(F)(F)F)nc1] |
| Ar-7 | 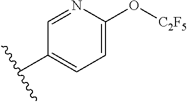 |
| Ar-8 | 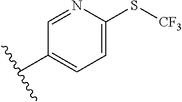 [SMILES: *c1ccc(SC(F)(F)F)nc1] |
| Ar-9 | 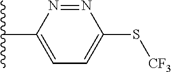 [SMILES: *c1ccc(SC(F)(F)F)nn1] |
| Ar-10 | 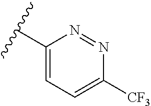 [SMILES: *c1ccc(C(F)(F)F)nn1] |
| Ar-11 | 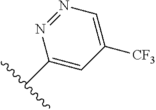 [SMILES: *c1cc(C(F)(F)F)cnn1] |
| Ar-12 | 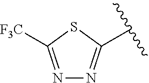 [SMILES: *c1nnc(C(F)(F)F)s1] |

In one embodiment, R¹ is X—Y—Z-T-R¹¹.

In another embodiment, R¹ is X—Y—Z-T-R¹².

In one embodiment, X is —CR^{xa}R^{xb}—.

In another embodiment, X is —O—.

In another embodiment, X is —S—.

In another embodiment, X is —NR^{xc}—.

In another embodiment, X is —CR^{xa}═CR^{xb}—.

In another embodiment, X is —CR^{xa}R^{xb}—CR^{xa}R^{xb}—.

In another embodiment, X is —O—CR^{xa}R^{xb-}.

In another embodiment, X is —S—CR^{xa}R^{xb}—.

In another embodiment, X is —N═CR^{xa}—.

In another embodiment, X is —NR^{xc}—CR^{xa}R^{xb}—, wherein CR^{xa}R^{xb} is bound to Y.

In another embodiment, X is —NR^{xc}—C(═S)—.

In another embodiment, X is —N═C(S—R^{e})—.

In another embodiment, X is —NR^{xc}—C(═O)—.

In one embodiment, Y is —CR^{ya}═N—, wherein the N is bound to Z.

In another embodiment, Y is —NR^{yc}—C(═S)—, wherein C(═S) is bound to Z.

In another embodiment, Y is —NR^{yc}—C(═O)—, wherein C(═O) is bound to Z.

In one embodiment, Z is —NR^{zc}—C(═S)—, wherein C(═S) is bound to T.

In another embodiment, Z is —NR^{zc}—C(═O)—, wherein C(═O) is bound to T.

In another embodiment, Z is-N═C(S—R^{za})—, wherein T is bound to the carbon atom.

In another embodiment, Z is —O—C(═O)—, wherein T is bound to the carbon atom.

In another embodiment, Z is —O—C(═S)—, wherein T is bound to the carbon atom.

In another embodiment, Z is —NR^{zc}—C(S—R^{za})═, wherein T is bound to the carbon atom.

In another embodiment, Z is a single bond.

In one embodiment, T is O.

In another embodiment, T is N—R^{T}.

In another embodiment, T is N.

In one embodiment, R^{xa}, R^{xb}, R^{ya} are H, halogen, C₁-C₆-alkyl, C₁-C₆-alkoxy, C₁-C₆-haloalkyl, C₁-C₆-haloalkoxy, which are unsubstituted or substituted with halogen,

phenyl, or benzyl, wherein the rings are unsubstituted or substituted with R^{f}.

In more preferred embodiment, R^{xa}, R^{xb}, R^{ya} are H, halogen, C₁-C₆-alkyl, C₁-C₆-alkoxy, which are unsubstituted or substituted with halogen,

or phenyl which is unsubstituted or substituted with R^{f}.

In most preferred embodiment, R^{xa}, R^{xb}, R^{ya} are H, F, Cl, Br, CH₃, C₂H₅, n-C₃H₇, isopropyl, CH₂F, CHF₂, CF₃, CH₂CF₃, CF₂CHF₂, C₂F₅, CH₂CH₂CF₃, CH₂CF₂CHF₂, CH₂CF₂CF₃, OCH₃, OC₂H₅, n- propyloxy, isopropyloxy, OCH₂F, OCHF₂, OCF₃, OCH₂CF₃, OCF₂CHF₂, OC₂F₅, OCH₂CH₂CF₃, OCH₂CF₂CHF₂, OCH₂CF₂CF₃, or phenyl which is unsubstituted or substituted with R^{f}.

In one embodiment, R^{xc}, R^{yc}, R^{zc} are H, C₁-C₆-alkyl, C₁-C₆-haloalkyl, C₃-C₆-cycloalkyl, which are unsubstituted or substituted with halogen,

phenyl, or benzyl, wherein the rings are unsubstituted or substituted with R^{f}.

In more preferred embodiment, R^{xc}, R^{yc}, R^{zc} are H, C₁-C₆-alkyl, which are unsubstituted or substituted with halogen,

or phenyl which is unsubstituted or substituted with R^{f}.

In most preferred embodiment, R^{xc}, R^{yc}, R^{zc} are H, CH₃, C₂H₅, n-C₃H₇, isopropyl, CH₂F, CHF₂, CF₃, CH₂CF₃, CF₂CHF₂, C₂F₅, CH₂CH₂CF₃, CH₂CF₂CHF₂, CH₂CF₂CF₃, or phenyl which is unsubstituted or substituted with R^{f}.

In one embodiment, R^{T} is H, C₁-C₆-alkyl, C₁-C₆-haloalkyl, C₂-C₆-alkenyl, C₂-C₆-alkynyl, C₁-C₄-alkyl-C₁-C₆-alkoxy, which are unsubstituted or substituted with halogen,

C(O)—NR^{b}R^{c}, C(O)—R^{d}, SO₂NR^{b}R^{c}, S(═O)ₘRe, phenyl, or benzyl, wherein the rings are unsubstituted or substituted with R^{f}.

In another embodiment, R^{zc} together with R^{T} if present, forms C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene may be unsubstituted or substituted with R^{h}.

In more preferred embodiment, R^{zc} together with R^{T} if present, forms C₁-C₆-alkylene or a linear C₂-C₆-alkenylene group, where in the linear C₁-C₆-alkylene and the linear C₂-C₆-alkenylene a CH₂ moiety is replaced by a carbonyl group.

In another more preferred embodiment, R^{zc} together with R^{T} if present, forms C₁-C₆-alkylene or a linear C₂-C₆-alkenylene group, where in the linear C₁-C₆-alkylene and the linear C₂-C₆-alkenylene a CH₂ moiety is replaced by a C═N—R′ and wherein 1 or 2 CH₂ moieties may be replaced by O or S and/or wherein the linear C₁-C₆-alkylene and the linear C₂-C₆-alkenylene may be unsubstituted or substituted with R^{h}.

In another more preferred embodiment, R^{zc} together with R^{T} if present, forms C₁-C₆-alkylene or a linear C₂-C₆-alkenylene group, where in the linear C₁-C₆-alkylene and the linear C₂-C₆-alkenylene 1 or 2 CH₂ moieties are replaced by O or S and/or wherein the linear C₁-C₆-alkylene and the linear C₂-C₆-alkenylene may be unsubstituted or substituted with R^{h}.

In one embodiment, R^{za} is H, C₁-C₆-alkyl, C₁-C₆-haloalkyl, C₁-C₆-alkylen-NR^{b}R^{c}, C₁-C₆—C(O)—R^{d}, phenyl, phenylcarbonyl, or benzyl, wherein the rings are unsubstituted or substituted with R^{f}.

In another embodiment, R^{za} together with R^{T} if present, forms C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene may be unsubstituted or substituted with R^{h}.

In more preferred embodiment, R^{za} together with R^{T} if present, forms C₁-C₆-alkylene or a linear C₂-C₆-alkenylene group, where in the linear C₁-C₆-alkylene and the linear C₂-C₆-alkenylene a CH₂ moiety is replaced by a carbonyl group.

In another more preferred embodiment, R^{za} together with R^{T} if present, forms C₁-C₆-alkylene or a linear C₂-C₆-alkenylene group, where in the linear C₁-C₆-alkylene and the linear C₂-C₆-alkenylene a CH₂ moiety is replaced by a C═N—R′ and wherein 1 or 2 CH₂ moieties may be replaced by O or S and/or wherein the linear C₁-C₆-alkylene and the linear C₂-C₆-alkenylene may be unsubstituted or substituted with R^{h}.

In another more preferred embodiment, R^{za} together with R^{T} if present, forms C₁-C₆-alkylene or a linear C₂-C₆-alkenylene group, where in the linear C₁-C₆-alkylene and the linear C₂-C₆-alkenylene 1 or 2 CH₂ moieties are replaced by O or S and/or wherein the linear C₁-C₆-alkylene and the linear C₂-C₆-alkenylene may be unsubstituted or substituted with R^{h}.

In a preferred embodiment, R^{a}, R^{b} and R^{c} are H, C₁-C₆-alkyl, C₁-C₆-haloalkyl, C₂-C₆-alkenyl, C₂-C₆-alkynyl, which are unsubstituted or substituted with halogen,

C₁-C₆-alkylen-CN, phenyl, or benzyl, wherein the rings are unsubstituted or substituted with R^{f};

In more preferred embodiment, R^{a}, R^{b} and R^{c} are H, C₁-C₆-alkyl, C₁-C₆-haloalkyl, C₂-C₆-alkenyl, C₂-C₆-alkynyl, which are unsubstituted or substituted with halogen,

phenyl, or benzyl, wherein the rings are unsubstituted or substituted with R^{f}.

In a preferred embodiment, R^{d} is H, C₁-C₆-alkyl, C₁-C₆-haloalkyl, C₂-C₆-alkenyl, C₂-C₆-alkynyl, which are unsubstituted or substituted with halogen,

phenyl, or benzyl, wherein the rings are unsubstituted or substituted with R^{f}.

In more preferred embodiment, R^{d} is H, C₁-C₆-alkyl, C₁-C₆-haloalkyl, or phenyl which is unsubstituted or substituted with R^{f}.

In one embodiment, R^{e} is C₁-C₆-alkyl, C₁-C₆-haloalkyl, C₃-C₆-cycloalkyl, which are unsubstituted or substituted with halogen,

phenyl, or benzyl, wherein the rings are unsubstituted or substituted with R^{f}.

In more preferred embodiment, R^{e} is H, C₁-C₆-alkyl, C₁-C₆-haloalkyl, or phenyl which is unsubstituted or substituted with R^{f}.

In one embodiment, R^{f} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₀₆-alkenyl, C₂-C₀₆-alkynyl, C₃-C₆-cycloalkyl, C₃-C₆-cycloalkoxy, which are unsubstituted or substituted with halogen,

C(O)—OR^{a}, NR^{b}R^{c}, C₁-C₆-alkylen-NR^{b}R^{c}, C₁-C₆-alkylen-CN, C(O)—NR^{b}R^{c}, C(O)—R^{d}, SO₂NR^{b}R^{c}, or S(═O)ₘRe.

In more preferred embodiment, R^{f} is halogen, N₃, OH, CN, C₁-C₆-alkyl, C₁-C₆-haloalkyl, C₁-C₆-alkoxy, C₁-C₆-haloalkoxy, C₂-C₆-alkenyl, C₂-C₆-alkynyl, C₃-C₆-cycloalkyl, C₃-C₆-cycloalkoxy, which are unsubstituted or substituted with halogen,

C(O)—OR^{a}, NR^{b}R^{c}, C₁-C₆-alkylen-NR^{b}R^{c}, C₁-C₆-alkylen-CN, C(O)—NR^{b}R^{c}, C(O)—R^{d}, SO₂NR^{b}R^{c}, or S(═O)ₘRe.

In a preferred embodiment, R^{f} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₀₆-alkenyl, C₂-C₆-alkynyl, C₃-C₆-cycloalkyl, C₃-C₆-cycloalkoxy, which are unsubstituted or substituted with halogen,

C(O)—OR^{a}, NR^{b}R^{c}, C₁-C₆-alkylen-NR^{b}R^{c}, NH—C₁-C₆-alkylen-NR^{b}R^{c}, C(O)—NR^{b}R^{c}, C(O)—R^{d}, SO₂NR^{b}R^{c}, or S(═O)ₘRe.

In more preferred embodiment, R^{g} is halogen, N₃, OH, CN, NO₂, C₁-C₆-alkyl, C₁-C₆-haloalkyl, C₁-C₆-alkoxy, C₁-C₆-haloalkoxy, C₂-C₀₆-alkenyl, C₃-C₆-cycloalkyl, C₃-C₆-cycloalkoxy, which are unsubstituted or substituted with halogen,

C(O)—OR^{a}, NR^{b}R^{c}, C₁-C₆-alkylen-NR^{b}R^{c}, C(O)—NR^{b}R^{c}, C(O)—R^{d}, SO₂NR^{b}R^{c}, or S(═O)ₘR^{e}.

In one embodiment, m is 0.

In another embodiment, m is 1.

In another embodiment, m is 2.

Particularly preferred X—Y—Z-T are formulas XYZT-1 to XYZT-19 wherein

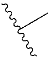

denotes attachment to the 6 membered hetaryl and # denotes attachment to R¹¹ or R¹², and wherein R^{e}, xa, xb, xy and xc are as defined in compounds of formula I.

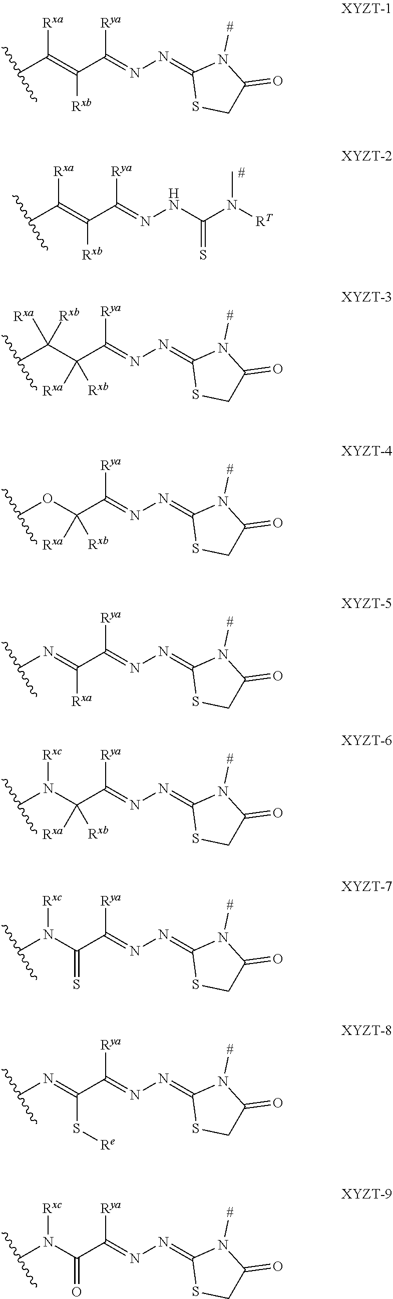

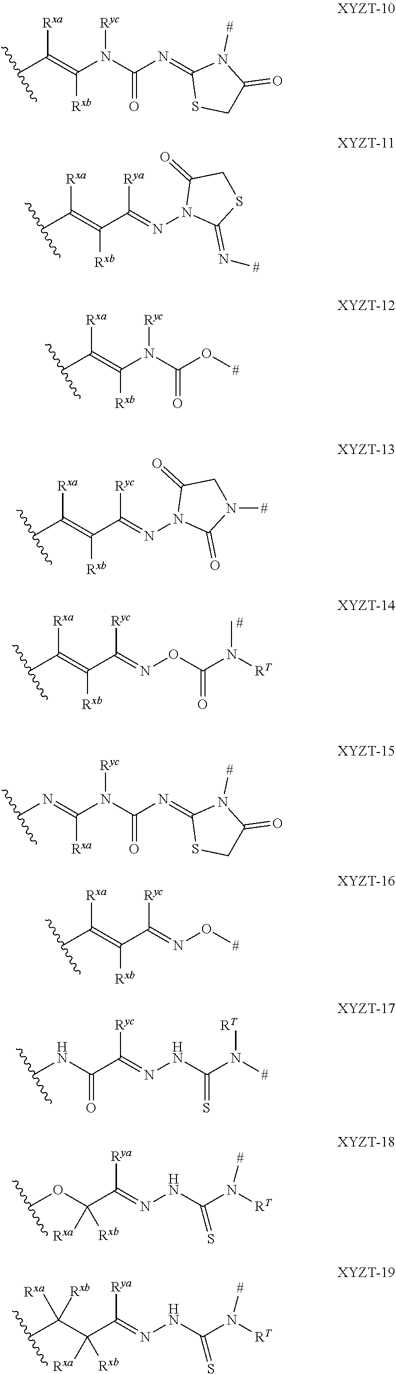

Also particularly preferred X—Y—Z-T are formulas XYZT-1 to XYZT-16;

Also particularly preferred X—Y—Z-T are formulas XYZT-17 to XYZT-19;

In one embodiment, R¹¹C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₁-C₄-alkyl-C₃-C₆-cycloalkoxy, which are unsubstituted or substituted with halogen,

aryl, arylcarbonyl, aryl-C₁-C₄-alkyl, aryloxy-C₁-C₄-alkyl, hetaryl, carbonylhetaryl, C₁-C₄-alkyl-hetaryl and C—C₄-alkyl-hetaryloxy, where the rings are unsubstituted or substituted with R^{g} and wherein the hetaryl is a 5- or 6-membered monocyclic hetaryl or a 8-, 9- or 10-membered bicyclic hetaryl.

In more preferred embodiment, R¹¹ C₁-C₆-alkyl, C₂-C₆-alkenyl, C₂-C₆-alkynyl, C₃-C₆-cycloalkyl, which are unsubstituted or substituted with halogen,

aryl, arylcarbonyl, aryl-C₁-C₄-alkyl, aryloxy-C₁-C₄-alkyl, hetaryl, carbonylhetaryl, C₁-C₄-alkyl-hetaryl and C—C₄-alkyl-hetaryloxy, where the rings are unsubstituted or substituted with R^{g} and wherein the hetaryl is a 5- or 6-membered monocyclic hetaryl or a 8-, 9- or 10-membered bicyclic hetaryl.

In most preferred embodiment, R¹¹ aryl, aryl-C₁-C₄-alkyl, hetaryl, or hetaryl-C₁-C₄-alkyl, wherein the rings are unsubstituted or substituted with R^{g} and where hetaryl in hetaryl or hetaryl-C₁-C₄-alkyl, is preferably a 5- or 6-membered monocyclic hetaryl such as pyridyl, pyrimidinyl, pyridazinyl, pyrrolyl, pyrazolyl, imidazolyl, oxazolyl, thiazolyl, isoxazolyl or isothiazolyl which is unsubstituted or substituted with R^{g}.

Examples of particularly preferred radicals R¹¹ are the radicals R¹¹-1 to R¹¹-29 summarized in Table B below.

TABLE B
Examples of radicals R¹¹

| | |
|---|---|
| R¹¹-1 | 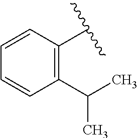 [SMILES: *c1ccccc1C(C)C] |
| R¹¹-2 | 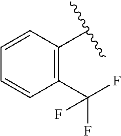 [SMILES: *c1ccccc1C(F)(F)F] |
| R¹¹-3 | 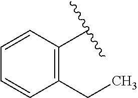 [SMILES: *c1ccccc1CC] |
| R¹¹-4 | 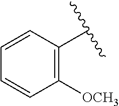 [SMILES: *c1ccccc1OC] |
| R¹¹-5 | 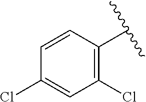 [SMILES: *c1ccc(Cl)cc1Cl] |
| R¹¹-6 | 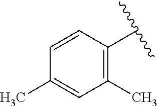 |
| R¹¹-7 | 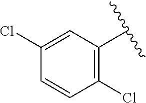 [SMILES: *c1cc(Cl)ccc1Cl] |
| R¹¹-8 | 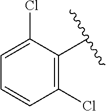 [SMILES: *c1c(Cl)cccc1Cl] |
| R¹¹-9 | 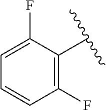 [SMILES: *c1c(F)cccc1F] |
| R¹¹-10 | 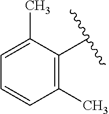 [SMILES: *c1c(C)cccc1C] |
| R¹¹-11 | 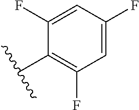 [SMILES: *c1c(F)cc(F)cc1F] |
| R¹¹-12 | 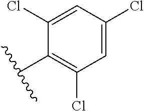 [SMILES: *c1c(Cl)cc(Cl)cc1Cl] |
| R¹¹-13 | 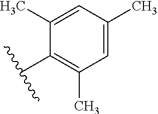 |
| R¹¹-14 | 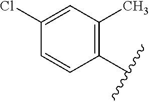 [SMILES: *c1ccc(Cl)cc1C] |
| R¹¹-15 | 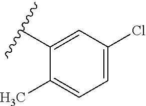 [SMILES: *c1cc(Cl)ccc1C] |
| R¹¹-16 | 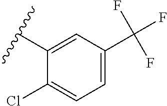 [SMILES: *c1cc(C(F)(F)F)ccc1Cl] |
| R¹¹-17 | 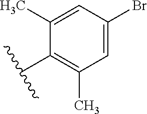 [SMILES: *c1c(C)cc(Br)cc1C] |
| R¹¹-18 | 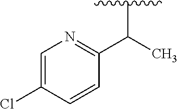 [SMILES: *C(C)c1ccc(Cl)cn1] |
| R¹¹-19 | 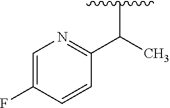 [SMILES: *C(C)c1ccc(F)cn1] |
| R¹¹-20 | 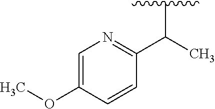 [SMILES: *C(C)c1ccc(OC)cn1] |
| R¹¹-21 | 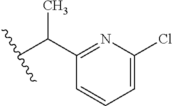 [SMILES: *C(C)c1cccc(Cl)n1] |
| R¹¹-22 | 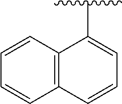 [SMILES: *c1cccc2ccccc12] |
| R¹¹-23 | 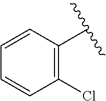 [SMILES: *c1ccccc1Cl] |
| R¹¹-24 | 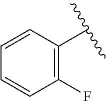 [SMILES: *c1ccccc1F] |
| R¹¹-25 | 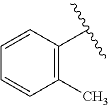 [SMILES: *c1ccccc1C] |
| R¹¹-26 | 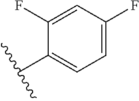 [SMILES: *c1ccc(F)cc1F] |
| R¹¹-27 | 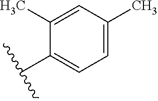 |
| R¹¹-28 | 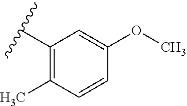 |
| R¹¹-29 | 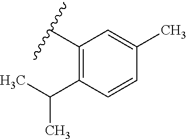 [SMILES: *c1cc(C)ccc1C(C)C] |

In one embodiment, R¹² is a radical of the formula (A¹),

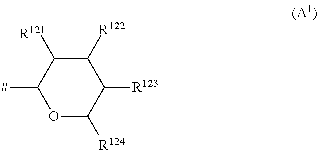

wherein # indicates the point of attachment to T and wherein R¹²¹, R¹²², R¹²³ and R¹²⁴ are as defined above and wherein R¹²¹, R¹²², R¹²³ and R¹²⁴ independently of each other and especially in combination preferably have the following meanings:
R¹²¹ is C₁-C₄-alkoxy, in particular OCH₃, OC₂H₅;
R¹²² is C₁-C₄-alkoxy, such as OCH₃, OC₂H₅, n-propoxyx or isopropoxy, or C₃-C₄-alkenyloxy, such as allyloxy, with R¹²² in particular being OCH₃, OC₂H₅, or n-propoxy;
R¹²³ is OH, C₁-C₄-alkoxy, such as OCH₃, OC₂H₅, or C₃-C₄-alkenyloxy, such as allyloxy, with R¹²³ in particular being OCH₃, OC₂H₅;
R¹²⁴ is C₁-C₄-alkyl, such as CH₃ or C₂H₅, or C₁-C₄-alkoxy-C₁-C₄-alkyl, such as methoxymethyl, ethoxymethyl, 2-methoxyethyl or 2-ethoxyethyl, with R¹²⁴ in particular being methyl.

In more preferred embodiment, R¹² is in particular a radical of the formula (A¹¹), e.g. (A¹¹-a) or (A¹¹-b)

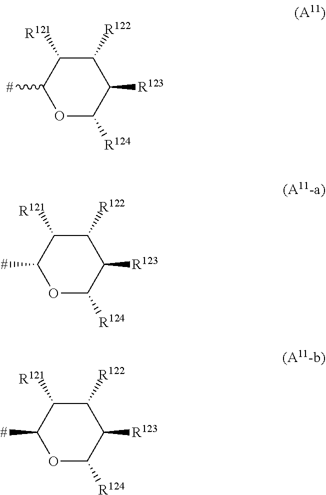

wherein # indicates the point of attachment to T and wherein R¹²¹, R¹²², R¹²³ and R¹²⁴ are as defined above and wherein R¹²¹, R¹²², R¹²³and R¹²⁴ independently of each other and especially in combination preferably have the following meanings:
R¹²¹ is C₁-C₄-alkoxy, in particular OCH₃ or OC₂H₅;
R¹²² is C₁-C₄-alkoxy, such as OCH₃, OC₂H₅, n-propoxyx or isopropoxy, or C₃-C₄-alkenyloxy, such as allyloxy, with R¹²² in particular being OCH₃, OC₂H₅ or n-propoxy;
R¹²³ is OH, C₁-C₄-alkoxy, such as OCH₃ or OC₂H₅, or C₃-C₄-alkenyloxy, such as allyloxy, with R¹²³ in particular being OCH₃ or OC₂H₅;
R¹²⁴ is C₁-C₄-alkyl, such as CH₃ or C₂H₅, or C₁-C₄-alkoxy-C₁-C₄-alkyl, such as methoxymethyl, ethoxymethyl, 2-methoxyethyl or 2-ethoxyethyl, with R¹²⁴ in particular being methyl.

Particular examples of radicals R¹² are the following radicals A¹¹-1, A¹¹-1a, A¹¹-1b, A¹¹-2, A¹¹-2a, A¹¹-2b, A¹¹-3, A¹¹-3a and A¹¹-3b:

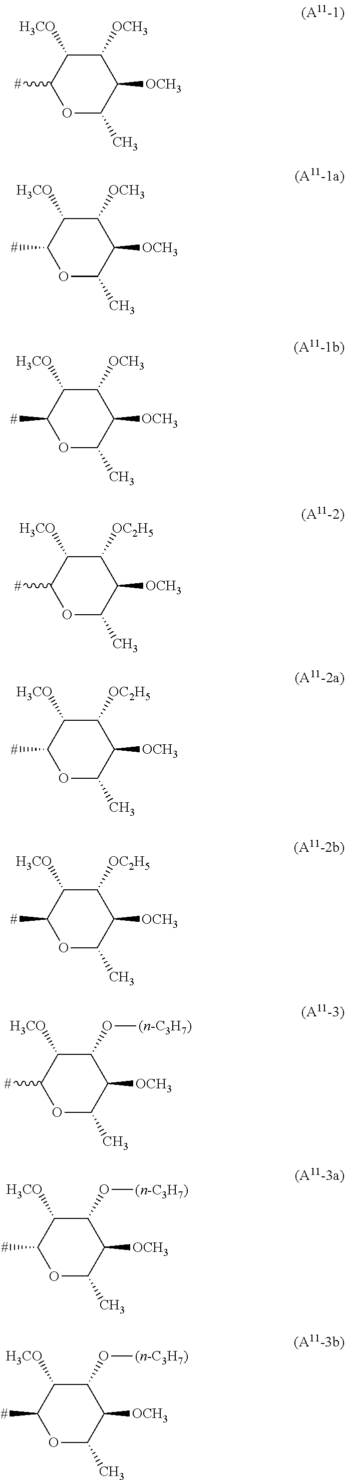

Particularly preferred compounds of formula I are compounds wherein,

A is N or CR^{A};

G is N or CR^{B};

Q is NH or NCH₃

R is H or C₁-C₆-alkyl, preferably CH₃;

R^{A} is H or N(CH₃)₂;

R^{B} is H or CH₃;

Ar is Ar-2;

R¹ is a moiety of formula X—Y—Z-T-R¹¹ or X—Y—Z-T-R¹²; wherein X—Y—Z-T is selected from X—Y—Z-T-1, X—Y—Z-T-2, X—Y—Z-T-3, X—Y—Z-T-4, X—Y—Z-T, X—Y—Z-T-9, X—Y—Z-T-13, X—Y—Z-T-16, X—Y—Z-T-17, X—Y—Z-T-18, and X—Y—Z-T-19;

R¹¹ is R¹¹-1 or R¹¹-10;

R¹² is formula A¹¹-1;

Also particularly preferred compounds of formula I are compounds of formula I.a to I.p, wherein D is R¹¹ or R¹², wherein R¹¹ is selected from R¹¹-1 to R¹¹-29, and R¹² is selected from (A¹¹-la), (A¹¹-1 b), (A¹¹-2a), (A¹¹-2b), (A¹¹-3a), and (A¹¹-3b).

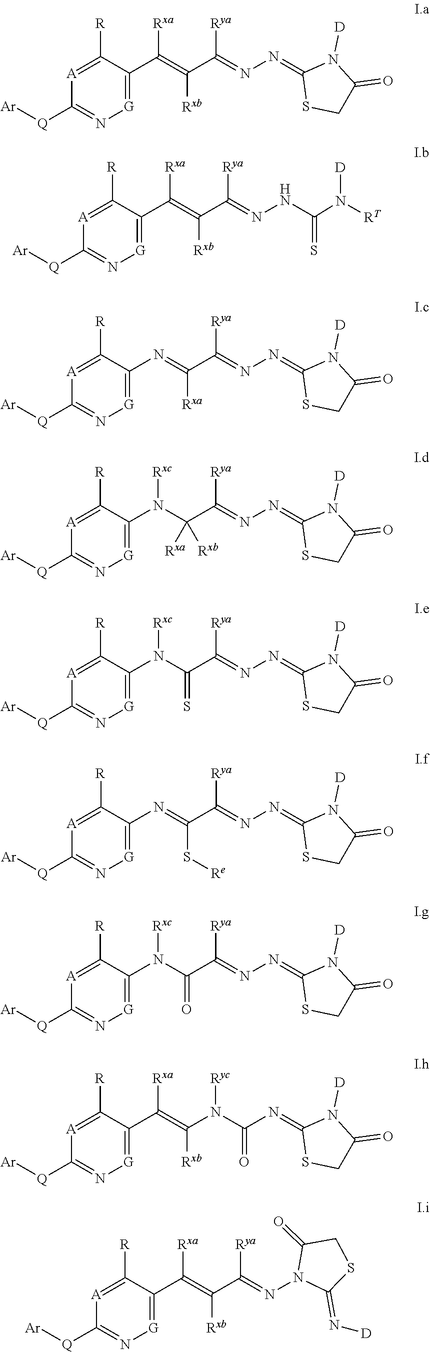

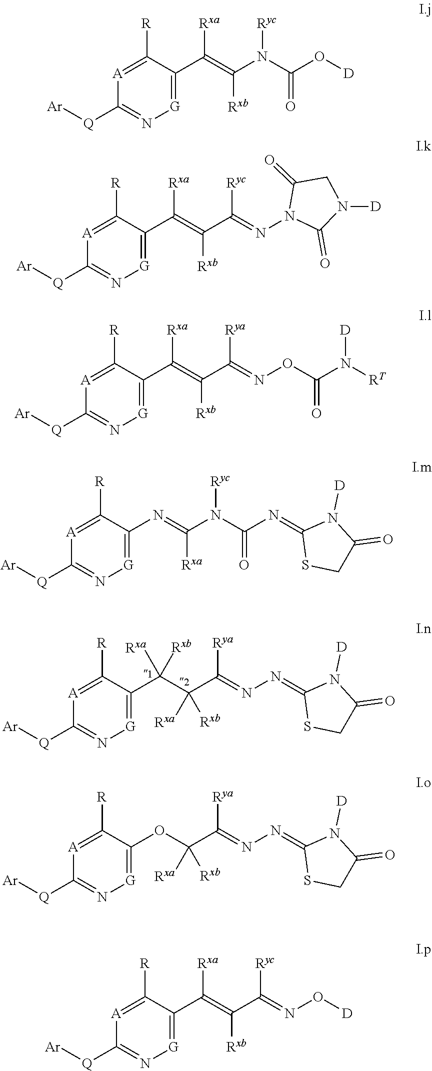

wherein,

Ar is Ar¹, Ar², Ar³, Ar⁴, Ar⁵, Ar⁶, Ar⁷, Ar⁸, Ar⁹, Ar¹⁰, Ar¹¹, or Ar¹²;

Q is NH, NCH₃, or O;

A is N or CH;

G is N, CH, C—CH₃, or C—Cl;

R is H, CH₃, and Cl;

D is R¹¹-1, R¹¹-2, R¹¹-3, R¹¹-4, R¹¹-5, R¹¹-6, R¹¹-7, R¹¹-8, R¹¹-9, R¹¹-10, R¹¹-11, R¹¹-12, R¹¹-13, R¹¹-14, R¹¹-15, R¹¹-16, R¹¹-17, R¹¹-18, R¹¹-19, R¹¹-20, R¹¹-21, R¹¹-22, R¹¹-23, R¹¹-24, R¹¹- 25, R¹¹-26, R¹¹-27, R¹¹-28, R¹¹-29, (A¹¹-1a), (A¹¹⁻¹ b), (A¹¹-2a), (A¹¹-2b), (A¹¹-3a), or (A¹¹-3b);

R^{xa} is H or CH₃;

R^{xb} is H or CH₃;

R^{xc} is H or CH₃;

R^{ya} is H or CH₃;

R^{yc} is H or CH₃;

R^{T} is H or CH₃; and

R^{e} is CH₃ Or CH₂Ph.

Particular compounds of formula I are the compounds of the formulae I.a to I.o that are compiled in the following tables. Each of the groups mentioned for a substituent in the tables is furthermore per se, independently of the combination in which it is mentioned, a particularly preferred aspect of the substituent in question.

Table a.1. Compounds of formula I.a in which R^{xa} is H, R^{xb} is H, R^{ya} is H, A is CH, G is CH, and the combination of R, Q, Ar and D for a compound corresponds to each line of Table A.

Table a.2. Compounds of formula I.a in which R^{xa} is H, R^{xb} is H, R^{ya} is H, A is N, G is CH, and the combination of R, Q, Ar and D for a compound corresponds to each line of Table A.

Table a.3. Compounds of formula I.a in which R^{xa} is H, R^{xb} is H, R^{ya} is H, A is CH, G is N, and the combination of R, Q, Ar and D for a compound corresponds to each line of Table A.

Table a.4. Compounds of formula I.a in which R^{xa} is H, R^{xb} is H, R^{ya} is H, A is N, G is N, and the combination of R, Q, Ar and D for a compound corresponds to each line of Table A.

Table a.5. Compounds of formula I.a in which R^{xa} is H, R^{xb} is H, R^{ya} is H, A is CH, G is C—CH₃, and the combination of R, Q, Ar and D for a compound corresponds to each line of Table A.

Table a.6. Compounds of formula I.a in which R^{xa} is H, R^{xb} is H, R^{ya} is H, A is N, G is C—CH₃, and the combination of R, Q, Ar and D for a compound corresponds to each line of Table A.

Table a.7. Compounds of formula I.a in which R^{xa} is H, R^{xb} is H, R^{ya} is H, A is CH, G is C—Cl, and the combination of R, Q, Ar and D for a compound corresponds to each line of Table A.

Table a.8. Compounds of formula I.a in which R^{xa} is H, R^{xb} is H, R^{ya} is H, A is N, G is C—Cl, and the combination of R, Q, Ar and D for a compound corresponds to each line of Table A.

Table a.9. Compounds of formula I.a in which R^{xa} is CH₃, R^{xb} is H, R^{ya} is H, A is CH, G is CH, and the combination of R, Q, Ar and D for a compound corresponds to each line of Table A.

Table a.10. Compounds of formula I.a in which R^{xa} is CH₃, R^{xb} is H, R^{ya} is H, A is N, G is CH, and the combination of R, Q, Ar and D for a compound corresponds to each line of Table A.

Table a.11. Compounds of formula I.a in which R^{xa} is CH₃, R^{xb} is H, R^{ya} is H, A is CH, G is N, and the combination of R, Q, Ar and D for a compound corresponds to each line of Table A.

Table a.12. Compounds of formula I.a in which R^{xa} is CH₃, R^{xb} is H, R^{ya} is H, A is N, G is N, and the combination of R, Q, Ar and D for a compound corresponds to each line of Table A.

Table a.13. Compounds of formula I.a in which R^{xa} is CH₃, R^{xb} is H, R^{ya} is H, A is CH, G is C—CH₃, and the combination of R, Q, Ar and D for a compound corresponds to each line of Table A.

Table a.14. Compounds of formula I.a in which R^{xa} is CH₃, R^{xb} is H, R^{ya} is H, A is N, G is C—CH₃, and the combination of R, Q, Ar and D for a compound corresponds to each line of Table A.

Table a.15. Compounds of formula I.a in which R^{xa} is CH₃, R^{xb} is H, R^{ya} is H, A is CH, G is C—Cl, and the combination of R, Q, Ar and D for a compound corresponds to each line of Table A.

Table a.16. Compounds of formula I.a in which R^{xa} is CH₃, R^{xb} is H, R^{ya} is H, A is N, G is C—Cl, and the combination of R, Q, Ar and D for a compound corresponds to each line of Table A.

Table a.17. Compounds of formula I.a in which R^{xa} is H, R^{xb} is CH₃, R^{ya} is H, A is CH, G is CH, and the combination of R, Q, Ar and D for a compound corresponds to each line of Table A.

Table a.18. Compounds of formula I.a in which R^{xa} is H, R^{xb} is CH₃, R^{ya} is H, A is N, G is CH, and the combination of R, Q, Ar and D for a compound corresponds to each line of Table A.

Table a.19. Compounds of formula I.a in which R^{xa} is H, R^{xb} is CH₃, R^{ya} is H, A is CH, G is N, and the combination of R, Q, Ar and D for a compound corresponds to each line of Table A.

Table a.20. Compounds of formula I.a in which R^{xa} is H, R^{xb} is CH₃, R^{ya} is H, A is N, G is N, and the combination of R, Q, Ar and D for a compound corresponds to each line of Table A.

Table a.21. Compounds of formula I.a in which R^{xa} is H, R^{xb} is CH₃, R^{ya} is H, A is CH, G is C—C₂H₅, and the combination of R, Q, Ar and D for a compound corresponds to each line of Table A.

Table a.22. Compounds of formula I.a in which R^{xa} is H, R^{xb} is CH₃, R^{ya} is H, A is N, G is C—CH₃, and the combination of R, Q, Ar and D for a compound corresponds to each line of Table A.

Table a.23. Compounds of formula I.a in which R^{xa} is H, R^{xb} is CH₃, R^{ya} is H, A is CH, G is C—Cl, and the combination of R, Q, Ar and D for a compound corresponds to each line of Table A.

Table a.24. Compounds of formula I.a in which R^{xa} is H, R^{xb} is CH₃, R^{ya} is H, A is N, G is C—Cl, and the combination of R, Q, Ar and D for a compound corresponds to each line of Table A.

Table a.25. Compounds of formula I.a in which R^{xa} is H, R^{xb} is H, R^{ya} is CH₃, A is CH, G is CH, and the combination of R, Q, Ar and D for a compound corresponds to each line of Table A.

Table a.26. Compounds of formula I.a in which R^{xa} is H, R^{xb} is H, R^{ya} is CH₃, A is N, G is CH, and the combination of R, Q, Ar and D for a compound corresponds to each line of Table A.

Table a.27. Compounds of formula I.a in which R^{xa} is H, R^{xb} is H, R^{ya} is CH₃, A is CH, G is N, and the combination of R, Q, Ar and D for a compound corresponds to each line of Table A.

Table a.28. Compounds of formula I.a in which R^{xa} is H, R^{xb} is H, R^{ya} is CH₃, A is N, G is N, and the combination of R, Q, Ar and D for a compound corresponds to each line of Table A.

Table a.29. Compounds of formula I.a in which R^{xa} is H, R^{xb} is H, R^{ya} is CH₃, A is CH, G is C—CH₃, and the combination of R, Q, Ar and D for a compound corresponds to each line of Table A.

Table a.30. Compounds of formula I.a in which R^{xa} is H, R^{xb} is H, R^{ya} is CH₃, A is N, G is C—CH₃, and the combination of R, Q, Ar and D for a compound corresponds to each line of Table A.

Table a.31. Compounds of formula I.a in which R^{xa} is H, R^{xb} is H, R^{ya} is CH₃, A is CH, G is C—Cl, and the combination of R, Q, Ar and D for a compound corresponds to each line of Table A.

Table a.32. Compounds of formula I.a in which R^{xa} is H, R^{xb} is H, R^{ya} is CH₃, A is N, G is C—Cl, and the combination of R, Q, Ar and D for a compound corresponds to each line of Table A.

Table a.33. Compounds of formula I.a in which R^{xa} is CH₃, R^{xb} is CH₃, R^{ya} is H, A is CH, G is CH, and the combination of R, Q, Ar and D for a compound corresponds to each line of Table A.

Table a.34. Compounds of formula I.a in which R^{xa} is CH₃, R^{xb} is CH₃, R^{ya} is H, A is N, G is CH, and the combination of R, Q, Ar and D for a compound corresponds to each line of Table A.

Table a.35. Compounds of formula I.a in which R^{xa} is CH₃, R^{xb} is CH₃, R^{ya} is H, A is CH, G is N, and the combination of R, Q, Ar and D for a compound corresponds to each line of Table A.

Table a.36. Compounds of formula I.a in which R^{xa} is CH₃, R^{xb} is CH₃, R^{ya} is H, A is N, G is N, and the combination of R, Q, Ar and D for a compound corresponds to each line of Table A.

Table a.37. Compounds of formula I.a in which R^{xa} is CH₃, R^{xb} is CH₃, R^{ya} is H, A is CH, G is C—CH₃, and the combination of R, Q, Ar and D for a compound corresponds to each line of Table A.

Table a.38. Compounds of formula I.a in which R^{xa} is CH₃, R^{xb} is CH₃, R^{ya} is H, A is N, G is C—CH₃, and the combination of R, Q, Ar and D for a compound corresponds to each line of Table A.

Table a.39. Compounds of formula I.a in which R^{xa} is CH₃, R^{xb} is CH₃, R^{ya} is H, A is CH, G is C—Cl, and the combination of R, Q, Ar and D for a compound corresponds to each line of Table A.

Table a.40. Compounds of formula I.a in which R^{xa} is CH₃, R^{xb} is CH₃, R^{ya} is H, A is N, G is C—Cl, and the combination of R, Q, Ar and D for a compound corresponds to each line of Table A.

Table a.41. Compounds of formula I.a in which R^{xa} is CH₃, R^{xb} is CH₃, R^{ya} is CH₃, A is CH, G is CH, and the combination of R, Q, Ar and D for a compound corresponds to each line of Table A.

Table a.42. Compounds of formula I.a in which R^{xa} is CH₃, R^{xb} is CH₃, R^{ya} is CH₃, A is N, G is CH, and the combination of R, Q, Ar and D for a compound corresponds to each line of Table A.

Table a.43. Compounds of formula I.a in which R^{xa} is CH₃, R^{xb} is CH₃, R^{ya} is CH₃, A is CH, G is N, and the combination of R, Q, Ar and D for a compound corresponds to each line of Table A.

Table a.44. Compounds of formula I.a in which R^{xa} is CH₃, R^{xb} is CH₃, R^{ya} is CH₃, A is N, G is N, and the combination of R, Q, Ar and D for a compound corresponds to each line of Table A.

Table a.45. Compounds of formula I.a in which R^{xa} is CH₃, R^{xb} is CH₃, R^{ya} is CH₃, A is CH, G is C—CH₃, and the combination of R, Q, Ar and D for a compound corresponds to each line of Table A.

Table a.46. Compounds of formula I.a in which R^{xa} is CH₃, R^{xb} is CH₃, R^{ya} is CH₃, A is N, G is C—CH₃, and the combination of R, Q, Ar and D for a compound corresponds to each line of Table A.

Table a.47. Compounds of formula I.a in which R^{xa} is CH₃, R^{xb} is CH₃, R^{ya} is CH₃, A is CH, G is C—Cl, and the combination of R, Q, Ar and D for a compound corresponds to each line of Table A.

Table a.48. Compounds of formula I.a in which R^{xa} is CH₃, R^{xb} is CH₃, R^{ya} is CH₃, A is N, G is C—Cl, and the combination of R, Q, Ar and D for a compound corresponds to each line of Table A.

Table b.1. Compounds of formula I.b in which R^{xa} is H, R^{xb} is H, R^{ya} is H, A is CH, G is CH, R^{T} is H, and the combination of R, Q, Ar and D for a compound corresponds to each line of Table A.

Table b.2. Compounds of formula I.b in which R^{xa} is H, R^{xb} is H, R^{ya} is H, A is N, G is CH, R^{T} is H, and the combination of R, Q, Ar and D for a compound corresponds to each line of Table A.

Table b.3. Compounds of formula I.b in which R^{xa} is H, R^{xb} is H, R^{ya} is H, A is CH, G is N, R^{T} is H, and the combination of R, Q, Ar and D for a compound corresponds to each line of Table A.

Table b.4. Compounds of formula I.b in which R^{xa} is H, R^{xb} is H, R^{ya} is H, A is N, G is N, R^{T} is H, and the combination of R, Q, Ar and D for a compound corresponds to each line of Table A.

Table b.5. Compounds of formula I.b in which R^{xa} is H, R^{xb} is H, R^{ya} is H, A is CH, G is C—CH₃, R^{T} is H, and the combination of R, Q, Ar and D for a compound corresponds to each line of Table A.

Table b.6. Compounds of formula I.b in which R^{xa} is H, R^{xb} is H, R^{ya} is H, A is N, G is C—CH₃, R^{T} is H, and the combination of R, Q, Ar and D for a compound corresponds to each line of Table A.

Table b.7. Compounds of formula I.b in which R^{xa} is H, R^{xb} is H, R^{ya} is H, A is CH, G is C—Cl, R^{T} is H, and the combination of R, Q, Ar and D for a compound corresponds to each line of Table A.

Table b.8. Compounds of formula I.b in which R^{xa} is H, R^{xb} is H, R^{ya} is H, A is N, G is C—Cl, R^{T} is H, and the combination of R, Q, Ar and D for a compound corresponds to each line of Table A.

Table b.9. Compounds of formula I.b in which R^{xa} is CH₃, R^{xb} is H, R^{ya} is H, A is CH, G is CH, R^{T} is H, and the combination of R, Q, Ar and D for a compound corresponds to each line of Table A.

Table b.10. Compounds of formula I.b in which R^{xa} is CH₃, R^{xb} is H, R^{ya} is H, A is N, G is CH, R^{T} is H, and the combination of R, Q, Ar and D for a compound corresponds to each line of Table A.

Table b.11. Compounds of formula I.b in which R^{xa} is CH₃, R^{xb} is H, R^{ya} is H, A is CH, G is N, R^{T} is H, and the combination of R, Q, Ar and D for a compound corresponds to each line of Table A.

Table b.12. Compounds of formula I.b in which R^{xa} is CH₃, R^{xb} is H, R^{ya} is H, A is N, G is N, R^{T} is H, and the combination of R, Q, Ar and D for a compound corresponds to each line of Table A.

Table b.13. Compounds of formula I.b in which R^{xa} is CH₃, R^{xb} is H, R^{ya} is H, A is CH, G is C—CH₃, R^{T} is H, and the combination of R, Q, Ar and D for a compound corresponds to each line of Table A.

Table b.14. Compounds of formula I.b in which R^{xa} is CH₃, R^{xb} is H, R^{ya} is H, A is N, G is C—CH₃, R^{T} is H, and the combination of R, Q, Ar and D for a compound corresponds to each line of Table A.

Table b.15. Compounds of formula I.b in which R^{xa} is CH₃, R^{xb} is H, R^{ya} is H, A is CH, G is C—Cl, R^{T} is H, and the combination of R, Q, Ar and D for a compound corresponds to each line of Table A.

Table b.16. Compounds of formula I.b in which R^{xa} is CH₃, R^{xb} is H, R^{ya} is H, A is N, G is C—Cl, R^{T} is H, and the combination of R, Q, Ar and D for a compound corresponds to each line of Table A.

Table b.17. Compounds of formula I.b in which R^{xa} is H, R^{xb} is CH₃, R^{ya} is H, A is CH, G is CH, R^{T} is H, and the combination of R, Q, Ar and D for a compound corresponds to each line of Table A.

Table b.18. Compounds of formula I.b in which R^{xa} is H, R^{xb} is CH₃, R^{ya} is H, A is N, G is CH, R^{T} is H, and the combination of R, Q, Ar and D for a compound corresponds to each line of Table A.

Table b.19. Compounds of formula I.b in which R^{xa} is H, R^{xb} is CH₃, R^{ya} is H, A is CH, G is N, R^{T} is H, and the combination of R, Q, Ar and D for a compound corresponds to each line of Table A.

Table b.20. Compounds of formula I.b in which R^{xa} is H, R^{xb} is CH₃, R^{ya} is H, A is N, G is N, R^{T} is H, and the combination of R, Q, Ar and D for a compound corresponds to each line of Table A.

Table b.21. Compounds of formula I.b in which R^{xa} is H, R^{xb} is CH₃, R^{ya} is H, A is CH, G is C—CH₃, R^{T} is H, and the combination of R, Q, Ar and D for a compound corresponds to each line of Table A.

Table b.22. Compounds of formula I.b in which R^{xa} is H, R^{xb} is CH₃, R^{ya} is H, A is N, G is C—CH₃, R^{T} is H, and the combination of R, Q, Ar and D for a compound corresponds to each line of Table A.

Table b.23. Compounds of formula I.b in which R^{xa} is H, R^{xb} is CH₃, R^{ya} is H, A is CH, G is C—Cl, R^{T} is H, and the combination of R, Q, Ar and D for a compound corresponds to each line of Table A.

Table b.24. Compounds of formula I.b in which R^{xa} is H, R^{xb} is CH₃, R^{ya} is H, A is N, G is C—Cl, R^{T} is H, and the combination of R, Q, Ar and D for a compound corresponds to each line of Table A.

Table b.25. Compounds of formula I.b in which R^{xa} is H, R^{xb} is H, R^{ya} is CH₃, A is CH, G is CH, R^{T} is H, and the combination of R, Q, Ar and D for a compound corresponds to each line of Table A.

Table b.26. Compounds of formula I.b in which R^{xa} is H, R^{xb} is H, R^{ya} is CH₃, A is N, G is CH, R^{T} is H, and the combination of R, Q, Ar and D for a compound corresponds to each line of Table A.

Table b.27. Compounds of formula I.b in which R^{xa} is H, R^{xb} is H, R^{ya} is CH₃, A is CH, G is N, R^{T} is H, and the combination of R, Q, Ar and D for a compound corresponds to each line of Table A.

Table b.28. Compounds of formula I.b in which R^{xa} is H, R^{xb} is H, R^{ya} is CH₃, A is N, G is N, R^{T} is H, and the combination of R, Q, Ar and D for a compound corresponds to each line of Table A.

Table b.29. Compounds of formula I.b in which R^{xa} is H, R^{xb} is H, R^{ya} is CH₃, A is CH, G is C—CH₃, R^{T} is H, and the combination of R, Q, Ar and D for a compound corresponds to each line of Table A.

Table b.30. Compounds of formula I.b in which R^{xa} is H, R^{xb} is H, R^{ya} is CH₃, A is N, G is C—CH₃, R^{T} is H, and the combination of R, Q, Ar and D for a compound corresponds to each line of Table A.

Table b.31. Compounds of formula I.b in which R^{xa} is H, R^{xb} is H, R^{ya} is CH₃, A is CH, G is C—Cl, R^{T} is H, and the combination of R, Q, Ar and D for a compound corresponds to each line of Table A.

Table b.32. Compounds of formula I.b in which R^{xa} is H, R^{xb} is H, R^{ya} is CH₃, A is N, G is C—Cl, R^{T} is H, and the combination of R, Q, Ar and D for a compound corresponds to each line of Table A.

Table b.33. Compounds of formula I.b in which R^{xa} is CH₃, R^{xb} is CH₃, R^{ya} is H, A is CH, G is CH, R^{T} is H, and the combination of R, Q, Ar and D for a compound corresponds to each line of Table A.

Table b.34. Compounds of formula I.b in which R^{xa} is CH₃, R^{xb} is CH₃, R^{ya} is H, A is N, G is CH, R^{T} is H, and the combination of R, Q, Ar and D for a compound corresponds to each line of Table A.

Table b.35. Compounds of formula I.b in which R^{xa} is CH₃, R^{xb} is CH₃, R^{ya} is H, A is CH, G is N, R^{T} is H, and the combination of R, Q, Ar and D for a compound corresponds to each line of Table A.

Table b.36. Compounds of formula I.b in which R^{xa} is CH₃, R^{xb} is CH₃, R^{ya} is H, A is N, G is N, R^{T} is H, and the combination of R, Q, Ar and D for a compound corresponds to each line of Table A.

Table b.37. Compounds of formula I.b in which R^{xa} is CH₃, R^{xb} is CH₃, R^{ya} is H, A is CH, G is C—CH₃, R^{T} is H, and the combination of R, Q, Ar and D for a compound corresponds to each line of Table A.

Table b.38. Compounds of formula I.b in which R^{xa} is CH₃, R^{xb} is CH₃, R^{ya} is H, A is N, G is C—CH₃, R^{T} is H, and the combination of R, Q, Ar and D for a compound corresponds to each line of Table A.

Table b.39. Compounds of formula I.b in which R^{xa} is CH₃, R^{xb} is CH₃, R^{ya} is H, A is CH, G is C—Cl, R^{T} is H, and the combination of R, Q, Ar and D for a compound corresponds to each line of Table A.

Table b.40. Compounds of formula I.b in which R^{xa} is CH₃, R^{xb} is CH₃, R^{ya} is H, A is N, G is C—Cl, R^{T} is H, and the combination of R, Q, Ar and D for a compound corresponds to each line of Table A.

Table b.41. Compounds of formula I.b in which R^{xa} is CH₃, R^{xb} is CH₃, R^{ya} is CH₃, A is CH, G is CH, R^{T} is H, and the combination of R, Q, Ar and D for a compound corresponds to each line of Table A.

Table b.42. Compounds of formula I.b in which R^{xa} is CH₃, R^{xb} is CH₃, R^{ya} is CH₃, A is N, G is CH, R^{T} is H, and the combination of R, Q, Ar and D for a compound corresponds to each line of Table A.

Table b.43. Compounds of formula I.b in which R^{xa} is CH₃, R^{xb} is CH₃, R^{ya} is CH₃, A is CH, G is N, R^{T} is H, and the combination of R, Q, Ar and D for a compound corresponds to each line of Table A.

Table b.44. Compounds of formula I.b in which R^{xa} is CH₃, R^{xb} is CH₃, R^{ya} is CH₃, A is N, G is N, R^{T} is H, and the combination of R, Q, Ar and D for a compound corresponds to each line of Table A.

Table b.45. Compounds of formula I.b in which R^{xa} is CH₃, R^{xb} is CH₃, R^{ya} is CH₃, A is CH, G is CH₃, R^{T} is H, and the combination of R, Q, Ar and D for a compound corresponds to each line of Table A.

Table b.46. Compounds of formula I.b in which R^{xa} is CH₃, R^{xb} is CH₃, R^{ya} is CH₃, A is N, G is CH₃, R^{T} is H, and the combination of R, Q, Ar and D for a compound corresponds to each line of Table A.

Table b.47. Compounds of formula I.b in which R^{xa} is CH₃, R^{xb} is CH₃, R^{ya} is CH₃, A is CH, G is C—Cl, R^{T} is H, and the combination of R, Q, Ar and D for a compound corresponds to each line of Table A.

Table b.48. Compounds of formula I.b in which R^{xa} is CH₃, R^{xb} is CH₃, R^{ya} is CH₃, A is N, G is C—Cl, R^{T} is H, and the combination of R, Q, Ar and D for a compound corresponds to each line of Table A.

Table b.49. Compounds of formula I.b in which R^{xa} is H, R^{xb} is H, R^{ya} is H, A is CH, G is CH, R^{T} is CH₃, and the combination of R, Q, Ar and D for a compound corresponds to each line of Table A.

Table b.50. Compounds of formula I.b in which R^{xa} is H, R^{xb} is H, R^{ya} is H, A is N, G is CH, R^{T} is CH₃, and the combination of R, Q, Ar and D for a compound corresponds to each line of Table A.

Table b.51. Compounds of formula I.b in which R^{xa} is H, R^{xb} is H, R^{ya} is H, A is CH, G is N, R^{T} is CH₃, and the combination of R, Q, Ar and D for a compound corresponds to each line of Table A.

Table b.52. Compounds of formula I.b in which R^{xa} is H, R^{xb} is H, R^{ya} is H, A is N, G is N, R^{T} is CH₃, and the combination of R, Q, Ar and D for a compound corresponds to each line of Table A.

Table b.53. Compounds of formula I.b in which R^{xa} is H, R^{xb} is H, R^{ya} is H, A is CH, G is C—CH₃, R^{T} is CH₃, and the combination of R, Q, Ar and D for a compound corresponds to each line of Table A.

Table b.54. Compounds of formula I.b in which R^{xa} is H, R^{xb} is H, R^{ya} is H, A is N, G is C—CH₃, R^{T} is CH₃, and the combination of R, Q, Ar and D for a compound corresponds to each line of Table A.

Table b.55. Compounds of formula I.b in which R^{xa} is H, R^{xb} is H, R^{ya} is H, A is CH, G is C—Cl, R^{T} is CH₃, and the combination of R, Q, Ar and D for a compound corresponds to each line of Table A.

Table b.56. Compounds of formula I.b in which R^{xa} is H, R^{xb} is H, R^{ya} is H, A is N, G is C—Cl, R^{T} is CH₃, and the combination of R, Q, Ar and D for a compound corresponds to each line of Table A.

Table b.57. Compounds of formula I.b in which R^{xa} is CH₃, R^{xb} is H, R^{ya} is H, A is CH, G is CH, R^{T} is CH₃, and the combination of R, Q, Ar and D for a compound corresponds to each line of Table A.

Table b.58. Compounds of formula I.b in which R^{xa} is CH₃, R^{xb} is H, R^{ya} is H, A is N, G is CH, R^{T} is CH₃, and the combination of R, Q, Ar and D for a compound corresponds to each line of Table A.

Table b.59. Compounds of formula I.b in which R^{xa} is CH₃, R^{xb} is H, R^{ya} is H, A is CH, G is N, R^{T} is CH₃, and the combination of R, Q, Ar and D for a compound corresponds to each line of Table A.

Table b.60. Compounds of formula I.b in which R^{xa} is CH₃, R^{xb} is H, R^{ya} is H, A is N, G is N, R^{T} is CH₃, and the combination of R, Q, Ar and D for a compound corresponds to each line of Table A.

Table b.61. Compounds of formula I.b in which R^{xa} is CH₃, R^{xb} is H, R^{ya} is H, A is CH, G is C—CH₃, R^{T} is CH₃, and the combination of R, Q, Ar and D for a compound corresponds to each line of Table A.

Table b.62. Compounds of formula I.b in which R^{xa} is CH₃, R^{xb} is H, R^{ya} is H, A is N, G is C—CH₃, R^{T} is CH₃, and the combination of R, Q, Ar and D for a compound corresponds to each line of Table A.

Table b.63. Compounds of formula I.b in which R^{xa} is CH₃, R^{xb} is H, R^{ya} is H, A is CH, G is C—Cl, R^{T} is CH₃, and the combination of R, Q, Ar and D for a compound corresponds to each line of Table A.

Table b.64. Compounds of formula I.b in which R^{xa} is CH₃, R^{xb} is H, R^{ya} is H, A is N, G is C—Cl, R^{T} is CH₃, and the combination of R, Q, Ar and D for a compound corresponds to each line of Table A.

Table b.65. Compounds of formula I.b in which R^{xa} is H, R^{xb} is CH₃, R^{ya} is H, A is CH, G is CH, R^{T} is CH₃, and the combination of R, Q, Ar and D for a compound corresponds to each line of Table A.

Table b.66. Compounds of formula I.b in which R^{xa} is H, R^{xb} is CH₃, R^{ya} is H, A is N, G is CH, R^{T} is CH₃, and the combination of R, Q, Ar and D for a compound corresponds to each line of Table A.

Table b.67. Compounds of formula I.b in which R^{xa} is H, R^{xb} is CH₃, R^{ya} is H, A is CH, G is N, R^{T} is CH₃, and the combination of R, Q, Ar and D for a compound corresponds to each line of Table A.

Table b.68. Compounds of formula I.b in which R^{xa} is H, R^{xb} is CH₃, R^{ya} is H, A is N, G is N, R^{T} is CH₃, and the combination of R, Q, Ar and D for a compound corresponds to each line of Table A.

Table b.69. Compounds of formula I.b in which R^{xa} is H, R^{xb} is CH₃, R^{ya} is H, A is CH, G is C—CH₃, R^{T} is CH₃, and the combination of R, Q, Ar and D for a compound corresponds to each line of Table A.

Table b.70. Compounds of formula I.b in which R^{xa} is H, R^{xb} is CH₃, R^{ya} is H, A is N, G is C—CH₃, R^{T} is CH₃, and the combination of R, Q, Ar and D for a compound corresponds to each line of Table A.

Table b.71. Compounds of formula I.b in which R^{xa} is H, R^{xb} is CH₃, R^{ya} is H, A is CH, G is C—Cl, R^{T}is CH₃, and the combination of R, Q, Ar and D for a compound corresponds to each line of Table A.

Table b.72. Compounds of formula I.b in which R^{xa} is H, R^{xb} is CH₃, R^{ya} is H, A is N, G is C—Cl, R^{T} is CH₃, and the combination of R, Q, Ar and D for a compound corresponds to each line of Table A.

Table b.73. Compounds of formula I.b in which R^{xa} is H, R^{xb} is H, R^{ya} is CH₃, A is CH, G is CH, R^{T} is CH₃, and the combination of R, Q, Ar and D for a compound corresponds to each line of Table A.

Table b.74. Compounds of formula I.b in which R^{xa} is H, R^{xb} is H, R^{ya} is CH₃, A is N, G is CH, R^{T} is CH₃, and the combination of R, Q, Ar and D for a compound corresponds to each line of Table A.

Table b.75. Compounds of formula I.b in which R^{xa} is H, R^{xb} is H, R^{ya} is CH₃, A is CH, G is N, R^{T} is CH₃, and the combination of R, Q, Ar and D for a compound corresponds to each line of Table A.

Table b.76. Compounds of formula I.b in which R^{xa} is H, R^{xb} is H, R^{ya} is CH₃, A is N, G is N, R^{T} is CH₃, and the combination of R, Q, Ar and D for a compound corresponds to each line of Table A.

Table b.77. Compounds of formula I.b in which R^{xa} is H, R^{xb} is H, R^{ya} is CH₃, A is CH, G is C—CH₃, R^{T} is CH₃, and the combination of R, Q, Ar and D for a compound corresponds to each line of Table A.

Table b.78. Compounds of formula I.b in which R^{xa} is H, R^{xb} is H, R^{ya} is CH₃, A is N, G is C—CH₃, R^{T} is CH₃, and the combination of R, Q, Ar and D for a compound corresponds to each line of Table A.

Table b.79. Compounds of formula I.b in which R^{xa} is H, R^{xb} is H, R^{ya} is CH₃, A is CH, G is C—Cl, R^{T} is CH₃, and the combination of R, Q, Ar and D for a compound corresponds to each line of Table A.

Table b.80. Compounds of formula I.b in which R^{xa} is H, R^{xb} is H, R^{ya} is CH₃, A is N, G is C—Cl, R^{T} is CH₃, and the combination of R, Q, Ar and D for a compound corresponds to each line of Table A.

Table b.81. Compounds of formula I.b in which R^{xa} is CH₃, R^{xb} is CH₃, R^{ya} is H, A is CH, G is CH, R^{T} is CH₃, and the combination of R, Q, Ar and D for a compound corresponds to each line of Table A.

Table b.82. Compounds of formula I.b in which R^{xa} is CH₃, R^{xb} is CH₃, R^{ya} is H, A is N, G is CH, R^{T} is CH₃, and the combination of R, Q, Ar and D for a compound corresponds to each line of Table A.

Table b.83. Compounds of formula I.b in which R^{xa} is CH₃, R^{xb} is CH₃, R^{ya} is H, A is CH, G is N, R^{T} is CH₃, and the combination of R, Q, Ar and D for a compound corresponds to each line of Table A.

Table b.84. Compounds of formula I.b in which R^{xa} is CH₃, R^{xb} is CH₃, R^{ya} is H, A is N, G is N, R^{T} is CH₃, and the combination of R, Q, Ar and D for a compound corresponds to each line of Table A.

Table b.85. Compounds of formula I.b in which R^{xa} is CH₃, R^{xb} is CH₃, R^{ya} is H, A is CH, G is C—CH₃, R^{T} is CH₃, and the combination of R, Q, Ar and D for a compound corresponds to each line of Table A.

Table b.86. Compounds of formula I.b in which R^{xa} is CH₃, R^{xb} is CH₃, R^{ya} is H, A is N, G is C—CH₃, R^{T} is CH₃, and the combination of R, Q, Ar and D for a compound corresponds to each line of Table A.

Table b.87. Compounds of formula I.b in which R^{xa} is CH₃, R^{xb} is CH₃, R^{ya} is H, A is CH, G is C—Cl, R^{T} is CH₃, and the combination of R, Q, Ar and D for a compound corresponds to each line of Table A.

Table b.88. Compounds of formula I.b in which R^{xa} is CH₃, R^{xb} is CH₃, R^{ya} is H, A is N, G is C—Cl, R^{T} is CH₃, and the combination of R, Q, Ar and D for a compound corresponds to each line of Table A.

Table b.89. Compounds of formula I.b in which R^{xa} is CH₃, R^{xb} is CH₃, R^{ya} is CH₃, A is CH, G is CH, R^{T} is CH₃, and the combination of R, Q, Ar and D for a compound corresponds to each line of Table A.

Table b.90. Compounds of formula I.b in which R^{xa} is CH₃, R^{xb} is CH₃, R^{ya} is CH₃, A is N, G is CH, R^{T} is CH₃, and the combination of R, Q, Ar and D for a compound corresponds to each line of Table A.

Table b.91. Compounds of formula I.b in which R^{xa} is CH₃, R^{xb} is CH₃, R^{ya} is CH₃, A is CH, G is N, R^{T} is CH₃, and the combination of R, Q, Ar and D for a compound corresponds to each line of Table A.

Table b.92. Compounds of formula I.b in which R^{xa} is CH₃, R^{xb} is CH₃, R^{ya} is CH₃, A is N, G is N, R^{T} is CH₃, and the combination of R, Q, Ar and D for a compound corresponds to each line of Table A.

Table b.93. Compounds of formula I.b in which R^{xa} is CH₃, R^{xb} is CH₃, R^{ya} is CH₃, A is CH, G is C—CH₃, R^{T} is CH₃, and the combination of R, Q, Ar and D for a compound corresponds to each line of Table A.

Table b.94. Compounds of formula I.b in which R^{xa} is CH₃, R^{xb} is CH₃, R^{ya} is CH₃, A is N, G is C—CH₃, R^{T} is CH₃, and the combination of R, Q, Ar and D for a compound corresponds to each line of Table A.

Table b.95. Compounds of formula I.b in which R^{xa} is CH₃, R^{xb} is CH₃, R^{ya} is CH₃, A is CH, G is C—Cl, R^{T} is CH₃, and the combination of R, Q, Ar and D for a compound corresponds to each line of Table A.

Table b.96. Compounds of formula I.b in which R^{xa} is CH₃, R^{xb} is CH₃, R^{ya} is CH₃, A is N, G is C—Cl, R^{T} is CH₃, and the combination of R, Q, Ar and D for a compound corresponds to each line of Table A.

Table c.1. Compounds of formula I.c in which R^{xa} is H, R^{ya} is H, A is CH, G is CH, and the combination of R, Q, Ar and D for a compound corresponds to each line of Table A.

Table c.2. Compounds of formula I.c in which R^{xa} is H, R^{ya} is H, A is N, G is CH, and the combination of R, Q, Ar and D for a compound corresponds to each line of Table A.

Table c.3. Compounds of formula I.c in which R^{xa} is H, R^{ya} is H, A is CH, G is N, and the combination of R, Q, Ar and D for a compound corresponds to each line of Table A.

Table c.4. Compounds of formula I.c in which R^{xa} is H, R^{ya} is H, A is N, G is N, and the combination of R, Q, Ar and D for a compound corresponds to each line of Table A.

Table c.5. Compounds of formula I.c in which R^{xa} is H, R^{ya} is H, A is CH, G is C—CH₃, and the combination of R, Q, Ar and D for a compound corresponds to each line of Table A.

Table c.6. Compounds of formula I.c in which R^{xa} is H, R^{ya} is H, A is N, G is C—CH₃, and the combination of R, Q, Ar and D for a compound corresponds to each line of Table A.

Table c.7. Compounds of formula I.c in which R^{xa} is H, R^{ya} is H, A is CH, G is C—Cl, and the combination of R, Q, Ar and D for a compound corresponds to each line of Table A.

Table c.8. Compounds of formula I.c in which R^{xa} is H, R^{ya} is H, A is N, G is C—Cl, and the combination of R, Q, Ar and D for a compound corresponds to each line of Table A.

Table c.9. Compounds of formula I.c in which R^{xa} is CH₃, R^{ya} is H, A is CH, G is CH, and the combination of R, Q, Ar and D for a compound corresponds to each line of Table A.

Table c.10. Compounds of formula I.c in which R^{xa} is CH₃, R^{ya} is H, A is N, G is CH, and the combination of R, Q, Ar and D for a compound corresponds to each line of Table A.

Table c.11. Compounds of formula I.c in which R^{xa} is CH₃, R^{ya} is H, A is CH, G is N, and the combination of R, Q, Ar and D for a compound corresponds to each line of Table A.

Table c.12. Compounds of formula I.c in which R^{xa} is CH₃, R^{ya} is H, A is N, G is N, and the combination of R, Q, Ar and D for a compound corresponds to each line of Table A.

Table c.13. Compounds of formula I.c in which R^{xa} is CH₃, R^{ya} is H, A is CH, G is C—CH₃, and the combination of R, Q, Ar and D for a compound corresponds to each line of Table A.

Table c.14. Compounds of formula I.c in which R^{xa} is CH₃, R^{ya} is H, A is N, G is C—CH₃, and the combination of R, Q, Ar and D for a compound corresponds to each line of Table A.

Table c.15. Compounds of formula I.c in which R^{xa} is CH₃, R^{ya} is H, A is CH, G is C—Cl, and the combination of R, Q, Ar and D for a compound corresponds to each line of Table A.

Table c.16. Compounds of formula I.c in which R^{xa} is CH₃, R^{ya} is H, A is N, G is C—Cl, and the combination of R, Q, Ar and D for a compound corresponds to each line of Table A.

Table c.17. Compounds of formula I.c in which R^{xa} is H, R^{ya} is CH₃, A is CH, G is CH, and the combination of R, Q, Ar and D for a compound corresponds to each line of Table A.

Table c.18. Compounds of formula I.c in which R^{xa} is H, R^{ya} is CH₃, A is N, G is CH, and the combination of R, Q, Ar and D for a compound corresponds to each line of Table A.

Table c.19. Compounds of formula I.c in which R^{xa} is H, R^{ya} is CH₃, A is CH, G is N, and the combination of R, Q, Ar and D for a compound corresponds to each line of Table A.

Table c.20. Compounds of formula I.c in which R^{xa} is H, R^{ya} is CH₃, A is N, G is N, and the combination of R, Q, Ar and D for a compound corresponds to each line of Table A.

Table c.21. Compounds of formula I.c in which R^{xa} is H, R^{ya} is CH₃, A is CH, G is C—CH₃, and the combination of R, Q, Ar and D for a compound corresponds to each line of Table A.

Table c.22. Compounds of formula I.c in which R^{xa} is H, R^{ya} is CH₃, A is N, G is C—CH₃, and the combination of R, Q, Ar and D for a compound corresponds to each line of Table A.

Table c.23. Compounds of formula I.c in which R^{xa} is H, R^{ya} is CH₃, A is CH, G is C—Cl, and the combination of R, Q, Ar and D for a compound corresponds to each line of Table A.

Table c.24. Compounds of formula I.c in which R^{xa} is H, R^{ya} is CH₃, A is N, G is C—Cl, and the combination of R, Q, Ar and D for a compound corresponds to each line of Table A.

Table d.1. Compounds of formula I.d in which R^{xa} is H, R^{xb} is H, R^{xc} is H, R^{ya} is H, A is CH, G is CH, and the combination of R, Q, Ar and D for a compound corresponds to each line of Table A.

Table d.2. Compounds of formula I.d in which R^{xa} is H, R^{xb} is H, R^{xc} is H, R^{ya} is H, A is N, G is CH, and the combination of R, Q, Ar and D for a compound corresponds to each line of Table A.

Table d.3. Compounds of formula I.d in which R^{xa} is H, R^{xb} is H, R^{xc} is H, R^{ya} is H, A is CH, G is N, and the combination of R, Q, Ar and D for a compound corresponds to each line of Table A.

Table d.4. Compounds of formula I.d in which R^{xa} is H, R^{xb} is H, R^{xc} is H, R^{ya} is H, A is N, G is N, and the combination of R, Q, Ar and D for a compound corresponds to each line of Table A.

Table d.5. Compounds of formula I.d in which R^{xa} is H, R^{xb} is H, R^{xc} is H, R^{ya} is H, A is CH, G is C—CH₃, and the combination of R, Q, Ar and D for a compound corresponds to each line of Table A.

Table d.6. Compounds of formula I.d in which R^{xa} is H, R^{xb} is H, R^{xc} is H, R^{ya} is H, A is N, G is C—CH₃, and the combination of R, Q, Ar and D for a compound corresponds to each line of Table A.

Table d.7. Compounds of formula I.d in which R^{xa} is H, R^{xb} is H, R^{xc} is H, R^{ya} is H, A is CH, G is C—Cl, and the combination of R, Q, Ar and D for a compound corresponds to each line of Table A.

Table d.8. Compounds of formula I.d in which R^{xa} is H, R^{xb} is H, R^{xc} is H, R^{ya} is H, A is N, G is C—Cl, and the combination of R, Q, Ar and D for a compound corresponds to each line of Table A.

Table d.9. Compounds of formula I.d in which R^{xa} is CH₃, R^{xb} is H, R^{xc} is H, R^{ya} is H, A is CH, G is CH, and the combination of R, Q, Ar and D for a compound corresponds to each line of Table A.

Table d.10. Compounds of formula I.d in which R^{xa} is CH₃, R^{xb} is H, R^{xc} is H, R^{ya} is H, A is N, G is CH, and the combination of R, Q, Ar and D for a compound corresponds to each line of Table A.

Table d.11. Compounds of formula I.d in which R^{xa} is CH₃, R^{xb} is H, R^{xc} is H, R^{ya} is H, A is CH, G is N, and the combination of R, Q, Ar and D for a compound corresponds to each line of Table A.

Table d.12. Compounds of formula I.d in which R^{xa} is CH₃, R^{xb} is H, R^{xc} is H, R^{ya} is H, A is N, G is N, and the combination of R, Q, Ar and D for a compound corresponds to each line of Table A.

Table d.13. Compounds of formula I.d in which R^{xa} is CH₃, R^{xb} is H, R^{xc} is H, R^{ya} is H, A is CH, G is C—CH₃, and the combination of R, Q, Ar and D for a compound corresponds to each line of Table A.

Table d.14. Compounds of formula I.d in which R^{xa} is CH₃, R^{xb} is H, R^{xc} is H, R^{ya} is H, A is N, G is C—CH₃, and the combination of R, Q, Ar and D for a compound corresponds to each line of Table A.

Table d.15. Compounds of formula I.d in which R^{xa} is CH₃, R^{xb} is H, R^{xc} is H, R^{ya} is H, A is CH, G is C—Cl, and the combination of R, Q, Ar and D for a compound corresponds to each line of Table A.

Table d.16. Compounds of formula I.d in which R^{xa} is CH₃, R^{xb} is H, R^{xc} is H, R^{ya} is H, A is N, G is C—Cl, and the combination of R, Q, Ar and D for a compound corresponds to each line of Table A.

Table d.17. Compounds of formula I.d in which R^{xa} is H, R^{xb} is H, R^{xc} is H, R^{ya} is CH₃, A is CH, G is CH, and the combination of R, Q, Ar and D for a compound corresponds to each line of Table A.

Table d.18. Compounds of formula I.d in which R^{xa} is H, R^{xb} is H, R^{xc} is H, R^{ya} is CH₃, A is N, G is CH, and the combination of R, Q, Ar and D for a compound corresponds to each line of Table A.

Table d.19. Compounds of formula I.d in which R^{xa} is H, R^{xb} is H, R^{xc} is H, R^{ya} is CH₃, A is CH, G is N, and the combination of R, Q, Ar and D for a compound corresponds to each line of Table A.

Table d.20. Compounds of formula I.d in which R^{xa} is H, R^{xb} is H, R^{xc} is H, R^{ya} is CH₃, A is N, G is N, and the combination of R, Q, Ar and D for a compound corresponds to each line of Table A.

Table d.21. Compounds of formula I.d in which R^{xa} is H, R^{xb} is H, R^{xc} is H, R^{ya} is CH₃, A is CH, G is C—CH₃, and the combination of R, Q, Ar and D for a compound corresponds to each line of Table A.

Table d.22. Compounds of formula I.d in which R^{xa} is H, R^{xb} is H, R^{xc} is H, R^{ya} is CH₃, A is N, G is C—CH₃, and the combination of R, Q, Ar and D for a compound corresponds to each line of Table A.

Table d.23. Compounds of formula I.d in which R^{xa} is H, R^{xb} is H, R^{ya} is CH₃, A is CH, G is C—Cl, and the combination of R, Q, Ar and D for a compound corresponds to each line of Table A.

Table d.24. Compounds of formula I.d in which R^{xa} is H, R^{xb} is H, R^{xc} is H, R^{ya} is CH₃, A is N, G is C—Cl, and the combination of R, Q, Ar and D for a compound corresponds to each line of Table A.

Table d.25. Compounds of formula I.d in which R^{xa} is CH₃, R^{xb} is CH₃, R^{xc} is H, R^{ya} is H, A is CH, G is CH, and the combination of R, Q, Ar and D for a compound corresponds to each line of Table A.

Table d.26. Compounds of formula I.d in which R^{xa} is CH₃, R^{xb} is CH₃, R^{xc} is H, R^{ya} is H, A is N, G is CH, and the combination of R, Q, Ar and D for a compound corresponds to each line of Table A.

Table d.27. Compounds of formula I.d in which R^{xa} is CH₃, R^{xb} is CH₃, R^{xc} is H, R^{ya} is H, A is CH, G is N, and the combination of R, Q, Ar and D for a compound corresponds to each line of Table A.

Table d.28. Compounds of formula I.d in which R^{xa} is CH₃, R^{xb} is CH₃, R^{xc} is H, R^{ya} is H, A is N, G is N, and the combination of R, Q, Ar and D for a compound corresponds to each line of Table A.

Table d.29. Compounds of formula I.d in which R^{xa} is CH₃, R^{xb} is CH₃, R^{xc} is H, R^{ya} is H, A is CH, G is C—CH₃, and the combination of R, Q, Ar and D for a compound corresponds to each line of Table A.

Table d.30. Compounds of formula I.d in which R^{xa} is CH₃, R^{xb} is CH₃, R^{xc} is H, R^{ya} is H, A is N, G is C—CH₃, and the combination of R, Q, Ar and D for a compound corresponds to each line of Table A.

Table d.31. Compounds of formula I.d in which R^{xa} is CH₃, R^{xb} is CH₃, R^{xc} is H, R^{ya} is H, A is CH, G is C—Cl, and the combination of R, Q, Ar and D for a compound corresponds to each line of Table A.

Table d.32. Compounds of formula I.d in which R^{xa} is CH₃, R^{xb} is CH₃, R^{xc} is H, R^{ya} is H, A is N, G is C—Cl, and the combination of R, Q, Ar and D for a compound corresponds to each line of Table A.

Table d.33. Compounds of formula I.d in which R^{xa} is CH₃, R^{xb} is CH₃, R^{xc} is H, R^{ya} is CH₃, A is CH, G is CH, and the combination of R, Q, Ar and D for a compound corresponds to each line of Table A.

Table d.34. Compounds of formula I.d in which R^{xa} is CH₃, R^{xb} is CH₃, R^{xc} is H, R^{ya} is CH₃, A is N, G is CH, and the combination of R, Q, Ar and D for a compound corresponds to each line of Table A.

Table d.35. Compounds of formula I.d in which R^{xa} is CH₃, R^{xb} is CH₃, R^{xc} is H, R^{ya} is CH₃, A is CH, G is N, and the combination of R, Q, Ar and D for a compound corresponds to each line of Table A.

Table d.36. Compounds of formula I.d in which R^{xa} is CH₃, R^{xb} is CH₃, R^{xc} is H, R^{ya} is CH₃, A is N, G is N, and the combination of R, Q, Ar and D for a compound corresponds to each line of Table A.

Table d.37. Compounds of formula I.d in which R^{xa} is CH₃, R^{xb} is CH₃, R^{xc} is H, R^{ya} is CH₃, A is CH, G is C—CH₃, and the combination of R, Q, Ar and D for a compound corresponds to each line of Table A.

Table d.38. Compounds of formula I.d in which R^{xa} is CH₃, R^{xb} is CH₃, R^{xc} is H, R^{ya} is CH₃, A is N, G is C—CH₃, and the combination of R, Q, Ar and D for a compound corresponds to each line of Table A.

Table d.39. Compounds of formula I.d in which R^{xa} is CH₃, R^{xb} is CH₃, R^{xc} is H, R^{ya} is CH₃, A is CH, G is C—Cl, and the combination of R, Q, Ar and D for a compound corresponds to each line of Table A.

Table d.40. Compounds of formula I.d in which R^{xa} is CH₃, R^{xb} is CH₃, R^{xc} is H, R^{ya} is CH₃, A is N, G is C—Cl, and the combination of R, Q, Ar and D for a compound corresponds to each line of Table A.

Table d.41. pounds of the formula I.d in which R^{xa} is H, R^{xb} is H, R^{xc} is CH₃, R^{ya} is CH₃, A is CH, G is CH, and the combination of R, Q, Ar and D for a compound corresponds to each line of Table A.

Table d.42. Compounds of formula I.d in which R^{xa} is H, R^{xb} is H, R^{xc} is CH₃, R^{ya} is H, A is N, G is CH, and the combination of R, Q, Ar and D for a compound corresponds to each line of Table A.

Table d.43. Compounds of formula I.d in which R^{xa} is H, R^{xb} is H, R^{xc} is CH₃, R^{ya} is H, A is CH, G is N, and the combination of R, Q, Ar and D for a compound corresponds to each line of Table A.

Table d.44. Compounds of formula I.d in which R^{xa} is H, R^{xb} is H, R^{xc} is CH₃, R^{ya} is H, A is N, G is N, and the combination of R, Q, Ar and D for a compound corresponds to each line of Table A.

Table d.45. Compounds of formula I.d in which R^{xa} is H, R^{xb} is H, R^{xc} is CH₃, R^{ya} is H, A is CH, G is C—CH₃, and the combination of R, Q, Ar and D for a compound corresponds to each line of Table A.

Table d.46. Compounds of formula I.d in which R^{xa} is H, R^{xb} is H, R^{xc} is CH₃, R^{ya} is H, A is N, G is C—CH₃, and the combination of R, Q, Ar and D for a compound corresponds to each line of Table A.

Table d.47. Compounds of formula I.d in which R^{xa} is H, R^{xb} is H, R^{xc} is CH₃, R^{ya} is H, A is CH, G is C—Cl, and the combination of R, Q, Ar and D for a compound corresponds to each line of Table A.

Table d.48. Compounds of formula I.d in which R^{xa} is H, R^{xb} is H, R^{xc} is CH₃, R^{ya} is H, A is N, G is C—Cl, and the combination of R, Q, Ar and D for a compound corresponds to each line of Table A.

Table d.49. Compounds of formula I.d in which R^{xa} is CH₃, R^{xb} is H, R^{xc} is CH₃, R^{ya} is H, A is CH, G is CH, and the combination of R, Q, Ar and D for a compound corresponds to each line of Table A.

Table d.50. Compounds of formula I.d in which R^{xa} is CH₃, R^{xb} is H, R^{xc} is CH₃, R^{ya} is H, A is N, G is CH, and the combination of R, Q, Ar and D for a compound corresponds to each line of Table A.

Table d.51. Compounds of formula I.d in which R^{xa} is CH₃, R^{xb} is H, R^{xc} is CH₃, R^{ya} is H, A is CH, G is N, and the combination of R, Q, Ar and D for a compound corresponds to each line of Table A.

Table d.52. Compounds of formula I.d in which R^{xa} is CH₃, R^{xb} is H, R^{xc} is CH₃, R^{ya} is H, A is N, G is N, and the combination of R, Q, Ar and D for a compound corresponds to each line of Table A.

Table d.53. Compounds of formula I.d in which R^{xa} is CH₃, R^{xb} is H, R^{xc} is CH₃, R^{ya} is H, A is CH, G is C—CH₃, and the combination of R, Q, Ar and D for a compound corresponds to each line of Table A.

Table d.54. Compounds of formula I.d in which R^{xa} is CH₃, R^{xb} is H, R^{xc} is CH₃, R^{ya} is H, A is N, G is C—CH₃, and the combination of R, Q, Ar and D for a compound corresponds to each line of Table A.

Table d.55. Compounds of formula I.d in which R^{xa} is CH₃, R^{xb} is H, R^{xc} is CH₃, R^{ya} is H, A is CH, G is C—Cl, and the combination of R, Q, Ar and D for a compound corresponds to each line of Table A.

Table d.56. Compounds of formula I.d in which R^{xa} is CH₃, R^{xb} is H, R^{xc} is CH₃, R^{ya} is H, A is N, G is C—Cl, and the combination of R, Q, Ar and D for a compound corresponds to each line of Table A.

Table d.57. Compounds of formula I.d in which R^{xa} is H, R^{xb} is H, R^{xc} is CH₃, R^{ya} is CH₃, A is CH, G is CH, and the combination of R, Q, Ar and D for a compound corresponds to each line of Table A.

Table d.58. Compounds of formula I.d in which R^{xa} is H, R^{xb} is H, R^{xc} is CH₃, R^{ya} is CH₃, A is N, G is CH, and the combination of R, Q, Ar and D for a compound corresponds to each line of Table A.

Table d.59. Compounds of formula I.d in which R^{xa} is H, R^{xb} is H, R^{xc} is CH₃, R^{ya} is CH₃, A is CH, G is N, and the combination of R, Q, Ar and D for a compound corresponds to each line of Table A.

Table d.60. Compounds of formula I.d in which R^{xa} is H, R^{xb} is H, R^{xc} is CH₃, R^{ya} is CH₃, A is N, G is N, and the combination of R, Q, Ar and D for a compound corresponds to each line of Table A.

Table d.61. Compounds of formula I.d in which R^{xa} is H, R^{xb} is H, R^{xc} is CH₃, R^{ya} is CH₃, A is CH, G is C—CH₃, and the combination of R, Q, Ar and D for a compound corresponds to each line of Table A.

Table d.62. Compounds of formula I.d in which R^{xa} is H, R^{xb} is H, R^{xc} is CH₃, R^{ya} is CH₃, A is N, G is C—CH₃, and the combination of R, Q, Ar and D for a compound corresponds to each line of Table A.

Table d.63. Compounds of formula I.d in which R^{xa} is H, R^{xb} is H, R^{xc} is CH₃, R^{ya} is CH₃, A is CH, G is C—Cl, and the combination of R, Q, Ar and D for a compound corresponds to each line of Table A.

Table d.64. Compounds of formula I.d in which R^{xa} is H, R^{xb} is H, R^{xc} is CH₃, R^{ya} is CH₃, A is N, G is C—Cl, and the combination of R, Q, Ar and D for a compound corresponds to each line of Table A.

Table d.65. Compounds of formula I.d in which R^{xa} is CH₃, R^{xb} is CH₃, R^{xc} is CH₃, R^{ya} is H, A is CH, G is CH, and the combination of R, Q, Ar and D for a compound corresponds to each line of Table A.

Table d.66. Compounds of formula I.d in which R^{xa} is CH₃, R^{xb} is CH₃, R^{xc} is CH₃, R^{ya} is H, A is N, G is CH, and the combination of R, Q, Ar and D for a compound corresponds to each line of Table A.

Table d.67. Compounds of formula I.d in which R^{xa} is CH₃, R^{xb} is CH₃, R^{xc} is CH₃, R^{ya} is H, A is CH, G is N, and the combination of R, Q, Ar and D for a compound corresponds to each line of Table A.

Table d.68. Compounds of formula I.d in which R^{xa} is CH₃, R^{xb} is CH₃, R^{xc} is CH₃, R^{ya} is H, A is N, G is N, and the combination of R, Q, Ar and D for a compound corresponds to each line of Table A.

Table d.69. Compounds of formula I.d in which R^{xa} is CH₃, R^{xb} is CH₃, R^{xc} is CH₃, R^{ya} is H, A is CH, G is C—CH₃, and the combination of R, Q, Ar and D for a compound corresponds to each line of Table A.

Table d.70. Compounds of formula I.d in which R^{xa} is CH₃, R^{xb} is CH₃, R^{xc} is CH₃, R^{ya} is H, A is N, G is C—CH₃, and the combination of R, Q, Ar and D for a compound corresponds to each line of Table A.

Table d.71. Compounds of formula I.d in which R^{xa} is CH₃, R^{xb} is CH₃, R^{xc} is CH₃, R^{ya} is H, A is CH, G is C—Cl, and the combination of R, Q, Ar and D for a compound corresponds to each line of Table A.

Table d.72. Compounds of formula I.d in which R^{xa} is CH₃, R^{xb} is CH₃, R^{xc} is CH₃, R^{ya} is H, A is N, G is C—Cl, and the combination of R, Q, Ar and D for a compound corresponds to each line of Table A.

Table d.73. Compounds of formula I.d in which R^{xa} is CH₃, R^{xb} is CH₃, R^{xc} is CH₃, R^{ya} is CH₃, A is CH, G is CH, and the combination of R, Q, Ar and D for a compound corresponds to each line of Table A.

Table d.74. Compounds of formula I.d in which R^{xa} is CH₃, R^{xb} is CH₃, R^{xc} is CH₃, R^{ya} is CH₃, A is N, G is CH, and the combination of R, Q, Ar and D for a compound corresponds to each line of Table A.

Table d.75. Compounds of formula I.d in which R^{xa} is CH₃, R^{xb} is CH₃, R^{xc} is CH₃, R^{ya} is CH₃, A is CH, G is N, and the combination of R, Q, Ar and D for a compound corresponds to each line of Table A.

Table d.76. Compounds of formula I.d in which R^{xa} is CH₃, R^{xb} is CH₃, R^{xc} is CH₃, R^{ya} is CH₃, A is N, G is N, and the combination of R, Q, Ar and D for a compound corresponds to each line of Table A.

Table d.77. Compounds of formula I.d in which R^{xa} is CH₃, R^{xb} is CH₃, R^{xc} is CH₃, R^{ya} is CH₃, A is CH, G is C—CH₃, and the combination of R, Q, Ar and D for a compound corresponds to each line of Table A.

Table d.78. Compounds of formula I.d in which R^{xa} is CH₃, R^{xb} is CH₃, R^{xc} is CH₃, R^{ya} is CH₃, A is N, G is C—CH₃, and the combination of R, Q, Ar and D for a compound corresponds to each line of Table A.

Table d.79. Compounds of formula I.d in which R^{xa} is CH₃, R^{xb} is CH₃, R^{xc} is CH₃, R^{ya} is CH₃, A is CH, G is C—Cl, and the combination of R, Q, Ar and D for a compound corresponds to each line of Table A.

Table d.80. Compounds of formula I.d in which R^{xa} is CH₃, R^{xb} is CH₃, R^{xc} is CH₃, R^{ya} is CH₃, A is N, G is C—Cl, and the combination of R, Q, Ar and D for a compound corresponds to each line of Table A.

Table e.1. Compounds of formula I.e in which R^{xc} is H, R^{ya} is H, A is CH, G is CH, and the combination of R, Q, Ar and D for a compound corresponds to each line of Table A.

Table e.2. Compounds of formula I.e in which R^{xc} is H, R^{ya} is H, A is N, G is CH, and the combination of R, Q, Ar and D for a compound corresponds to each line of Table A.

Table e.3. Compounds of formula I.e in which R^{xc} is H, R^{ya} is H, A is CH, G is N, and the combination of R, Q, Ar and D for a compound corresponds to each line of Table A.

Table e.4. Compounds of formula I.e in which R^{xc} is H, R^{ya} is H, A is N, G is N, and the combination of R, Q, Ar and D for a compound corresponds to each line of Table A.

Table e.5. Compounds of formula I.e in which R^{xc} is H, R^{ya} is H, A is CH, G is C—CH₃, and the combination of R, Q, Ar and D for a compound corresponds to each line of Table A.

Table e.6. Compounds of formula I.e in which R^{xc} is H, R^{ya} is H, A is N, G is C—CH₃, and the combination of R, Q, Ar and D for a compound corresponds to each line of Table A.

Table e.7. Compounds of formula I.e in which R^{xc} is H, R^{ya} is H, A is CH, G is C—Cl, and the combination of R, Q, Ar and D for a compound corresponds to each line of Table A.

Table e.8. Compounds of formula I.e in which R^{xc} is H, R^{ya} is H, A is N, G is C—Cl, and the combination of R, Q, Ar and D for a compound corresponds to each line of Table A.

Table e.9. Compounds of formula I.e in which R^{xc} is CH₃, R^{ya} is H, A is CH, G is CH, and the combination of R, Q, Ar and D for a compound corresponds to each line of Table A.

Table e.10. Compounds of formula I.e in which R^{xc} is CH₃, R^{ya} is H, A is N, G is CH, and the combination of R, Q, Ar and D for a compound corresponds to each line of Table A.

Table e.11. Compounds of formula I.e in which R^{xc} is CH₃, R^{ya} is H, A is CH, G is N, and the combination of R, Q, Ar and D for a compound corresponds to each line of Table A.

Table e.12. Compounds of formula I.e in which R^{xc} is CH₃, R^{ya} is H, A is N, G is N, and the combination of R, Q, Ar and D for a compound corresponds to each line of Table A.

Table e.13. Compounds of formula I.e in which R^{xc} is CH₃, R^{ya} is H, A is CH, G is C—CH₃, and the combination of R, Q, Ar and D for a compound corresponds to each line of Table A.

Table e.14. Compounds of formula I.e in which R^{xc} is CH₃, R^{ya} is H, A is N, G is C—CH₃, and the combination of R, Q, Ar and D for a compound corresponds to each line of Table A.

Table e.15. Compounds of formula I.e in which R^{xc} is CH₃, R^{ya} is H, A is CH, G is C—Cl, and the combination of R, Q, Ar and D for a compound corresponds to each line of Table A.

Table e.16. Compounds of formula I.e in which R^{xc} is CH₃, R^{ya} is H, A is N, G is C—Cl, and the combination of R, Q, Ar and D for a compound corresponds to each line of Table A.

Table e.17. Compounds of formula I.e in which R^{xc} is H, R^{ya} is CH₃, A is CH, G is CH, and the combination of R, Q, Ar and D for a compound corresponds to each line of Table A.

Table e.18. Compounds of formula I.e in which R^{xc} is H, R^{ya} is CH₃, A is N, G is CH, and the combination of R, Q, Ar and D for a compound corresponds to each line of Table A.

Table e.19. Compounds of formula I.e in which R^{xc} is H, R^{ya} is CH₃, A is CH, G is N, and the combination of R, Q, Ar and D for a compound corresponds to each line of Table A.

Table e.20. Compounds of formula I.e in which R^{xc} is H, R^{ya} is CH₃, A is N, G is N, and the combination of R, Q, Ar and D for a compound corresponds to each line of Table A.

Table e.21. Compounds of formula I.e in which R^{xc} is H, R^{ya} is CH₃, A is CH, G is C—CH₃, and the combination of R, Q, Ar and D for a compound corresponds to each line of Table A.

Table e.22. Compounds of formula I.e in which R^{xc} is H, R^{ya} is CH₃, A is N, G is C—CH₃, and the combination of R, Q, Ar and D for a compound corresponds to each line of Table A.

Table e.23. Compounds of formula I.e in which R^{xc} is H, R^{ya} is CH₃, A is CH, G is C—Cl, and the combination of R, Q, Ar and D for a compound corresponds to each line of Table A.

Table e.24. Compounds of formula I.e in which R^{xc} is H, R^{ya} is CH₃, A is N, G is C—Cl, and the combination of R, Q, Ar and D for a compound corresponds to each line of Table A.

Table f.1. Compounds of formula I.f in which R^{e} is CH₂Ph, R^{ya} is H, A is CH, G is CH, and the combination of R, Q, Ar and D for a compound corresponds to each line of Table A.

Table f.2. Compounds of formula I.f in which R^{e} is CH₂Ph, R^{ya} is H, A is N, G is CH, and the combination of R, Q, Ar and D for a compound corresponds to each line of Table A.

Table f.3. Compounds of formula I.f in which R^{e} Re is CH₂Ph, R^{ya} is H, A is CH, G is N, and the combination of R, Q, Ar and D for a compound corresponds to each line of Table A.

Table f.4. Compounds of formula I.f in which R^{e} is CH₂Ph, R^{ya} is H, A is N, G is N, and the combination of R, Q, Ar and D for a compound corresponds to each line of Table A.

Table f.5. Compounds of formula I.f in which R^{e} is CH₂Ph, R^{ya} is H, A is CH, G is C—CH₃, and the combination of R, Q, Ar and D for a compound corresponds to each line of Table A.

Table f.6. Compounds of formula I.f in which R^{e} is CH₂Ph, R^{ya} is H, A is N, G is C—CH₃, and the combination of R, Q, Ar and D for a compound corresponds to each line of Table A.

Table f.7. Compounds of formula I.f in which R^{e} is CH₂Ph, R^{ya} is H, A is CH, G is C—Cl, and the combination of R, Q, Ar and D for a compound corresponds to each line of Table A.

Table f.8. Compounds of formula I.f in which R^{e} Re is CH₂Ph, R^{ya} is H, A is N, G is C—Cl, and the combination of R, Q, Ar and D for a compound corresponds to each line of Table A.

Table f.9. Compounds of formula I.f in which R^{e} is CH₃, R^{ya} is H, A is CH, G is CH, and the combination of R, Q, Ar and D for a compound corresponds to each line of Table A.

Table f.10. Compounds of formula I.f in which R^{e} is CH₃, R^{ya} is H, A is N, G is CH, and the combination of R, Q, Ar and D for a compound corresponds to each line of Table A.

Table f.11. Compounds of formula I.f in which R^{e} is CH₃, R^{ya} is H, A is CH, G is N, and the combination of R, Q, Ar and D for a compound corresponds to each line of Table A.

Table f.12. Compounds of formula I.f in which R^{e} is CH₃, R^{ya} is H, A is N, G is N, and the combination of R, Q, Ar and D for a compound corresponds to each line of Table A.

Table f.13. Compounds of formula I.f in which R^{e} is CH₃, R^{ya} is H, A is CH, G is C—CH₃, and the combination of R, Q, Ar and D for a compound corresponds to each line of Table A.

Table f.14. Compounds of formula I.f in which R^{e} is CH₃, R^{ya} is H, A is N, G is C—CH₃, and the combination of R, Q, Ar and D for a compound corresponds to each line of Table A.

Table f.15. Compounds of formula I.f in which R^{e} is CH₃, R^{ya} is H, A is CH, G is C—Cl, and the combination of R, Q, Ar and D for a compound corresponds to each line of Table A.

Table f.16. Compounds of formula I.f in which R^{e} is CH₃, R^{ya} is H, A is N, G is C—Cl, and the combination of R, Q, Ar and D for a compound corresponds to each line of Table A.

Table f.17. Compounds of formula I.f in which R^{e} is CH₂Ph, R^{ya} is CH₃, A is CH, G is CH, and the combination of R, Q, Ar and D for a compound corresponds to each line of Table A.

Table f.18. Compounds of formula I.f in which R^{e} is CH₂Ph, R^{ya} is CH₃, A is N, G is CH, and the combination of R, Q, Ar and D for a compound corresponds to each line of Table A.

Table f.19. Compounds of formula I.f in which R^{e} is CH₂Ph, R^{ya} is CH₃, A is CH, G is N, and the combination of R, Q, Ar and D for a compound corresponds to each line of Table A.

Table f.20. Compounds of formula I.f in which R^{e} is CH₂Ph, R^{ya} is CH₃, A is N, G is N, and the combination of R, Q, Ar and D for a compound corresponds to each line of Table A.

Table f.21. Compounds of formula I.f in which R^{e} is CH₂Ph, R^{ya} is CH₃, A is CH, G is C—CH₃, and the combination of R, Q, Ar and D for a compound corresponds to each line of Table A.

Table f.22. Compounds of formula I.f in which R^{e} is CH₂Ph, R^{ya} is CH₃, A is N, G is C—CH₃, and the combination of R, Q, Ar and D for a compound corresponds to each line of Table A.

Table f.23. Compounds of formula I.f in which R^{e} is CH₂Ph, R^{ya} is CH₃, A is CH, G is C—Cl, and the combination of R, Q, Ar and D for a compound corresponds to each line of Table A.

Table f.24. Compounds of formula I.f in which R^{e} is CH₂Ph, R^{ya} is CH₃, A is N, G is C—Cl, and the combination of R, Q, Ar and D for a compound corresponds to each line of Table A.

Table g.1. Compounds of formula I.g in which R^{xc} is H, R^{ya} is H, A is CH, G is CH, and the combination of R, Q, Ar and D for a compound corresponds to each line of Table A.

Table g.2. Compounds of formula I.g in which R^{xc} is H, R^{ya} is H, A is N, G is CH, and the combination of R, Q, Ar and D for a compound corresponds to each line of Table A.

Table g.3. Compounds of formula I.g in which R^{xc} is H, R^{ya} is H, A is CH, G is N, and the combination of R, Q, Ar and D for a compound corresponds to each line of Table A.

Table g.4. Compounds of formula I.g in which R^{xc} is H, R^{ya} is H, A is N, G is N, and the combination of R, Q, Ar and D for a compound corresponds to each line of Table A.

Table g.5. Compounds of formula I.g in which R^{xc} is H, R^{ya} is H, A is CH, G is C—CH₃, and the combination of R, Q, Ar and D for a compound corresponds to each line of Table A.

Table g.6. Compounds of formula I.g in which R^{xc} is H, R^{ya} is H, A is N, G is C—CH₃, and the combination of R, Q, Ar and D for a compound corresponds to each line of Table A.

Table g.7. Compounds of formula I.g in which R^{xc} is H, R^{ya} is H, A is CH, G is C—Cl, and the combination of R, Q, Ar and D for a compound corresponds to each line of Table A.

Table g.8. Compounds of formula I.g in which R^{xc} is H, R^{ya} is H, A is N, G is C—Cl, and the combination of R, Q, Ar and D for a compound corresponds to each line of Table A.

Table g.9. Compounds of formula I.g in which R^{xc} is CH₃, R^{ya} is H, A is CH, G is CH, and the combination of R, Q, Ar and D for a compound corresponds to each line of Table A.

Table g.10. Compounds of formula I.g in which R^{xc} is CH₃, R^{ya} is H, A is N, G is CH, and the combination of R, Q, Ar and D for a compound corresponds to each line of Table A.

Table g.11. Compounds of formula I.g in which R^{xc} is CH₃, R^{ya} is H, A is CH, G is N, and the combination of R, Q, Ar and D for a compound corresponds to each line of Table A.

Table g.12. Compounds of formula I.g in which R^{xc} is CH₃, R^{ya} is H, A is N, G is N, and the combination of R, Q, Ar and D for a compound corresponds to each line of Table A.

Table g.13. Compounds of formula I.g in which R^{xc} is CH₃, R^{ya} is H, A is CH, G is C—CH₃, and the combination of R, Q, Ar and D for a compound corresponds to each line of Table A.

Table g.14. Compounds of formula I.g in which R^{xc} is CH₃, R^{ya} is H, A is N, G is C—CH₃, and the combination of R, Q, Ar and D for a compound corresponds to each line of Table A.

Table g.15. Compounds of formula I.g in which R^{xc} is CH₃, R^{ya} is H, A is CH, G is C—Cl, and the combination of R, Q, Ar and D for a compound corresponds to each line of Table A.

Table g.16. Compounds of formula I.g in which R^{xc} is CH₃, R^{ya} is H, A is N, G is C—Cl, and the combination of R, Q, Ar and D for a compound corresponds to each line of Table A.

Table g.17. Compounds of formula I.g in which R^{xc} is H, R^{ya} is CH₃, A is CH, G is CH, and the combination of R, Q, Ar and D for a compound corresponds to each line of Table A.

Table g.18. Compounds of formula I.g in which R^{xc} is H, R^{ya} is CH₃, A is N, G is CH, and the combination of R, Q, Ar and D for a compound corresponds to each line of Table A.

Table g.19. Compounds of formula I.g in which R^{xc} is H, R^{ya} is CH₃, A is CH, G is N, and the combination of R, Q, Ar and D for a compound corresponds to each line of Table A.

Table g.20. Compounds of formula I.g in which R^{xc} is H, R^{ya} is CH₃, A is N, G is N, and the combination of R, Q, Ar and D for a compound corresponds to each line of Table A.

Table g.21. Compounds of formula I.g in which R^{xc} is H, R^{ya} is CH₃, A is CH, G is C—CH₃, and the combination of R, Q, Ar and D for a compound corresponds to each line of Table A.

Table g.22. Compounds of formula I.g in which R^{xc} is H, R^{ya} is CH₃, A is N, G is C—CH₃, and the combination of R, Q, Ar and D for a compound corresponds to each line of Table A.

Table g.23. Compounds of formula I.g in which R^{xc} is H, R^{ya} is CH₃, A is CH, G is C—Cl, and the combination of R, Q, Ar and D for a compound corresponds to each line of Table A.

Table g.24. Compounds of formula I.g in which R^{xc} is H, R^{ya} is CH₃, A is N, G is C—Cl, and the combination of R, Q, Ar and D for a compound corresponds to each line of Table A.

Table h.1. Compounds of formula I.h in which R^{xa} is H, R^{xb} is H, R^{yc} is H, A is CH, G is CH, and the combination of R, Q, Ar and D for a compound corresponds to each line of Table A.

Table h.2. Compounds of formula I.h in which R^{xa} is H, R^{xb} is H, R^{yc} is H, A is N, G is CH, and the combination of R, Q, Ar and D for a compound corresponds to each line of Table A.

Table h.3. Compounds of formula I.h in which R^{xa} is H, R^{xb} is H, R^{yc} is H, A is CH, G is N, and the combination of R, Q, Ar and D for a compound corresponds to each line of Table A.

Table h.4. Compounds of formula I.h in which R^{xa} is H, R^{xb} is H, R^{yc} is H, A is N, G is N, and the combination of R, Q, Ar and D for a compound corresponds to each line of Table A.

Table h.5. Compounds of formula I.h in which R^{xa} is H, R^{xb} is H, R^{yc} is H, A is CH, G is C—CH₃, and the combination of R, Q, Ar and D for a compound corresponds to each line of Table A.

Table h.6. Compounds of formula I.h in which R^{xa} is H, R^{xb} is H, R^{yc} is H, A is N, G is C—CH₃, and the combination of R, Q, Ar and D for a compound corresponds to each line of Table A.

Table h.7. Compounds of formula I.h in which R^{xa} is H, R^{xb} is H, R^{yc} is H, A is CH, G is C—Cl, and the combination of R, Q, Ar and D for a compound corresponds to each line of Table A.

Table h.8. Compounds of formula I.h in which R^{xa} is H, R^{xb} is H, R^{yc} is H, A is N, G is C—Cl, and the combination of R, Q, Ar and D for a compound corresponds to each line of Table A.

Table h.9. Compounds of formula I.h in which R^{xa} is CH₃, R^{xb} is H, R^{yc} is H, A is CH, G is CH, and the combination of R, Q, Ar and D for a compound corresponds to each line of Table A.

Table h.10. Compounds of formula I.h in which R^{xa} is CH₃, R^{xb} is H, R^{yc} is H, A is N, G is CH, and the combination of R, Q, Ar and D for a compound corresponds to each line of Table A.

Table h.11. Compounds of formula I.h in which R^{xa} is CH₃, R^{xb} is H, R^{yc} is H, A is CH, G is N, and the combination of R, Q, Ar and D for a compound corresponds to each line of Table A.

Table h.12. Compounds of formula I.h in which R^{xa} is CH₃, R^{xb} is H, R^{yc} is H, A is N, G is N, and the combination of R, Q, Ar and D for a compound corresponds to each line of Table A.

Table h.13. Compounds of formula I.h in which R^{xa} is CH₃, R^{xb} is H, R^{yc} is H, A is CH, G is C—CH₃, and the combination of R, Q, Ar and D for a compound corresponds to each line of Table A.

Table h.14. Compounds of formula I.h in which R^{xa} is CH₃, R^{xb} is H, R^{yc} is H, A is N, G is C—CH₃, and the combination of R, Q, Ar and D for a compound corresponds to each line of Table A.

Table h.15. Compounds of formula I.h in which R^{xa} is CH₃, R^{xb} is H, R^{yc} is H, A is CH, G is C—Cl, and the combination of R, Q, Ar and D for a compound corresponds to each line of Table A.

Table h.16. Compounds of formula I.h in which R^{xa} is CH₃, R^{xb} is H, R^{yc} is H, A is N, G is C—Cl, and the combination of R, Q, Ar and D for a compound corresponds to each line of Table A.

Table h.17. Compounds of formula I.h in which R^{xa} is H, R^{xb} is CH₃, R^{yc} is H, A is CH, G is CH, and the combination of R, Q, Ar and D for a compound corresponds to each line of Table A.

Table h.18. Compounds of formula I.h in which R^{xa} is H, R^{xb} is CH₃, R^{yc} is H, A is N, G is CH, and the combination of R, Q, Ar and D for a compound corresponds to each line of Table A.

Table h.19. Compounds of formula I.h in which R^{xa} is H, R^{xb} is CH₃, R^{yc} is H, A is CH, G is N, and the combination of R, Q, Ar and D for a compound corresponds to each line of Table A.

Table h.20. Compounds of formula I.h in which R^{xa} is H, R^{xb} is CH₃, R^{yc} is H, A is N, G is N, and the combination of R, Q, Ar and D for a compound corresponds to each line of Table A.

Table h.21. Compounds of formula I.h in which R^{xa} is H, R^{xb} is CH₃, R^{yc} is H, A is CH, G is C—C₂H₅, and the combination of R, Q, Ar and D for a compound corresponds to each line of Table A.

Table h.22. Compounds of formula I.h in which R^{xa} is H, R^{xb} is CH₃, R^{yc} is H, A is N, G is C—CH₃, and the combination of R, Q, Ar and D for a compound corresponds to each line of Table A.

Table h.23. Compounds of formula I.h in which R^{xa} is H, R^{xb} is CH₃, R^{yc} is H, A is CH, G is C—Cl, and the combination of R, Q, Ar and D for a compound corresponds to each line of Table A.

Table h.24. Compounds of formula I.h in which R^{xa} is H, R^{xb} is CH₃, R^{yc} is H, A is N, G is C—Cl, and the combination of R, Q, Ar and D for a compound corresponds to each line of Table A.

Table h.25. Compounds of formula I.h in which R^{xa} is H, R^{xb} is H, R^{yc} is CH₃, A is CH, G is CH, and the combination of R, Q, Ar and D for a compound corresponds to each line of Table A.

Table h.26. Compounds of formula I.h in which R^{xa} is H, R^{xb} is H, R^{yc} is CH₃, A is N, G is CH, and the combination of R, Q, Ar and D for a compound corresponds to each line of Table A.

Table h.27. Compounds of formula I.h in which R^{xa} is H, R^{xb} is H, R^{yc} is CH₃, A is CH, G is N, and the combination of R, Q, Ar and D for a compound corresponds to each line of Table A.

Table h.28. Compounds of formula I.h in which R^{xa} is H, R^{xb} is H, R^{yc} is CH₃, A is N, G is N, and the combination of R, Q, Ar and D for a compound corresponds to each line of Table A.

Table h.29. Compounds of formula I.h in which R^{xa} is H, R^{xb} is H, R^{yc} is CH₃, A is CH, G is C—CH₃, and the combination of R, Q, Ar and D for a compound corresponds to each line of Table A.

Table h.30. Compounds of formula I.h in which R^{xa} is H, R^{xb} is H, R^{yc} is CH₃, A is N, G is C—CH₃, and the combination of R, Q, Ar and D for a compound corresponds to each line of Table A.

Table h.31. Compounds of formula I.h in which R^{xa} is H, R^{xb} is H, R^{yc} is CH₃, A is CH, G is C—Cl, and the combination of R, Q, Ar and D for a compound corresponds to each line of Table A.

Table h.32. Compounds of formula I.h in which R^{xa} is H, R^{xb} is H, R^{yc} is CH₃, A is N, G is C—Cl, and the combination of R, Q, Ar and D for a compound corresponds to each line of Table A.

Table h.33. Compounds of formula I.h in which R^{xa} is CH₃, R^{xb} is CH₃, R^{yc} is H, A is CH, G is CH, and the combination of R, Q, Ar and D for a compound corresponds to each line of Table A.

Table h.34. Compounds of formula I.h in which R^{xa} is CH₃, R^{xb} is CH₃, R^{yc} is H, A is N, G is CH, and the combination of R, Q, Ar and D for a compound corresponds to each line of Table A.

Table h.35. Compounds of formula I.h in which R^{xa} is CH₃, R^{xb} is CH₃, R^{yc} is H, A is CH, G is N, and the combination of R, Q, Ar and D for a compound corresponds to each line of Table A.

Table h.36. Compounds of formula I.h in which R^{xa} is CH₃, R^{xb} is CH₃, R^{yc} is H, A is N, G is N, and the combination of R, Q, Ar and D for a compound corresponds to each line of Table A.

Table h.37. Compounds of formula I.h in which R^{xa} is CH₃, R^{xb} is CH₃, R^{yc} is H, A is CH, G is C—CH₃, and the combination of R, Q, Ar and D for a compound corresponds to each line of Table A.

Table h.38. Compounds of formula I.h in which R^{xa} is CH₃, R^{xb} is CH₃, R^{yc} is H, A is N, G is C—CH₃, and the combination of R, Q, Ar and D for a compound corresponds to each line of Table A.

Table h.39. Compounds of formula I.h in which R^{xa} is CH₃, R^{xb} is CH₃, R^{yc} is H, A is CH, G is C—Cl, and the combination of R, Q, Ar and D for a compound corresponds to each line of Table A.

Table h.40. Compounds of formula I.h in which R^{xa} is CH₃, R^{xb} is CH₃, R^{yc} is H, A is N, G is C—Cl, and the combination of R, Q, Ar and D for a compound corresponds to each line of Table A.

Table h.41. Compounds of formula I.h in which R^{xa} is CH₃, R^{xb} is CH₃, R^{yc} is CH₃, A is CH, G is CH, and the combination of R, Q, Ar and D for a compound corresponds to each line of Table A.

Table h.42. Compounds of formula I.h in which R^{xa} is CH₃, R^{xb} is CH₃, R^{yc} is CH₃, A is N, G is CH, and the combination of R, Q, Ar and D for a compound corresponds to each line of Table A.

Table h.43. Compounds of formula I.h in which R^{xa} is CH₃, R^{xb} is CH₃, R^{yc} is CH₃, A is CH, G is N, and the combination of R, Q, Ar and D for a compound corresponds to each line of Table A.

Table h.44. Compounds of formula I.h in which R^{xa} is CH₃, R^{xb} is CH₃, R^{yc} is CH₃, A is N, G is N, and the combination of R, Q, Ar and D for a compound corresponds to each line of Table A.

Table h.45. Compounds of formula I.h in which R^{xa} is CH₃, R^{xb} is CH₃, R^{yc} is CH₃, A is CH, G is C—CH₃, and the combination of R, Q, Ar and D for a compound corresponds to each line of Table A.

Table h.46. Compounds of formula I.h in which R^{xa} is CH₃, R^{xb} is CH₃, R^{yc} is CH₃, A is N, G is C—CH₃, and the combination of R, Q, Ar and D for a compound corresponds to each line of Table A.

Table h.47. Compounds of formula I.h in which R^{xa} is CH₃, R^{xb} is CH₃, R^{yc} is CH₃, A is CH, G is C—Cl, and the combination of R, Q, Ar and D for a compound corresponds to each line of Table A.

Table h.48. Compounds of formula I.h in which R^{xa} is CH₃, R^{xb} is CH₃, R^{yc} is CH₃, A is N, G is C—Cl, and the combination of R, Q, Ar and D for a compound corresponds to each line of Table A.

Table i.1. Compounds of formula I.i in which R^{xa} is H, R^{xb} is H, R^{ya} is H, A is CH, G is CH, and the combination of R, Q, Ar and D for a compound corresponds to each line of Table A.

Table i.2. Compounds of formula I.i in which R^{xa} is H, R^{xb} is H, R^{ya} is H, A is N, G is CH, and the combination of R, Q, Ar and D for a compound corresponds to each line of Table A.

Table i.3. Compounds of formula I.i in which R^{xa} is H, R^{xb} is H, R^{ya} is H, A is CH, G is N, and the combination of R, Q, Ar and D for a compound corresponds to each line of Table A.

Table i.4. Compounds of formula I.i in which R^{xa} is H, R^{xb} is H, R^{ya} is H, A is N, G is N, and the combination of R, Q, Ar and D for a compound corresponds to each line of Table A.

Table i.5. Compounds of formula I.i in which R^{xa} is H, R^{xb} is H, R^{ya} is H, A is CH, G is C—CH₃, and the combination of R, Q, Ar and D for a compound corresponds to each line of Table A.

Table i.6. Compounds of formula I.i in which R^{xa} is H, R^{xb} is H, R^{ya} is H, A is N, G is C—CH₃, and the combination of R, Q, Ar and D for a compound corresponds to each line of Table A.

Table i.7. Compounds of formula I.i in which R^{xa} is H, R^{xb} is H, R^{ya} is H, A is CH, G is C—Cl, and the combination of R, Q, Ar and D for a compound corresponds to each line of Table A.

Table i.8. Compounds of formula I.i in which R^{xa} is H, R^{xb} is H, R^{ya} is H, A is N, G is C—Cl, and the combination of R, Q, Ar and D for a compound corresponds to each line of Table A.

Table i.9. Compounds of formula I.i in which R^{xa} is CH₃, R^{xb} is H, R^{ya} is H, A is CH, G is CH, and the combination of R, Q, Ar and D for a compound corresponds to each line of Table A.

Table i.10. Compounds of formula I.i in which R^{xa} is CH₃, R^{xb} is H, R^{ya} is H, A is N, G is CH, and the combination of R, Q, Ar and D for a compound corresponds to each line of Table A.

Table i.11. Compounds of formula I.i in which R^{xa} is CH₃, R^{xb} is H, R^{ya} is H, A is CH, G is N, and the combination of R, Q, Ar and D for a compound corresponds to each line of Table A.

Table i.12. Compounds of formula I.i in which R^{xa} is CH₃, R^{xb} is H, R^{ya} is H, A is N, G is N, and the combination of R, Q, Ar and D for a compound corresponds to each line of Table A.

Table i.13. Compounds of formula I.i in which R^{xa} is CH₃, R^{xb} is H, R^{ya} is H, A is CH, G is C—CH₃, and the combination of R, Q, Ar and D for a compound corresponds to each line of Table A.

Table i.14. Compounds of formula I.i in which R^{xa} is CH₃, R^{xb} is H, R^{ya} is H, A is N, G is C—CH₃, and the combination of R, Q, Ar and D for a compound corresponds to each line of Table A.

Table i.15. Compounds of formula I.i in which R^{xa} is CH₃, R^{xb} is H, R^{ya} is H, A is CH, G is C—Cl, and the combination of R, Q, Ar and D for a compound corresponds to each line of Table A.

Table i.16. Compounds of formula I.i in which R^{xa} is CH₃, R^{xb} is H, R^{ya} is H, A is N, G is C—Cl, and the combination of R, Q, Ar and D for a compound corresponds to each line of Table A.

Table i.17. Compounds of formula I.i in which R^{xa} is H, R^{xb} is CH₃, R^{ya} is H, A is CH, G is CH, and the combination of R, Q, Ar and D for a compound corresponds to each line of Table A.

Table i.18. Compounds of formula I.i in which R^{xa} is H, R^{xb} is CH₃, R^{ya} is H, A is N, G is CH, and the combination of R, Q, Ar and D for a compound corresponds to each line of Table A.

Table i.19. Compounds of formula I.i in which R^{xa} is H, R^{xb} is CH₃, R^{ya} is H, A is CH, G is N, and the combination of R, Q, Ar and D for a compound corresponds to each line of Table A.

Table i.20. Compounds of formula I.i in which R^{xa} is H, R^{xb} is CH₃, R^{ya} is H, A is N, G is N, and the combination of R, Q, Ar and D for a compound corresponds to each line of Table A.

Table i.21. Compounds of formula I.i in which R^{xa} is H, R^{xb} is CH₃, R^{ya} is H, A is CH, G is C—C₂H₅, and the combination of R, Q, Ar and D for a compound corresponds to each line of Table A.

Table i.22. Compounds of formula I.i in which R^{xa} is H, R^{xb} is CH₃, R^{ya} is H, A is N, G is C—CH₃, and the combination of R, Q, Ar and D for a compound corresponds to each line of Table A.

Table i.23. Compounds of formula I.i in which R^{xa} is H, R^{xb} is CH₃, R^{ya} is H, A is CH, G is C—Cl, and the combination of R, Q, Ar and D for a compound corresponds to each line of Table A.

Table i.24. Compounds of formula I.i in which R^{xa} is H, R^{xb} is CH₃, R^{ya} is H, A is N, G is C—Cl, and the combination of R, Q, Ar and D for a compound corresponds to each line of Table A.

Table i.25. Compounds of formula I.i in which R^{xa} is H, R^{xb} is H, R^{ya} is CH₃, A is CH, G is CH, and the combination of R, Q, Ar and D for a compound corresponds to each line of Table A.

Table i.26. Compounds of formula I.i in which R^{xa} is H, R^{xb} is H, R^{ya} is CH₃, A is N, G is CH, and the combination of R, Q, Ar and D for a compound corresponds to each line of Table A.

Table i.27. Compounds of formula I.i in which R^{xa} is H, R^{xb} is H, R^{ya} is CH₃, A is CH, G is N, and the combination of R, Q, Ar and D for a compound corresponds to each line of Table A.

Table i.28. Compounds of formula I.i in which R^{xa} is H, R^{xb} is H, R^{ya} is CH₃, A is N, G is N, and the combination of R, Q, Ar and D for a compound corresponds to each line of Table A.

Table i.29. Compounds of formula I.i in which R^{xa} is H, R^{xb} is H, R^{ya} is CH₃, A is CH, G is C—CH₃, and the combination of R, Q, Ar and D for a compound corresponds to each line of Table A.

Table i.30. Compounds of formula I.i in which R^{xa} is H, R^{xb} is H, R^{ya} is CH₃, A is N, G is C—CH₃, and the combination of R, Q, Ar and D for a compound corresponds to each line of Table A.

Table i.31. Compounds of formula I.i in which R^{xa} is H, R^{xb} is H, R^{ya} is CH₃, A is CH, G is C—Cl, and the combination of R, Q, Ar and D for a compound corresponds to each line of Table A.

Table i.32. Compounds of formula I.i in which R^{xa} is H, R^{xb} is H, R^{ya} is CH₃, A is N, G is C—Cl, and the combination of R, Q, Ar and D for a compound corresponds to each line of Table A.

Table i.33. Compounds of formula I.i in which R^{xa} is CH₃, R^{xb} is CH₃, R^{ya} is H, A is CH, G is CH, and the combination of R, Q, Ar and D for a compound corresponds to each line of Table A.

Table i.34. Compounds of formula I.i in which R^{xa} is CH₃, R^{xb} is CH₃, R^{ya} is H, A is N, G is CH, and the combination of R, Q, Ar and D for a compound corresponds to each line of Table A.

Table i.35. Compounds of formula I.i in which R^{xa} is CH₃, R^{xb} is CH₃, R^{ya} is H, A is CH, G is N, and the combination of R, Q, Ar and D for a compound corresponds to each line of Table A.

Table i.36. Compounds of formula I.i in which R^{xa} is CH₃, R^{xb} is CH₃, R^{ya} is H, A is N, G is N, and the combination of R, Q, Ar and D for a compound corresponds to each line of Table A.

Table i.37. Compounds of formula I.i in which R^{xa} is CH₃, R^{xb} is CH₃, R^{ya} is H, A is CH, G is C—CH₃, and the combination of R, Q, Ar and D for a compound corresponds to each line of Table A.

Table i.38. Compounds of formula I.i in which R^{xa} is CH₃, R^{xb} is CH₃, R^{ya} is H, A is N, G is C—CH₃, and the combination of R, Q, Ar and D for a compound corresponds to each line of Table A.

Table i.39. Compounds of formula I.i in which R^{xa} is CH₃, R^{xb} is CH₃, R^{ya} is H, A is CH, G is C—Cl, and the combination of R, Q, Ar and D for a compound corresponds to each line of Table A.

Table i.40. Compounds of formula I.i in which R^{xa} is CH₃, R^{xb} is CH₃, R^{ya} is H, A is N, G is C—Cl, and the combination of R, Q, Ar and D for a compound corresponds to each line of Table A.

Table i.41. Compounds of formula I.i in which R^{xa} is CH₃, R^{xb} is CH₃, R^{ya} is CH₃, A is CH, G is CH, and the combination of R, Q, Ar and D for a compound corresponds to each line of Table A.

Table i.42. Compounds of formula I.i in which R^{xa} is CH₃, R^{xb} is CH₃, R^{ya} is CH₃, A is N, G is CH, and the combination of R, Q, Ar and D for a compound corresponds to each line of Table A.

Table i.43. Compounds of formula I.i in which R^{xa} is CH₃, R^{xb} is CH₃, R^{ya} is CH₃, A is CH, G is N, and the combination of R, Q, Ar and D for a compound corresponds to each line of Table A.

Table i.44. Compounds of formula I.i in which R^{xa} is CH₃, R^{xb} is CH₃, R^{ya} is CH₃, A is N, G is N, and the combination of R, Q, Ar and D for a compound corresponds to each line of Table A.

Table i.45. Compounds of formula I.i in which R^{xa} is CH₃, R^{xb} is CH₃, R^{ya} is CH₃, A is CH, G is C—CH₃, and the combination of R, Q, Ar and D for a compound corresponds to each line of Table A.

Table i.46. Compounds of formula I.i in which R^{xa} is CH₃, R^{xb} is CH₃, R^{ya} is CH₃, A is N, G is C—CH₃, and the combination of R, Q, Ar and D for a compound corresponds to each line of Table A.

Table i.47. Compounds of formula I.i in which R^{xa} is CH₃, R^{xb} is CH₃, R^{ya} is CH₃, A is CH, G is C—Cl, and the combination of R, Q, Ar and D for a compound corresponds to each line of Table A.

Table i.48. Compounds of formula I.i in which R^{xa} is CH₃, R^{xb} is CH₃, R^{ya} is CH₃, A is N, G is C—Cl, and the combination of R, Q, Ar and D for a compound corresponds to each line of Table A.

Table j.1. Compounds of formula I.j in which R^{xa} is H, R^{xb} is H, R^{yc} is H, A is CH, G is CH, and the combination of R, Q, Ar and D for a compound corresponds to each line of Table A.

Table j.2. Compounds of formula I.j in which R^{xa} is H, R^{xb} is H, R^{yc} is H, A is N, G is CH, and the combination of R, Q, Ar and D for a compound corresponds to each line of Table A.

Table j.3. Compounds of formula I.j in which R^{xa} is H, R^{xb} is H, R^{yc} is H, A is CH, G is N, and the combination of R, Q, Ar and D for a compound corresponds to each line of Table A.

Table j.4. Compounds of formula I.j in which R^{xa} is H, R^{xb} is H, R^{yc} is H, A is N, G is N, and the combination of R, Q, Ar and D for a compound corresponds to each line of Table A.

Table j.5. Compounds of formula I.j in which R^{xa} is H, R^{xb} is H, R^{yc} is H, A is CH, G is C—CH₃, and the combination of R, Q, Ar and D for a compound corresponds to each line of Table A.

Table j.6. Compounds of formula I.j in which R^{xa} is H, R^{xb} is H, R^{yc} is H, A is N, G is C—CH₃, and the combination of R, Q, Ar and D for a compound corresponds to each line of Table A.

Table j.7. Compounds of formula I.j in which R^{xa} is H, R^{xb} is H, R^{yc} is H, A is CH, G is C—Cl, and the combination of R, Q, Ar and D for a compound corresponds to each line of Table A.

Table j.8. Compounds of formula I.j in which R^{xa} is H, R^{xb} is H, R^{yc} is H, A is N, G is C—Cl, and the combination of R, Q, Ar and D for a compound corresponds to each line of Table A.

Table j.9. Compounds of formula I.j in which R^{xa} is CH₃, R^{xb} is H, R^{yc} is H, A is CH, G is CH, and the combination of R, Q, Ar and D for a compound corresponds to each line of Table A.

Table j.10. Compounds of formula I.j in which R^{xa} is CH₃, R^{xb} is H, R^{yc} is H, A is N, G is CH, and the combination of R, Q, Ar and D for a compound corresponds to each line of Table A.

Table j.11. Compounds of formula I.j in which R^{xa} is CH₃, R^{xb} is H, R^{yc} is H, A is CH, G is N, and the combination of R, Q, Ar and D for a compound corresponds to each line of Table A.

Table j.12. Compounds of formula I.j in which R^{xa} is CH₃, R^{xb} is H, R^{yc} is H, A is N, G is N, and the combination of R, Q, Ar and D for a compound corresponds to each line of Table A.

Table j.13. Compounds of formula I.j in which R^{xa} is CH₃, R^{xb} is H, R^{yc} is H, A is CH, G is C—CH₃, and the combination of R, Q, Ar and D for a compound corresponds to each line of Table A.

Table j.14. Compounds of formula I.j in which R^{xa} is CH₃, R^{xb} is H, R^{yc} is H, A is N, G is C—CH₃, and the combination of R, Q, Ar and D for a compound corresponds to each line of Table A.

Table j.15. Compounds of formula I.j in which R^{xa} is CH₃, R^{xb} is H, R^{yc} is H, A is CH, G is C—Cl, and the combination of R, Q, Ar and D for a compound corresponds to each line of Table A.

Table j.16. Compounds of formula I.j in which R^{xa} is CH₃, R^{xb} is H, R^{yc} is H, A is N, G is C—Cl, and the combination of R, Q, Ar and D for a compound corresponds to each line of Table A.

Table j.17. Compounds of formula I.j in which R^{xa} is H, R^{xb} is CH₃, R^{yc} is H, A is CH, G is CH, and the combination of R, Q, Ar and D for a compound corresponds to each line of Table A.

Table j.18. Compounds of formula I.j in which R^{xa} is H, R^{xb} is CH₃, R^{yc} is H, A is N, G is CH, and the combination of R, Q, Ar and D for a compound corresponds to each line of Table A.

Table j.19. Compounds of formula I.j in which R^{xa} is H, R^{xb} is CH₃, R^{yc} is H, A is CH, G is N, and the combination of R, Q, Ar and D for a compound corresponds to each line of Table A.

Table j.20. Compounds of formula I.j in which R^{xa} is H, R^{xb} is CH₃, R^{yc} is H, A is N, G is N, and the combination of R, Q, Ar and D for a compound corresponds to each line of Table A.

Table j.21. Compounds of formula I.j in which R^{xa} is H, R^{xb} is CH₃, R^{yc} is H, A is CH, G is C—C₂H₅, and the combination of R, Q, Ar and D for a compound corresponds to each line of Table A.

Table j.22. Compounds of formula I.j in which R^{xa} is H, R^{xb} is CH₃, R^{yc} is H, A is N, G is C—CH₃, and the combination of R, Q, Ar and D for a compound corresponds to each line of Table A.

Table j.23. Compounds of formula I.j in which R^{xa} is H, R^{xb} is CH₃, R^{yc} is H, A is CH, G is C—Cl, and the combination of R, Q, Ar and D for a compound corresponds to each line of Table A.

Table j.24. Compounds of formula I.j in which R^{xa} is H, R^{xb} is CH₃, R^{yc} is H, A is N, G is C—Cl, and the combination of R, Q, Ar and D for a compound corresponds to each line of Table A.

Table j.25. Compounds of formula I.j in which R^{xa} is H, R^{xb} is H, R^{yc} is CH₃, A is CH, G is CH, and the combination of R, Q, Ar and D for a compound corresponds to each line of Table A.

Table j.26. Compounds of formula I.j in which R^{xa} is H, R^{xb} is H, R^{yc} is CH₃, A is N, G is CH, and the combination of R, Q, Ar and D for a compound corresponds to each line of Table A.

Table j.27. Compounds of formula I.j in which R^{xa} is H, R^{xb} is H, R^{yc} is CH₃, A is CH, G is N, and the combination of R, Q, Ar and D for a compound corresponds to each line of Table A.

Table j.28. Compounds of formula I.j in which R^{xa} is H, R^{xb} is H, R^{yc} is CH₃, A is N, G is N, and the combination of R, Q, Ar and D for a compound corresponds to each line of Table A.

Table j.29. Compounds of formula I.j in which R^{xa} is H, R^{xb} is H, R^{yc} is CH₃, A is CH, G is C—CH₃, and the combination of R, Q, Ar and D for a compound corresponds to each line of Table A.

Table j.30. Compounds of formula I.j in which R^{xa} is H, R^{xb} is H, R^{yc} is CH₃, A is N, G is C—CH₃, and the combination of R, Q, Ar and D for a compound corresponds to each line of Table A.

Table j.31. Compounds of formula I.j in which R^{xa} is H, R^{xb} is H, R^{yc} is CH₃, A is CH, G is C—Cl, and the combination of R, Q, Ar and D for a compound corresponds to each line of Table A.

Table j.32. Compounds of formula I.j in which R^{xa} is H, R^{xb} is H, R^{yc} is CH₃, A is N, G is C—Cl, and the combination of R, Q, Ar and D for a compound corresponds to each line of Table A.

Table j.33. Compounds of formula I.j in which R^{xa} is CH₃, R^{xb} is CH₃, R^{yc} is H, A is CH, G is CH, and the combination of R, Q, Ar and D for a compound corresponds to each line of Table A.

Table j.34. Compounds of formula I.j in which R^{xa} is CH₃, R^{xb} is CH₃, R^{yc} is H, A is N, G is CH, and the combination of R, Q, Ar and D for a compound corresponds to each line of Table A.

Table j.35. Compounds of formula I.j in which R^{xa} is CH₃, R^{xb} is CH₃, R^{yc} is H, A is CH, G is N, and the combination of R, Q, Ar and D for a compound corresponds to each line of Table A.

Table j.36. Compounds of formula I.j in which R^{xa} is CH₃, R^{xb} is CH₃, R^{yc} is H, A is N, G is N, and the combination of R, Q, Ar and D for a compound corresponds to each line of Table A.

Table j.37. Compounds of formula I.j in which R^{xa} is CH₃, R^{xb} is CH₃, R^{yc} is H, A is CH, G is C—CH₃, and the combination of R, Q, Ar and D for a compound corresponds to each line of Table A.

Table j.38. Compounds of formula I.j in which R^{xa} is CH₃, R^{xb} is CH₃, R^{yc} is H, A is N, G is C—CH₃, and the combination of R, Q, Ar and D for a compound corresponds to each line of Table A.

Table j.39. Compounds of formula I.j in which R^{xa} is CH₃, R^{xb} is CH₃, R^{yc} is H, A is CH, G is C—Cl, and the combination of R, Q, Ar and D for a compound corresponds to each line of Table A.

Table j.40. Compounds of formula I.j in which R^{xa} is CH₃, R^{xb} is CH₃, R^{yc} is H, A is N, G is C—Cl, and the combination of R, Q, Ar and D for a compound corresponds to each line of Table A.

Table j.41. Compounds of formula I.j in which R^{xa} is CH₃, R^{xb} is CH₃, R^{yc} is CH₃, A is CH, G is CH, and the combination of R, Q, Ar and D for a compound corresponds to each line of Table A.

Table j.42. Compounds of formula I.j in which R^{xa} is CH₃, R^{xb} is CH₃, R^{yc} is CH₃, A is N, G is CH, and the combination of R, Q, Ar and D for a compound corresponds to each line of Table A.

Table j.43. Compounds of formula I.j in which R^{xa} is CH₃, R^{xb} is CH₃, R^{yc} is CH₃, A is CH, G is N, and the combination of R, Q, Ar and D for a compound corresponds to each line of Table A.

Table j.44. Compounds of formula I.j in which R^{xa} is CH₃, R^{xb} is CH₃, R^{yc} is CH₃, A is N, G is N, and the combination of R, Q, Ar and D for a compound corresponds to each line of Table A.

Table j.45. Compounds of formula I.j in which R^{xa} is CH₃, R^{xb} is CH₃, R^{yc} is CH₃, A is CH, G is C—CH₃, and the combination of R, Q, Ar and D for a compound corresponds to each line of Table A.

Table j.46. Compounds of formula I.j in which R^{xa} is CH₃, R^{xb} is CH₃, R^{yc} is CH₃, A is N, G is C—CH₃, and the combination of R, Q, Ar and D for a compound corresponds to each line of Table A.

Table j.47. Compounds of formula I.j in which R^{xa} is CH₃, R^{xb} is CH₃, R^{yc} is CH₃, A is CH, G is C—Cl, and the combination of R, Q, Ar and D for a compound corresponds to each line of Table A.

Table j.48. Compounds of formula I.j in which R^{xa} is CH₃, R^{xb} is CH₃, R^{yc} is CH₃, A is N, G is C—Cl, and the combination of R, Q, Ar and D for a compound corresponds to each line of Table A.

Table k.1. Compounds of formula I.k in which R^{xa} is H, R^{xb} is H, R^{yc} is H, A is CH, G is CH, and the combination of R, Q, Ar and D for a compound corresponds to each line of Table A.

Table k.2. Compounds of formula I.k in which R^{xa} is H, R^{xb} is H, R^{yc} is H, A is N, G is CH, and the combination of R, Q, Ar and D for a compound corresponds to each line of Table A.

Table k.3. Compounds of formula I.k in which R^{xa} is H, R^{xb} is H, R^{yc} is H, A is CH, G is N, and the combination of R, Q, Ar and D for a compound corresponds to each line of Table A.

Table k.4. Compounds of formula I.k in which R^{xa} is H, R^{xb} is H, R^{yc} is H, A is N, G is N, and the combination of R, Q, Ar and D for a compound corresponds to each line of Table A.

Table k.5. Compounds of formula I.k in which R^{xa} is H, R^{xb} is H, R^{yc} is H, A is CH, G is C—CH₃, and the combination of R, Q, Ar and D for a compound corresponds to each line of Table A.

Table k.6. Compounds of formula I.k in which R^{xa} is H, R^{xb} is H, R^{yc} is H, A is N, G is C—CH₃, and the combination of R, Q, Ar and D for a compound corresponds to each line of Table A.

Table k.7. Compounds of formula I.k in which R^{xa} is H, R^{xb} is H, R^{yc} is H, A is CH, G is C—Cl, and the combination of R, Q, Ar and D for a compound corresponds to each line of Table A.

Table k.8. Compounds of formula I.k in which R^{xa} is H, R^{xb} is H, R^{yc} is H, A is N, G is C—Cl, and the combination of R, Q, Ar and D for a compound corresponds to each line of Table A.

Table k.9. Compounds of formula I.k in which R^{xa} is CH₃, R^{xb} is H, R^{yc} is H, A is CH, G is CH, and the combination of R, Q, Ar and D for a compound corresponds to each line of Table A.

Table k.10. Compounds of formula I.k in which R^{xa} is CH₃, R^{xb} is H, R^{yc} is H, A is N, G is CH, and the combination of R, Q, Ar and D for a compound corresponds to each line of Table A.

Table k.11. Compounds of formula I.k in which R^{xa} is CH₃, R^{xb} is H, R^{yc} is H, A is CH, G is N, and the combination of R, Q, Ar and D for a compound corresponds to each line of Table A.

Table k.12. Compounds of formula I.k in which R^{xa} is CH₃, R^{xb} is H, R^{yc} is H, A is N, G is N, and the combination of R, Q, Ar and D for a compound corresponds to each line of Table A.

Table k.13. Compounds of formula I.k in which R^{xa} is CH₃, R^{xb} is H, R^{yc} is H, A is CH, G is C—CH₃, and the combination of R, Q, Ar and D for a compound corresponds to each line of Table A.

Table k.14. Compounds of formula I.k in which R^{xa} is CH₃, R^{xb} is H, R^{yc} is H, A is N, G is C—CH₃, and the combination of R, Q, Ar and D for a compound corresponds to each line of Table A.

Table k.15. Compounds of formula I.k in which R^{xa} is CH₃, R^{xb} is H, R^{yc} is H, A is CH, G is C—Cl, and the combination of R, Q, Ar and D for a compound corresponds to each line of Table A.

Table k.16. Compounds of formula I.k in which R^{xa} is CH₃, R^{xb} is H, R^{yc} is H, A is N, G is C—Cl, and the combination of R, Q, Ar and D for a compound corresponds to each line of Table A.

Table k.17. Compounds of formula I.k in which R^{xa} is H, R^{xb} is CH₃, R^{yc} is H, A is CH, G is CH, and the combination of R, Q, Ar and D for a compound corresponds to each line of Table A.

Table k.18. Compounds of formula I.k in which R^{xa} is H, R^{xb} is CH₃, R^{yc} is H, A is N, G is CH, and the combination of R, Q, Ar and D for a compound corresponds to each line of Table A.

Table k.19. Compounds of formula I.k in which R^{xa} is H, R^{xb} is CH₃, R^{yc} is H, A is CH, G is N, and the combination of R, Q, Ar and D for a compound corresponds to each line of Table A.

Table k.20. Compounds of formula I.k in which R^{xa} is H, R^{xb} is CH₃, R^{yc} is H, A is N, G is N, and the combination of R, Q, Ar and D for a compound corresponds to each line of Table A.

Table k.21. Compounds of formula I.k in which R^{xa} is H, R^{xb} is CH₃, R^{yc} is H, A is CH, G is C—CH₃CH₃, and the combination of R, Q, Ar and D for a compound corresponds to each line of Table A.

Table k.22. Compounds of formula I.k in which R^{xa} is H, R^{xb} is CH₃, R^{yc} is H, A is N, G is C—CH₃, and the combination of R, Q, Ar and D for a compound corresponds to each line of Table A.

Table k.23. Compounds of formula I.k in which R^{xa} is H, R^{xb} is CH₃, R^{yc} is H, A is CH, G is C—Cl, and the combination of R, Q, Ar and D for a compound corresponds to each line of Table A.

Table k.24. Compounds of formula I.k in which R^{xa} is H, R^{xb} is CH₃, R^{yc} is H, A is N, G is C—Cl, and the combination of R, Q, Ar and D for a compound corresponds to each line of Table A.

Table k.25. Compounds of formula I.k in which R^{xa} is H, R^{xb} is H, R^{yc} is CH₃, A is CH, G is CH, and the combination of R, Q, Ar and D for a compound corresponds to each line of Table A.

Table k.26. Compounds of formula I.k in which R^{xa} is H, R^{xb} is H, R^{yc} is CH₃, A is N, G is CH, and the combination of R, Q, Ar and D for a compound corresponds to each line of Table A.

Table k.27. Compounds of formula I.k in which R^{xa} is H, R^{xb} is H, R^{yc} is CH₃, A is CH, G is N, and the combination of R, Q, Ar and D for a compound corresponds to each line of Table A.

Table k.28. Compounds of formula I.k in which R^{xa} is H, R^{xb} is H, R^{yc} is CH₃, A is N, G is N, and the combination of R, Q, Ar and D for a compound corresponds to each line of Table A.

Table k.29. Compounds of formula I.k in which R^{xa} is H, R^{xb} is H, R^{yc} is CH₃, A is CH, G is C—CH₃, and the combination of R, Q, Ar and D for a compound corresponds to each line of Table A.

Table k.30. Compounds of formula I.k in which R^{xa} is H, R^{xb} is H, R^{yc} is CH₃, A is N, G is C—CH₃, and the combination of R, Q, Ar and D for a compound corresponds to each line of Table A.

Table k.31. Compounds of formula I.k in which R^{xa} is H, R^{xb} is H, R^{yc} is CH₃, A is CH, G is C—Cl, and the combination of R, Q, Ar and D for a compound corresponds to each line of Table A.

Table k.32. Compounds of formula I.k in which R^{xa} is H, R^{xb} is H, R^{yc} is CH₃, A is N, G is C—Cl, and the combination of R, Q, Ar and D for a compound corresponds to each line of Table A.

Table k.33. Compounds of formula I.k in which R^{xa} is CH₃, R^{xb} is CH₃, R^{yc} is H, A is CH, G is CH, and the combination of R, Q, Ar and D for a compound corresponds to each line of Table A.

Table k.34. Compounds of formula I.k in which R^{xa} is CH₃, R^{xb} is CH₃, R^{yc} is H, A is N, G is CH, and the combination of R, Q, Ar and D for a compound corresponds to each line of Table A.

Table k.35. Compounds of formula I.k in which R^{xa} is CH₃, R^{xb} is CH₃, R^{yc} is H, A is CH, G is N, and the combination of R, Q, Ar and D for a compound corresponds to each line of Table A.

Table k.36. Compounds of formula I.k in which R^{xa} is CH₃, R^{xb} is CH₃, R^{yc} is H, A is N, G is N, and the combination of R, Q, Ar and D for a compound corresponds to each line of Table A.

Table k.37. Compounds of formula I.k in which R^{xa} is CH₃, R^{xb} is CH₃, R^{yc} is H, A is CH, G is C—CH₃, and the combination of R, Q, Ar and D for a compound corresponds to each line of Table A.

Table k.38. Compounds of formula I.k in which R^{xa} is CH₃, R^{xb} is CH₃, R^{yc} is H, A is N, G is C—CH₃, and the combination of R, Q, Ar and D for a compound corresponds to each line of Table A.

Table k.39. Compounds of formula I.k in which R^{xa} is CH₃, R^{xb} is CH₃, R^{yc} is H, A is CH, G is C—Cl, and the combination of R, Q, Ar and D for a compound corresponds to each line of Table A.

Table k.40. Compounds of formula I.k in which R^{xa} is CH₃, R^{xb} is CH₃, R^{yc} is H, A is N, G is C—Cl, and the combination of R, Q, Ar and D for a compound corresponds to each line of Table A.

Table k.41. Compounds of formula I.k in which R^{xa} is CH₃, R^{xb} is CH₃, R^{yc} is CH₃, A is CH, G is CH, and the combination of R, Q, Ar and D for a compound corresponds to each line of Table A.

Table k.42. Compounds of formula I.k in which R^{xa} is CH₃, R^{xb} is CH₃, R^{yc} is CH₃, A is N, G is CH, and the combination of R, Q, Ar and D for a compound corresponds to each line of Table A.

Table k.43. Compounds of formula I.k in which R^{xa} is CH₃, R^{xb} is CH₃, R^{yc} is CH₃, A is CH, G is N, and the combination of R, Q, Ar and D for a compound corresponds to each line of Table A.

Table k.44. Compounds of formula I.k in which R^{xa} is CH₃, R^{xb} is CH₃, R^{yc} is CH₃, A is N, G is N, and the combination of R, Q, Ar and D for a compound corresponds to each line of Table A.

Table k.45. Compounds of formula I.k in which R^{xa} is CH₃, R^{xb} is CH₃, R^{yc} is CH₃, A is CH, G is C—CH₃, and the combination of R, Q, Ar and D for a compound corresponds to each line of Table A.

Table k.46. Compounds of formula I.k in which R^{xa} is CH₃, R^{xb} is CH₃, R^{yc} is CH₃, A is N, G is C—CH₃, and the combination of R, Q, Ar and D for a compound corresponds to each line of Table A.

Table k.47. Compounds of formula I.k in which R^{xa} is CH₃, R^{xb} is CH₃, R^{yc} is CH₃, A is CH, G is C—Cl, and the combination of R, Q, Ar and D for a compound corresponds to each line of Table A.

Table k.48. Compounds of formula I.k in which R^{xa} is CH₃, R^{xb} is CH₃, R^{yc} is CH₃, A is N, G is C—Cl, and the combination of R, Q, Ar and D for a compound corresponds to each line of Table A.

Table l.1. Compounds of formula I.l in which R^{xa} is H, R^{xb} is H, R^{ya} is H, A is CH, G is CH, R^{T} is H, and the combination of R, Q, Ar and D for a compound corresponds to each line of Table A.

Table l.2. Compounds of formula I.l in which R^{xa} is H, R^{xb} is H, R^{ya} is H, A is N, G is CH, R^{T} is H, and the combination of R, Q, Ar and D for a compound corresponds to each line of Table A.

Table l.3. Compounds of formula I.l in which R^{xa} is H, R^{xb} is H, R^{ya} is H, A is CH, G is N, R^{T} is H, and the combination of R, Q, Ar and D for a compound corresponds to each line of Table A.

Table l.4. Compounds of formula I.l in which R^{xa} is H, R^{xb} is H, R^{ya} is H, A is N, G is N, R^{T} is H, and the combination of R, Q, Ar and D for a compound corresponds to each line of Table A.

Table l.5. Compounds of formula I.l in which R^{xa} is H, R^{xb} is H, R^{ya} is H, A is CH, G is C—CH₃, R^{T} is H, and the combination of R, Q, Ar and D for a compound corresponds to each line of Table A.

Table l.6. Compounds of formula I.l in which R^{xa} is H, R^{xb} is H, R^{ya} is H, A is N, G is C—CH₃, R^{T} is H, and the combination of R, Q, Ar and D for a compound corresponds to each line of Table A.

Table l.7. Compounds of formula I.l in which R^{xa} is H, R^{xb} is H, R^{ya} is H, A is CH, G is C—Cl, R^{T} is H, and the combination of R, Q, Ar and D for a compound corresponds to each line of Table A.

Table l.8. Compounds of formula I.l in which R^{xa} is H, R^{xb} is H, R^{ya} is H, A is N, G is C—Cl, R^{T} is H, and the combination of R, Q, Ar and D for a compound corresponds to each line of Table A.

Table l.9. Compounds of formula I.l in which R^{xa} is CH₃, R^{xb} is H, R^{ya} is H, A is CH, G is CH, R^{T} is H, and the combination of R, Q, Ar and D for a compound corresponds to each line of Table A.

Table l.10. Compounds of formula I.l in which R^{xa} is CH₃, R^{xb} is H, R^{ya} is H, A is N, G is CH, R^{T} is H, and the combination of R, Q, Ar and D for a compound corresponds to each line of Table A.

Table l.11. Compounds of formula I.l in which R^{xa} is CH₃, R^{xb} is H, R^{ya} is H, A is CH, G is N, R^{T} is H, and the combination of R, Q, Ar and D for a compound corresponds to each line of Table A.

Table l.12. Compounds of formula I.l in which R^{xa} is CH₃, R^{xb} is H, R^{ya} is H, A is N, G is N, R^{T} is H, and the combination of R, Q, Ar and D for a compound corresponds to each line of Table A.

Table l.13. Compounds of formula I.l in which R^{xa} is CH₃, R^{xb} is H, R^{ya} is H, A is CH, G is C—CH₃, R^{T} is H, and the combination of R, Q, Ar and D for a compound corresponds to each line of Table A.

Table l.14. Compounds of formula I.l in which R^{xa} is CH₃, R^{xb} is H, R^{ya} is H, A is N, G is C—CH₃, R^{T} is H, and the combination of R, Q, Ar and D for a compound corresponds to each line of Table A.

Table l.15. Compounds of formula I.l in which R^{xa} is CH₃, R^{xb} is H, R^{ya} is H, A is CH, G is C—Cl, R^{T} is H, and the combination of R, Q, Ar and D for a compound corresponds to each line of Table A.

Table l.16. Compounds of formula I.l in which R^{xa} is CH₃, R^{xb} is H, R^{ya} is H, A is N, G is C—Cl, R^{T} is H, and the combination of R, Q, Ar and D for a compound corresponds to each line of Table A.

Table l.17. Compounds of formula I.l in which R^{xa} is H, R^{xb} is CH₃, R^{ya} is H, A is CH, G is CH, R^{T} is H, and the combination of R, Q, Ar and D for a compound corresponds to each line of Table A.

Table l.18. Compounds of formula I.l in which R^{xa} is H, R^{xb} is CH₃, R^{ya} is H, A is N, G is CH, R^{T} is H, and the combination of R, Q, Ar and D for a compound corresponds to each line of Table A.

Table l.19. Compounds of formula I.l in which R^{xa} is H, R^{xb} is CH₃, R^{ya} is H, A is CH, G is N, R^{T} is H, and the combination of R, Q, Ar and D for a compound corresponds to each line of Table A.

Table l.20. Compounds of formula I.l in which R^{xa} is H, R^{xb} is CH₃, R^{ya} is H, A is N, G is N, R^{T} is H, and the combination of R, Q, Ar and D for a compound corresponds to each line of Table A.

Table l.21. Compounds of formula I.l in which R^{xa} is H, R^{xb} is CH₃, R^{ya} is H, A is CH, G is C—CH₃, R^{T} is H, and the combination of R, Q, Ar and D for a compound corresponds to each line of Table A.

Table l.22. Compounds of formula I.l in which R^{xa} is H, R^{xb} is CH₃, R^{ya} is H, A is N, G is C—CH₃, R^{T} is H, and the combination of R, Q, Ar and D for a compound corresponds to each line of Table A.

Table l.23. Compounds of formula I.l in which R^{xa} is H, R^{xb} is CH₃, R^{ya} is H, A is CH, G is C—Cl, R^{T} is H, and the combination of R, Q, Ar and D for a compound corresponds to each line of Table A.

Table l.24. Compounds of formula I.l in which R^{xa} is H, R^{xb} is CH₃, R^{ya} is H, A is N, G is C—Cl, R^{T} is H, and the combination of R, Q, Ar and D for a compound corresponds to each line of Table A.

Table l.25. Compounds of formula I.l in which R^{xa} is H, R^{xb} is H, R^{ya} is CH₃, A is CH, G is CH, R^{T} is H, and the combination of R, Q, Ar and D for a compound corresponds to each line of Table A.

Table l.26. Compounds of formula I.l in which R^{xa} is H, R^{xb} is H, R^{ya} is CH₃, A is N, G is CH, R^{T} is H, and the combination of R, Q, Ar and D for a compound corresponds to each line of Table A.

Table l.27. Compounds of formula I.l in which R^{xa} is H, R^{xb} is H, R^{ya} is CH₃, A is CH, G is N, R^{T} is H, and the combination of R, Q, Ar and D for a compound corresponds to each line of Table A.

Table l.28. Compounds of formula I.l in which R^{xa} is H, R^{xb} is H, R^{ya} is CH₃, A is N, G is N, R^{T} is H, and the combination of R, Q, Ar and D for a compound corresponds to each line of Table A.

Table l.29. Compounds of formula I.l in which R^{xa} is H, R^{xb} is H, R^{ya} is CH₃, A is CH, G is C—CH₃, R^{T} is H, and the combination of R, Q, Ar and D for a compound corresponds to each line of Table A.

Table l.30. Compounds of formula I.l in which R^{xa} is H, R^{xb} is H, R^{ya} is CH₃, A is N, G is C—CH₃, R^{T} is H, and the combination of R, Q, Ar and D for a compound corresponds to each line of Table A.

Table l.31. Compounds of formula I.l in which R^{xa} is H, R^{xb} is H, R^{ya} is CH₃, A is CH, G is C—Cl, R^{T} is H, and the combination of R, Q, Ar and D for a compound corresponds to each line of Table A.

Table l.32. Compounds of formula I.l in which R^{xa} is H, R^{xb} is H, R^{ya} is CH₃, A is N, G is C—Cl, R^{T} is H, and the combination of R, Q, Ar and D for a compound corresponds to each line of Table A.

Table l.33. Compounds of formula I.l in which R^{xa} is CH₃, R^{xb} is CH₃, R^{ya} is H, A is CH, G is CH, R^{T}is H, and the combination of R, Q, Ar and D for a compound corresponds to each line of Table A.

Table l.34. Compounds of formula I.l in which R^{xa} is CH₃, R^{xb} is CH₃, R^{ya} is H, A is N, G is CH, R^{T} is H, and the combination of R, Q, Ar and D for a compound corresponds to each line of Table A.

Table l.35. Compounds of formula I.l in which R^{xa} is CH₃, R^{xb} is CH₃, R^{ya} is H, A is CH, G is N, R^{T} is H, and the combination of R, Q, Ar and D for a compound corresponds to each line of Table A.

Table l.36. Compounds of formula I.l in which R^{xa} is CH₃, R^{xb} is CH₃, R^{ya} is H, A is N, G is N, R^{T} is H, and the combination of R, Q, Ar and D for a compound corresponds to each line of Table A.

Table l.37. Compounds of formula I.l in which R^{xa} is CH₃, R^{xb} is CH₃, R^{ya} is H, A is CH, G is C—CH₃, R^{T} is H, and the combination of R, Q, Ar and D for a compound corresponds to each line of Table A.

Table l.38. Compounds of formula I.l in which R^{xa} is CH₃, R^{xb} is CH₃, R^{ya} is H, A is N, G is C—CH₃, R^{T} is H, and the combination of R, Q, Ar and D for a compound corresponds to each line of Table A.

Table l.39. Compounds of formula I.l in which R^{xa} is CH₃, R^{xb} is CH₃, R^{ya} is H, A is CH, G is C—Cl, R^{T} is H, and the combination of R, Q, Ar and D for a compound corresponds to each line of Table A.

Table l.40. Compounds of formula I.l in which R^{xa} is CH₃, R^{xb} is CH₃, R^{ya} is H, A is N, G is C—Cl, R^{T} is H, and the combination of R, Q, Ar and D for a compound corresponds to each line of Table A.

Table l.41. Compounds of formula I.l in which R^{xa} is CH₃, R^{xb} is CH₃, R^{ya} is CH₃, A is CH, G is CH, R^{T} is H, and the combination of R, Q, Ar and D for a compound corresponds to each line of Table A.

Table l.42. Compounds of formula I.l in which R^{xa} is CH₃, R^{xb} is CH₃, R^{ya} is CH₃, A is N, G is CH, R^{T} is H, and the combination of R, Q, Ar and D for a compound corresponds to each line of Table A.

Table l.43. Compounds of formula I.l in which R^{xa} is CH₃, R^{xb} is CH₃, R^{ya} is CH₃, A is CH, G is N, R^{T} is H, and the combination of R, Q, Ar and D for a compound corresponds to each line of Table A.

Table l.44. Compounds of formula I.l in which R^{xa} is CH₃, R^{xb} is CH₃, R^{ya} is CH₃, A is N, G is N, R^{T} is H, and the combination of R, Q, Ar and D for a compound corresponds to each line of Table A.

Table l.45. Compounds of formula I.l in which R^{xa} is CH₃, R^{xb} is CH₃, R^{ya} is CH₃, A is CH, G is CH₃, R^{T} is H, and the combination of R, Q, Ar and D for a compound corresponds to each line of Table A.

Table l.46. Compounds of formula I.l in which R^{xa} is CH₃, R^{xb} is CH₃, R^{ya} is CH₃, A is N, G is CH₃, R^{T} is H, and the combination of R, Q, Ar and D for a compound corresponds to each line of Table A.

Table l.47. Compounds of formula I.l in which R^{xa} is CH₃, R^{xb} is CH₃, R^{ya} is CH₃, A is CH, G is C—Cl, R^{T} is H, and the combination of R, Q, Ar and D for a compound corresponds to each line of Table A.

Table l.48. Compounds of formula I.l in which R^{xa} is CH₃, R^{xb} is CH₃, R^{ya} is CH₃, A is N, G is C—Cl, R^{T} is H, and the combination of R, Q, Ar and D for a compound corresponds to each line of Table A.

Table l.49. Compounds of formula I.l in which R^{xa} is H, R^{xb} is H, R^{ya} is H, A is CH, G is CH, R^{T} is CH₃, and the combination of R, Q, Ar and D for a compound corresponds to each line of Table A.

Table l.50. Compounds of formula I.l in which R^{xa} is H, R^{xb} is H, R^{ya} is H, A is N, G is CH, R^{T} is CH₃, and the combination of R, Q, Ar and D for a compound corresponds to each line of Table A.

Table l.51. Compounds of formula I.l in which R^{xa} is H, R^{xb} is H, R^{ya} is H, A is CH, G is N, R^{T} is CH₃, and the combination of R, Q, Ar and D for a compound corresponds to each line of Table A.

Table l.52. Compounds of formula I.l in which R^{xa} is H, R^{xb} is H, R^{ya} is H, A is N, G is N, R^{T} is CH₃, and the combination of R, Q, Ar and D for a compound corresponds to each line of Table A.

Table l.53. Compounds of formula I.l in which R^{xa} is H, R^{xb} is H, R^{ya} is H, A is CH, G is C—CH₃, R^{T} is CH₃, and the combination of R, Q, Ar and D for a compound corresponds to each line of Table A.

Table l.54. Compounds of formula I.l in which R^{xa} is H, R^{xb} is H, R^{ya} is H, A is N, G is C—CH₃, R^{T} is CH₃, and the combination of R, Q, Ar and D for a compound corresponds to each line of Table A.

Table l.55. Compounds of formula I.l in which R^{xa} is H, R^{xb} is H, R^{ya} is H, A is CH, G is C—Cl, R^{T} is CH₃, and the combination of R, Q, Ar and D for a compound corresponds to each line of Table A.

Table l.56. Compounds of formula I.l in which R^{xa} is H, R^{xb} is H, R^{ya} is H, A is N, G is C—Cl, R^{T} is CH₃, and the combination of R, Q, Ar and D for a compound corresponds to each line of Table A.

Table l.57. Compounds of formula I.l in which R^{xa} is CH₃, R^{xb} is H, R^{ya} is H, A is CH, G is CH, R^{T} is CH₃, and the combination of R, Q, Ar and D for a compound corresponds to each line of Table A.

Table l.58. Compounds of formula I.l in which R^{xa} is CH₃, R^{xb} is H, R^{ya} is H, A is N, G is CH, R^{T} is CH₃, and the combination of R, Q, Ar and D for a compound corresponds to each line of Table A.

Table l.59. Compounds of formula I.l in which R^{xa} is CH₃, R^{xb} is H, R^{ya} is H, A is CH, G is N, R^{T} is CH₃, and the combination of R, Q, Ar and D for a compound corresponds to each line of Table A.

Table l.60. Compounds of formula I.l in which R^{xa} is CH₃, R^{xb} is H, R^{ya} is H, A is N, G is N, R^{T} is CH₃, and the combination of R, Q, Ar and D for a compound corresponds to each line of Table A.

Table l.61. Compounds of formula I.l in which R^{xa} is CH₃, R^{xb} is H, R^{ya} is H, A is CH, G is C—CH₃, R^{T} is CH₃, and the combination of R, Q, Ar and D for a compound corresponds to each line of Table A.

Table l.62. Compounds of formula I.l in which R^{xa} is CH₃, R^{xb} is H, R^{ya} is H, A is N, G is C—CH₃, R^{T} is CH₃, and the combination of R, Q, Ar and D for a compound corresponds to each line of Table A.

Table l.63. Compounds of formula I.l in which R^{xa} is CH₃, R^{xb} is H, R^{ya} is H, A is CH, G is C—Cl, R^{T} is CH₃, and the combination of R, Q, Ar and D for a compound corresponds to each line of Table A.

Table l.64. Compounds of formula I.l in which R^{xa} is CH₃, R^{xb} is H, R^{ya} is H, A is N, G is C—Cl, R^{T} is CH₃, and the combination of R, Q, Ar and D for a compound corresponds to each line of Table A.

Table l.65. Compounds of formula I.l in which R^{xa} is H, R^{xb} is CH₃, R^{ya} is H, A is CH, G is CH, R^{T} is CH₃, and the combination of R, Q, Ar and D for a compound corresponds to each line of Table A.

Table l.66. Compounds of formula I.l in which R^{xa} is H, R^{xb} is CH₃, R^{ya} is H, A is N, G is CH, R^{T} is CH₃, and the combination of R, Q, Ar and D for a compound corresponds to each line of Table A.

Table l.67. Compounds of formula I.l in which R^{xa} is H, R^{xb} is CH₃, R^{ya} is H, A is CH, G is N, R^{T} is CH₃, and the combination of R, Q, Ar and D for a compound corresponds to each line of Table A.

Table l.68. Compounds of formula I.l in which R^{xa} is H, R^{xb} is CH₃, R^{ya} is H, A is N, G is N, R^{T} is CH₃, and the combination of R, Q, Ar and D for a compound corresponds to each line of Table A.

Table l.69. Compounds of formula I.l in which R^{xa} is H, R^{xb} is CH₃, R^{ya} is H, A is CH, G is C—CH₃, R^{T} is CH₃, and the combination of R, Q, Ar and D for a compound corresponds to each line of Table A.

Table l.70. Compounds of formula I.l in which R^{xa} is H, R^{xb} is CH₃, R^{ya} is H, A is N, G is C—CH₃, R^{T} is CH₃, and the combination of R, Q, Ar and D for a compound corresponds to each line of Table A.

Table l.71. Compounds of formula I.l in which R^{xa} is H, R^{xb} is CH₃, R^{ya} is H, A is CH, G is C—Cl, R^{T} is CH₃, and the combination of R, Q, Ar and D for a compound corresponds to each line of Table A.

Table l.72. Compounds of formula I.l in which R^{xa} is H, R^{xb} is CH₃, R^{ya} is H, A is N, G is C—Cl, R^{T} is CH₃, and the combination of R, Q, Ar and D for a compound corresponds to each line of Table A.

Table l.73. Compounds of formula I.l in which R^{xa} is H, R^{xb} is H, R^{ya} is CH₃, A is CH, G is CH, R^{T} is CH₃, and the combination of R, Q, Ar and D for a compound corresponds to each line of Table A.

Table l.74. Compounds of formula I.l in which R^{xa} is H, R^{xb} is H, R^{ya} is CH₃, A is N, G is CH, R^{T} is CH₃, and the combination of R, Q, Ar and D for a compound corresponds to each line of Table A.

Table l.75. Compounds of formula I.l in which R^{xa} is H, R^{xb} is H, R^{ya} is CH₃, A is CH, G is N, R^{T} is CH₃, and the combination of R, Q, Ar and D for a compound corresponds to each line of Table A.

Table l.76. Compounds of formula I.l in which R^{xa} is H, R^{xb} is H, R^{ya} is CH₃, A is N, G is N, R^{T} is CH₃, and the combination of R, Q, Ar and D for a compound corresponds to each line of Table A.

Table l.77. Compounds of formula I.l in which R^{xa} is H, R^{xb} is H, R^{ya} is CH₃, A is CH, G is C—CH₃, R^{T} is CH₃, and the combination of R, Q, Ar and D for a compound corresponds to each line of Table A.

Table l.78. Compounds of formula I.l in which R^{xa} is H, R^{xb} is H, R^{ya} is CH₃, A is N, G is C—CH₃, R^{T} is CH₃, and the combination of R, Q, Ar and D for a compound corresponds to each line of Table A.

Table l.79. Compounds of formula I.l in which R^{xa} is H, R^{xb} is H, R^{ya} is CH₃, A is CH, G is C—Cl, R^{T} is CH₃, and the combination of R, Q, Ar and D for a compound corresponds to each line of Table A.

Table l.80. Compounds of formula I.l in which R^{xa} is H, R^{xb} is H, R^{ya} is CH₃, A is N, G is C—Cl, R^{T} is CH₃, and the combination of R, Q, Ar and D for a compound corresponds to each line of Table A.

Table l.81. Compounds of formula I.l in which R^{xa} is CH₃, R^{xb} is CH₃, R^{ya} is H, A is CH, G is CH, R^{T} is CH₃, and the combination of R, Q, Ar and D for a compound corresponds to each line of Table A.

Table l.82. Compounds of formula I.l in which R^{xa} is CH₃, R^{xb} is CH₃, R^{ya} is H, A is N, G is CH, R^{T} is CH₃, and the combination of R, Q, Ar and D for a compound corresponds to each line of Table A.

Table l.83. Compounds of formula I.l in which R^{xa} is CH₃, R^{xb} is CH₃, R^{ya} is H, A is CH, G is N, R^{T} is CH₃, and the combination of R, Q, Ar and D for a compound corresponds to each line of Table A.

Table l.84. Compounds of formula I.l in which R^{xa} is CH₃, R^{xb} is CH₃, R^{ya} is H, A is N, G is N, R^{T} is CH₃, and the combination of R, Q, Ar and D for a compound corresponds to each line of Table A.

Table l.85. Compounds of formula I.l in which R^{xa} is CH₃, R^{xb} is CH₃, R^{ya} is H, A is CH, G is C—CH₃, R^{T} is CH₃, and the combination of R, Q, Ar and D for a compound corresponds to each line of Table A.

Table l.86. Compounds of formula I.l in which R^{xa} is CH₃, R^{xb} is CH₃, R^{ya} is H, A is N, G is C—CH₃, R^{T} is CH₃, and the combination of R, Q, Ar and D for a compound corresponds to each line of Table A.

Table l.87. Compounds of formula I.l in which R^{xa} is CH₃, R^{xb} is CH₃, R^{ya} is H, A is CH, G is C—Cl, R^{T} is CH₃, and the combination of R, Q, Ar and D for a compound corresponds to each line of Table A.

Table l.88. Compounds of formula I.l in which R^{xa} is CH₃, R^{xb} is CH₃, R^{ya} is H, A is N, G is C—Cl, R^{T} is CH₃, and the combination of R, Q, Ar and D for a compound corresponds to each line of Table A.

Table l.89. Compounds of formula I.l in which R^{xa} is CH₃, R^{xb} is CH₃, R^{ya} is CH₃, A is CH, G is CH, R^{T} is CH₃, and the combination of R, Q, Ar and D for a compound corresponds to each line of Table A.

Table l.90. Compounds of formula I.l in which R^{xa} is CH₃, R^{xb} is CH₃, R^{ya} is CH₃, A is N, G is CH, R^{T} is CH₃, and the combination of R, Q, Ar and D for a compound corresponds to each line of Table A.

Table l.91. Compounds of formula I.l in which R^{xa} is CH₃, R^{xb} is CH₃, R^{ya} is CH₃, A is CH, G is N, R^{T} is CH₃, and the combination of R, Q, Ar and D for a compound corresponds to each line of Table A.

Table l.92. Compounds of formula I.l in which R^{xa} is CH₃, R^{xb} is CH₃, R^{ya} is CH₃, A is N, G is N, R^{T}is CH₃, and the combination of R, Q, Ar and D for a compound corresponds to each line of Table A.

Table l.93. Compounds of formula I.l in which R^{xa} is CH₃, R^{xb} is CH₃, R^{ya} is CH₃, A is CH, G is C—CH₃, R^{T} is CH₃, and the combination of R, Q, Ar and D for a compound corresponds to each line of Table A.

Table l.94. Compounds of formula I.l in which R^{xa} is CH₃, R^{xb} is CH₃, R^{ya} is CH₃, A is N, G is C—CH₃, R^{T} is CH₃, and the combination of R, Q, Ar and D for a compound corresponds to each line of Table A.

Table l.95. Compounds of formula I.l in which R^{xa} is CH₃, R^{xb} is CH₃, R^{ya} is CH₃, A is CH, G is C—Cl, R^{T} is CH₃, and the combination of R, Q, Ar and D for a compound corresponds to each line of Table A.

Table l.96. Compounds of formula I.l in which R^{xa} is CH₃, R^{xb} is CH₃, R^{ya} is CH₃, A is N, G is C—Cl, R^{T} is CH₃, and the combination of R, Q, Ar and D for a compound corresponds to each line of Table A.

Table m.1. Compounds of formula I.m in which R^{xa} is H, R^{yc} is H, A is CH, G is CH, and the combination of R, Q, Ar and D for a compound corresponds to each line of Table A.

Table m.2. Compounds of formula I.m in which R^{xa} is H, R^{yc} is H, A is N, G is CH, and the combination of R, Q, Ar and D for a compound corresponds to each line of Table A.

Table m.3. Compounds of formula I.m in which R^{xa} is H, R^{yc} is H, A is CH, G is N, and the combination of R, Q, Ar and D for a compound corresponds to each line of Table A.

Table m.4. Compounds of formula I.m in which R^{xa} is H, R^{yc} is H, A is N, G is N, and the combination of R, Q, Ar and D for a compound corresponds to each line of Table A.

Table m.5. Compounds of formula I.m in which R^{xa} is H, R^{yc} is H, A is CH, G is C—CH₃, and the combination of R, Q, Ar and D for a compound corresponds to each line of Table A.

Table m.6. Compounds of formula I.m in which R^{xa} is H, R^{yc} is H, A is N, G is C—CH₃, and the combination of R, Q, Ar and D for a compound corresponds to each line of Table A.

Table m.7. Compounds of formula I.m in which R^{xa} is H, R^{yc} is H, A is CH, G is C—Cl, and the combination of R, Q, Ar and D for a compound corresponds to each line of Table A.

Table m.8. Compounds of formula I.m in which R^{xa} is H, R^{yc} is H, A is N, G is C—Cl, and the combination of R, Q, Ar and D for a compound corresponds to each line of Table A.

Table m.9. Compounds of formula I.m in which R^{xa} is CH₃, R^{yc} is H, A is CH, G is CH, and the combination of R, Q, Ar and D for a compound corresponds to each line of Table A.

Table m.10. Compounds of formula I.m in which R^{xa} is CH₃, R^{yc} is H, A is N, G is CH, and the combination of R, Q, Ar and D for a compound corresponds to each line of Table A.

Table m.11. Compounds of formula I.m in which R^{xa} is CH₃, R^{yc} is H, A is CH, G is N, and the combination of R, Q, Ar and D for a compound corresponds to each line of Table A.

Table m.12. Compounds of formula I.m in which R^{xa} is CH₃, R^{yc} is H, A is N, G is N, and the combination of R, Q, Ar and D for a compound corresponds to each line of Table A.

Table m.13. Compounds of formula I.m in which R^{xa} is CH₃, R^{yc} is H, A is CH, G is C—CH₃, and the combination of R, Q, Ar and D for a compound corresponds to each line of Table A.

Table m.14. Compounds of formula I.m in which R^{xa} is CH₃, R^{yc} is H, A is N, G is C—CH₃, and the combination of R, Q, Ar and D for a compound corresponds to each line of Table A.

Table m.15. Compounds of formula I.m in which R^{xa} is CH₃, R^{yc} is H, A is CH, G is C—Cl, and the combination of R, Q, Ar and D for a compound corresponds to each line of Table A.

Table m.16. Compounds of formula I.m in which R^{xa} is CH₃, R^{yc} is H, A is N, G is C—Cl, and the combination of R, Q, Ar and D for a compound corresponds to each line of Table A.

Table m.17. Compounds of formula I.m in which R^{xa} is H, R^{yc} is CH₃, A is CH, G is CH, and the combination of R, Q, Ar and D for a compound corresponds to each line of Table A.

Table m.18. Compounds of formula I.m in which R^{xa} is H, R^{yc} is CH₃, A is N, G is CH, and the combination of R, Q, Ar and D for a compound corresponds to each line of Table A.

Table m.19. Compounds of formula I.m in which R^{xa} is H, R^{yc} is CH₃, A is CH, G is N, and the combination of R, Q, Ar and D for a compound corresponds to each line of Table A.

Table m.20. Compounds of formula I.m in which R^{xa} is H, R^{yc} is CH₃, A is N, G is N, and the combination of R, Q, Ar and D for a compound corresponds to each line of Table A.

Table m.21. Compounds of formula I.m in which R^{xa} is H, R^{yc} is CH₃, A is CH, G is C—CH₃, and the combination of R, Q, Ar and D for a compound corresponds to each line of Table A.

Table m.22. Compounds of formula I.m in which R^{xa} is H, R^{yc} is CH₃, A is N, G is C—CH₃, and the combination of R, Q, Ar and D for a compound corresponds to each line of Table A.

Table m.23. Compounds of formula I.m in which R^{xa} is H, R^{yc} is CH₃, A is CH, G is C—Cl, and the combination of R, Q, Ar and D for a compound corresponds to each line of Table A.

Table m.24. Compounds of formula I.m in which R^{xa} is H, R^{yc} is CH₃, A is N, G is C—Cl, and the combination of R, Q, Ar and D for a compound corresponds to each line of Table A.

Table n.1. Compounds of formula I.n in which “1-R^{xa} is H, “1-R^{xb} is H, “2-R^{xa} is H, “2-R^{xb} is H, R^{ya} is H, A is CH, G is CH, and the combination of R, Q, Ar and D for a compound corresponds to each line of Table A.

Table n.2. Compounds of formula I.n in which “1-R^{xa} is H, “1-R^{xb} is H, “2-R^{xa} is H, “2-R^{xb} is H, R^{ya} is H, A is N, G is CH, and the combination of R, Q, Ar and D for a compound corresponds to each line of Table A.

Table n.3. Compounds of formula I.n in which “1-R^{xa} is H, “1-R^{xb} is H, “2-R^{xa} is H, “2-R^{xb} is H, R^{ya} is H, A is CH, G is N, and the combination of R, Q, Ar and D for a compound corresponds to each line of Table A.

Table n.4. Compounds of formula I.n in which “1-R^{xa} is H, “1-R^{xb} is H, “2-R^{xa} is H, “2-R^{xb} is H, R^{ya} is H, A is N, G is N, and the combination of R, Q, Ar and D for a compound corresponds to each line of Table A.

Table n.5. Compounds of formula I.n in which “1-R^{xa} is H, “1-R^{xb} is H, “2-R^{xa} is H, “2-R^{xb} is H, R^{ya} is H, A is CH, G is C—CH₃, and the combination of R, Q, Ar and D for a compound corresponds to each line of Table A.

Table n.6. Compounds of formula I.n in which “1-R^{xa} is H, “1-R^{xb} is H, “2-R^{xa} is H, “2-R^{xb} is H, R^{ya} is H, A is N, G is C—CH₃, and the combination of R, Q, Ar and D for a compound corresponds to each line of Table A.

Table n.7. Compounds of formula I.n in which “1-R^{xa} is H, “1-R^{xb} is H, “2-R^{xa} is H, “2-R^{xb} is H, R^{ya} is H, A is CH, G is C—Cl, and the combination of R, Q, Ar and D for a compound corresponds to each line of Table A.

Table n.8. Compounds of formula I.n in which “1-R^{xa} is H, “1-R^{xb} is H, “2-R^{xa} is H, “2-R^{xb} is H, R^{ya} is H, A is N, G is C—Cl, and the combination of R, Q, Ar and D for a compound corresponds to each line of Table A.

Table n.9. Compounds of formula I.n in which “1-R^{xa} is CH₃, “1-R^{xb} is H, “2-R^{xa} is H, “2-R^{xb} is H, R^{ya} is H, A is CH, G is CH, and the combination of R, Q, Ar and D for a compound corresponds to each line of Table A.

Table n.10. Compounds of formula I.n in which “1-R^{xa} is CH₃, “1-R^{xb} is H, “2-R^{xa} is H, “2-R^{xb} is H, R^{ya} is H, R^{ya} is H, A is N, G is CH, and the combination of R, Q, Ar and D for a compound corresponds to each line of Table A.

Table n.11. Compounds of formula I.n in which “1-R^{xa} is CH₃, “1-R^{xb} is H, “2-R^{xa} is H, “2-R^{xb} is H, R^{ya} is H, R^{ya} is H, A is CH, G is N, and the combination of R, Q, Ar and D for a compound corresponds to each line of Table A.

Table n.12. Compounds of formula I.n in which “1-R^{xa} is CH₃, “1-R^{xb} is H, “2-R^{xa} is H, “2-R^{xb} is H, R^{ya} is H, R^{ya} is H, A is N, G is N, and the combination of R, Q, Ar and D for a compound corresponds to each line of Table A.

Table n.13. Compounds of formula I.n in which “1-R^{xa} is CH₃, “1-R^{xb} is H, “2-R^{xa} is H, “2-R^{xb} is H, R^{ya} is H, R^{ya} is H, A is CH, G is C—CH₃, and the combination of R, Q, Ar and D for a compound corresponds to each line of Table A.

Table n.14. Compounds of formula I.n in which “1-R^{xa} is CH₃, “1-R^{xb} is H, “2-R^{xa} is H, “2-R^{xb} is H, R^{ya} is H, R^{ya} is H, A is N, G is C—CH₃, and the combination of R, Q, Ar and D for a compound corresponds to each line of Table A.

Table n.15. Compounds of formula I.n in which “1-R^{xa} is CH₃, “1-R^{xb} is H, “2-R^{xa} is H, “2-R^{xb} is H, R^{ya} is H, R^{ya} is H, A is CH, G is C—Cl, and the combination of R, Q, Ar and D for a compound corresponds to each line of Table A.

Table n.16. Compounds of formula I.n in which “1-R^{xa} is CH₃, “1-R^{xb} is H, “2-R^{xa} is H, “2-R^{xb} is H, R^{ya} is H, R^{ya} is H, A is N, G is C—Cl, and the combination of R, Q, Ar and D for a compound corresponds to each line of Table A.

Table n.17. Compounds of formula I.n in which “1-R^{xa} is H, “1-R^{xb} is H, “2-R^{xa} is CH₃, “2-R^{xb} is H, R^{ya} is H, R^{ya} is H, A is CH, G is CH, and the combination of R, Q, Ar and D for a compound corresponds to each line of Table A.

Table n.18. Compounds of formula I.n in which “1-R^{xa} is H, “1-R^{xb} is H, “2-R^{xa} is CH₃, “2-R^{xb} is H, R^{ya} is H, A is N, G is CH, and the combination of R, Q, Ar and D for a compound corresponds to each line of Table A.

Table n.19. Compounds of formula I.n in which “1-R^{xa} is H, “1-R^{xb} is H, “2-R^{xa} is CH₃, “2-R^{xb} is H, R^{ya} is H, A is CH, G is N, and the combination of R, Q, Ar and D for a compound corresponds to each line of Table A.

Table n.20. Compounds of formula I.n in which “1-R^{xa} is H, “1-R^{xb} is H, “2-R^{xa} is CH₃, “2-R^{xb} is H, R^{ya} is H, A is N, G is N, and the combination of R, Q, Ar and D for a compound corresponds to each line of Table A.

Table n.21. Compounds of formula I.n in which “1-R^{xa} is H, “1-R^{xb} is H, “2-R^{xa} is CH₃, “2-R^{xb} is H, R^{ya} is H, A is CH, G is C—CH₃, and the combination of R, Q, Ar and D for a compound corresponds to each line of Table A.

Table n.22. Compounds of formula I.n in which “1-R^{xa} is H, “1-R^{xb} is H, “2-R^{xa} is CH₃, “2-R^{xb} is H, R^{ya} is H, A is N, G is C—CH₃, and the combination of R, Q, Ar and D for a compound corresponds to each line of Table A.

Table n.23. Compounds of formula I.n in which “1-R^{xa} is H, “1-R^{xb} is H, “2-R^{xa} is CH₃, “2-R^{xb} is H, R^{ya} is H, A is CH, G is C—Cl, and the combination of R, Q, Ar and D for a compound corresponds to each line of Table A.

Table n.24. Compounds of formula I.n in which “1-R^{xa} is H, “1-R^{xb} is H, “2-R^{xa} is CH₃, “2-R^{xb} is H, R^{ya} is H, A is N, G is C—Cl, and the combination of R, Q, Ar and D for a compound corresponds to each line of Table A.

Table n.25. Compounds of formula I.n in which “1-R^{xa} is H, “1-R^{xb} is H, “2-R^{xa} is H, “2-R^{xb} is H, R^{ya} is CH₃, A is CH, G is CH, and the combination of R, Q, Ar and D for a compound corresponds to each line of Table A.

Table n.26. Compounds of formula I.n in which “1-R^{xa} is H, “1-R^{xb} is H, “2-R^{xa} is H, “2-R^{xb} is H, R^{ya} is CH₃, A is N, G is CH, and the combination of R, Q, Ar and D for a compound corresponds to each line of Table A.

Table n.27. Compounds of formula I.n in which “1-R^{xa} is H, “1-R^{xb} is H, “2-R^{xa} is H, “2-R^{xb} is H, R^{ya} is CH₃, A is CH, G is N, and the combination of R, Q, Ar and D for a compound corresponds to each line of Table A.

Table n.28. Compounds of formula I.n in which “1-R^{xa} is H, “1-R^{xb} is H, “2-R^{xa} is H, “2-R^{xb} is H, R^{ya} is CH₃, A is N, G is N, and the combination of R, Q, Ar and D for a compound corresponds to each line of Table A.

Table n.29. Compounds of formula I.n in which “1-R^{xa} is H, “1-R^{xb} is H, “2-R^{xa} is H, “2-R^{xb} is H, R^{ya} is CH₃, A is CH, G is C—CH₃, and the combination of R, Q, Ar and D for a compound corresponds to each line of Table A.

Table n.30. Compounds of formula I.n in which “1-R^{xa} is H, “1-R^{xb} is H, “2-R^{xa} is H, “2-R^{xb} is H, R^{ya} is CH₃, A is N, G is C—CH₃, and the combination of R, Q, Ar and D for a compound corresponds to each line of Table A.

Table n.31. Compounds of formula I.n in which “1-R^{xa} is H, “1-R^{xb} is H, “2-R^{xa} is H, “2-R^{xb} is H, R^{ya} is CH₃, A is CH, G is C—Cl, and the combination of R, Q, Ar and D for a compound corresponds to each line of Table A.

Table n.32. Compounds of formula I.n in which “1-R^{xa} is H, “1-R^{xb} is H, “2-R^{xa} is H, “2-R^{xb} is H, R^{ya} is CH₃, A is N, G is C—Cl, and the combination of R, Q, Ar and D for a compound corresponds to each line of Table A.

Table n.33. Compounds of formula I.n in which “1-R^{xa} is CH₃, “1-R^{xb} is CH₃, “2-R^{xa} is CH₃, “2-R^{xb} is CH₃, R^{ya} is H, A is CH, G is CH, and the combination of R, Q, Ar and D for a compound corresponds to each line of Table A.

Table n.34. Compounds of formula I.n in which “1-R^{xa} is CH₃, “1-R^{xb} is CH₃, “2-R^{xa} is CH₃, “2-R^{xb} is CH₃, R^{ya} is H, A is N, G is CH, and the combination of R, Q, Ar and D for a compound corresponds to each line of Table A.

Table n.35. Compounds of formula I.n in which “1-R^{xa} is CH₃, “1-R^{xb} is CH₃, “2-R^{xa} is CH₃, “2-R^{xb} is CH₃, R^{ya} is H, A is CH, G is N, and the combination of R, Q, Ar and D for a compound corresponds to each line of Table A.

Table n.36. Compounds of formula I.n in which “1-R^{xa} is CH₃, “1-R^{xb} is CH₃, “2-R^{xa} is CH₃, “2-R^{xb} is CH₃, R^{ya} is H, A is N, G is N, and the combination of R, Q, Ar and D for a compound corresponds to each line of Table A.

Table n.37. Compounds of formula I.n in which “1-R^{xa} is CH₃, “1-R^{xb} is CH₃, “2-R^{xa} is CH₃, “2-R^{xb} is CH₃, R^{ya} is H, A is CH, G is C—CH₃, and the combination of R, Q, Ar and D for a compound corresponds to each line of Table A.

Table n.38. Compounds of formula I.n in which “1-R^{xa} is CH₃, “1-R^{xb} is CH₃, “2-R^{xa} is CH₃, “2-R^{xb} is CH₃, R^{ya} is H, A is N, G is C—CH₃, and the combination of R, Q, Ar and D for a compound corresponds to each line of Table A.

Table n.39. Compounds of formula I.n in which “1-R^{xa} is CH₃, “1-R^{xb} is CH₃, “2-R^{xa} is CH₃, “2-R^{xb} is CH₃, R^{ya} is H, A is CH, G is C—Cl, and the combination of R, Q, Ar and D for a compound corresponds to each line of Table A.

Table n.40. Compounds of formula I.n in which “1-R^{xa} is CH₃, “1-R^{xb} is CH₃, “2-R^{xa} is CH₃, “2-R^{xb} is CH₃, R^{ya} is H, A is N, G is C—Cl, and the combination of R, Q, Ar and D for a compound corresponds to each line of Table A.

Table n.41. Compounds of formula I.n in which “1-R^{xa} is CH₃, “1-R^{xb} is CH₃, “2-R^{xa} is CH₃, “2-R^{xb} is CH₃, R^{ya} is CH₃, A is CH, G is CH, and the combination of R, Q, Ar and D for a compound corresponds to each line of Table A.

Table n.42. Compounds of formula I.n in which “1-R^{xa} is CH₃, “1-R^{xb} is CH₃, “2-R^{xa} is CH₃, “2-R^{xb} is CH₃, R^{ya} is CH₃, A is N, G is CH, and the combination of R, Q, Ar and D for a compound corresponds to each line of Table A.

Table n.43. Compounds of formula I.n in which “1-R^{xa} is CH₃, “1-R^{xb} is CH₃, “2-R^{xa} is CH₃, “2-R^{xb} is CH₃, R^{ya} is CH₃, A is CH, G is N, and the combination of R, Q, Ar and D for a compound corresponds to each line of Table A.

Table n.44. Compounds of formula I.n in which “1-R^{xa} is CH₃, “1-R^{xb} is CH₃, “2-R^{xa} is CH₃, “2-R^{xb} is CH₃, R^{ya} is CH₃, A is N, G is N, and the combination of R, Q, Ar and D for a compound corresponds to each line of Table A.

Table n.45. Compounds of formula I.n in which “1-R^{xa} is CH₃, “1-R^{xb} is CH₃, “2-R^{xa} is CH₃, “2-R^{xb} is CH₃, R^{ya} is CH₃, A is CH, G is CH₃, and the combination of R, Q, Ar and D for a compound corresponds to each line of Table A.

Table n.46. Compounds of formula I.n in which “1-R^{xa} is CH₃, “1-R^{xb} is CH₃, “2-R^{xa} is CH₃, “2-R^{xb} is CH₃, R^{ya} is CH₃, A is N, G is CH₃, and the combination of R, Q, Ar and D for a compound corresponds to each line of Table A.

Table n.47. Compounds of formula I.n in which “1-R^{xa} is CH₃, “1-R^{xb} is CH₃, “2-R^{xa} is CH₃, “2-R^{xb} is CH₃, R^{ya} is CH₃, A is CH, G is C—Cl, and the combination of R, Q, Ar and D for a compound corresponds to each line of Table A.

Table n.48. Compounds of formula I.n in which “1-R^{xa} is CH₃, “1-R^{xb} is CH₃, “2-R^{xa} is CH₃, “2-R^{xb} is CH₃, R^{ya} is CH₃, A is N, G is C—Cl, and the combination of R, Q, Ar and D for a compound corresponds to each line of Table A.

Table n.49. Compounds of formula I.n in which “1-R^{xa} is CH₃, “1-R^{xb} is CH₃, “2-R^{xa} is H, “2-R^{xb} is H, R^{ya} is H, A is CH, G is CH, and the combination of R, Q, Ar and D for a compound corresponds to each line of Table A.

Table n.50. Compounds of formula I.n in which “1-R^{xa} is CH₃, “1-R^{xb} is CH₃, “2-R^{xa} is H, “2-R^{xb} is H, R^{ya} is H, R^{ya} is H, A is N, G is CH, and the combination of R, Q, Ar and D for a compound corresponds to each line of Table A.

Table n.51. Compounds of formula I.n in which “1-R^{xa} is CH₃, “1-R^{xb} is CH₃, “2-R^{xa} is H, “2-R^{xb} is H, R^{ya} is H, R^{ya} is H, A is CH, G is N, and the combination of R, Q, Ar and D for a compound corresponds to each line of Table A.

Table n.52. Compounds of formula I.n in which “1-R^{xa} is CH₃, “1-R^{xb} is CH₃, “2-R^{xa} is H, “2-R^{xb} is H, R^{ya} is H, R^{ya} is H, A is N, G is N, and the combination of R, Q, Ar and D for a compound corresponds to each line of Table A.

Table n.53. Compounds of formula I.n in which “1-R^{xa} is CH₃, “1-R^{xb} is CH₃, “2-R^{xa} is H, “2-R^{xb} is H, R^{ya} is H, R^{ya} is H, A is CH, G is C—CH₃, and the combination of R, Q, Ar and D for a compound corresponds to each line of Table A.

Table n.54. Compounds of formula I.n in which “1-R^{xa} is CH₃, “1-R^{xb} is CH₃, “2-R^{xa} is H, “2-R^{xb} is H, R^{ya} is H, R^{ya} is H, A is N, G is C—CH₃, and the combination of R, Q, Ar and D for a compound corresponds to each line of Table A.

Table n.55. Compounds of formula I.n in which “1-R^{xa} is CH₃, “1-R^{xb} is CH₃, “2-R^{xa} is H, “2-R^{xb} is H, R^{ya} is H, R^{ya} is H, A is CH, G is C—Cl, and the combination of R, Q, Ar and D for a compound corresponds to each line of Table A.

Table n.56. Compounds of formula I.n in which “1-R^{xa} is CH₃, “1-R^{xb} is CH₃, “2-R^{xa} is H, “2-R^{xb} is H, R^{ya} is H, R^{ya} is H, A is N, G is C—Cl, and the combination of R, Q, Ar and D for a compound corresponds to each line of Table A.

Table n.57. Compounds of formula I.n in which “1-R^{xa} is H, “1-R^{xb} is H, “2-R^{xa} is CH₃, “2-R^{xb} is CH₃, R^{ya} is H, R^{ya} is H, A is CH, G is CH, and the combination of R, Q, Ar and D for a compound corresponds to each line of Table A.

Table n.58. Compounds of formula I.n in which “1-R^{xa} is H, “1-R^{xb} is H, “2-R^{xa} is CH₃, “2-R^{xb} is CH₃, R^{ya} is H, A is N, G is CH, and the combination of R, Q, Ar and D for a compound corresponds to each line of Table A.

Table n.59. Compounds of formula I.n in which “1-R^{xa} is H, “1-R^{xb} is H, “2-R^{xa} is CH₃, “2-R^{xb} is CH₃, R^{ya} is H, A is CH, G is N, and the combination of R, Q, Ar and D for a compound corresponds to each line of Table A.

Table n.60. Compounds of formula I.n in which “1-R^{xa} is H, “1-R^{xb} is H, “2-R^{xa} is CH₃, “2-R^{xb} is CH₃, R^{ya} is H, A is N, G is N, and the combination of R, Q, Ar and D for a compound corresponds to each line of Table A.

Table n.61. Compounds of formula I.n in which “1-R^{xa} is H, “1-R^{xb} is H, “2-R^{xa} is CH₃, “2-R^{xb} is CH₃, R^{ya} is H, A is CH, G is C—CH₃, and the combination of R, Q, Ar and D for a compound corresponds to each line of Table A.

Table n.62. Compounds of formula I.n in which “1-R^{xa} is H, “1-R^{xb} is H, “2-R^{xa} is CH₃, “2-R^{xb} is CH₃, R^{ya} is H, A is N, G is C—CH₃, and the combination of R, Q, Ar and D for a compound corresponds to each line of Table A.

Table n.63. Compounds of formula I.n in which “1-R^{xa} is H, “1-R^{xb} is H, “2-R^{xa} is CH₃, “2-R^{xb} is CH₃, R^{ya} is H, A is CH, G is C—Cl, and the combination of R, Q, Ar and D for a compound corresponds to each line of Table A.

Table n.64. Compounds of formula I.n in which “1-R^{xa} is H, “1-R^{xb} is H, “2-R^{xa} is CH₃, “2-R^{xb} is CH₃, R^{ya} is H, A is N, G is C—Cl, and the combination of R, Q, Ar and D for a compound corresponds to each line of Table A.

Table n.65. Compounds of formula I.n in which “1-R^{xa} is CH₃, “1-R^{xb} is CH₃, “2-R^{xa} is H, “2-R^{xb} is H, R^{ya} is CH₃, A is CH, G is CH, and the combination of R, Q, Ar and D for a compound corresponds to each line of Table A.

Table n.66. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is N, G is CH, and the combination of R, Q, Ar and D for a compound corresponds to each line of Table A.

Table n.67. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is CH, G is N, and the combination of R, Q, Ar and D for a compound corresponds to each line of Table A.

Table n.68. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is N, G is N, and the combination of R, Q, Ar and D for a compound corresponds to each line of Table A.

Table n.69. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is CH, G is C—CH₃, and the combination of R, Q, Ar and D for a compound corresponds to each line of Table A.

Table n.70. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is N, G is C—CH₃, and the combination of R, Q, Ar and D for a compound corresponds to each line of Table A.

Table n.71. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is CH, G is C—Cl, and the combination of R, Q, Ar and D for a compound corresponds to each line of Table A.

Table n.72. Compounds of formula I.n in which “1-R^{xa} is CH₃, “1-R^{xb} is CH₃, “2-R^{xa} is H, “2-R^{xb} is H, R^{ya} is CH₃, R^{ya} is CH₃, A is N, G is C—Cl, and the combination of R, Q, Ar and D for a compound corresponds to each line of Table A.

Table n.73. Compounds of formula I.n in which “1-R^{xa} is H, “1-R^{xb} is H, “2-R^{xa} is CH₃, “2-R^{xb} is CH₃, R^{ya} is CH₃, R^{ya} is CH₃, A is CH, G is CH, and the combination of R, Q, Ar and D for a compound corresponds to each line of Table A.

Table n.74. Compounds of formula I.n in which “1-R^{xa} is H, “1-R^{xb} is H, “2-R^{xa} is CH₃, “2-R^{xb} is CH₃, R^{ya} is CH₃, A is N, G is CH, and the combination of R, Q, Ar and D for a compound corresponds to each line of Table A.

Table n.75. Compounds of formula I.n in which “1-R^{xa} is H, “1-R^{xb} is H, “2-R^{xa} is CH₃, “2-R^{xb} is CH₃, R^{ya} is CH₃, A is CH, G is N, and the combination of R, Q, Ar and D for a compound corresponds to each line of Table A.

Table n.76. Compounds of formula I.n in which “1-R^{xa} is H, “1-R^{xb} is H, “2-R^{xa} is CH₃, “2-R^{xb} is CH₃, R^{ya} is CH₃, A is N, G is N, and the combination of R, Q, Ar and D for a compound corresponds to each line of Table A.

Table n.77. Compounds of formula I.n in which “1-R^{xa} is H, “1-R^{xb} is H, “2-R^{xa} is CH₃, “2-R^{xb} is CH₃, R^{ya} is CH₃, A is CH, G is C—CH₃, and the combination of R, Q, Ar and D for a compound corresponds to each line of Table A.

Table n.78. Compounds of formula I.n in which “1-R^{xa} is H, “1-R^{xb} is H, “2-R^{xa} is CH₃, “2-R^{xb} is CH₃, R^{ya} is CH₃, A is N, G is C—CH₃, and the combination of R, Q, Ar and D for a compound corresponds to each line of Table A.

Table n.79. Compounds of formula I.n in which “1-R^{xa} is H, “1-R^{xb} is H, “2-R^{xa} is CH₃, “2-R^{xb} is CH₃, R^{ya} is CH₃, A is CH, G is C—Cl, and the combination of R, Q, Ar and D for a compound corresponds to each line of Table A.

Table n.80. Compounds of formula I.n in which “1-R^{xa} is H, “1-R^{xb} is H, “2-R^{xa} is CH₃, “2-R^{xb} is CH₃, R^{ya} is CH₃, A is N, G is C—Cl, and the combination of R, Q, Ar and D for a compound corresponds to each line of Table A.

Table o.1. Compounds of formula I.o in which R^{xa} is H, R^{xb} is H, R^{ya} is H, A is CH, G is CH, and the combination of R, Q, Ar and D for a compound corresponds to each line of Table A.

Table o.2. Compounds of formula I.o in which R^{xa} is H, R^{xb} is H, R^{ya} is H, A is N, G is CH, and the combination of R, Q, Ar and D for a compound corresponds to each line of Table A.

Table o.3. Compounds of formula I.o in which R^{xa} is H, R^{xb} is H, R^{ya} is H, A is CH, G is N, and the combination of R, Q, Ar and D for a compound corresponds to each line of Table A.

Table o.4. Compounds of formula I.o in which R^{xa} is H, R^{xb} is H, R^{ya} is H, A is N, G is N, and the combination of R, Q, Ar and D for a compound corresponds to each line of Table A.

Table o.5. Compounds of formula I.o in which R^{xa} is H, R^{xb} is H, R^{ya} is H, A is CH, G is C—CH₃, and the combination of R, Q, Ar and D for a compound corresponds to each line of Table A.

Table o.6. Compounds of formula I.o in which R^{xa} is H, R^{xb} is H, R^{ya} is H, A is N, G is C—CH₃, and the combination of R, Q, Ar and D for a compound corresponds to each line of Table A.

Table o.7. Compounds of formula I.o in which R^{xa} is H, R^{xb} is H, R^{ya} is H, A is CH, G is C—Cl, and the combination of R, Q, Ar and D for a compound corresponds to each line of Table A.

Table o.8. Compounds of formula I.o in which R^{xa} is H, R^{xb} is H, R^{ya} is H, A is N, G is C—Cl, and the combination of R, Q, Ar and D for a compound corresponds to each line of Table A.

Table o.9. Compounds of formula I.o in which R^{xa} is CH₃, R^{xb} is H, R^{ya} is H, A is CH, G is CH, and the combination of R, Q, Ar and D for a compound corresponds to each line of Table A.

Table o.10. Compounds of formula I.o in which R^{xa} is CH₃, R^{xb} is H, R^{ya} is H, A is N, G is CH, and the combination of R, Q, Ar and D for a compound corresponds to each line of Table A.

Table o.11. Compounds of formula I.o in which R^{xa} is CH₃, R^{xb} is H, R^{ya} is H, A is CH, G is N, and the combination of R, Q, Ar and D for a compound corresponds to each line of Table A.

Table o.12. Compounds of formula I.o in which R^{xa} is CH₃, R^{xb} is H, R^{ya} is H, A is N, G is N, and the combination of R, Q, Ar and D for a compound corresponds to each line of Table A.

Table o.13. Compounds of formula I.o in which R^{xa} is CH₃, R^{xb} is H, R^{ya} is H, A is CH, G is C—CH₃, and the combination of R, Q, Ar and D for a compound corresponds to each line of Table A.

Table o.14. Compounds of formula I.o in which R^{xa} is CH₃, R^{xb} is H, R^{ya} is H, A is N, G is C—CH₃, and the combination of R, Q, Ar and D for a compound corresponds to each line of Table A.

Table o.15. Compounds of formula I.o in which R^{xa} is CH₃, R^{xb} is H, R^{ya} is H, A is CH, G is C—Cl, and the combination of R, Q, Ar and D for a compound corresponds to each line of Table A.

Table o.16. Compounds of formula I.o in which R^{xa} is CH₃, R^{xb} is H, R^{ya} is H, A is N, G is C—Cl, and the combination of R, Q, Ar and D for a compound corresponds to each line of Table A.

Table o.17. Compounds of formula I.o in which R^{xa} is H, R^{xb} is CH₃, R^{ya} is H, A is CH, G is CH, and the combination of R, Q, Ar and D for a compound corresponds to each line of Table A.

Table o.18. Compounds of formula I.o in which R^{xa} is H, R^{xb} is CH₃, R^{ya} is H, A is N, G is CH, and the combination of R, Q, Ar and D for a compound corresponds to each line of Table A.

Table o.19. Compounds of formula I.o in which R^{xa} is H, R^{xb} is CH₃, R^{ya} is H, A is CH, G is N, and the combination of R, Q, Ar and D for a compound corresponds to each line of Table A.

Table o.20. Compounds of formula I.o in which R^{xa} is H, R^{xb} is CH₃, R^{ya} is H, A is N, G is N, and the combination of R, Q, Ar and D for a compound corresponds to each line of Table A.

Table o.21. Compounds of formula I.o in which R^{xa} is H, R^{xb} is CH₃, R^{ya} is H, A is CH, G is C—C₂H₅, and the combination of R, Q, Ar and D for a compound corresponds to each line of Table A.

Table o.22. Compounds of formula I.o in which R^{xa} is H, R^{xb} is CH₃, R^{ya} is H, A is N, G is C—CH₃, and the combination of R, Q, Ar and D for a compound corresponds to each line of Table A.

Table o.23. Compounds of formula I.o in which R^{xa} is H, R^{xb} is CH₃, R^{ya} is H, A is CH, G is C—Cl, and the combination of R, Q, Ar and D for a compound corresponds to each line of Table A.

Table o.24. Compounds of formula I.o in which R^{xa} is H, R^{xb} is CH₃, R^{ya} is H, A is N, G is C—Cl, and the combination of R, Q, Ar and D for a compound corresponds to each line of Table A.

Table o.25. Compounds of formula I.o in which R^{xa} is H, R^{xb} is H, R^{ya} is CH₃, A is CH, G is CH, and the combination of R, Q, Ar and D for a compound corresponds to each line of Table A.

Table o.26. Compounds of formula I.o in which R^{xa} is H, R^{xb} is H, R^{ya} is CH₃, A is N, G is CH, and the combination of R, Q, Ar and D for a compound corresponds to each line of Table A.

Table o.27. Compounds of formula I.o in which R^{xa} is H, R^{xb} is H, R^{ya} is CH₃, A is CH, G is N, and the combination of R, Q, Ar and D for a compound corresponds to each line of Table A.

Table o.28. Compounds of formula I.o in which R^{xa} is H, R^{xb} is H, R^{ya} is CH₃, A is N, G is N, and the combination of R, Q, Ar and D for a compound corresponds to each line of Table A.

Table o.29. Compounds of formula I.o in which R^{xa} is H, R^{xb} is H, R^{ya} is CH₃, A is CH, G is C—CH₃, and the combination of R, Q, Ar and D for a compound corresponds to each line of Table A.

Table o.30. Compounds of formula I.o in which R^{xa} is H, R^{xb} is H, R^{ya} is CH₃, A is N, G is C—CH₃, and the combination of R, Q, Ar and D for a compound corresponds to each line of Table A.

Table o.31. Compounds of formula I.o in which R^{xa} is H, R^{xb} is H, R^{ya} is CH₃, A is CH, G is C—Cl, and the combination of R, Q, Ar and D for a compound corresponds to each line of Table A.

Table o.32. Compounds of formula I.o in which R^{xa} is H, R^{xb} is H, R^{ya} is CH₃, A is N, G is C—Cl, and the combination of R, Q, Ar and D for a compound corresponds to each line of Table A.

Table o.33. Compounds of formula I.o in which R^{xa} is CH₃, R^{xb} is CH₃, R^{ya} is H, A is CH, G is CH, and the combination of R, Q, Ar and D for a compound corresponds to each line of Table A.

Table o.34. Compounds of formula I.o in which R^{xa} is CH₃, R^{xb} is CH₃, R^{ya} is H, A is N, G is CH, and the combination of R, Q, Ar and D for a compound corresponds to each line of Table A.

Table o.35. Compounds of formula I.o in which R^{xa} is CH₃, R^{xb} is CH₃, R^{ya} is H, A is CH, G is N, and the combination of R, Q, Ar and D for a compound corresponds to each line of Table A.

Table o.36. Compounds of formula I.o in which R^{xa} is CH₃, R^{xb} is CH₃, R^{ya} is H, A is N, G is N, and the combination of R, Q, Ar and D for a compound corresponds to each line of Table A.

Table o.37. Compounds of formula I.o in which R^{xa} is CH₃, R^{xb} is CH₃, R^{ya} is H, A is CH, G is C—CH₃, and the combination of R, Q, Ar and D for a compound corresponds to each line of Table A.

Table o.38. Compounds of formula I.o in which R^{xa} is CH₃, R^{xb} is CH₃, R^{ya} is H, A is N, G is C—CH₃, and the combination of R, Q, Ar and D for a compound corresponds to each line of Table A.

Table o.39. Compounds of formula I.o in which R^{xa} is CH₃, R^{xb} is CH₃, R^{ya} is H, A is CH, G is C—Cl, and the combination of R, Q, Ar and D for a compound corresponds to each line of Table A.

Table o.40. Compounds of formula I.o in which R^{xa} is CH₃, R^{xb} is CH₃, R^{ya} is H, A is N, G is C—Cl, and the combination of R, Q, Ar and D for a compound corresponds to each line of Table A.

Table o.41. Compounds of formula I.o in which R^{xa} is CH₃, R^{xb} is CH₃, R^{ya} is CH₃, A is CH, G is CH, and the combination of R, Q, Ar and D for a compound corresponds to each line of Table A.

Table o.42. Compounds of formula I.o in which R^{xa} is CH₃, R^{xb} is CH₃, R^{ya} is CH₃, A is N, G is CH, and the combination of R, Q, Ar and D for a compound corresponds to each line of Table A.

Table o.43. Compounds of formula I.o in which R^{xa} is CH₃, R^{xb} is CH₃, R^{ya} is CH₃, A is CH, G is N, and the combination of R, Q, Ar and D for a compound corresponds to each line of Table A.

Table o.44. Compounds of formula I.o in which R^{xa} is CH₃, R^{xb} is CH₃, R^{ya} is CH₃, A is N, G is N, and the combination of R, Q, Ar and D for a compound corresponds to each line of Table A.

Table o.45. Compounds of formula I.o in which R^{xa} is CH₃, R^{xb} is CH₃, R^{ya} is CH₃, A is CH, G is C—CH₃, and the combination of R, Q, Ar and D for a compound corresponds to each line of Table A.

Table o.46. Compounds of formula I.o in which R^{xa} is CH₃, R^{xb} is CH₃, R^{ya} is CH₃, A is N, G is C—CH₃, and the combination of R, Q, Ar and D for a compound corresponds to each line of Table A.

Table o.47. Compounds of formula I.o in which R^{xa} is CH₃, R^{xb} is CH₃, R^{ya} is CH₃, A is CH, G is C—Cl, and the combination of R, Q, Ar and D for a compound corresponds to each line of Table A.

Table o.48. Compounds of formula I.o in which R^{xa} is CH₃, R^{xb} is CH₃, R^{ya} is CH₃, A is N, G is C—Cl, and the combination of R, Q, Ar and D for a compound corresponds to each line of Table A.

Table p.1. Compounds of formula I.p in which R^{xa} is H, R^{xb} is H, R^{yc} is H, A is CH, G is CH, and the combination of R, Q, Ar and D for a compound corresponds to each line of Table A.

Table p.2. Compounds of formula I.p in which R^{xa} is H, R^{xb} is H, R^{yc} is H, A is N, G is CH, and the combination of R, Q, Ar and D for a compound corresponds to each line of Table A.

Table p.3. Compounds of formula I.p in which R^{xa} is H, R^{xb} is H, R^{yc} is H, A is CH, G is N, and the combination of R, Q, Ar and D for a compound corresponds to each line of Table A.

Table p.4. Compounds of formula I.p in which R^{xa} is H, R^{xb} is H, R^{yc} is H, A is N, G is N, and the combination of R, Q, Ar and D for a compound corresponds to each line of Table A.

Table p.5. Compounds of formula I.p in which R^{xa} is H, R^{xb} is H, R^{yc} is H, A is CH, G is C—CH₃, and the combination of R, Q, Ar and D for a compound corresponds to each line of Table A.

Table p.6. Compounds of formula I.p in which R^{xa} is H, R^{xb} is H, R^{yc} is H, A is N, G is C—CH₃, and the combination of R, Q, Ar and D for a compound corresponds to each line of Table A.

Table p.7. Compounds of formula I.p in which R^{xa} is H, R^{xb} is H, R^{yc} is H, A is CH, G is C—Cl, and the combination of R, Q, Ar and D for a compound corresponds to each line of Table A.

Table p.8. Compounds of formula I.p in which R^{xa} is H, R^{xb} is H, R^{yc} is H, A is N, G is C—Cl, and the combination of R, Q, Ar and D for a compound corresponds to each line of Table A.

Table p.9. Compounds of formula I.p in which R^{xa} is CH₃, R^{xb} is H, R^{yc} is H, A is CH, G is CH, and the combination of R, Q, Ar and D for a compound corresponds to each line of Table A.

Table p.10. Compounds of formula I.p in which R^{xa} is CH₃, R^{xb} is H, R^{yc} is H, A is N, G is CH, and the combination of R, Q, Ar and D for a compound corresponds to each line of Table A.

Table p.11. Compounds of formula I.p in which R^{xa} is CH₃, R^{xb} is H, R^{yc} is H, A is CH, G is N, and the combination of R, Q, Ar and D for a compound corresponds to each line of Table A.

Table p.12. Compounds of formula I.p in which R^{xa} is CH₃, R^{xb} is H, R^{yc} is H, A is N, G is N, and the combination of R, Q, Ar and D for a compound corresponds to each line of Table A.

Table p.13. Compounds of formula I.p in which R^{xa} is CH₃, R^{xb} is H, R^{yc} is H, A is CH, G is C—CH₃, and the combination of R, Q, Ar and D for a compound corresponds to each line of Table A.

Table p.14. Compounds of formula I.p in which R^{xa} is CH₃, R^{xb} is H, R^{yc} is H, A is N, G is C—CH₃, and the combination of R, Q, Ar and D for a compound corresponds to each line of Table A.

Table p.15. Compounds of formula I.p in which R^{xa} is CH₃, R^{xb} is H, R^{yc} is H, A is CH, G is C—Cl, and the combination of R, Q, Ar and D for a compound corresponds to each line of Table A.

Table p.16. Compounds of formula I.p in which R^{xa} is CH₃, R^{xb} is H, R^{yc} is H, A is N, G is C—Cl, and the combination of R, Q, Ar and D for a compound corresponds to each line of Table A.

Table p.17. Compounds of formula I.p in which R^{xa} is H, R^{xb} is CH₃, R^{yc} is H, A is CH, G is CH, and the combination of R, Q, Ar and D for a compound corresponds to each line of Table A.

Table p.18. Compounds of formula I.p in which R^{xa} is H, R^{xb} is CH₃, R^{yc} is H, A is N, G is CH, and the combination of R, Q, Ar and D for a compound corresponds to each line of Table A.

Table p.19. Compounds of formula I.p in which R^{xa} is H, R^{xb} is CH₃, R^{yc} is H, A is CH, G is N, and the combination of R, Q, Ar and D for a compound corresponds to each line of Table A.

Table p.20. Compounds of formula I.p in which R^{xa} is H, R^{xb} is CH₃, R^{yc} is H, A is N, G is N, and the combination of R, Q, Ar and D for a compound corresponds to each line of Table A.

Table p.21. Compounds of formula I.p in which R^{xa} is H, R^{xb} is CH₃, R^{yc} is H, A is CH, G is C—CH₃CH₃, and the combination of R, Q, Ar and D for a compound corresponds to each line of Table A.

Table p.22. Compounds of formula I.p in which R^{xa} is H, R^{xb} is CH₃, R^{yc} is H, A is N, G is C—CH₃, and the combination of R, Q, Ar and D for a compound corresponds to each line of Table A.

Table p.23. Compounds of formula I.p in which R^{xa} is H, R^{xb} is CH₃, R^{yc} is H, A is CH, G is C—Cl, and the combination of R, Q, Ar and D for a compound corresponds to each line of Table A.

Table p.24. Compounds of formula I.p in which R^{xa} is H, R^{xb} is CH₃, R^{yc} is H, A is N, G is C—Cl, and the combination of R, Q, Ar and D for a compound corresponds to each line of Table A.

Table p.25. Compounds of formula I.p in which R^{xa} is H, R^{xb} is H, R^{yc} is CH₃, A is CH, G is CH, and the combination of R, Q, Ar and D for a compound corresponds to each line of Table A.

Table p.26. Compounds of formula I.p in which R^{xa} is H, R^{xb} is H, R^{yc} is CH₃, A is N, G is CH, and the combination of R, Q, Ar and D for a compound corresponds to each line of Table A.

Table p.27. Compounds of formula I.p in which R^{xa} is H, R^{xb} is H, R^{yc} is CH₃, A is CH, G is N, and the combination of R, Q, Ar and D for a compound corresponds to each line of Table A.

Table p.28. Compounds of formula I.p in which R^{xa} is H, R^{xb} is H, R^{yc} is CH₃, A is N, G is N, and the combination of R, Q, Ar and D for a compound corresponds to each line of Table A.

Table p.29. Compounds of formula I.p in which R^{xa} is H, R^{xb} is H, R^{yc} is CH₃, A is CH, G is C—CH₃, and the combination of R, Q, Ar and D for a compound corresponds to each line of Table A.

Table p.30. Compounds of formula I.p in which R^{xa} is H, R^{xb} is H, R^{yc} is CH₃, A is N, G is C—CH₃, and the combination of R, Q, Ar and D for a compound corresponds to each line of Table A.

Table p.31. Compounds of formula I.p in which R^{xa} is H, R^{xb} is H, R^{yc} is CH₃, A is CH, G is C—Cl, and the combination of R, Q, Ar and D for a compound corresponds to each line of Table A.

Table p.32. Compounds of formula I.p in which R^{xa} is H, R^{xb} is H, R^{yc} is CH₃, A is N, G is C—Cl, and the combination of R, Q, Ar and D for a compound corresponds to each line of Table A.

Table p.33. Compounds of formula I.p in which R^{xa} is CH₃, R^{xb} is CH₃, R^{yc} is H, A is CH, G is CH, and the combination of R, Q, Ar and D for a compound corresponds to each line of Table A.

Table p.34. Compounds of formula I.p in which R^{xa} is CH₃, R^{xb} is CH₃, R^{yc} is H, A is N, G is CH, and the combination of R, Q, Ar and D for a compound corresponds to each line of Table A.

Table p.35. Compounds of formula I.p in which R^{xa} is CH₃, R^{xb} is CH₃, R^{yc} is H, A is CH, G is N, and the combination of R, Q, Ar and D for a compound corresponds to each line of Table A.

Table p.36. Compounds of formula I.p in which R^{xa} is CH₃, R^{xb} is CH₃, R^{yc} is H, A is N, G is N, and the combination of R, Q, Ar and D for a compound corresponds to each line of Table A.

Table p.37. Compounds of formula I.p in which R^{xa} is CH₃, R^{xb} is CH₃, R^{yc} is H, A is CH, G is C—CH₃, and the combination of R, Q, Ar and D for a compound corresponds to each line of Table A.

Table p.38. Compounds of formula I.p in which R^{xa} is CH₃, R^{xb} is CH₃, R^{yc} is H, A is N, G is C—CH₃, and the combination of R, Q, Ar and D for a compound corresponds to each line of Table A.

Table p.39. Compounds of formula I.p in which R^{xa} is CH₃, R^{xb} is CH₃, R^{yc} is H, A is CH, G is C—Cl, and the combination of R, Q, Ar and D for a compound corresponds to each line of Table A.

Table p.40. Compounds of formula I.p in which R^{xa} is CH₃, R^{xb} is CH₃, R^{yc} is H, A is N, G is C—Cl, and the combination of R, Q, Ar and D for a compound corresponds to each line of Table A.

Table p.41. Compounds of formula I.p in which R^{xa} is CH₃, R^{xb} is CH₃, R^{yc} is CH₃, A is CH, G is CH, and the combination of R, Q, Ar and D for a compound corresponds to each line of Table A.

Table p.42. Compounds of formula I.p in which R^{xa} is CH₃, R^{xb} is CH₃, R^{yc} is CH₃, A is N, G is CH, and the combination of R, Q, Ar and D for a compound corresponds to each line of Table A.

Table p.43. Compounds of formula I.p in which R^{xa} is CH₃, R^{xb} is CH₃, R^{yc} is CH₃, A is CH, G is N, and the combination of R, Q, Ar and D for a compound corresponds to each line of Table A.

Table p.44. Compounds of formula I.p in which R^{xa} is CH₃, R^{xb} is CH₃, R^{yc} is CH₃, A is N, G is N, and the combination of R, Q, Ar and D for a compound corresponds to each line of Table A.

Table p.45. Compounds of formula I.p in which R^{xa} is CH₃, R^{xb} is CH₃, R^{yc} is CH₃, A is CH, G is C—CH₃, and the combination of R, Q, Ar and D for a compound corresponds to each line of Table A.

Table p.46. Compounds of formula I.p in which R^{xa} is CH₃, R^{xb} is CH₃, R^{yc} is CH₃, A is N, G is C—CH₃, and the combination of R, Q, Ar and D for a compound corresponds to each line of Table A.

Table p.47. Compounds of formula I.p in which R^{xa} is CH₃, R^{xb} is CH₃, R^{yc} is CH₃, A is CH, G is C—Cl, and the combination of R, Q, Ar and D for a compound corresponds to each line of Table A.

Table p.48. Compounds of formula I.p in which R^{xa} is CH₃, R^{xb} is CH₃, R^{yc} is CH₃, A is N, G is C—Cl, and the combination of R, Q, Ar and D for a compound corresponds to each line of Table A.

TABLE A

| Line | R | Q | Ar | D |
|---|---|---|---|---|
| 1 | H | NH | Ar¹ | R¹¹-1 |
| 2 | H | NH | Ar¹ | R¹¹-2 |
| 3 | H | NH | Ar¹ | R¹¹-3 |
| 4 | H | NH | Ar¹ | R¹¹-4 |
| 5 | H | NH | Ar¹ | R¹¹-5 |
| 6 | H | NH | Ar¹ | R¹¹-6 |
| 7 | H | NH | Ar¹ | R¹¹-7 |
| 8 | H | NH | Ar¹ | R¹¹-8 |
| 9 | H | NH | Ar¹ | R¹¹-9 |
| 10 | H | NH | Ar¹ | R¹¹-10 |
| 11 | H | NH | Ar¹ | R¹¹-11 |
| 12 | H | NH | Ar¹ | R¹¹-12 |
| 13 | H | NH | Ar¹ | R¹¹-13 |
| 14 | H | NH | Ar¹ | R¹¹-14 |
| 15 | H | NH | Ar¹ | R¹¹-15 |
| 16 | H | NH | Ar¹ | R¹¹-16 |
| 17 | H | NH | Ar¹ | R¹¹-17 |
| 18 | H | NH | Ar¹ | R¹¹-18 |
| 19 | H | NH | Ar¹ | R¹¹-19 |
| 20 | H | NH | Ar¹ | R¹¹-20 |
| 21 | H | NH | Ar¹ | R¹¹-21 |
| 22 | H | NH | Ar¹ | R¹¹-22 |
| 23 | H | NH | Ar¹ | R¹¹-23 |
| 24 | H | NH | Ar¹ | R¹¹-24 |
| 25 | H | NH | Ar¹ | R¹¹-25 |
| 26 | H | NH | Ar¹ | R¹¹-26 |
| 27 | H | NH | Ar¹ | R¹¹-27 |
| 28 | H | NH | Ar¹ | R¹¹-28 |
| 29 | H | NH | Ar¹ | R¹¹-29 |
| 30 | H | NH | Ar¹ | A¹¹-1a |
| 31 | H | NH | Ar¹ | A¹¹-1b |
| 32 | H | NH | Ar¹ | A¹¹-2a |
| 33 | H | NH | Ar¹ | A¹¹-2b |
| 34 | H | NH | Ar¹ | A¹¹-3a |
| 35 | H | NH | Ar¹ | A¹¹-3b |
| 36 | H | NH | Ar² | R¹¹-1 |
| 37 | H | NH | Ar² | R¹¹-2 |
| 38 | H | NH | Ar² | R¹¹-3 |
| 39 | H | NH | Ar² | R¹¹-4 |
| 40 | H | NH | Ar² | R¹¹-5 |
| 41 | H | NH | Ar² | R¹¹-6 |
| 42 | H | NH | Ar² | R¹¹-7 |
| 43 | H | NH | Ar² | R¹¹-8 |
| 44 | H | NH | Ar² | R¹¹-9 |
| 45 | H | NH | Ar² | R¹¹-10 |
| 46 | H | NH | Ar² | R¹¹-11 |
| 47 | H | NH | Ar² | R¹¹-12 |
| 48 | H | NH | Ar² | R¹¹-13 |
| 49 | H | NH | Ar² | R¹¹-14 |
| 50 | H | NH | Ar² | R¹¹-15 |
| 51 | H | NH | Ar² | R¹¹-16 |
| 52 | H | NH | Ar² | R¹¹-17 |
| 53 | H | NH | Ar² | R¹¹-18 |
| 54 | H | NH | Ar² | R¹¹-19 |
| 55 | H | NH | Ar² | R¹¹-20 |
| 56 | H | NH | Ar² | R¹¹-21 |
| 57 | H | NH | Ar² | R¹¹-22 |
| 58 | H | NH | Ar² | R¹¹-23 |
| 59 | H | NH | Ar² | R¹¹-24 |
| 60 | H | NH | Ar² | R¹¹-25 |
| 61 | H | NH | Ar² | R¹¹-26 |
| 62 | H | NH | Ar² | R¹¹-27 |
| 63 | H | NH | Ar² | R¹¹-28 |
| 64 | H | NH | Ar² | R¹¹-29 |
| 65 | H | NH | Ar² | A¹¹-1a |
| 66 | H | NH | Ar² | A¹¹-1b |
| 67 | H | NH | Ar² | A¹¹-2a |
| 68 | H | NH | Ar² | A¹¹-2b |
| 69 | H | NH | Ar² | A¹¹-3a |
| 70 | H | NH | Ar² | A¹¹-3b |
| 71 | H | NH | Ar³ | R¹¹-1 |
| 72 | H | NH | Ar³ | R¹¹-2 |
| 73 | H | NH | Ar³ | R¹¹-3 |
| 74 | H | NH | Ar³ | R¹¹-4 |
| 75 | H | NH | Ar³ | R¹¹-5 |
| 76 | H | NH | Ar³ | R¹¹-6 |
| 77 | H | NH | Ar³ | R¹¹-7 |
| 78 | H | NH | Ar³ | R¹¹-8 |
| 79 | H | NH | Ar³ | R¹¹-9 |
| 80 | H | NH | Ar³ | R¹¹-10 |
| 81 | H | NH | Ar³ | R¹¹-11 |
| 82 | H | NH | Ar³ | R¹¹-12 |
| 83 | H | NH | Ar³ | R¹¹-13 |
| 84 | H | NH | Ar³ | R¹¹-14 |
| 85 | H | NH | Ar³ | R¹¹-15 |
| 86 | H | NH | Ar³ | R¹¹-16 |
| 87 | H | NH | Ar³ | R¹¹-17 |
| 88 | H | NH | Ar³ | R¹¹-18 |
| 89 | H | NH | Ar³ | R¹¹-19 |
| 90 | H | NH | Ar³ | R¹¹-20 |
| 91 | H | NH | Ar³ | R¹¹-21 |
| 92 | H | NH | Ar³ | R¹¹-22 |
| 93 | H | NH | Ar³ | R¹¹-23 |
| 94 | H | NH | Ar³ | R¹¹-24 |
| 95 | H | NH | Ar³ | R¹¹-25 |
| 96 | H | NH | Ar³ | R¹¹-26 |
| 97 | H | NH | Ar³ | R¹¹-27 |
| 98 | H | NH | Ar³ | R¹¹-28 |
| 99 | H | NH | Ar³ | R¹¹-29 |
| 100 | H | NH | Ar³ | A¹¹-1a |
| 101 | H | NH | Ar³ | A¹¹-1b |
| 102 | H | NH | Ar³ | A¹¹-2a |
| 103 | H | NH | Ar³ | A¹¹-2b |
| 104 | H | NH | Ar³ | A¹¹-3a |
| 105 | H | NH | Ar⁴ | A¹¹-3b |
| 106 | H | NH | Ar⁴ | R¹¹-1 |
| 107 | H | NH | Ar⁴ | R¹¹-2 |
| 108 | H | NH | Ar⁴ | R¹¹-3 |
| 109 | H | NH | Ar⁴ | R¹¹-4 |
| 110 | H | NH | Ar⁴ | R¹¹-5 |
| 111 | H | NH | Ar⁴ | R¹¹-6 |
| 112 | H | NH | Ar⁴ | R¹¹-7 |
| 113 | H | NH | Ar⁴ | R¹¹-8 |
| 114 | H | NH | Ar⁴ | R¹¹-9 |
| 115 | H | NH | Ar⁴ | R¹¹-10 |
| 116 | H | NH | Ar⁴ | R¹¹-11 |
| 117 | H | NH | Ar⁴ | R¹¹-12 |
| 118 | H | NH | Ar⁴ | R¹¹-13 |
| 119 | H | NH | Ar⁴ | R¹¹-14 |
| 120 | H | NH | Ar⁴ | R¹¹-15 |
| 121 | H | NH | Ar⁴ | R¹¹-16 |
| 122 | H | NH | Ar⁴ | R¹¹-17 |
| 123 | H | NH | Ar⁴ | R¹¹-18 |
| 124 | H | NH | Ar⁴ | R¹¹-19 |
| 125 | H | NH | Ar⁴ | R¹¹-20 |
| 126 | H | NH | Ar⁴ | R¹¹-21 |
| 127 | H | NH | Ar⁴ | R¹¹-22 |
| 128 | H | NH | Ar⁴ | R¹¹-23 |
| 129 | H | NH | Ar⁴ | R¹¹-24 |
| 130 | H | NH | Ar⁴ | R¹¹-25 |
| 131 | H | NH | Ar⁴ | R¹¹-26 |
| 132 | H | NH | Ar⁴ | R¹¹-27 |
| 133 | H | NH | Ar⁴ | R¹¹-28 |
| 134 | H | NH | Ar⁴ | R¹¹-29 |
| 135 | H | NH | Ar⁴ | A¹¹-1a |
| 136 | H | NH | Ar⁴ | A¹¹-1b |
| 137 | H | NH | Ar⁴ | A¹¹-2a |
| 138 | H | NH | Ar⁴ | A¹¹-2b |
| 139 | H | NH | Ar⁴ | A¹¹-3a |
| 140 | H | NH | Ar⁴ | A¹¹-3b |
| 141 | H | NH | Ar⁵ | R¹¹-1 |
| 142 | H | NH | Ar⁵ | R¹¹-2 |
| 143 | H | NH | Ar⁵ | R¹¹-3 |
| 144 | H | NH | Ar⁵ | R¹¹-4 |
| 145 | H | NH | Ar⁵ | R¹¹-5 |
| 146 | H | NH | Ar⁵ | R¹¹-6 |
| 147 | H | NH | Ar⁵ | R¹¹-7 |
| 148 | H | NH | Ar⁵ | R¹¹-8 |
| 149 | H | NH | Ar⁵ | R¹¹-9 |
| 150 | H | NH | Ar⁵ | R¹¹-10 |
| 151 | H | NH | Ar⁵ | R¹¹-11 |
| 152 | H | NH | Ar⁵ | R¹¹-12 |
| 153 | H | NH | Ar⁵ | R¹¹-13 |
| 154 | H | NH | Ar⁵ | R¹¹-14 |
| 155 | H | NH | Ar⁵ | R¹¹-15 |
| 156 | H | NH | Ar⁵ | R¹¹-16 |
| 157 | H | NH | Ar⁵ | R¹¹-17 |
| 158 | H | NH | Ar⁵ | R¹¹-18 |
| 159 | H | NH | Ar⁵ | R¹¹-19 |
| 160 | H | NH | Ar⁵ | R¹¹-20 |
| 161 | H | NH | Ar⁵ | R¹¹-21 |
| 162 | H | NH | Ar⁵ | R¹¹-22 |
| 163 | H | NH | Ar⁵ | R¹¹-23 |
| 164 | H | NH | Ar⁵ | R¹¹-24 |
| 165 | H | NH | Ar⁵ | R¹¹-25 |
| 166 | H | NH | Ar⁵ | R¹¹-26 |
| 167 | H | NH | Ar⁵ | R¹¹-27 |
| 168 | H | NH | Ar⁵ | R¹¹-28 |
| 169 | H | NH | Ar⁵ | R¹¹-29 |
| 170 | H | NH | Ar⁵ | A¹¹-1a |
| 171 | H | NH | Ar⁵ | A¹¹-1b |
| 172 | H | NH | Ar⁵ | A¹¹-2a |
| 173 | H | NH | Ar⁵ | A¹¹-2b |
| 174 | H | NH | Ar⁵ | A¹¹-3a |
| 175 | H | NH | Ar⁵ | A¹¹-3b |
| 176 | H | NH | Ar⁶ | R¹¹-1 |
| 177 | H | NH | Ar⁶ | R¹¹-2 |
| 178 | H | NH | Ar⁶ | R¹¹-3 |
| 179 | H | NH | Ar⁶ | R¹¹-4 |
| 180 | H | NH | Ar⁶ | R¹¹-5 |
| 181 | H | NH | Ar⁶ | R¹¹-6 |
| 182 | H | NH | Ar⁶ | R¹¹-7 |
| 183 | H | NH | Ar⁶ | R¹¹-8 |
| 184 | H | NH | Ar⁶ | R¹¹-9 |
| 185 | H | NH | Ar⁶ | R¹¹-10 |
| 186 | H | NH | Ar⁶ | R¹¹-11 |
| 187 | H | NH | Ar⁶ | R¹¹-12 |
| 188 | H | NH | Ar⁶ | R¹¹-13 |
| 189 | H | NH | Ar⁶ | R¹¹-14 |
| 190 | H | NH | Ar⁶ | R¹¹-15 |
| 191 | H | NH | Ar⁶ | R¹¹-16 |
| 192 | H | NH | Ar⁶ | R¹¹-17 |
| 193 | H | NH | Ar⁶ | R¹¹-18 |
| 194 | H | NH | Ar⁶ | R¹¹-19 |
| 195 | H | NH | Ar⁶ | R¹¹-20 |
| 196 | H | NH | Ar⁶ | R¹¹-21 |
| 197 | H | NH | Ar⁶ | R¹¹-22 |
| 198 | H | NH | Ar⁶ | R¹¹-23 |
| 199 | H | NH | Ar⁶ | R¹¹-24 |
| 200 | H | NH | Ar⁶ | R¹¹-25 |
| 201 | H | NH | Ar⁶ | R¹¹-26 |
| 202 | H | NH | Ar⁶ | R¹¹-27 |
| 203 | H | NH | Ar⁶ | R¹¹-28 |
| 204 | H | NH | Ar⁶ | R¹¹-29 |
| 205 | H | NH | Ar⁶ | A¹¹-1a |
| 206 | H | NH | Ar⁶ | A¹¹-1b |
| 207 | H | NH | Ar⁶ | A¹¹-2a |
| 208 | H | NH | Ar⁶ | A¹¹-2b |
| 209 | H | NH | Ar⁶ | A¹¹-3a |
| 210 | H | NH | Ar⁶ | A¹¹-3b |
| 211 | H | NH | Ar⁷ | R¹¹-1 |
| 212 | H | NH | Ar⁷ | R¹¹-2 |
| 213 | H | NH | Ar⁷ | R¹¹-3 |
| 214 | H | NH | Ar⁷ | R¹¹-4 |
| 215 | H | NH | Ar⁷ | R¹¹-5 |
| 216 | H | NH | Ar⁷ | R¹¹-6 |
| 217 | H | NH | Ar⁷ | R¹¹-7 |
| 218 | H | NH | Ar⁷ | R¹¹-8 |
| 219 | H | NH | Ar⁷ | R¹¹-9 |
| 220 | H | NH | Ar⁷ | R¹¹-10 |
| 221 | H | NH | Ar⁷ | R¹¹-11 |
| 222 | H | NH | Ar⁷ | R¹¹-12 |
| 223 | H | NH | Ar⁷ | R¹¹-13 |
| 224 | H | NH | Ar⁷ | R¹¹-14 |
| 225 | H | NH | Ar⁷ | R¹¹-15 |
| 226 | H | NH | Ar⁷ | R¹¹-16 |
| 227 | H | NH | Ar⁷ | R¹¹-17 |
| 228 | H | NH | Ar⁷ | R¹¹-18 |
| 229 | H | NH | Ar⁷ | R¹¹-19 |
| 230 | H | NH | Ar⁷ | R¹¹-20 |
| 231 | H | NH | Ar⁷ | R¹¹-21 |
| 232 | H | NH | Ar⁷ | R¹¹-22 |
| 233 | H | NH | Ar⁷ | R¹¹-23 |
| 234 | H | NH | Ar⁷ | R¹¹-24 |
| 235 | H | NH | Ar⁷ | R¹¹-25 |
| 236 | H | NH | Ar⁷ | R¹¹-26 |
| 237 | H | NH | Ar⁷ | R¹¹-27 |
| 238 | H | NH | Ar⁷ | R¹¹-28 |
| 239 | H | NH | Ar⁷ | R¹¹-29 |
| 240 | H | NH | Ar⁷ | A¹¹-1a |
| 241 | H | NH | Ar⁷ | A¹¹-1b |
| 242 | H | NH | Ar⁷ | A¹¹-2a |
| 243 | H | NH | Ar⁷ | A¹¹-2b |
| 244 | H | NH | Ar⁷ | A¹¹-3a |
| 245 | H | NH | Ar⁷ | A¹¹-3b |
| 246 | H | NH | Ar⁸ | R¹¹-1 |
| 247 | H | NH | Ar⁸ | R¹¹-2 |
| 248 | H | NH | Ar⁸ | R¹¹-3 |
| 249 | H | NH | Ar⁸ | R¹¹-4 |
| 250 | H | NH | Ar⁸ | R¹¹-5 |
| 251 | H | NH | Ar⁸ | R¹¹-6 |
| 252 | H | NH | Ar⁸ | R¹¹-7 |
| 253 | H | NH | Ar⁸ | R¹¹-8 |
| 254 | H | NH | Ar⁸ | R¹¹-9 |
| 255 | H | NH | Ar⁸ | R¹¹-10 |
| 256 | H | NH | Ar⁸ | R¹¹-11 |
| 257 | H | NH | Ar⁸ | R¹¹-12 |
| 258 | H | NH | Ar⁸ | R¹¹-13 |
| 259 | H | NH | Ar⁸ | R¹¹-14 |
| 260 | H | NH | Ar⁸ | R¹¹-15 |
| 261 | H | NH | Ar⁸ | R¹¹-16 |
| 262 | H | NH | Ar⁸ | R¹¹-17 |
| 263 | H | NH | Ar⁸ | R¹¹-18 |
| 264 | H | NH | Ar⁸ | R¹¹-19 |
| 265 | H | NH | Ar⁸ | R¹¹-20 |
| 266 | H | NH | Ar⁸ | R¹¹-21 |
| 267 | H | NH | Ar⁸ | R¹¹-22 |
| 268 | H | NH | Ar⁸ | R¹¹-23 |
| 269 | H | NH | Ar⁸ | R¹¹-24 |
| 270 | H | NH | Ar⁸ | R¹¹-25 |
| 271 | H | NH | Ar⁸ | R¹¹-26 |
| 272 | H | NH | Ar⁸ | R¹¹-27 |
| 273 | H | NH | Ar⁸ | R¹¹-28 |
| 274 | H | NH | Ar⁸ | R¹¹-29 |
| 275 | H | NH | Ar⁸ | A¹¹-1a |
| 276 | H | NH | Ar⁸ | A¹¹-1b |
| 277 | H | NH | Ar⁸ | A¹¹-2a |
| 278 | H | NH | Ar⁸ | A¹¹-2b |
| 279 | H | NH | Ar⁸ | A¹¹-3a |
| 280 | H | NH | Ar⁸ | A¹¹-3b |
| 281 | H | NH | Ar⁹ | R¹¹-1 |
| 282 | H | NH | Ar⁹ | R¹¹-2 |
| 283 | H | NH | Ar⁹ | R¹¹-3 |
| 284 | H | NH | Ar⁹ | R¹¹-4 |
| 285 | H | NH | Ar⁹ | R¹¹-5 |
| 286 | H | NH | Ar⁹ | R¹¹-6 |
| 287 | H | NH | Ar⁹ | R¹¹-7 |
| 288 | H | NH | Ar⁹ | R¹¹-8 |
| 289 | H | NH | Ar⁹ | R¹¹-9 |
| 290 | H | NH | Ar⁹ | R¹¹-10 |
| 291 | H | NH | Ar⁹ | R¹¹-11 |
| 292 | H | NH | Ar⁹ | R¹¹-12 |
| 293 | H | NH | Ar⁹ | R¹¹-13 |
| 294 | H | NH | Ar⁹ | R¹¹-14 |
| 295 | H | NH | Ar⁹ | R¹¹-15 |
| 296 | H | NH | Ar⁹ | R¹¹-16 |
| 297 | H | NH | Ar⁹ | R¹¹-17 |
| 298 | H | NH | Ar⁹ | R¹¹-18 |
| 299 | H | NH | Ar⁹ | R¹¹-19 |
| 300 | H | NH | Ar⁹ | R¹¹-20 |
| 301 | H | NH | Ar⁹ | R¹¹-21 |
| 302 | H | NH | Ar⁹ | R¹¹-22 |
| 303 | H | NH | Ar⁹ | R¹¹-23 |
| 304 | H | NH | Ar⁹ | R¹¹-24 |
| 305 | H | NH | Ar⁹ | R¹¹-25 |
| 306 | H | NH | Ar⁹ | R¹¹-26 |
| 307 | H | NH | Ar⁹ | R¹¹-27 |
| 308 | H | NH | Ar⁹ | R¹¹-28 |
| 309 | H | NH | Ar⁹ | R¹¹-29 |
| 310 | H | NH | Ar⁹ | A¹¹-1a |
| 311 | H | NH | Ar⁹ | A¹¹-1b |
| 312 | H | NH | Ar⁹ | A¹¹-2a |
| 313 | H | NH | Ar⁹ | A¹¹-2b |
| 314 | H | NH | Ar⁹ | A¹¹-3a |
| 315 | H | NH | Ar⁹ | A¹¹-3b |
| 316 | H | NH | Ar¹⁰ | R¹¹-1 |
| 317 | H | NH | Ar¹⁰ | R¹¹-2 |
| 318 | H | NH | Ar¹⁰ | R¹¹-3 |
| 319 | H | NH | Ar¹⁰ | R¹¹-4 |
| 320 | H | NH | Ar¹⁰ | R¹¹-5 |
| 321 | H | NH | Ar¹⁰ | R¹¹-6 |
| 322 | H | NH | Ar¹⁰ | R¹¹-7 |
| 323 | H | NH | Ar¹⁰ | R¹¹-8 |
| 324 | H | NH | Ar¹⁰ | R¹¹-9 |
| 325 | H | NH | Ar¹⁰ | R¹¹-10 |
| 326 | H | NH | Ar¹⁰ | R¹¹-11 |
| 327 | H | NH | Ar¹⁰ | R¹¹-12 |
| 328 | H | NH | Ar¹⁰ | R¹¹-13 |
| 329 | H | NH | Ar¹⁰ | R¹¹-14 |
| 330 | H | NH | Ar¹⁰ | R¹¹-15 |
| 331 | H | NH | Ar¹⁰ | R¹¹-16 |
| 332 | H | NH | Ar¹⁰ | R¹¹-17 |
| 333 | H | NH | Ar¹⁰ | R¹¹-18 |
| 334 | H | NH | Ar¹⁰ | R¹¹-19 |
| 335 | H | NH | Ar¹⁰ | R¹¹-20 |
| 336 | H | NH | Ar¹⁰ | R¹¹-21 |
| 337 | H | NH | Ar¹⁰ | R¹¹-22 |
| 338 | H | NH | Ar¹⁰ | R¹¹-23 |
| 339 | H | NH | Ar¹⁰ | R¹¹-24 |
| 340 | H | NH | Ar¹⁰ | R¹¹-25 |
| 341 | H | NH | Ar¹⁰ | R¹¹-26 |
| 342 | H | NH | Ar¹⁰ | R¹¹-27 |
| 343 | H | NH | Ar¹⁰ | R¹¹-28 |
| 344 | H | NH | Ar¹⁰ | R¹¹-29 |
| 345 | H | NH | Ar¹⁰ | A¹¹-1a |
| 346 | H | NH | Ar¹⁰ | A¹¹-1b |
| 347 | H | NH | Ar¹⁰ | A¹¹-2a |
| 348 | H | NH | Ar¹⁰ | A¹¹-2b |
| 349 | H | NH | Ar¹⁰ | A¹¹-3a |
| 350 | H | NH | Ar¹⁰ | A¹¹-3b |
| 351 | H | NH | Ar¹¹ | R¹¹-1 |
| 352 | H | NH | Ar¹¹ | R¹¹-2 |
| 353 | H | NH | Ar¹¹ | R¹¹-3 |
| 354 | H | NH | Ar¹¹ | R¹¹-4 |
| 355 | H | NH | Ar¹¹ | R¹¹-5 |
| 356 | H | NH | Ar¹¹ | R¹¹-6 |
| 357 | H | NH | Ar¹¹ | R¹¹-7 |
| 358 | H | NH | Ar¹¹ | R¹¹-8 |
| 359 | H | NH | Ar¹¹ | R¹¹-9 |
| 360 | H | NH | Ar¹¹ | R¹¹-10 |
| 361 | H | NH | Ar¹¹ | R¹¹-11 |
| 362 | H | NH | Ar¹¹ | R¹¹-12 |
| 363 | H | NH | Ar¹¹ | R¹¹-13 |
| 364 | H | NH | Ar¹¹ | R¹¹-14 |
| 365 | H | NH | Ar¹¹ | R¹¹-15 |
| 366 | H | NH | Ar¹¹ | R¹¹-16 |
| 367 | H | NH | Ar¹¹ | R¹¹-17 |
| 368 | H | NH | Ar¹¹ | R¹¹-18 |
| 369 | H | NH | Ar¹¹ | R¹¹-19 |
| 370 | H | NH | Ar¹¹ | R¹¹-20 |
| 371 | H | NH | Ar¹¹ | R¹¹-21 |
| 372 | H | NH | Ar¹¹ | R¹¹-22 |
| 373 | H | NH | Ar¹¹ | R¹¹-23 |
| 374 | H | NH | Ar¹¹ | R¹¹-24 |
| 375 | H | NH | Ar¹¹ | R¹¹-25 |
| 376 | H | NH | Ar¹¹ | R¹¹-26 |
| 377 | H | NH | Ar¹¹ | R¹¹-27 |
| 378 | H | NH | Ar¹¹ | R¹¹-28 |
| 379 | H | NH | Ar¹¹ | R¹¹-29 |
| 380 | H | NH | Ar¹¹ | A¹¹-1a |
| 381 | H | NH | Ar¹¹ | A¹¹-1b |
| 382 | H | NH | Ar¹¹ | A¹¹-2a |
| 383 | H | NH | Ar¹¹ | A¹¹-2b |
| 384 | H | NH | Ar¹¹ | A¹¹-3a |
| 385 | H | NH | Ar¹¹ | A¹¹-3b |
| 386 | H | NH | Ar¹² | R¹¹-1 |
| 387 | H | NH | Ar¹² | R¹¹-2 |
| 388 | H | NH | Ar¹² | R¹¹-3 |
| 389 | H | NH | Ar¹² | R¹¹-4 |
| 390 | H | NH | Ar¹² | R¹¹-5 |
| 391 | H | NH | Ar¹² | R¹¹-6 |
| 392 | H | NH | Ar¹² | R¹¹-7 |
| 393 | H | NH | Ar¹² | R¹¹-8 |
| 394 | H | NH | Ar¹² | R¹¹-9 |
| 395 | H | NH | Ar¹² | R¹¹-10 |
| 396 | H | NH | Ar¹² | R¹¹-11 |
| 397 | H | NH | Ar¹² | R¹¹-12 |
| 398 | H | NH | Ar¹² | R¹¹-13 |
| 399 | H | NH | Ar¹² | R¹¹-14 |
| 400 | H | NH | Ar¹² | R¹¹-15 |
| 401 | H | NH | Ar¹² | R¹¹-16 |
| 402 | H | NH | Ar¹² | R¹¹-17 |
| 403 | H | NH | Ar¹² | R¹¹-18 |
| 404 | H | NH | Ar¹² | R¹¹-19 |
| 405 | H | NH | Ar¹² | R¹¹-20 |
| 406 | H | NH | Ar¹² | R¹¹-21 |
| 407 | H | NH | Ar¹² | R¹¹-22 |
| 408 | H | NH | Ar¹² | R¹¹-23 |
| 409 | H | NH | Ar¹² | R¹¹-24 |
| 410 | H | NH | Ar¹² | R¹¹-25 |
| 411 | H | NH | Ar¹² | R¹¹-26 |
| 412 | H | NH | Ar¹² | R¹¹-27 |
| 413 | H | NH | Ar¹² | R¹¹-28 |
| 414 | H | NH | Ar¹² | R¹¹-29 |
| 415 | H | NH | Ar¹² | A¹¹-1a |
| 416 | H | NH | Ar¹² | A¹¹-1b |
| 417 | H | NH | Ar¹² | A¹¹-2a |
| 418 | H | NH | Ar¹² | A¹¹-2b |
| 419 | H | NH | Ar¹² | A¹¹-3a |
| 420 | H | NH | Ar¹² | A¹¹-3b |
| 421 | H | NCH₃ | Ar¹ | R¹¹-1 |
| 422 | H | NCH₃ | Ar¹ | R¹¹-2 |
| 423 | H | NCH₃ | Ar¹ | R¹¹-3 |
| 424 | H | NCH₃ | Ar¹ | R¹¹-4 |
| 425 | H | NCH₃ | Ar¹ | R¹¹-5 |
| 426 | H | NCH₃ | Ar¹ | R¹¹-6 |
| 427 | H | NCH₃ | Ar¹ | R¹¹-7 |
| 428 | H | NCH₃ | Ar¹ | R¹¹-8 |
| 429 | H | NCH₃ | Ar¹ | R¹¹-9 |
| 430 | H | NCH₃ | Ar¹ | R¹¹-10 |
| 431 | H | NCH₃ | Ar¹ | R¹¹-11 |
| 432 | H | NCH₃ | Ar¹ | R¹¹-12 |
| 433 | H | NCH₃ | Ar¹ | R¹¹-13 |
| 434 | H | NCH₃ | Ar¹ | R¹¹-14 |
| 435 | H | NCH₃ | Ar¹ | R¹¹-15 |
| 436 | H | NCH₃ | Ar¹ | R¹¹-16 |
| 437 | H | NCH₃ | Ar¹ | R¹¹-17 |
| 438 | H | NCH₃ | Ar¹ | R¹¹-18 |
| 439 | H | NCH₃ | Ar¹ | R¹¹-19 |
| 440 | H | NCH₃ | Ar¹ | R¹¹-20 |
| 441 | H | NCH₃ | Ar¹ | R¹¹-21 |
| 442 | H | NCH₃ | Ar¹ | R¹¹-22 |
| 443 | H | NCH₃ | Ar¹ | R¹¹-23 |
| 444 | H | NCH₃ | Ar¹ | R¹¹-24 |
| 445 | H | NCH₃ | Ar¹ | R¹¹-25 |
| 446 | H | NCH₃ | Ar¹ | R¹¹-26 |
| 447 | H | NCH₃ | Ar¹ | R¹¹-27 |
| 448 | H | NCH₃ | Ar¹ | R¹¹-28 |
| 449 | H | NCH₃ | Ar¹ | R¹¹-29 |
| 450 | H | NCH₃ | Ar¹ | A¹¹-1a |
| 451 | H | NCH₃ | Ar¹ | A¹¹-1b |
| 452 | H | NCH₃ | Ar¹ | A¹¹-2a |
| 453 | H | NCH₃ | Ar¹ | A¹¹-2b |
| 454 | H | NCH₃ | Ar¹ | A¹¹-3a |
| 455 | H | NCH₃ | Ar¹ | A¹¹-3b |
| 456 | H | NCH₃ | Ar² | R¹¹-1 |
| 457 | H | NCH₃ | Ar² | R¹¹-2 |
| 458 | H | NCH₃ | Ar² | R¹¹-3 |
| 459 | H | NCH₃ | Ar² | R¹¹-4 |
| 460 | H | NCH₃ | Ar² | R¹¹-5 |
| 461 | H | NCH₃ | Ar² | R¹¹-6 |
| 462 | H | NCH₃ | Ar² | R¹¹-7 |
| 463 | H | NCH₃ | Ar² | R¹¹-8 |
| 464 | H | NCH₃ | Ar² | R¹¹-9 |
| 465 | H | NCH₃ | Ar² | R¹¹-10 |
| 466 | H | NCH₃ | Ar² | R¹¹-11 |
| 467 | H | NCH₃ | Ar² | R¹¹-12 |
| 468 | H | NCH₃ | Ar² | R¹¹-13 |
| 469 | H | NCH₃ | Ar² | R¹¹-14 |
| 470 | H | NCH₃ | Ar² | R¹¹-15 |
| 471 | H | NCH₃ | Ar² | R¹¹-16 |
| 472 | H | NCH₃ | Ar² | R¹¹-17 |
| 473 | H | NCH₃ | Ar² | R¹¹-18 |
| 474 | H | NCH₃ | Ar² | R¹¹-19 |
| 475 | H | NCH₃ | Ar² | R¹¹-20 |
| 476 | H | NCH₃ | Ar² | R¹¹-21 |
| 477 | H | NCH₃ | Ar² | R¹¹-22 |
| 478 | H | NCH₃ | Ar² | R¹¹-23 |
| 479 | H | NCH₃ | Ar² | R¹¹-24 |
| 480 | H | NCH₃ | Ar² | R¹¹-25 |
| 481 | H | NCH₃ | Ar² | R¹¹-26 |
| 482 | H | NCH₃ | Ar² | R¹¹-27 |
| 483 | H | NCH₃ | Ar² | R¹¹-28 |
| 484 | H | NCH₃ | Ar² | R¹¹-29 |
| 485 | H | NCH₃ | Ar² | A¹¹-1a |
| 486 | H | NCH₃ | Ar² | A¹¹-1b |
| 487 | H | NCH₃ | Ar² | A¹¹-2a |
| 488 | H | NCH₃ | Ar² | A¹¹-2b |
| 489 | H | NCH₃ | Ar² | A¹¹-3a |
| 490 | H | NCH₃ | Ar² | A¹¹-3b |
| 491 | H | NCH₃ | Ar³ | R¹¹-1 |
| 492 | H | NCH₃ | Ar³ | R¹¹-2 |
| 493 | H | NCH₃ | Ar³ | R¹¹-3 |
| 494 | H | NCH₃ | Ar³ | R¹¹-4 |
| 495 | H | NCH₃ | Ar³ | R¹¹-5 |
| 496 | H | NCH₃ | Ar³ | R¹¹-6 |
| 497 | H | NCH₃ | Ar³ | R¹¹-7 |
| 498 | H | NCH₃ | Ar³ | R¹¹-8 |
| 499 | H | NCH₃ | Ar³ | R¹¹-9 |
| 500 | H | NCH₃ | Ar³ | R¹¹-10 |
| 501 | H | NCH₃ | Ar³ | R¹¹-11 |
| 502 | H | NCH₃ | Ar³ | R¹¹-12 |
| 503 | H | NCH₃ | Ar³ | R¹¹-13 |
| 504 | H | NCH₃ | Ar³ | R¹¹-14 |
| 505 | H | NCH₃ | Ar³ | R¹¹-15 |
| 506 | H | NCH₃ | Ar³ | R¹¹-16 |
| 507 | H | NCH₃ | Ar³ | R¹¹-17 |
| 508 | H | NCH₃ | Ar³ | R¹¹-18 |
| 509 | H | NCH₃ | Ar³ | R¹¹-19 |
| 510 | H | NCH₃ | Ar³ | R¹¹-20 |
| 511 | H | NCH₃ | Ar³ | R¹¹-21 |
| 512 | H | NCH₃ | Ar³ | R¹¹-22 |
| 513 | H | NCH₃ | Ar³ | R¹¹-23 |
| 514 | H | NCH₃ | Ar³ | R¹¹-24 |
| 515 | H | NCH₃ | Ar³ | R¹¹-25 |
| 516 | H | NCH₃ | Ar³ | R¹¹-26 |
| 517 | H | NCH₃ | Ar³ | R¹¹-27 |
| 518 | H | NCH₃ | Ar³ | R¹¹-28 |
| 519 | H | NCH₃ | Ar³ | R¹¹-29 |
| 520 | H | NCH₃ | Ar³ | A¹¹-1a |
| 521 | H | NCH₃ | Ar³ | A¹¹-1b |
| 522 | H | NCH₃ | Ar³ | A¹¹-2a |
| 523 | H | NCH₃ | Ar³ | A¹¹-2b |
| 524 | H | NCH₃ | Ar³ | A¹¹-3a |
| 525 | H | NCH₃ | Ar⁴ | A¹¹-3b |
| 526 | H | NCH₃ | Ar⁴ | R¹¹-1 |
| 527 | H | NCH₃ | Ar⁴ | R¹¹-2 |
| 528 | H | NCH₃ | Ar⁴ | R¹¹-3 |
| 529 | H | NCH₃ | Ar⁴ | R¹¹-4 |
| 530 | H | NCH₃ | Ar⁴ | R¹¹-5 |
| 531 | H | NCH₃ | Ar⁴ | R¹¹-6 |
| 532 | H | NCH₃ | Ar⁴ | R¹¹-7 |
| 533 | H | NCH₃ | Ar⁴ | R¹¹-8 |
| 534 | H | NCH₃ | Ar⁴ | R¹¹-9 |
| 535 | H | NCH₃ | Ar⁴ | R¹¹-10 |
| 536 | H | NCH₃ | Ar⁴ | R¹¹-11 |
| 537 | H | NCH₃ | Ar⁴ | R¹¹-12 |
| 538 | H | NCH₃ | Ar⁴ | R¹¹-13 |
| 539 | H | NCH₃ | Ar⁴ | R¹¹-14 |
| 540 | H | NCH₃ | Ar⁴ | R¹¹-15 |
| 541 | H | NCH₃ | Ar⁴ | R¹¹-16 |
| 542 | H | NCH₃ | Ar⁴ | R¹¹-17 |
| 543 | H | NCH₃ | Ar⁴ | R¹¹-18 |
| 544 | H | NCH₃ | Ar⁴ | R¹¹-19 |
| 545 | H | NCH₃ | Ar⁴ | R¹¹-20 |
| 546 | H | NCH₃ | Ar⁴ | R¹¹-21 |
| 547 | H | NCH₃ | Ar⁴ | R¹¹-22 |
| 548 | H | NCH₃ | Ar⁴ | R¹¹-23 |
| 549 | H | NCH₃ | Ar⁴ | R¹¹-24 |
| 550 | H | NCH₃ | Ar⁴ | R¹¹-25 |
| 551 | H | NCH₃ | Ar⁴ | R¹¹-26 |
| 552 | H | NCH₃ | Ar⁴ | R¹¹-27 |
| 553 | H | NCH₃ | Ar⁴ | R¹¹-28 |
| 554 | H | NCH₃ | Ar⁴ | R¹¹-29 |
| 555 | H | NCH₃ | Ar⁴ | A¹¹-1a |
| 556 | H | NCH₃ | Ar⁴ | A¹¹-1b |
| 557 | H | NCH₃ | Ar⁴ | A¹¹-2a |
| 558 | H | NCH₃ | Ar⁴ | A¹¹-2b |
| 559 | H | NCH₃ | Ar⁴ | A¹¹-3a |
| 560 | H | NCH₃ | Ar⁴ | A¹¹-3b |
| 561 | H | NCH₃ | Ar⁵ | R¹¹-1 |
| 562 | H | NCH₃ | Ar⁵ | R¹¹-2 |
| 563 | H | NCH₃ | Ar⁵ | R¹¹-3 |
| 564 | H | NCH₃ | Ar⁵ | R¹¹-4 |
| 565 | H | NCH₃ | Ar⁵ | R¹¹-5 |
| 566 | H | NCH₃ | Ar⁵ | R¹¹-6 |
| 567 | H | NCH₃ | Ar⁵ | R¹¹-7 |
| 568 | H | NCH₃ | Ar⁵ | R¹¹-8 |
| 569 | H | NCH₃ | Ar⁵ | R¹¹-9 |
| 570 | H | NCH₃ | Ar⁵ | R¹¹-10 |
| 571 | H | NCH₃ | Ar⁵ | R¹¹-11 |
| 572 | H | NCH₃ | Ar⁵ | R¹¹-12 |
| 573 | H | NCH₃ | Ar⁵ | R¹¹-13 |
| 574 | H | NCH₃ | Ar⁵ | R¹¹-14 |
| 575 | H | NCH₃ | Ar⁵ | R¹¹-15 |
| 576 | H | NCH₃ | Ar⁵ | R¹¹-16 |
| 577 | H | NCH₃ | Ar⁵ | R¹¹-17 |
| 578 | H | NCH₃ | Ar⁵ | R¹¹-18 |
| 579 | H | NCH₃ | Ar⁵ | R¹¹-19 |
| 580 | H | NCH₃ | Ar⁵ | R¹¹-20 |
| 581 | H | NCH₃ | Ar⁵ | R¹¹-21 |
| 582 | H | NCH₃ | Ar⁵ | R¹¹-22 |
| 583 | H | NCH₃ | Ar⁵ | R¹¹-23 |
| 584 | H | NCH₃ | Ar⁵ | R¹¹-24 |
| 585 | H | NCH₃ | Ar⁵ | R¹¹-25 |
| 586 | H | NCH₃ | Ar⁵ | R¹¹-26 |
| 587 | H | NCH₃ | Ar⁵ | R¹¹-27 |
| 588 | H | NCH₃ | Ar⁵ | R¹¹-28 |
| 589 | H | NCH₃ | Ar⁵ | R¹¹-29 |
| 590 | H | NCH₃ | Ar⁵ | A¹¹-1a |
| 591 | H | NCH₃ | Ar⁵ | A¹¹-1b |
| 592 | H | NCH₃ | Ar⁵ | A¹¹-2a |
| 593 | H | NCH₃ | Ar⁵ | A¹¹-2b |
| 594 | H | NCH₃ | Ar⁵ | A¹¹-3a |
| 595 | H | NCH₃ | Ar⁵ | A¹¹-3b |
| 596 | H | NCH₃ | Ar⁶ | R¹¹-1 |
| 597 | H | NCH₃ | Ar⁶ | R¹¹-2 |
| 598 | H | NCH₃ | Ar⁶ | R¹¹-3 |
| 599 | H | NCH₃ | Ar⁶ | R¹¹-4 |
| 600 | H | NCH₃ | Ar⁶ | R¹¹-5 |
| 601 | H | NCH₃ | Ar⁶ | R¹¹-6 |
| 602 | H | NCH₃ | Ar⁶ | R¹¹-7 |
| 603 | H | NCH₃ | Ar⁶ | R¹¹-8 |
| 604 | H | NCH₃ | Ar⁶ | R¹¹-9 |
| 605 | H | NCH₃ | Ar⁶ | R¹¹-10 |
| 606 | H | NCH₃ | Ar⁶ | R¹¹-11 |
| 607 | H | NCH₃ | Ar⁶ | R¹¹-12 |
| 608 | H | NCH₃ | Ar⁶ | R¹¹-13 |
| 609 | H | NCH₃ | Ar⁶ | R¹¹-14 |
| 610 | H | NCH₃ | Ar⁶ | R¹¹-15 |
| 611 | H | NCH₃ | Ar⁶ | R¹¹-16 |
| 612 | H | NCH₃ | Ar⁶ | R¹¹-17 |
| 613 | H | NCH₃ | Ar⁶ | R¹¹-18 |
| 614 | H | NCH₃ | Ar⁶ | R¹¹-19 |
| 615 | H | NCH₃ | Ar⁶ | R¹¹-20 |
| 616 | H | NCH₃ | Ar⁶ | R¹¹-21 |
| 617 | H | NCH₃ | Ar⁶ | R¹¹-22 |
| 618 | H | NCH₃ | Ar⁶ | R¹¹-23 |
| 619 | H | NCH₃ | Ar⁶ | R¹¹-24 |
| 620 | H | NCH₃ | Ar⁶ | R¹¹-25 |
| 621 | H | NCH₃ | Ar⁶ | R¹¹-26 |
| 622 | H | NCH₃ | Ar⁶ | R¹¹-27 |
| 623 | H | NCH₃ | Ar⁶ | R¹¹-28 |
| 624 | H | NCH₃ | Ar⁶ | R¹¹-29 |
| 625 | H | NCH₃ | Ar⁶ | A¹¹-1a |
| 626 | H | NCH₃ | Ar⁶ | A¹¹-1b |
| 627 | H | NCH₃ | Ar⁶ | A¹¹-2a |
| 628 | H | NCH₃ | Ar⁶ | A¹¹-2b |
| 629 | H | NCH₃ | Ar⁶ | A¹¹-3a |
| 630 | H | NCH₃ | Ar⁶ | A¹¹-3b |
| 631 | H | NCH₃ | Ar⁷ | R¹¹-1 |
| 632 | H | NCH₃ | Ar⁷ | R¹¹-2 |
| 633 | H | NCH₃ | Ar⁷ | R¹¹-3 |
| 634 | H | NCH₃ | Ar⁷ | R¹¹-4 |
| 635 | H | NCH₃ | Ar⁷ | R¹¹-5 |
| 636 | H | NCH₃ | Ar⁷ | R¹¹-6 |
| 637 | H | NCH₃ | Ar⁷ | R¹¹-7 |
| 638 | H | NCH₃ | Ar⁷ | R¹¹-8 |
| 639 | H | NCH₃ | Ar⁷ | R¹¹-9 |
| 640 | H | NCH₃ | Ar⁷ | R¹¹-10 |
| 641 | H | NCH₃ | Ar⁷ | R¹¹-11 |
| 642 | H | NCH₃ | Ar⁷ | R¹¹-12 |
| 643 | H | NCH₃ | Ar⁷ | R¹¹-13 |
| 644 | H | NCH₃ | Ar⁷ | R¹¹-14 |
| 645 | H | NCH₃ | Ar⁷ | R¹¹-15 |
| 646 | H | NCH₃ | Ar⁷ | R¹¹-16 |
| 647 | H | NCH₃ | Ar⁷ | R¹¹-17 |
| 648 | H | NCH₃ | Ar⁷ | R¹¹-18 |
| 649 | H | NCH₃ | Ar⁷ | R¹¹-19 |
| 650 | H | NCH₃ | Ar⁷ | R¹¹-20 |
| 651 | H | NCH₃ | Ar⁷ | R¹¹-21 |
| 652 | H | NCH₃ | Ar⁷ | R¹¹-22 |
| 653 | H | NCH₃ | Ar⁷ | R¹¹-23 |
| 654 | H | NCH₃ | Ar⁷ | R¹¹-24 |
| 655 | H | NCH₃ | Ar⁷ | R¹¹-25 |
| 656 | H | NCH₃ | Ar⁷ | R¹¹-26 |
| 657 | H | NCH₃ | Ar⁷ | R¹¹-27 |
| 658 | H | NCH₃ | Ar⁷ | R¹¹-28 |
| 659 | H | NCH₃ | Ar⁷ | R¹¹-29 |
| 660 | H | NCH₃ | Ar⁷ | A¹¹-1a |
| 661 | H | NCH₃ | Ar⁷ | A¹¹-1b |
| 662 | H | NCH₃ | Ar⁷ | A¹¹-2a |
| 663 | H | NCH₃ | Ar⁷ | A¹¹-2b |
| 664 | H | NCH₃ | Ar⁷ | A¹¹-3a |
| 665 | H | NCH₃ | Ar⁷ | A¹¹-3b |
| 666 | H | NCH₃ | Ar⁸ | R¹¹-1 |
| 667 | H | NCH₃ | Ar⁸ | R¹¹-2 |
| 668 | H | NCH₃ | Ar⁸ | R¹¹-3 |
| 669 | H | NCH₃ | Ar⁸ | R¹¹-4 |
| 670 | H | NCH₃ | Ar⁸ | R¹¹-5 |
| 671 | H | NCH₃ | Ar⁸ | R¹¹-6 |
| 672 | H | NCH₃ | Ar⁸ | R¹¹-7 |
| 673 | H | NCH₃ | Ar⁸ | R¹¹-8 |
| 674 | H | NCH₃ | Ar⁸ | R¹¹-9 |
| 675 | H | NCH₃ | Ar⁸ | R¹¹-10 |
| 676 | H | NCH₃ | Ar⁸ | R¹¹-11 |
| 677 | H | NCH₃ | Ar⁸ | R¹¹-12 |
| 678 | H | NCH₃ | Ar⁸ | R¹¹-13 |
| 679 | H | NCH₃ | Ar⁸ | R¹¹-14 |
| 680 | H | NCH₃ | Ar⁸ | R¹¹-15 |
| 681 | H | NCH₃ | Ar⁸ | R¹¹-16 |
| 682 | H | NCH₃ | Ar⁸ | R¹¹-17 |
| 683 | H | NCH₃ | Ar⁸ | R¹¹-18 |
| 684 | H | NCH₃ | Ar⁸ | R¹¹-19 |
| 685 | H | NCH₃ | Ar⁸ | R¹¹-20 |
| 686 | H | NCH₃ | Ar⁸ | R¹¹-21 |
| 687 | H | NCH₃ | Ar⁸ | R¹¹-22 |
| 688 | H | NCH₃ | Ar⁸ | R¹¹-23 |
| 689 | H | NCH₃ | Ar⁸ | R¹¹-24 |
| 690 | H | NCH₃ | Ar⁸ | R¹¹-25 |
| 691 | H | NCH₃ | Ar⁸ | R¹¹-26 |
| 692 | H | NCH₃ | Ar⁸ | R¹¹-27 |
| 693 | H | NCH₃ | Ar⁸ | R¹¹-28 |
| 694 | H | NCH₃ | Ar⁸ | R¹¹-29 |
| 695 | H | NCH₃ | Ar⁸ | A¹¹-1a |
| 696 | H | NCH₃ | Ar⁸ | A¹¹-1b |
| 697 | H | NCH₃ | Ar⁸ | A¹¹-2a |
| 698 | H | NCH₃ | Ar⁸ | A¹¹-2b |
| 699 | H | NCH₃ | Ar⁸ | A¹¹-3a |
| 700 | H | NCH₃ | Ar⁸ | A¹¹-3b |
| 701 | H | NCH₃ | Ar⁹ | R¹¹-1 |
| 702 | H | NCH₃ | Ar⁹ | R¹¹-2 |
| 703 | H | NCH₃ | Ar⁹ | R¹¹-3 |
| 704 | H | NCH₃ | Ar⁹ | R¹¹-4 |
| 705 | H | NCH₃ | Ar⁹ | R¹¹-5 |
| 706 | H | NCH₃ | Ar⁹ | R¹¹-6 |
| 707 | H | NCH₃ | Ar⁹ | R¹¹-7 |
| 708 | H | NCH₃ | Ar⁹ | R¹¹-8 |
| 709 | H | NCH₃ | Ar⁹ | R¹¹-9 |
| 710 | H | NCH₃ | Ar⁹ | R¹¹-10 |
| 711 | H | NCH₃ | Ar⁹ | R¹¹-11 |
| 712 | H | NCH₃ | Ar⁹ | R¹¹-12 |
| 713 | H | NCH₃ | Ar⁹ | R¹¹-13 |
| 714 | H | NCH₃ | Ar⁹ | R¹¹-14 |
| 715 | H | NCH₃ | Ar⁹ | R¹¹-15 |
| 716 | H | NCH₃ | Ar⁹ | R¹¹-16 |
| 717 | H | NCH₃ | Ar⁹ | R¹¹-17 |
| 718 | H | NCH₃ | Ar⁹ | R¹¹-18 |
| 719 | H | NCH₃ | Ar⁹ | R¹¹-19 |
| 720 | H | NCH₃ | Ar⁹ | R¹¹-20 |
| 721 | H | NCH₃ | Ar⁹ | R¹¹-21 |
| 722 | H | NCH₃ | Ar⁹ | R¹¹-22 |
| 723 | H | NCH₃ | Ar⁹ | R¹¹-23 |
| 724 | H | NCH₃ | Ar⁹ | R¹¹-24 |
| 725 | H | NCH₃ | Ar⁹ | R¹¹-25 |
| 726 | H | NCH₃ | Ar⁹ | R¹¹-26 |
| 727 | H | NCH₃ | Ar⁹ | R¹¹-27 |
| 728 | H | NCH₃ | Ar⁹ | R¹¹-28 |
| 729 | H | NCH₃ | Ar⁹ | R¹¹-29 |
| 730 | H | NCH₃ | Ar⁹ | A¹¹-1a |
| 731 | H | NCH₃ | Ar⁹ | A¹¹-1b |
| 732 | H | NCH₃ | Ar⁹ | A¹¹-2a |
| 733 | H | NCH₃ | Ar⁹ | A¹¹-2b |
| 734 | H | NCH₃ | Ar⁹ | A¹¹-3a |
| 735 | H | NCH₃ | Ar⁹ | A¹¹-3b |
| 736 | H | NCH₃ | Ar¹⁰ | R¹¹-1 |
| 737 | H | NCH₃ | Ar¹⁰ | R¹¹-2 |
| 738 | H | NCH₃ | Ar¹⁰ | R¹¹-3 |
| 739 | H | NCH₃ | Ar¹⁰ | R¹¹-4 |
| 740 | H | NCH₃ | Ar¹⁰ | R¹¹-5 |
| 741 | H | NCH₃ | Ar¹⁰ | R¹¹-6 |
| 742 | H | NCH₃ | Ar¹⁰ | R¹¹-7 |
| 743 | H | NCH₃ | Ar¹⁰ | R¹¹-8 |
| 744 | H | NCH₃ | Ar¹⁰ | R¹¹-9 |
| 745 | H | NCH₃ | Ar¹⁰ | R¹¹-10 |
| 746 | H | NCH₃ | Ar¹⁰ | R¹¹-11 |
| 747 | H | NCH₃ | Ar¹⁰ | R¹¹-12 |
| 748 | H | NCH₃ | Ar¹⁰ | R¹¹-13 |
| 749 | H | NCH₃ | Ar¹⁰ | R¹¹-14 |
| 750 | H | NCH₃ | Ar¹⁰ | R¹¹-15 |
| 751 | H | NCH₃ | Ar¹⁰ | R¹¹-16 |
| 752 | H | NCH₃ | Ar¹⁰ | R¹¹-17 |
| 753 | H | NCH₃ | Ar¹⁰ | R¹¹-18 |
| 754 | H | NCH₃ | Ar¹⁰ | R¹¹-19 |
| 755 | H | NCH₃ | Ar¹⁰ | R¹¹-20 |
| 756 | H | NCH₃ | Ar¹⁰ | R¹¹-21 |
| 757 | H | NCH₃ | Ar¹⁰ | R¹¹-22 |
| 758 | H | NCH₃ | Ar¹⁰ | R¹¹-23 |
| 759 | H | NCH₃ | Ar¹⁰ | R¹¹-24 |
| 760 | H | NCH₃ | Ar¹⁰ | R¹¹-25 |
| 761 | H | NCH₃ | Ar¹⁰ | R¹¹-26 |
| 762 | H | NCH₃ | Ar¹⁰ | R¹¹-27 |
| 763 | H | NCH₃ | Ar¹⁰ | R¹¹-28 |
| 764 | H | NCH₃ | Ar¹⁰ | R¹¹-29 |
| 765 | H | NCH₃ | Ar¹⁰ | A¹¹-1a |
| 766 | H | NCH₃ | Ar¹⁰ | A¹¹-1b |
| 767 | H | NCH₃ | Ar¹⁰ | A¹¹-2a |
| 768 | H | NCH₃ | Ar¹⁰ | A¹¹-2b |
| 769 | H | NCH₃ | Ar¹⁰ | A¹¹-3a |
| 770 | H | NCH₃ | Ar¹⁰ | A¹¹-3b |
| 771 | H | NCH₃ | Ar¹¹ | R¹¹-1 |
| 772 | H | NCH₃ | Ar¹¹ | R¹¹-2 |
| 773 | H | NCH₃ | Ar¹¹ | R¹¹-3 |
| 774 | H | NCH₃ | Ar¹¹ | R¹¹-4 |
| 775 | H | NCH₃ | Ar¹¹ | R¹¹-5 |
| 776 | H | NCH₃ | Ar¹¹ | R¹¹-6 |
| 777 | H | NCH₃ | Ar¹¹ | R¹¹-7 |
| 778 | H | NCH₃ | Ar¹¹ | R¹¹-8 |
| 779 | H | NCH₃ | Ar¹¹ | R¹¹-9 |
| 780 | H | NCH₃ | Ar¹¹ | R¹¹-10 |
| 781 | H | NCH₃ | Ar¹¹ | R¹¹-11 |
| 782 | H | NCH₃ | Ar¹¹ | R¹¹-12 |
| 783 | H | NCH₃ | Ar¹¹ | R¹¹-13 |
| 784 | H | NCH₃ | Ar¹¹ | R¹¹-14 |
| 785 | H | NCH₃ | Ar¹¹ | R¹¹-15 |
| 786 | H | NCH₃ | Ar¹¹ | R¹¹-16 |
| 787 | H | NCH₃ | Ar¹¹ | R¹¹-17 |
| 788 | H | NCH₃ | Ar¹¹ | R¹¹-18 |
| 789 | H | NCH₃ | Ar¹¹ | R¹¹-19 |
| 790 | H | NCH₃ | Ar¹¹ | R¹¹-20 |
| 791 | H | NCH₃ | Ar¹¹ | R¹¹-21 |
| 792 | H | NCH₃ | Ar¹¹ | R¹¹-22 |
| 793 | H | NCH₃ | Ar¹¹ | R¹¹-23 |
| 794 | H | NCH₃ | Ar¹¹ | R¹¹-24 |
| 795 | H | NCH₃ | Ar¹¹ | R¹¹-25 |
| 796 | H | NCH₃ | Ar¹¹ | R¹¹-26 |
| 797 | H | NCH₃ | Ar¹¹ | R¹¹-27 |
| 798 | H | NCH₃ | Ar¹¹ | R¹¹-28 |
| 799 | H | NCH₃ | Ar¹¹ | R¹¹-29 |
| 800 | H | NCH₃ | Ar¹¹ | A¹¹-1a |
| 801 | H | NCH₃ | Ar¹¹ | A¹¹-1b |
| 802 | H | NCH₃ | Ar¹¹ | A¹¹-2a |
| 803 | H | NCH₃ | Ar¹¹ | A¹¹-2b |
| 804 | H | NCH₃ | Ar¹¹ | A¹¹-3a |
| 805 | H | NCH₃ | Ar¹¹ | A¹¹-3b |
| 806 | H | NCH₃ | Ar¹² | R¹¹-1 |
| 807 | H | NCH₃ | Ar¹² | R¹¹-2 |
| 808 | H | NCH₃ | Ar¹² | R¹¹-3 |
| 809 | H | NCH₃ | Ar¹² | R¹¹-4 |
| 810 | H | NCH₃ | Ar¹² | R¹¹-5 |
| 811 | H | NCH₃ | Ar¹² | R¹¹-6 |
| 812 | H | NCH₃ | Ar¹² | R¹¹-7 |
| 813 | H | NCH₃ | Ar¹² | R¹¹-8 |
| 814 | H | NCH₃ | Ar¹² | R¹¹-9 |
| 815 | H | NCH₃ | Ar¹² | R¹¹-10 |
| 816 | H | NCH₃ | Ar¹² | R¹¹-11 |
| 817 | H | NCH₃ | Ar¹² | R¹¹-12 |
| 818 | H | NCH₃ | Ar¹² | R¹¹-13 |
| 819 | H | NCH₃ | Ar¹² | R¹¹-14 |
| 820 | H | NCH₃ | Ar¹² | R¹¹-15 |
| 821 | H | NCH₃ | Ar¹² | R¹¹-16 |
| 822 | H | NCH₃ | Ar¹² | R¹¹-17 |
| 823 | H | NCH₃ | Ar¹² | R¹¹-18 |
| 824 | H | NCH₃ | Ar¹² | R¹¹-19 |
| 825 | H | NCH₃ | Ar¹² | R¹¹-20 |
| 826 | H | NCH₃ | Ar¹² | R¹¹-21 |
| 827 | H | NCH₃ | Ar¹² | R¹¹-22 |
| 828 | H | NCH₃ | Ar¹² | R¹¹-23 |
| 829 | H | NCH₃ | Ar¹² | R¹¹-24 |
| 830 | H | NCH₃ | Ar¹² | R¹¹-25 |
| 831 | H | NCH₃ | Ar¹² | R¹¹-26 |
| 832 | H | NCH₃ | Ar¹² | R¹¹-27 |
| 833 | H | NCH₃ | Ar¹² | R¹¹-28 |
| 834 | H | NCH₃ | Ar¹² | R¹¹-29 |
| 835 | H | NCH₃ | Ar¹² | A¹¹-1a |
| 836 | H | NCH₃ | Ar¹² | A¹¹-1b |
| 837 | H | NCH₃ | Ar¹² | A¹¹-2a |
| 838 | H | NCH₃ | Ar¹² | A¹¹-2b |
| 839 | H | NCH₃ | Ar¹² | A¹¹-3a |
| 840 | H | NCH₃ | Ar¹² | A¹¹-3b |
| 841 | H | O | Ar¹ | R¹¹-1 |
| 842 | H | O | Ar¹ | R¹¹-2 |
| 843 | H | O | Ar¹ | R¹¹-3 |
| 844 | H | O | Ar¹ | R¹¹-4 |
| 845 | H | O | Ar¹ | R¹¹-5 |
| 846 | H | O | Ar¹ | R¹¹-6 |
| 847 | H | O | Ar¹ | R¹¹-7 |
| 848 | H | O | Ar¹ | R¹¹-8 |
| 849 | H | O | Ar¹ | R¹¹-9 |
| 850 | H | O | Ar¹ | R¹¹-10 |
| 851 | H | O | Ar¹ | R¹¹-11 |
| 852 | H | O | Ar¹ | R¹¹-12 |
| 853 | H | O | Ar¹ | R¹¹-13 |
| 854 | H | O | Ar¹ | R¹¹-14 |
| 855 | H | O | Ar¹ | R¹¹-15 |
| 856 | H | O | Ar¹ | R¹¹-16 |
| 857 | H | O | Ar¹ | R¹¹-17 |
| 858 | H | O | Ar¹ | R¹¹-18 |
| 859 | H | O | Ar¹ | R¹¹-19 |
| 860 | H | O | Ar¹ | R¹¹-20 |
| 861 | H | O | Ar¹ | R¹¹-21 |
| 862 | H | O | Ar¹ | R¹¹-22 |
| 863 | H | O | Ar¹ | R¹¹-23 |
| 864 | H | O | Ar¹ | R¹¹-24 |
| 865 | H | O | Ar¹ | R¹¹-25 |
| 866 | H | O | Ar¹ | R¹¹-26 |
| 867 | H | O | Ar¹ | R¹¹-27 |
| 868 | H | O | Ar¹ | R¹¹-28 |
| 869 | H | O | Ar¹ | R¹¹-29 |
| 870 | H | O | Ar¹ | A¹¹-1a |
| 871 | H | O | Ar¹ | A¹¹-1b |
| 872 | H | O | Ar¹ | A¹¹-2a |
| 873 | H | O | Ar¹ | A¹¹-2b |
| 874 | H | O | Ar¹ | A¹¹-3a |
| 875 | H | O | Ar¹ | A¹¹-3b |
| 876 | H | O | Ar² | R¹¹-1 |
| 877 | H | O | Ar² | R¹¹-2 |
| 878 | H | O | Ar² | R¹¹-3 |
| 879 | H | O | Ar² | R¹¹-4 |
| 880 | H | O | Ar² | R¹¹-5 |
| 881 | H | O | Ar² | R¹¹-6 |
| 882 | H | O | Ar² | R¹¹-7 |
| 883 | H | O | Ar² | R¹¹-8 |
| 884 | H | O | Ar² | R¹¹-9 |
| 885 | H | O | Ar² | R¹¹-10 |
| 886 | H | O | Ar² | R¹¹-11 |
| 887 | H | O | Ar² | R¹¹-12 |
| 888 | H | O | Ar² | R¹¹-13 |
| 889 | H | O | Ar² | R¹¹-14 |
| 890 | H | O | Ar² | R¹¹-15 |
| 891 | H | O | Ar² | R¹¹-16 |
| 892 | H | O | Ar² | R¹¹-17 |
| 893 | H | O | Ar² | R¹¹-18 |
| 894 | H | O | Ar² | R¹¹-19 |
| 895 | H | O | Ar² | R¹¹-20 |
| 896 | H | O | Ar² | R¹¹-21 |
| 897 | H | O | Ar² | R¹¹-22 |
| 898 | H | O | Ar² | R¹¹-23 |
| 899 | H | O | Ar² | R¹¹-24 |
| 900 | H | O | Ar² | R¹¹-25 |
| 901 | H | O | Ar² | R¹¹-26 |
| 902 | H | O | Ar² | R¹¹-27 |
| 903 | H | O | Ar² | R¹¹-28 |
| 904 | H | O | Ar² | R¹¹-29 |
| 905 | H | O | Ar² | A¹¹-1a |
| 906 | H | O | Ar² | A¹¹-1b |
| 907 | H | O | Ar² | A¹¹-2a |
| 908 | H | O | Ar² | A¹¹-2b |
| 909 | H | O | Ar² | A¹¹-3a |
| 910 | H | O | Ar² | A¹¹-3b |
| 911 | H | O | Ar³ | R¹¹-1 |
| 912 | H | O | Ar³ | R¹¹-2 |
| 913 | H | O | Ar³ | R¹¹-3 |
| 914 | H | O | Ar³ | R¹¹-4 |
| 915 | H | O | Ar³ | R¹¹-5 |
| 916 | H | O | Ar³ | R¹¹-6 |
| 917 | H | O | Ar³ | R¹¹-7 |
| 918 | H | O | Ar³ | R¹¹-8 |
| 919 | H | O | Ar³ | R¹¹-9 |
| 920 | H | O | Ar³ | R¹¹-10 |
| 921 | H | O | Ar³ | R¹¹-11 |
| 922 | H | O | Ar³ | R¹¹-12 |
| 923 | H | O | Ar³ | R¹¹-13 |
| 924 | H | O | Ar³ | R¹¹-14 |
| 925 | H | O | Ar³ | R¹¹-15 |
| 926 | H | O | Ar³ | R¹¹-16 |
| 927 | H | O | Ar³ | R¹¹-17 |
| 928 | H | O | Ar³ | R¹¹-18 |
| 929 | H | O | Ar³ | R¹¹-19 |
| 930 | H | O | Ar³ | R¹¹-20 |
| 931 | H | O | Ar³ | R¹¹-21 |
| 932 | H | O | Ar³ | R¹¹-22 |
| 933 | H | O | Ar³ | R¹¹-23 |
| 934 | H | O | Ar³ | R¹¹-24 |
| 935 | H | O | Ar³ | R¹¹-25 |
| 936 | H | O | Ar³ | R¹¹-26 |
| 937 | H | O | Ar³ | R¹¹-27 |
| 938 | H | O | Ar³ | R¹¹-28 |
| 939 | H | O | Ar³ | R¹¹-29 |
| 940 | H | O | Ar³ | A¹¹-1a |
| 941 | H | O | Ar³ | A¹¹-1b |
| 942 | H | O | Ar³ | A¹¹-2a |
| 943 | H | O | Ar³ | A¹¹-2b |
| 944 | H | O | Ar³ | A¹¹-3a |
| 945 | H | O | Ar⁴ | A¹¹-3b |
| 946 | H | O | Ar⁴ | R¹¹-1 |
| 947 | H | O | Ar⁴ | R¹¹-2 |
| 948 | H | O | Ar⁴ | R¹¹-3 |
| 949 | H | O | Ar⁴ | R¹¹-4 |
| 950 | H | O | Ar⁴ | R¹¹-5 |
| 951 | H | O | Ar⁴ | R¹¹-6 |
| 952 | H | O | Ar⁴ | R¹¹-7 |
| 953 | H | O | Ar⁴ | R¹¹-8 |
| 954 | H | O | Ar⁴ | R¹¹-9 |
| 955 | H | O | Ar⁴ | R¹¹-10 |
| 956 | H | O | Ar⁴ | R¹¹-11 |
| 957 | H | O | Ar⁴ | R¹¹-12 |
| 958 | H | O | Ar⁴ | R¹¹-13 |
| 959 | H | O | Ar⁴ | R¹¹-14 |
| 960 | H | O | Ar⁴ | R¹¹-15 |
| 961 | H | O | Ar⁴ | R¹¹-16 |
| 962 | H | O | Ar⁴ | R¹¹-17 |
| 963 | H | O | Ar⁴ | R¹¹-18 |
| 964 | H | O | Ar⁴ | R¹¹-19 |
| 965 | H | O | Ar⁴ | R¹¹-20 |
| 966 | H | O | Ar⁴ | R¹¹-21 |
| 967 | H | O | Ar⁴ | R¹¹-22 |
| 968 | H | O | Ar⁴ | R¹¹-23 |
| 969 | H | O | Ar⁴ | R¹¹-24 |
| 970 | H | O | Ar⁴ | R¹¹-25 |
| 971 | H | O | Ar⁴ | R¹¹-26 |
| 972 | H | O | Ar⁴ | R¹¹-27 |
| 973 | H | O | Ar⁴ | R¹¹-28 |
| 974 | H | O | Ar⁴ | R¹¹-29 |
| 975 | H | O | Ar⁴ | A¹¹-1a |
| 976 | H | O | Ar⁴ | A¹¹-1b |
| 977 | H | O | Ar⁴ | A¹¹-2a |
| 978 | H | O | Ar⁴ | A¹¹-2b |
| 979 | H | O | Ar⁴ | A¹¹-3a |
| 980 | H | O | Ar⁴ | A¹¹-3b |
| 981 | H | O | Ar⁵ | R¹¹-1 |
| 982 | H | O | Ar⁵ | R¹¹-2 |
| 983 | H | O | Ar⁵ | R¹¹-3 |
| 984 | H | O | Ar⁵ | R¹¹-4 |
| 985 | H | O | Ar⁵ | R¹¹-5 |
| 986 | H | O | Ar⁵ | R¹¹-6 |
| 987 | H | O | Ar⁵ | R¹¹-7 |
| 988 | H | O | Ar⁵ | R¹¹-8 |
| 989 | H | O | Ar⁵ | R¹¹-9 |
| 990 | H | O | Ar⁵ | R¹¹-10 |
| 991 | H | O | Ar⁵ | R¹¹-11 |
| 992 | H | O | Ar⁵ | R¹¹-12 |
| 993 | H | O | Ar⁵ | R¹¹-13 |
| 994 | H | O | Ar⁵ | R¹¹-14 |
| 995 | H | O | Ar⁵ | R¹¹-15 |
| 996 | H | O | Ar⁵ | R¹¹-16 |
| 997 | H | O | Ar⁵ | R¹¹-17 |
| 998 | H | O | Ar⁵ | R¹¹-18 |
| 999 | H | O | Ar⁵ | R¹¹-19 |
| 1000 | H | O | Ar⁵ | R¹¹-20 |
| 1001 | H | O | Ar⁵ | R¹¹-21 |
| 1002 | H | O | Ar⁵ | R¹¹-22 |
| 1003 | H | O | Ar⁵ | R¹¹-23 |
| 1004 | H | O | Ar⁵ | R¹¹-24 |
| 1005 | H | O | Ar⁵ | R¹¹-25 |
| 1006 | H | O | Ar⁵ | R¹¹-26 |
| 1007 | H | O | Ar⁵ | R¹¹-27 |
| 1008 | H | O | Ar⁵ | R¹¹-28 |
| 1009 | H | O | Ar⁵ | R¹¹-29 |
| 1010 | H | O | Ar⁵ | A¹¹-1a |
| 1011 | H | O | Ar⁵ | A¹¹-1b |
| 1012 | H | O | Ar⁵ | A¹¹-2a |
| 1013 | H | O | Ar⁵ | A¹¹-2b |
| 1014 | H | O | Ar⁵ | A¹¹-3a |
| 1015 | H | O | Ar⁵ | A¹¹-3b |
| 1016 | H | O | Ar⁶ | R¹¹-1 |
| 1017 | H | O | Ar⁶ | R¹¹-2 |
| 1018 | H | O | Ar⁶ | R¹¹-3 |
| 1019 | H | O | Ar⁶ | R¹¹-4 |
| 1020 | H | O | Ar⁶ | R¹¹-5 |
| 1021 | H | O | Ar⁶ | R¹¹-6 |
| 1022 | H | O | Ar⁶ | R¹¹-7 |
| 1023 | H | O | Ar⁶ | R¹¹-8 |
| 1024 | H | O | Ar⁶ | R¹¹-9 |
| 1025 | H | O | Ar⁶ | R¹¹-10 |
| 1026 | H | O | Ar⁶ | R¹¹-11 |
| 1027 | H | O | Ar⁶ | R¹¹-12 |
| 1028 | H | O | Ar⁶ | R¹¹-13 |
| 1029 | H | O | Ar⁶ | R¹¹-14 |
| 1030 | H | O | Ar⁶ | R¹¹-15 |
| 1031 | H | O | Ar⁶ | R¹¹-16 |
| 1032 | H | O | Ar⁶ | R¹¹-17 |
| 1033 | H | O | Ar⁶ | R¹¹-18 |
| 1034 | H | O | Ar⁶ | R¹¹-19 |
| 1035 | H | O | Ar⁶ | R¹¹-20 |
| 1036 | H | O | Ar⁶ | R¹¹-21 |
| 1037 | H | O | Ar⁶ | R¹¹-22 |
| 1038 | H | O | Ar⁶ | R¹¹-23 |
| 1039 | H | O | Ar⁶ | R¹¹-24 |
| 1040 | H | O | Ar⁶ | R¹¹-25 |
| 1041 | H | O | Ar⁶ | R¹¹-26 |
| 1042 | H | O | Ar⁶ | R¹¹-27 |
| 1043 | H | O | Ar⁶ | R¹¹-28 |
| 1044 | H | O | Ar⁶ | R¹¹-29 |
| 1045 | H | O | Ar⁶ | A¹¹-1a |
| 1046 | H | O | Ar⁶ | A¹¹-1b |
| 1047 | H | O | Ar⁶ | A¹¹-2a |
| 1048 | H | O | Ar⁶ | A¹¹-2b |
| 1049 | H | O | Ar⁶ | A¹¹-3a |
| 1050 | H | O | Ar⁶ | A¹¹-3b |
| 1051 | H | O | Ar⁷ | R¹¹-1 |
| 1052 | H | O | Ar⁷ | R¹¹-2 |
| 1053 | H | O | Ar⁷ | R¹¹-3 |
| 1054 | H | O | Ar⁷ | R¹¹-4 |
| 1055 | H | O | Ar⁷ | R¹¹-5 |
| 1056 | H | O | Ar⁷ | R¹¹-6 |
| 1057 | H | O | Ar⁷ | R¹¹-7 |
| 1058 | H | O | Ar⁷ | R¹¹-8 |
| 1059 | H | O | Ar⁷ | R¹¹-9 |
| 1060 | H | O | Ar⁷ | R¹¹-10 |
| 1061 | H | O | Ar⁷ | R¹¹-11 |
| 1062 | H | O | Ar⁷ | R¹¹-12 |
| 1063 | H | O | Ar⁷ | R¹¹-13 |
| 1064 | H | O | Ar⁷ | R¹¹-14 |
| 1065 | H | O | Ar⁷ | R¹¹-15 |
| 1066 | H | O | Ar⁷ | R¹¹-16 |
| 1067 | H | O | Ar⁷ | R¹¹-17 |
| 1068 | H | O | Ar⁷ | R¹¹-18 |
| 1069 | H | O | Ar⁷ | R¹¹-19 |
| 1070 | H | O | Ar⁷ | R¹¹-20 |
| 1071 | H | O | Ar⁷ | R¹¹-21 |
| 1072 | H | O | Ar⁷ | R¹¹-22 |
| 1073 | H | O | Ar⁷ | R¹¹-23 |
| 1074 | H | O | Ar⁷ | R¹¹-24 |
| 1075 | H | O | Ar⁷ | R¹¹-25 |
| 1076 | H | O | Ar⁷ | R¹¹-26 |
| 1077 | H | O | Ar⁷ | R¹¹-27 |
| 1078 | H | O | Ar⁷ | R¹¹-28 |
| 1079 | H | O | Ar⁷ | R¹¹-29 |
| 1080 | H | O | Ar⁷ | A¹¹-1a |
| 1081 | H | O | Ar⁷ | A¹¹-1b |
| 1082 | H | O | Ar⁷ | A¹¹-2a |
| 1083 | H | O | Ar⁷ | A¹¹-2b |
| 1084 | H | O | Ar⁷ | A¹¹-3a |
| 1085 | H | O | Ar⁷ | A¹¹-3b |
| 1086 | H | O | Ar⁸ | R¹¹-1 |
| 1087 | H | O | Ar⁸ | R¹¹-2 |
| 1088 | H | O | Ar⁸ | R¹¹-3 |
| 1089 | H | O | Ar⁸ | R¹¹-4 |
| 1090 | H | O | Ar⁸ | R¹¹-5 |
| 1091 | H | O | Ar⁸ | R¹¹-6 |
| 1092 | H | O | Ar⁸ | R¹¹-7 |
| 1093 | H | O | Ar⁸ | R¹¹-8 |
| 1094 | H | O | Ar⁸ | R¹¹-9 |
| 1095 | H | O | Ar⁸ | R¹¹-10 |
| 1096 | H | O | Ar⁸ | R¹¹-11 |
| 1097 | H | O | Ar⁸ | R¹¹-12 |
| 1098 | H | O | Ar⁸ | R¹¹-13 |
| 1099 | H | O | Ar⁸ | R¹¹-14 |
| 1100 | H | O | Ar⁸ | R¹¹-15 |
| 1101 | H | O | Ar⁸ | R¹¹-16 |
| 1102 | H | O | Ar⁸ | R¹¹-17 |
| 1103 | H | O | Ar⁸ | R¹¹-18 |
| 1104 | H | O | Ar⁸ | R¹¹-19 |
| 1105 | H | O | Ar⁸ | R¹¹-20 |
| 1106 | H | O | Ar⁸ | R¹¹-21 |
| 1107 | H | O | Ar⁸ | R¹¹-22 |
| 1108 | H | O | Ar⁸ | R¹¹-23 |
| 1109 | H | O | Ar⁸ | R¹¹-24 |
| 1110 | H | O | Ar⁸ | R¹¹-25 |
| 1111 | H | O | Ar⁸ | R¹¹-26 |
| 1112 | H | O | Ar⁸ | R¹¹-27 |
| 1113 | H | O | Ar⁸ | R¹¹-28 |
| 1114 | H | O | Ar⁸ | R¹¹-29 |
| 1115 | H | O | Ar⁸ | A¹¹-1a |
| 1116 | H | O | Ar⁸ | A¹¹-1b |
| 1117 | H | O | Ar⁸ | A¹¹-2a |
| 1118 | H | O | Ar⁸ | A¹¹-2b |
| 1119 | H | O | Ar⁸ | A¹¹-3a |
| 1120 | H | O | Ar⁸ | A¹¹-3b |
| 1121 | H | O | Ar⁹ | R¹¹-1 |
| 1122 | H | O | Ar⁹ | R¹¹-2 |
| 1123 | H | O | Ar⁹ | R¹¹-3 |
| 1124 | H | O | Ar⁹ | R¹¹-4 |
| 1125 | H | O | Ar⁹ | R¹¹-5 |
| 1126 | H | O | Ar⁹ | R¹¹-6 |
| 1127 | H | O | Ar⁹ | R¹¹-7 |
| 1128 | H | O | Ar⁹ | R¹¹-8 |
| 1129 | H | O | Ar⁹ | R¹¹-9 |
| 1130 | H | O | Ar⁹ | R¹¹-10 |
| 1131 | H | O | Ar⁹ | R¹¹-11 |
| 1132 | H | O | Ar⁹ | R¹¹-12 |
| 1133 | H | O | Ar⁹ | R¹¹-13 |
| 1134 | H | O | Ar⁹ | R¹¹-14 |
| 1135 | H | O | Ar⁹ | R¹¹-15 |
| 1136 | H | O | Ar⁹ | R¹¹-16 |
| 1137 | H | O | Ar⁹ | R¹¹-17 |
| 1138 | H | O | Ar⁹ | R¹¹-18 |
| 1139 | H | O | Ar⁹ | R¹¹-19 |
| 1140 | H | O | Ar⁹ | R¹¹-20 |
| 1141 | H | O | Ar⁹ | R¹¹-21 |
| 1142 | H | O | Ar⁹ | R¹¹-22 |
| 1143 | H | O | Ar⁹ | R¹¹-23 |
| 1144 | H | O | Ar⁹ | R¹¹-24 |
| 1145 | H | O | Ar⁹ | R¹¹-25 |
| 1146 | H | O | Ar⁹ | R¹¹-26 |
| 1147 | H | O | Ar⁹ | R¹¹-27 |
| 1148 | H | O | Ar⁹ | R¹¹-28 |
| 1149 | H | O | Ar⁹ | R¹¹-29 |
| 1150 | H | O | Ar⁹ | A¹¹-1a |
| 1151 | H | O | Ar⁹ | A¹¹-1b |
| 1152 | H | O | Ar⁹ | A¹¹-2a |
| 1153 | H | O | Ar⁹ | A¹¹-2b |
| 1154 | H | O | Ar⁹ | A¹¹-3a |
| 1155 | H | O | Ar⁹ | A¹¹-3b |
| 1156 | H | O | Ar¹⁰ | R¹¹-1 |
| 1157 | H | O | Ar¹⁰ | R¹¹-2 |
| 1158 | H | O | Ar¹⁰ | R¹¹-3 |
| 1159 | H | O | Ar¹⁰ | R¹¹-4 |
| 1160 | H | O | Ar¹⁰ | R¹¹-5 |
| 1161 | H | O | Ar¹⁰ | R¹¹-6 |
| 1162 | H | O | Ar¹⁰ | R¹¹-7 |
| 1163 | H | O | Ar¹⁰ | R¹¹-8 |
| 1164 | H | O | Ar¹⁰ | R¹¹-9 |
| 1165 | H | O | Ar¹⁰ | R¹¹-10 |
| 1166 | H | O | Ar¹⁰ | R¹¹-11 |
| 1167 | H | O | Ar¹⁰ | R¹¹-12 |
| 1168 | H | O | Ar¹⁰ | R¹¹-13 |
| 1169 | H | O | Ar¹⁰ | R¹¹-14 |
| 1170 | H | O | Ar¹⁰ | R¹¹-15 |
| 1171 | H | O | Ar¹⁰ | R¹¹-16 |
| 1172 | H | O | Ar¹⁰ | R¹¹-17 |
| 1173 | H | O | Ar¹⁰ | R¹¹-18 |
| 1174 | H | O | Ar¹⁰ | R¹¹-19 |
| 1175 | H | O | Ar¹⁰ | R¹¹-20 |
| 1176 | H | O | Ar¹⁰ | R¹¹-21 |
| 1177 | H | O | Ar¹⁰ | R¹¹-22 |
| 1178 | H | O | Ar¹⁰ | R¹¹-23 |
| 1179 | H | O | Ar¹⁰ | R¹¹-24 |
| 1180 | H | O | Ar¹⁰ | R¹¹-25 |
| 1181 | H | O | Ar¹⁰ | R¹¹-26 |
| 1182 | H | O | Ar¹⁰ | R¹¹-27 |
| 1183 | H | O | Ar¹⁰ | R¹¹-28 |
| 1184 | H | O | Ar¹⁰ | R¹¹-29 |
| 1185 | H | O | Ar¹⁰ | A¹¹-1a |
| 1186 | H | O | Ar¹⁰ | A¹¹-1b |
| 1187 | H | O | Ar¹⁰ | A¹¹-2a |
| 1188 | H | O | Ar¹⁰ | A¹¹-2b |
| 1189 | H | O | Ar¹⁰ | A¹¹-3a |
| 1190 | H | O | Ar¹⁰ | A¹¹-3b |
| 1191 | H | O | Ar¹¹ | R¹¹-1 |
| 1192 | H | O | Ar¹¹ | R¹¹-2 |
| 1193 | H | O | Ar¹¹ | R¹¹-3 |
| 1194 | H | O | Ar¹¹ | R¹¹-4 |
| 1195 | H | O | Ar¹¹ | R¹¹-5 |
| 1196 | H | O | Ar¹¹ | R¹¹-6 |
| 1197 | H | O | Ar¹¹ | R¹¹-7 |
| 1198 | H | O | Ar¹¹ | R¹¹-8 |
| 1199 | H | O | Ar¹¹ | R¹¹-9 |
| 1200 | H | O | Ar¹¹ | R¹¹-10 |
| 1201 | H | O | Ar¹¹ | R¹¹-11 |
| 1202 | H | O | Ar¹¹ | R¹¹-12 |
| 1203 | H | O | Ar¹¹ | R¹¹-13 |
| 1204 | H | O | Ar¹¹ | R¹¹-14 |
| 1205 | H | O | Ar¹¹ | R¹¹-15 |
| 1206 | H | O | Ar¹¹ | R¹¹-16 |
| 1207 | H | O | Ar¹¹ | R¹¹-17 |
| 1208 | H | O | Ar¹¹ | R¹¹-18 |
| 1209 | H | O | Ar¹¹ | R¹¹-19 |
| 1210 | H | O | Ar¹¹ | R¹¹-20 |
| 1211 | H | O | Ar¹¹ | R¹¹-21 |
| 1212 | H | O | Ar¹¹ | R¹¹-22 |
| 1213 | H | O | Ar¹¹ | R¹¹-23 |
| 1214 | H | O | Ar¹¹ | R¹¹-24 |
| 1215 | H | O | Ar¹¹ | R¹¹-25 |
| 1216 | H | O | Ar¹¹ | R¹¹-26 |
| 1217 | H | O | Ar¹¹ | R¹¹-27 |
| 1218 | H | O | Ar¹¹ | R¹¹-28 |
| 1219 | H | O | Ar¹¹ | R¹¹-29 |
| 1220 | H | O | Ar¹¹ | A¹¹-1a |
| 1221 | H | O | Ar¹¹ | A¹¹-1b |
| 1222 | H | O | Ar¹¹ | A¹¹-2a |
| 1223 | H | O | Ar¹¹ | A¹¹-2b |
| 1224 | H | O | Ar¹¹ | A¹¹-3a |
| 1225 | H | O | Ar¹¹ | A¹¹-3b |
| 1226 | H | O | Ar¹² | R¹¹-1 |
| 1227 | H | O | Ar¹² | R¹¹-2 |
| 1228 | H | O | Ar¹² | R¹¹-3 |
| 1229 | H | O | Ar¹² | R¹¹-4 |
| 1230 | H | O | Ar¹² | R¹¹-5 |
| 1231 | H | O | Ar¹² | R¹¹-6 |
| 1232 | H | O | Ar¹² | R¹¹-7 |
| 1233 | H | O | Ar¹² | R¹¹-8 |
| 1234 | H | O | Ar¹² | R¹¹-9 |
| 1235 | H | O | Ar¹² | R¹¹-10 |
| 1236 | H | O | Ar¹² | R¹¹-11 |
| 1237 | H | O | Ar¹² | R¹¹-12 |
| 1238 | H | O | Ar¹² | R¹¹-13 |
| 1239 | H | O | Ar¹² | R¹¹-14 |
| 1240 | H | O | Ar¹² | R¹¹-15 |
| 1241 | H | O | Ar¹² | R¹¹-16 |
| 1242 | H | O | Ar¹² | R¹¹-17 |
| 1243 | H | O | Ar¹² | R¹¹-18 |
| 1244 | H | O | Ar¹² | R¹¹-19 |
| 1245 | H | O | Ar¹² | R¹¹-20 |
| 1246 | H | O | Ar¹² | R¹¹-21 |
| 1247 | H | O | Ar¹² | R¹¹-22 |
| 1248 | H | O | Ar¹² | R¹¹-23 |
| 1249 | H | O | Ar¹² | R¹¹-24 |
| 1250 | H | O | Ar¹² | R¹¹-25 |
| 1251 | H | O | Ar¹² | R¹¹-26 |
| 1252 | H | O | Ar¹² | R¹¹-27 |
| 1253 | H | O | Ar¹² | R¹¹-28 |
| 1254 | H | O | Ar¹² | R¹¹-29 |
| 1255 | H | O | Ar¹² | A¹¹-1a |
| 1256 | H | O | Ar¹² | A¹¹-1b |
| 1257 | H | O | Ar¹² | A¹¹-2a |
| 1258 | H | O | Ar¹² | A¹¹-2b |
| 1259 | H | O | Ar¹² | A¹¹-3a |
| 1260 | H | O | Ar¹² | A¹¹-3b |
| 1261 | CH₃ | NH | Ar¹ | R¹¹-1 |
| 1262 | CH₃ | NH | Ar¹ | R¹¹-2 |
| 1263 | CH₃ | NH | Ar¹ | R¹¹-3 |
| 1264 | CH₃ | NH | Ar¹ | R¹¹-4 |
| 1265 | CH₃ | NH | Ar¹ | R¹¹-5 |
| 1266 | CH₃ | NH | Ar¹ | R¹¹-6 |
| 1267 | CH₃ | NH | Ar¹ | R¹¹-7 |
| 1268 | CH₃ | NH | Ar¹ | R¹¹-8 |
| 1269 | CH₃ | NH | Ar¹ | R¹¹-9 |
| 1270 | CH₃ | NH | Ar¹ | R¹¹-10 |
| 1271 | CH₃ | NH | Ar¹ | R¹¹-11 |
| 1272 | CH₃ | NH | Ar¹ | R¹¹-12 |
| 1273 | CH₃ | NH | Ar¹ | R¹¹-13 |
| 1274 | CH₃ | NH | Ar¹ | R¹¹-14 |
| 1275 | CH₃ | NH | Ar¹ | R¹¹-15 |
| 1276 | CH₃ | NH | Ar¹ | R¹¹-16 |
| 1277 | CH₃ | NH | Ar¹ | R¹¹-17 |
| 1278 | CH₃ | NH | Ar¹ | R¹¹-18 |
| 1279 | CH₃ | NH | Ar¹ | R¹¹-19 |
| 1280 | CH₃ | NH | Ar¹ | R¹¹-20 |
| 1281 | CH₃ | NH | Ar¹ | R¹¹-21 |
| 1282 | CH₃ | NH | Ar¹ | R¹¹-22 |
| 1283 | CH₃ | NH | Ar¹ | R¹¹-23 |
| 1284 | CH₃ | NH | Ar¹ | R¹¹-24 |
| 1285 | CH₃ | NH | Ar¹ | R¹¹-25 |
| 1286 | CH₃ | NH | Ar¹ | R¹¹-26 |
| 1287 | CH₃ | NH | Ar¹ | R¹¹-27 |
| 1288 | CH₃ | NH | Ar¹ | R¹¹-28 |
| 1289 | CH₃ | NH | Ar¹ | R¹¹-29 |
| 1290 | CH₃ | NH | Ar¹ | A¹¹-1a |
| 1291 | CH₃ | NH | Ar¹ | A¹¹-1b |
| 1292 | CH₃ | NH | Ar¹ | A¹¹-2a |
| 1293 | CH₃ | NH | Ar¹ | A¹¹-2b |
| 1294 | CH₃ | NH | Ar¹ | A¹¹-3a |
| 1295 | CH₃ | NH | Ar¹ | A¹¹-3b |
| 1296 | CH₃ | NH | Ar² | R¹¹-1 |
| 1297 | CH₃ | NH | Ar² | R¹¹-2 |
| 1298 | CH₃ | NH | Ar² | R¹¹-3 |
| 1299 | CH₃ | NH | Ar² | R¹¹-4 |
| 1300 | CH₃ | NH | Ar² | R¹¹-5 |
| 1301 | CH₃ | NH | Ar² | R¹¹-6 |
| 1302 | CH₃ | NH | Ar² | R¹¹-7 |
| 1303 | CH₃ | NH | Ar² | R¹¹-8 |
| 1304 | CH₃ | NH | Ar² | R¹¹-9 |
| 1305 | CH₃ | NH | Ar² | R¹¹-10 |
| 1306 | CH₃ | NH | Ar² | R¹¹-11 |
| 1307 | CH₃ | NH | Ar² | R¹¹-12 |
| 1308 | CH₃ | NH | Ar² | R¹¹-13 |
| 1309 | CH₃ | NH | Ar² | R¹¹-14 |
| 1310 | CH₃ | NH | Ar² | R¹¹-15 |
| 1311 | CH₃ | NH | Ar² | R¹¹-16 |
| 1312 | CH₃ | NH | Ar² | R¹¹-17 |
| 1313 | CH₃ | NH | Ar² | R¹¹-18 |
| 1314 | CH₃ | NH | Ar² | R¹¹-19 |
| 1315 | CH₃ | NH | Ar² | R¹¹-20 |
| 1316 | CH₃ | NH | Ar² | R¹¹-21 |
| 1317 | CH₃ | NH | Ar² | R¹¹-22 |
| 1318 | CH₃ | NH | Ar² | R¹¹-23 |
| 1319 | CH₃ | NH | Ar² | R¹¹-24 |
| 1320 | CH₃ | NH | Ar² | R¹¹-25 |
| 1321 | CH₃ | NH | Ar² | R¹¹-26 |
| 1322 | CH₃ | NH | Ar² | R¹¹-27 |
| 1323 | CH₃ | NH | Ar² | R¹¹-28 |
| 1324 | CH₃ | NH | Ar² | R¹¹-29 |
| 1325 | CH₃ | NH | Ar² | A¹¹-1a |
| 1326 | CH₃ | NH | Ar² | A¹¹-1b |
| 1327 | CH₃ | NH | Ar² | A¹¹-2a |
| 1328 | CH₃ | NH | Ar² | A¹¹-2b |
| 1329 | CH₃ | NH | Ar² | A¹¹-3a |
| 1330 | CH₃ | NH | Ar² | A¹¹-3b |
| 1331 | CH₃ | NH | Ar³ | R¹¹-1 |
| 1332 | CH₃ | NH | Ar³ | R¹¹-2 |
| 1333 | CH₃ | NH | Ar³ | R¹¹-3 |
| 1334 | CH₃ | NH | Ar³ | R¹¹-4 |
| 1335 | CH₃ | NH | Ar³ | R¹¹-5 |
| 1336 | CH₃ | NH | Ar³ | R¹¹-6 |
| 1337 | CH₃ | NH | Ar³ | R¹¹-7 |
| 1338 | CH₃ | NH | Ar³ | R¹¹-8 |
| 1339 | CH₃ | NH | Ar³ | R¹¹-9 |
| 1340 | CH₃ | NH | Ar³ | R¹¹-10 |
| 1341 | CH₃ | NH | Ar³ | R¹¹-11 |
| 1342 | CH₃ | NH | Ar³ | R¹¹-12 |
| 1343 | CH₃ | NH | Ar³ | R¹¹-13 |
| 1344 | CH₃ | NH | Ar³ | R¹¹-14 |
| 1345 | CH₃ | NH | Ar³ | R¹¹-15 |
| 1346 | CH₃ | NH | Ar³ | R¹¹-16 |
| 1347 | CH₃ | NH | Ar³ | R¹¹-17 |
| 1348 | CH₃ | NH | Ar³ | R¹¹-18 |
| 1349 | CH₃ | NH | Ar³ | R¹¹-19 |
| 1350 | CH₃ | NH | Ar³ | R¹¹-20 |
| 1351 | CH₃ | NH | Ar³ | R¹¹-21 |
| 1352 | CH₃ | NH | Ar³ | R¹¹-22 |
| 1353 | CH₃ | NH | Ar³ | R¹¹-23 |
| 1354 | CH₃ | NH | Ar³ | R¹¹-24 |
| 1355 | CH₃ | NH | Ar³ | R¹¹-25 |
| 1356 | CH₃ | NH | Ar³ | R¹¹-26 |
| 1357 | CH₃ | NH | Ar³ | R¹¹-27 |
| 1358 | CH₃ | NH | Ar³ | R¹¹-28 |
| 1359 | CH₃ | NH | Ar³ | R¹¹-29 |
| 1360 | CH₃ | NH | Ar³ | A¹¹-1a |
| 1361 | CH₃ | NH | Ar³ | A¹¹-1b |
| 1362 | CH₃ | NH | Ar³ | A¹¹-2a |
| 1363 | CH₃ | NH | Ar³ | A¹¹-2b |
| 1364 | CH₃ | NH | Ar³ | A¹¹-3a |
| 1365 | CH₃ | NH | Ar⁴ | A¹¹-3b |
| 1366 | CH₃ | NH | Ar⁴ | R¹¹-1 |
| 1367 | CH₃ | NH | Ar⁴ | R¹¹-2 |
| 1368 | CH₃ | NH | Ar⁴ | R¹¹-3 |
| 1369 | CH₃ | NH | Ar⁴ | R¹¹-4 |
| 1370 | CH₃ | NH | Ar⁴ | R¹¹-5 |
| 1371 | CH₃ | NH | Ar⁴ | R¹¹-6 |
| 1372 | CH₃ | NH | Ar⁴ | R¹¹-7 |
| 1373 | CH₃ | NH | Ar⁴ | R¹¹-8 |
| 1374 | CH₃ | NH | Ar⁴ | R¹¹-9 |
| 1375 | CH₃ | NH | Ar⁴ | R¹¹-10 |
| 1376 | CH₃ | NH | Ar⁴ | R¹¹-11 |
| 1377 | CH₃ | NH | Ar⁴ | R¹¹-12 |
| 1378 | CH₃ | NH | Ar⁴ | R¹¹-13 |
| 1379 | CH₃ | NH | Ar⁴ | R¹¹-14 |
| 1380 | CH₃ | NH | Ar⁴ | R¹¹-15 |
| 1381 | CH₃ | NH | Ar⁴ | R¹¹-16 |
| 1382 | CH₃ | NH | Ar⁴ | R¹¹-17 |
| 1383 | CH₃ | NH | Ar⁴ | R¹¹-18 |
| 1384 | CH₃ | NH | Ar⁴ | R¹¹-19 |
| 1385 | CH₃ | NH | Ar⁴ | R¹¹-20 |
| 1386 | CH₃ | NH | Ar⁴ | R¹¹-21 |
| 1387 | CH₃ | NH | Ar⁴ | R¹¹-22 |
| 1388 | CH₃ | NH | Ar⁴ | R¹¹-23 |
| 1389 | CH₃ | NH | Ar⁴ | R¹¹-24 |
| 1390 | CH₃ | NH | Ar⁴ | R¹¹-25 |
| 1391 | CH₃ | NH | Ar⁴ | R¹¹-26 |
| 1392 | CH₃ | NH | Ar⁴ | R¹¹-27 |
| 1393 | CH₃ | NH | Ar⁴ | R¹¹-28 |
| 1394 | CH₃ | NH | Ar⁴ | R¹¹-29 |
| 1395 | CH₃ | NH | Ar⁴ | A¹¹-1a |
| 1396 | CH₃ | NH | Ar⁴ | A¹¹-1b |
| 1397 | CH₃ | NH | Ar⁴ | A¹¹-2a |
| 1398 | CH₃ | NH | Ar⁴ | A¹¹-2b |
| 1399 | CH₃ | NH | Ar⁴ | A¹¹-3a |
| 1400 | CH₃ | NH | Ar⁴ | A¹¹-3b |
| 1401 | CH₃ | NH | Ar⁵ | R¹¹-1 |
| 1402 | CH₃ | NH | Ar⁵ | R¹¹-2 |
| 1403 | CH₃ | NH | Ar⁵ | R¹¹-3 |
| 1404 | CH₃ | NH | Ar⁵ | R¹¹-4 |
| 1405 | CH₃ | NH | Ar⁵ | R¹¹-5 |
| 1406 | CH₃ | NH | Ar⁵ | R¹¹-6 |
| 1407 | CH₃ | NH | Ar⁵ | R¹¹-7 |
| 1408 | CH₃ | NH | Ar⁵ | R¹¹-8 |
| 1409 | CH₃ | NH | Ar⁵ | R¹¹-9 |
| 1410 | CH₃ | NH | Ar⁵ | R¹¹-10 |
| 1411 | CH₃ | NH | Ar⁵ | R¹¹-11 |
| 1412 | CH₃ | NH | Ar⁵ | R¹¹-12 |
| 1413 | CH₃ | NH | Ar⁵ | R¹¹-13 |
| 1414 | CH₃ | NH | Ar⁵ | R¹¹-14 |
| 1415 | CH₃ | NH | Ar⁵ | R¹¹-15 |
| 1416 | CH₃ | NH | Ar⁵ | R¹¹-16 |
| 1417 | CH₃ | NH | Ar⁵ | R¹¹-17 |
| 1418 | CH₃ | NH | Ar⁵ | R¹¹-18 |
| 1419 | CH₃ | NH | Ar⁵ | R¹¹-19 |
| 1420 | CH₃ | NH | Ar⁵ | R¹¹-20 |
| 1421 | CH₃ | NH | Ar⁵ | R¹¹-21 |
| 1422 | CH₃ | NH | Ar⁵ | R¹¹-22 |
| 1423 | CH₃ | NH | Ar⁵ | R¹¹-23 |
| 1424 | CH₃ | NH | Ar⁵ | R¹¹-24 |
| 1425 | CH₃ | NH | Ar⁵ | R¹¹-25 |
| 1426 | CH₃ | NH | Ar⁵ | R¹¹-26 |
| 1427 | CH₃ | NH | Ar⁵ | R¹¹-27 |
| 1428 | CH₃ | NH | Ar⁵ | R¹¹-28 |
| 1429 | CH₃ | NH | Ar⁵ | R¹¹-29 |
| 1430 | CH₃ | NH | Ar⁵ | A¹¹-1a |
| 1431 | CH₃ | NH | Ar⁵ | A¹¹-1b |
| 1432 | CH₃ | NH | Ar⁵ | A¹¹-2a |
| 1433 | CH₃ | NH | Ar⁵ | A¹¹-2b |
| 1434 | CH₃ | NH | Ar⁵ | A¹¹-3a |
| 1435 | CH₃ | NH | Ar⁵ | A¹¹-3b |
| 1436 | CH₃ | NH | Ar⁶ | R¹¹-1 |
| 1437 | CH₃ | NH | Ar⁶ | R¹¹-2 |
| 1438 | CH₃ | NH | Ar⁶ | R¹¹-3 |
| 1439 | CH₃ | NH | Ar⁶ | R¹¹-4 |
| 1440 | CH₃ | NH | Ar⁶ | R¹¹-5 |
| 1441 | CH₃ | NH | Ar⁶ | R¹¹-6 |
| 1442 | CH₃ | NH | Ar⁶ | R¹¹-7 |
| 1443 | CH₃ | NH | Ar⁶ | R¹¹-8 |
| 1444 | CH₃ | NH | Ar⁶ | R¹¹-9 |
| 1445 | CH₃ | NH | Ar⁶ | R¹¹-10 |
| 1446 | CH₃ | NH | Ar⁶ | R¹¹-11 |
| 1447 | CH₃ | NH | Ar⁶ | R¹¹-12 |
| 1448 | CH₃ | NH | Ar⁶ | R¹¹-13 |
| 1449 | CH₃ | NH | Ar⁶ | R¹¹-14 |
| 1450 | CH₃ | NH | Ar⁶ | R¹¹-15 |
| 1451 | CH₃ | NH | Ar⁶ | R¹¹-16 |
| 1452 | CH₃ | NH | Ar⁶ | R¹¹-17 |
| 1453 | CH₃ | NH | Ar⁶ | R¹¹-18 |
| 1454 | CH₃ | NH | Ar⁶ | R¹¹-19 |
| 1455 | CH₃ | NH | Ar⁶ | R¹¹-20 |
| 1456 | CH₃ | NH | Ar⁶ | R¹¹-21 |
| 1457 | CH₃ | NH | Ar⁶ | R¹¹-22 |
| 1458 | CH₃ | NH | Ar⁶ | R¹¹-23 |
| 1459 | CH₃ | NH | Ar⁶ | R¹¹-24 |
| 1460 | CH₃ | NH | Ar⁶ | R¹¹-25 |
| 1461 | CH₃ | NH | Ar⁶ | R¹¹-26 |
| 1462 | CH₃ | NH | Ar⁶ | R¹¹-27 |
| 1463 | CH₃ | NH | Ar⁶ | R¹¹-28 |
| 1464 | CH₃ | NH | Ar⁶ | R¹¹-29 |
| 1465 | CH₃ | NH | Ar⁶ | A¹¹-1a |
| 1466 | CH₃ | NH | Ar⁶ | A¹¹-1b |
| 1467 | CH₃ | NH | Ar⁶ | A¹¹-2a |
| 1468 | CH₃ | NH | Ar⁶ | A¹¹-2b |
| 1469 | CH₃ | NH | Ar⁶ | A¹¹-3a |
| 1470 | CH₃ | NH | Ar⁶ | A¹¹-3b |
| 1471 | CH₃ | NH | Ar⁷ | R¹¹-1 |
| 1472 | CH₃ | NH | Ar⁷ | R¹¹-2 |
| 1473 | CH₃ | NH | Ar⁷ | R¹¹-3 |
| 1474 | CH₃ | NH | Ar⁷ | R¹¹-4 |
| 1475 | CH₃ | NH | Ar⁷ | R¹¹-5 |
| 1476 | CH₃ | NH | Ar⁷ | R¹¹-6 |
| 1477 | CH₃ | NH | Ar⁷ | R¹¹-7 |
| 1478 | CH₃ | NH | Ar⁷ | R¹¹-8 |
| 1479 | CH₃ | NH | Ar⁷ | R¹¹-9 |
| 1480 | CH₃ | NH | Ar⁷ | R¹¹-10 |
| 1481 | CH₃ | NH | Ar⁷ | R¹¹-11 |
| 1482 | CH₃ | NH | Ar⁷ | R¹¹-12 |
| 1483 | CH₃ | NH | Ar⁷ | R¹¹-13 |
| 1484 | CH₃ | NH | Ar⁷ | R¹¹-14 |
| 1485 | CH₃ | NH | Ar⁷ | R¹¹-15 |
| 1486 | CH₃ | NH | Ar⁷ | R¹¹-16 |
| 1487 | CH₃ | NH | Ar⁷ | R¹¹-17 |
| 1488 | CH₃ | NH | Ar⁷ | R¹¹-18 |
| 1489 | CH₃ | NH | Ar⁷ | R¹¹-19 |
| 1490 | CH₃ | NH | Ar⁷ | R¹¹-20 |
| 1491 | CH₃ | NH | Ar⁷ | R¹¹-21 |
| 1492 | CH₃ | NH | Ar⁷ | R¹¹-22 |
| 1493 | CH₃ | NH | Ar⁷ | R¹¹-23 |
| 1494 | CH₃ | NH | Ar⁷ | R¹¹-24 |
| 1495 | CH₃ | NH | Ar⁷ | R¹¹-25 |
| 1496 | CH₃ | NH | Ar⁷ | R¹¹-26 |
| 1497 | CH₃ | NH | Ar⁷ | R¹¹-27 |
| 1498 | CH₃ | NH | Ar⁷ | R¹¹-28 |
| 1499 | CH₃ | NH | Ar⁷ | R¹¹-29 |
| 1500 | CH₃ | NH | Ar⁷ | A¹¹-1a |
| 1501 | CH₃ | NH | Ar⁷ | A¹¹-1b |
| 1502 | CH₃ | NH | Ar⁷ | A¹¹-2a |
| 1503 | CH₃ | NH | Ar⁷ | A¹¹-2b |
| 1504 | CH₃ | NH | Ar⁷ | A¹¹-3a |
| 1505 | CH₃ | NH | Ar⁷ | A¹¹-3b |
| 1506 | CH₃ | NH | Ar⁸ | R¹¹-1 |
| 1507 | CH₃ | NH | Ar⁸ | R¹¹-2 |
| 1508 | CH₃ | NH | Ar⁸ | R¹¹-3 |
| 1509 | CH₃ | NH | Ar⁸ | R¹¹-4 |
| 1510 | CH₃ | NH | Ar⁸ | R¹¹-5 |
| 1511 | CH₃ | NH | Ar⁸ | R¹¹-6 |
| 1512 | CH₃ | NH | Ar⁸ | R¹¹-7 |
| 1513 | CH₃ | NH | Ar⁸ | R¹¹-8 |
| 1514 | CH₃ | NH | Ar⁸ | R¹¹-9 |
| 1515 | CH₃ | NH | Ar⁸ | R¹¹-10 |
| 1516 | CH₃ | NH | Ar⁸ | R¹¹-11 |
| 1517 | CH₃ | NH | Ar⁸ | R¹¹-12 |
| 1518 | CH₃ | NH | Ar⁸ | R¹¹-13 |
| 1519 | CH₃ | NH | Ar⁸ | R¹¹-14 |
| 1520 | CH₃ | NH | Ar⁸ | R¹¹-15 |
| 1521 | CH₃ | NH | Ar⁸ | R¹¹-16 |
| 1522 | CH₃ | NH | Ar⁸ | R¹¹-17 |
| 1523 | CH₃ | NH | Ar⁸ | R¹¹-18 |
| 1524 | CH₃ | NH | Ar⁸ | R¹¹-19 |
| 1525 | CH₃ | NH | Ar⁸ | R¹¹-20 |
| 1526 | CH₃ | NH | Ar⁸ | R¹¹-21 |
| 1527 | CH₃ | NH | Ar⁸ | R¹¹-22 |
| 1528 | CH₃ | NH | Ar⁸ | R¹¹-23 |
| 1529 | CH₃ | NH | Ar⁸ | R¹¹-24 |
| 1530 | CH₃ | NH | Ar⁸ | R¹¹-25 |
| 1531 | CH₃ | NH | Ar⁸ | R¹¹-26 |
| 1532 | CH₃ | NH | Ar⁸ | R¹¹-27 |
| 1533 | CH₃ | NH | Ar⁸ | R¹¹-28 |
| 1534 | CH₃ | NH | Ar⁸ | R¹¹-29 |
| 1535 | CH₃ | NH | Ar⁸ | A¹¹-1a |
| 1536 | CH₃ | NH | Ar⁸ | A¹¹-1b |
| 1537 | CH₃ | NH | Ar⁸ | A¹¹-2a |
| 1538 | CH₃ | NH | Ar⁸ | A¹¹-2b |
| 1539 | CH₃ | NH | Ar⁸ | A¹¹-3a |
| 1540 | CH₃ | NH | Ar⁸ | A¹¹-3b |
| 1541 | CH₃ | NH | Ar⁹ | R¹¹-1 |
| 1542 | CH₃ | NH | Ar⁹ | R¹¹-2 |
| 1543 | CH₃ | NH | Ar⁹ | R¹¹-3 |
| 1544 | CH₃ | NH | Ar⁹ | R¹¹-4 |
| 1545 | CH₃ | NH | Ar⁹ | R¹¹-5 |
| 1546 | CH₃ | NH | Ar⁹ | R¹¹-6 |
| 1547 | CH₃ | NH | Ar⁹ | R¹¹-7 |
| 1548 | CH₃ | NH | Ar⁹ | R¹¹-8 |
| 1549 | CH₃ | NH | Ar⁹ | R¹¹-9 |
| 1550 | CH₃ | NH | Ar⁹ | R¹¹-10 |
| 1551 | CH₃ | NH | Ar⁹ | R¹¹-11 |
| 1552 | CH₃ | NH | Ar⁹ | R¹¹-12 |
| 1553 | CH₃ | NH | Ar⁹ | R¹¹-13 |
| 1554 | CH₃ | NH | Ar⁹ | R¹¹-14 |
| 1555 | CH₃ | NH | Ar⁹ | R¹¹-15 |
| 1556 | CH₃ | NH | Ar⁹ | R¹¹-16 |
| 1557 | CH₃ | NH | Ar⁹ | R¹¹-17 |
| 1558 | CH₃ | NH | Ar⁹ | R¹¹-18 |
| 1559 | CH₃ | NH | Ar⁹ | R¹¹-19 |
| 1560 | CH₃ | NH | Ar⁹ | R¹¹-20 |
| 1561 | CH₃ | NH | Ar⁹ | R¹¹-21 |
| 1562 | CH₃ | NH | Ar⁹ | R¹¹-22 |
| 1563 | CH₃ | NH | Ar⁹ | R¹¹-23 |
| 1564 | CH₃ | NH | Ar⁹ | R¹¹-24 |
| 1565 | CH₃ | NH | Ar⁹ | R¹¹-25 |
| 1566 | CH₃ | NH | Ar⁹ | R¹¹-26 |
| 1567 | CH₃ | NH | Ar⁹ | R¹¹-27 |
| 1568 | CH₃ | NH | Ar⁹ | R¹¹-28 |
| 1569 | CH₃ | NH | Ar⁹ | R¹¹-29 |
| 1570 | CH₃ | NH | Ar⁹ | A¹¹-1a |
| 1571 | CH₃ | NH | Ar⁹ | A¹¹-1b |
| 1572 | CH₃ | NH | Ar⁹ | A¹¹-2a |
| 1573 | CH₃ | NH | Ar⁹ | A¹¹-2b |
| 1574 | CH₃ | NH | Ar⁹ | A¹¹-3a |
| 1575 | CH₃ | NH | Ar⁹ | A¹¹-3b |
| 1576 | CH₃ | NH | Ar¹⁰ | R¹¹-1 |
| 1577 | CH₃ | NH | Ar¹⁰ | R¹¹-2 |
| 1578 | CH₃ | NH | Ar¹⁰ | R¹¹-3 |
| 1579 | CH₃ | NH | Ar¹⁰ | R¹¹-4 |
| 1580 | CH₃ | NH | Ar¹⁰ | R¹¹-5 |
| 1581 | CH₃ | NH | Ar¹⁰ | R¹¹-6 |
| 1582 | CH₃ | NH | Ar¹⁰ | R¹¹-7 |
| 1583 | CH₃ | NH | Ar¹⁰ | R¹¹-8 |
| 1584 | CH₃ | NH | Ar¹⁰ | R¹¹-9 |
| 1585 | CH₃ | NH | Ar¹⁰ | R¹¹-10 |
| 1586 | CH₃ | NH | Ar¹⁰ | R¹¹-11 |
| 1587 | CH₃ | NH | Ar¹⁰ | R¹¹-12 |
| 1588 | CH₃ | NH | Ar¹⁰ | R¹¹-13 |
| 1589 | CH₃ | NH | Ar¹⁰ | R¹¹-14 |
| 1590 | CH₃ | NH | Ar¹⁰ | R¹¹-15 |
| 1591 | CH₃ | NH | Ar¹⁰ | R¹¹-16 |
| 1592 | CH₃ | NH | Ar¹⁰ | R¹¹-17 |
| 1593 | CH₃ | NH | Ar¹⁰ | R¹¹-18 |
| 1594 | CH₃ | NH | Ar¹⁰ | R¹¹-19 |
| 1595 | CH₃ | NH | Ar¹⁰ | R¹¹-20 |
| 1596 | CH₃ | NH | Ar¹⁰ | R¹¹-21 |
| 1597 | CH₃ | NH | Ar¹⁰ | R¹¹-22 |
| 1598 | CH₃ | NH | Ar¹⁰ | R¹¹-23 |
| 1599 | CH₃ | NH | Ar¹⁰ | R¹¹-24 |
| 1600 | CH₃ | NH | Ar¹⁰ | R¹¹-25 |
| 1601 | CH₃ | NH | Ar¹⁰ | R¹¹-26 |
| 1602 | CH₃ | NH | Ar¹⁰ | R¹¹-27 |
| 1603 | CH₃ | NH | Ar¹⁰ | R¹¹-28 |
| 1604 | CH₃ | NH | Ar¹⁰ | R¹¹-29 |
| 1605 | CH₃ | NH | Ar¹⁰ | A¹¹-1a |
| 1606 | CH₃ | NH | Ar¹⁰ | A¹¹-1b |
| 1607 | CH₃ | NH | Ar¹⁰ | A¹¹-2a |
| 1608 | CH₃ | NH | Ar¹⁰ | A¹¹-2b |
| 1609 | CH₃ | NH | Ar¹⁰ | A¹¹-3a |
| 1610 | CH₃ | NH | Ar¹⁰ | A¹¹-3b |
| 1611 | CH₃ | NH | Ar¹¹ | R¹¹-1 |
| 1612 | CH₃ | NH | Ar¹¹ | R¹¹-2 |
| 1613 | CH₃ | NH | Ar¹¹ | R¹¹-3 |
| 1614 | CH₃ | NH | Ar¹¹ | R¹¹-4 |
| 1615 | CH₃ | NH | Ar¹¹ | R¹¹-5 |
| 1616 | CH₃ | NH | Ar¹¹ | R¹¹-6 |
| 1617 | CH₃ | NH | Ar¹¹ | R¹¹-7 |
| 1618 | CH₃ | NH | Ar¹¹ | R¹¹-8 |
| 1619 | CH₃ | NH | Ar¹¹ | R¹¹-9 |
| 1620 | CH₃ | NH | Ar¹¹ | R¹¹-10 |
| 1621 | CH₃ | NH | Ar¹¹ | R¹¹-11 |
| 1622 | CH₃ | NH | Ar¹¹ | R¹¹-12 |
| 1623 | CH₃ | NH | Ar¹¹ | R¹¹-13 |
| 1624 | CH₃ | NH | Ar¹¹ | R¹¹-14 |
| 1625 | CH₃ | NH | Ar¹¹ | R¹¹-15 |
| 1626 | CH₃ | NH | Ar¹¹ | R¹¹-16 |
| 1627 | CH₃ | NH | Ar¹¹ | R¹¹-17 |
| 1628 | CH₃ | NH | Ar¹¹ | R¹¹-18 |
| 1629 | CH₃ | NH | Ar¹¹ | R¹¹-19 |
| 1630 | CH₃ | NH | Ar¹¹ | R¹¹-20 |
| 1631 | CH₃ | NH | Ar¹¹ | R¹¹-21 |
| 1632 | CH₃ | NH | Ar¹¹ | R¹¹-22 |
| 1633 | CH₃ | NH | Ar¹¹ | R¹¹-23 |
| 1634 | CH₃ | NH | Ar¹¹ | R¹¹-24 |
| 1635 | CH₃ | NH | Ar¹¹ | R¹¹-25 |
| 1636 | CH₃ | NH | Ar¹¹ | R¹¹-26 |
| 1637 | CH₃ | NH | Ar¹¹ | R¹¹-27 |
| 1638 | CH₃ | NH | Ar¹¹ | R¹¹-28 |
| 1639 | CH₃ | NH | Ar¹¹ | R¹¹-29 |
| 1640 | CH₃ | NH | Ar¹¹ | A¹¹-1a |
| 1641 | CH₃ | NH | Ar¹¹ | A¹¹-1b |
| 1642 | CH₃ | NH | Ar¹¹ | A¹¹-2a |
| 1643 | CH₃ | NH | Ar¹¹ | A¹¹-2b |
| 1644 | CH₃ | NH | Ar¹¹ | A¹¹-3a |
| 1645 | CH₃ | NH | Ar¹¹ | A¹¹-3b |
| 1646 | CH₃ | NH | Ar¹² | R¹¹-1 |
| 1647 | CH₃ | NH | Ar¹² | R¹¹-2 |
| 1648 | CH₃ | NH | Ar¹² | R¹¹-3 |
| 1649 | CH₃ | NH | Ar¹² | R¹¹-4 |
| 1650 | CH₃ | NH | Ar¹² | R¹¹-5 |
| 1651 | CH₃ | NH | Ar¹² | R¹¹-6 |
| 1652 | CH₃ | NH | Ar¹² | R¹¹-7 |
| 1653 | CH₃ | NH | Ar¹² | R¹¹-8 |
| 1654 | CH₃ | NH | Ar¹² | R¹¹-9 |
| 1655 | CH₃ | NH | Ar¹² | R¹¹-10 |
| 1656 | CH₃ | NH | Ar¹² | R¹¹-11 |
| 1657 | CH₃ | NH | Ar¹² | R¹¹-12 |
| 1658 | CH₃ | NH | Ar¹² | R¹¹-13 |
| 1659 | CH₃ | NH | Ar¹² | R¹¹-14 |
| 1660 | CH₃ | NH | Ar¹² | R¹¹-15 |
| 1661 | CH₃ | NH | Ar¹² | R¹¹-16 |
| 1662 | CH₃ | NH | Ar¹² | R¹¹-17 |
| 1663 | CH₃ | NH | Ar¹² | R¹¹-18 |
| 1664 | CH₃ | NH | Ar¹² | R¹¹-19 |
| 1665 | CH₃ | NH | Ar¹² | R¹¹-20 |
| 1666 | CH₃ | NH | Ar¹² | R¹¹-21 |
| 1667 | CH₃ | NH | Ar¹² | R¹¹-22 |
| 1668 | CH₃ | NH | Ar¹² | R¹¹-23 |
| 1669 | CH₃ | NH | Ar¹² | R¹¹-24 |
| 1670 | CH₃ | NH | Ar¹² | R¹¹-25 |
| 1671 | CH₃ | NH | Ar¹² | R¹¹-26 |
| 1672 | CH₃ | NH | Ar¹² | R¹¹-27 |
| 1673 | CH₃ | NH | Ar¹² | R¹¹-28 |
| 1674 | CH₃ | NH | Ar¹² | R¹¹-29 |
| 1675 | CH₃ | NH | Ar¹² | A¹¹-1a |
| 1676 | CH₃ | NH | Ar¹² | A¹¹-1b |
| 1677 | CH₃ | NH | Ar¹² | A¹¹-2a |
| 1678 | CH₃ | NH | Ar¹² | A¹¹-2b |
| 1679 | CH₃ | NH | Ar¹² | A¹¹-3a |
| 1680 | CH₃ | NH | Ar¹² | A¹¹-3b |
| 1681 | CH₃ | NCH₃ | Ar¹ | R¹¹-1 |
| 1682 | CH₃ | NCH₃ | Ar¹ | R¹¹-2 |
| 1683 | CH₃ | NCH₃ | Ar¹ | R¹¹-3 |
| 1684 | CH₃ | NCH₃ | Ar¹ | R¹¹-4 |
| 1685 | CH₃ | NCH₃ | Ar¹ | R¹¹-5 |
| 1686 | CH₃ | NCH₃ | Ar¹ | R¹¹-6 |
| 1687 | CH₃ | NCH₃ | Ar¹ | R¹¹-7 |
| 1688 | CH₃ | NCH₃ | Ar¹ | R¹¹-8 |
| 1689 | CH₃ | NCH₃ | Ar¹ | R¹¹-9 |
| 1690 | CH₃ | NCH₃ | Ar¹ | R¹¹-10 |
| 1691 | CH₃ | NCH₃ | Ar¹ | R¹¹-11 |
| 1692 | CH₃ | NCH₃ | Ar¹ | R¹¹-12 |
| 1693 | CH₃ | NCH₃ | Ar¹ | R¹¹-13 |
| 1694 | CH₃ | NCH₃ | Ar¹ | R¹¹-14 |
| 1695 | CH₃ | NCH₃ | Ar¹ | R¹¹-15 |
| 1696 | CH₃ | NCH₃ | Ar¹ | R¹¹-16 |
| 1697 | CH₃ | NCH₃ | Ar¹ | R¹¹-17 |
| 1698 | CH₃ | NCH₃ | Ar¹ | R¹¹-18 |
| 1699 | CH₃ | NCH₃ | Ar¹ | R¹¹-19 |
| 1700 | CH₃ | NCH₃ | Ar¹ | R¹¹-20 |
| 1701 | CH₃ | NCH₃ | Ar¹ | R¹¹-21 |
| 1702 | CH₃ | NCH₃ | Ar¹ | R¹¹-22 |
| 1703 | CH₃ | NCH₃ | Ar¹ | R¹¹-23 |
| 1704 | CH₃ | NCH₃ | Ar¹ | R¹¹-24 |
| 1705 | CH₃ | NCH₃ | Ar¹ | R¹¹-25 |
| 1706 | CH₃ | NCH₃ | Ar¹ | R¹¹-26 |
| 1707 | CH₃ | NCH₃ | Ar¹ | R¹¹-27 |
| 1708 | CH₃ | NCH₃ | Ar¹ | R¹¹-28 |
| 1709 | CH₃ | NCH₃ | Ar¹ | R¹¹-29 |
| 1710 | CH₃ | NCH₃ | Ar¹ | A¹¹-1a |
| 1711 | CH₃ | NCH₃ | Ar¹ | A¹¹-1b |
| 1712 | CH₃ | NCH₃ | Ar¹ | A¹¹-2a |
| 1713 | CH₃ | NCH₃ | Ar¹ | A¹¹-2b |
| 1714 | CH₃ | NCH₃ | Ar¹ | A¹¹-3a |
| 1715 | CH₃ | NCH₃ | Ar¹ | A¹¹-3b |
| 1716 | CH₃ | NCH₃ | Ar² | R¹¹-1 |
| 1717 | CH₃ | NCH₃ | Ar² | R¹¹-2 |
| 1718 | CH₃ | NCH₃ | Ar² | R¹¹-3 |
| 1719 | CH₃ | NCH₃ | Ar² | R¹¹-4 |
| 1720 | CH₃ | NCH₃ | Ar² | R¹¹-5 |
| 1721 | CH₃ | NCH₃ | Ar² | R¹¹-6 |
| 1722 | CH₃ | NCH₃ | Ar² | R¹¹-7 |
| 1723 | CH₃ | NCH₃ | Ar² | R¹¹-8 |
| 1724 | CH₃ | NCH₃ | Ar² | R¹¹-9 |
| 1725 | CH₃ | NCH₃ | Ar² | R¹¹-10 |
| 1726 | CH₃ | NCH₃ | Ar² | R¹¹-11 |
| 1727 | CH₃ | NCH₃ | Ar² | R¹¹-12 |
| 1728 | CH₃ | NCH₃ | Ar² | R¹¹-13 |
| 1729 | CH₃ | NCH₃ | Ar² | R¹¹-14 |
| 1730 | CH₃ | NCH₃ | Ar² | R¹¹-15 |
| 1731 | CH₃ | NCH₃ | Ar² | R¹¹-16 |
| 1732 | CH₃ | NCH₃ | Ar² | R¹¹-17 |
| 1733 | CH₃ | NCH₃ | Ar² | R¹¹-18 |
| 1734 | CH₃ | NCH₃ | Ar² | R¹¹-19 |
| 1735 | CH₃ | NCH₃ | Ar² | R¹¹-20 |
| 1736 | CH₃ | NCH₃ | Ar² | R¹¹-21 |
| 1737 | CH₃ | NCH₃ | Ar² | R¹¹-22 |
| 1738 | CH₃ | NCH₃ | Ar² | R¹¹-23 |
| 1739 | CH₃ | NCH₃ | Ar² | R¹¹-24 |
| 1740 | CH₃ | NCH₃ | Ar² | R¹¹-25 |
| 1741 | CH₃ | NCH₃ | Ar² | R¹¹-26 |
| 1742 | CH₃ | NCH₃ | Ar² | R¹¹-27 |
| 1743 | CH₃ | NCH₃ | Ar² | R¹¹-28 |
| 1744 | CH₃ | NCH₃ | Ar² | R¹¹-29 |
| 1745 | CH₃ | NCH₃ | Ar² | A¹¹-1a |
| 1746 | CH₃ | NCH₃ | Ar² | A¹¹-1b |
| 1747 | CH₃ | NCH₃ | Ar² | A¹¹-2a |
| 1748 | CH₃ | NCH₃ | Ar² | A¹¹-2b |
| 1749 | CH₃ | NCH₃ | Ar² | A¹¹-3a |
| 1750 | CH₃ | NCH₃ | Ar² | A¹¹-3b |
| 1751 | CH₃ | NCH₃ | Ar³ | R¹¹-1 |
| 1752 | CH₃ | NCH₃ | Ar³ | R¹¹-2 |
| 1753 | CH₃ | NCH₃ | Ar³ | R¹¹-3 |
| 1754 | CH₃ | NCH₃ | Ar³ | R¹¹-4 |
| 1755 | CH₃ | NCH₃ | Ar³ | R¹¹-5 |
| 1756 | CH₃ | NCH₃ | Ar³ | R¹¹-6 |
| 1757 | CH₃ | NCH₃ | Ar³ | R¹¹-7 |
| 1758 | CH₃ | NCH₃ | Ar³ | R¹¹-8 |
| 1759 | CH₃ | NCH₃ | Ar³ | R¹¹-9 |
| 1760 | CH₃ | NCH₃ | Ar³ | R¹¹-10 |
| 1761 | CH₃ | NCH₃ | Ar³ | R¹¹-11 |
| 1762 | CH₃ | NCH₃ | Ar³ | R¹¹-12 |
| 1763 | CH₃ | NCH₃ | Ar³ | R¹¹-13 |
| 1764 | CH₃ | NCH₃ | Ar³ | R¹¹-14 |
| 1765 | CH₃ | NCH₃ | Ar³ | R¹¹-15 |
| 1766 | CH₃ | NCH₃ | Ar³ | R¹¹-16 |
| 1767 | CH₃ | NCH₃ | Ar³ | R¹¹-17 |
| 1768 | CH₃ | NCH₃ | Ar³ | R¹¹-18 |
| 1769 | CH₃ | NCH₃ | Ar³ | R¹¹-19 |
| 1770 | CH₃ | NCH₃ | Ar³ | R¹¹-20 |
| 1771 | CH₃ | NCH₃ | Ar³ | R¹¹-21 |
| 1772 | CH₃ | NCH₃ | Ar³ | R¹¹-22 |
| 1773 | CH₃ | NCH₃ | Ar³ | R¹¹-23 |
| 1774 | CH₃ | NCH₃ | Ar³ | R¹¹-24 |
| 1775 | CH₃ | NCH₃ | Ar³ | R¹¹-25 |
| 1776 | CH₃ | NCH₃ | Ar³ | R¹¹-26 |
| 1777 | CH₃ | NCH₃ | Ar³ | R¹¹-27 |
| 1778 | CH₃ | NCH₃ | Ar³ | R¹¹-28 |
| 1779 | CH₃ | NCH₃ | Ar³ | R¹¹-29 |
| 1780 | CH₃ | NCH₃ | Ar³ | A¹¹-1a |
| 1781 | CH₃ | NCH₃ | Ar³ | A¹¹-1b |
| 1782 | CH₃ | NCH₃ | Ar³ | A¹¹-2a |
| 1783 | CH₃ | NCH₃ | Ar³ | A¹¹-2b |
| 1784 | CH₃ | NCH₃ | Ar³ | A¹¹-3a |
| 1785 | CH₃ | NCH₃ | Ar⁴ | A¹¹-3b |
| 1786 | CH₃ | NCH₃ | Ar⁴ | R¹¹-1 |
| 1787 | CH₃ | NCH₃ | Ar⁴ | R¹¹-2 |
| 1788 | CH₃ | NCH₃ | Ar⁴ | R¹¹-3 |
| 1789 | CH₃ | NCH₃ | Ar⁴ | R¹¹-4 |
| 1790 | CH₃ | NCH₃ | Ar⁴ | R¹¹-5 |
| 1791 | CH₃ | NCH₃ | Ar⁴ | R¹¹-6 |
| 1792 | CH₃ | NCH₃ | Ar⁴ | R¹¹-7 |
| 1793 | CH₃ | NCH₃ | Ar⁴ | R¹¹-8 |
| 1794 | CH₃ | NCH₃ | Ar⁴ | R¹¹-9 |
| 1795 | CH₃ | NCH₃ | Ar⁴ | R¹¹-10 |
| 1796 | CH₃ | NCH₃ | Ar⁴ | R¹¹-11 |
| 1797 | CH₃ | NCH₃ | Ar⁴ | R¹¹-12 |
| 1798 | CH₃ | NCH₃ | Ar⁴ | R¹¹-13 |
| 1799 | CH₃ | NCH₃ | Ar⁴ | R¹¹-14 |
| 1800 | CH₃ | NCH₃ | Ar⁴ | R¹¹-15 |
| 1801 | CH₃ | NCH₃ | Ar⁴ | R¹¹-16 |
| 1802 | CH₃ | NCH₃ | Ar⁴ | R¹¹-17 |
| 1803 | CH₃ | NCH₃ | Ar⁴ | R¹¹-18 |
| 1804 | CH₃ | NCH₃ | Ar⁴ | R¹¹-19 |
| 1805 | CH₃ | NCH₃ | Ar⁴ | R¹¹-20 |
| 1806 | CH₃ | NCH₃ | Ar⁴ | R¹¹-21 |
| 1807 | CH₃ | NCH₃ | Ar⁴ | R¹¹-22 |
| 1808 | CH₃ | NCH₃ | Ar⁴ | R¹¹-23 |
| 1809 | CH₃ | NCH₃ | Ar⁴ | R¹¹-24 |
| 1810 | CH₃ | NCH₃ | Ar⁴ | R¹¹-25 |
| 1811 | CH₃ | NCH₃ | Ar⁴ | R¹¹-26 |
| 1812 | CH₃ | NCH₃ | Ar⁴ | R¹¹-27 |
| 1813 | CH₃ | NCH₃ | Ar⁴ | R¹¹-28 |
| 1814 | CH₃ | NCH₃ | Ar⁴ | R¹¹-29 |
| 1815 | CH₃ | NCH₃ | Ar⁴ | A¹¹-1a |
| 1816 | CH₃ | NCH₃ | Ar⁴ | A¹¹-1b |
| 1817 | CH₃ | NCH₃ | Ar⁴ | A¹¹-2a |
| 1818 | CH₃ | NCH₃ | Ar⁴ | A¹¹-2b |
| 1819 | CH₃ | NCH₃ | Ar⁴ | A¹¹-3a |
| 1820 | CH₃ | NCH₃ | Ar⁴ | A¹¹-3b |
| 1821 | CH₃ | NCH₃ | Ar⁵ | R¹¹-1 |
| 1822 | CH₃ | NCH₃ | Ar⁵ | R¹¹-2 |
| 1823 | CH₃ | NCH₃ | Ar⁵ | R¹¹-3 |
| 1824 | CH₃ | NCH₃ | Ar⁵ | R¹¹-4 |
| 1825 | CH₃ | NCH₃ | Ar⁵ | R¹¹-5 |
| 1826 | CH₃ | NCH₃ | Ar⁵ | R¹¹-6 |
| 1827 | CH₃ | NCH₃ | Ar⁵ | R¹¹-7 |
| 1828 | CH₃ | NCH₃ | Ar⁵ | R¹¹-8 |
| 1829 | CH₃ | NCH₃ | Ar⁵ | R¹¹-9 |
| 1830 | CH₃ | NCH₃ | Ar⁵ | R¹¹-10 |
| 1831 | CH₃ | NCH₃ | Ar⁵ | R¹¹-11 |
| 1832 | CH₃ | NCH₃ | Ar⁵ | R¹¹-12 |
| 1833 | CH₃ | NCH₃ | Ar⁵ | R¹¹-13 |
| 1834 | CH₃ | NCH₃ | Ar⁵ | R¹¹-14 |
| 1835 | CH₃ | NCH₃ | Ar⁵ | R¹¹-15 |
| 1836 | CH₃ | NCH₃ | Ar⁵ | R¹¹-16 |
| 1837 | CH₃ | NCH₃ | Ar⁵ | R¹¹-17 |
| 1838 | CH₃ | NCH₃ | Ar⁵ | R¹¹-18 |
| 1839 | CH₃ | NCH₃ | Ar⁵ | R¹¹-19 |
| 1840 | CH₃ | NCH₃ | Ar⁵ | R¹¹-20 |
| 1841 | CH₃ | NCH₃ | Ar⁵ | R¹¹-21 |
| 1842 | CH₃ | NCH₃ | Ar⁵ | R¹¹-22 |
| 1843 | CH₃ | NCH₃ | Ar⁵ | R¹¹-23 |
| 1844 | CH₃ | NCH₃ | Ar⁵ | R¹¹-24 |
| 1845 | CH₃ | NCH₃ | Ar⁵ | R¹¹-25 |
| 1846 | CH₃ | NCH₃ | Ar⁵ | R¹¹-26 |
| 1847 | CH₃ | NCH₃ | Ar⁵ | R¹¹-27 |
| 1848 | CH₃ | NCH₃ | Ar⁵ | R¹¹-28 |
| 1849 | CH₃ | NCH₃ | Ar⁵ | R¹¹-29 |
| 1850 | CH₃ | NCH₃ | Ar⁵ | A¹¹-1a |
| 1851 | CH₃ | NCH₃ | Ar⁵ | A¹¹-1b |
| 1852 | CH₃ | NCH₃ | Ar⁵ | A¹¹-2a |
| 1853 | CH₃ | NCH₃ | Ar⁵ | A¹¹-2b |
| 1854 | CH₃ | NCH₃ | Ar⁵ | A¹¹-3a |
| 1855 | CH₃ | NCH₃ | Ar⁵ | A¹¹-3b |
| 1856 | CH₃ | NCH₃ | Ar⁶ | R¹¹-1 |
| 1857 | CH₃ | NCH₃ | Ar⁶ | R¹¹-2 |
| 1858 | CH₃ | NCH₃ | Ar⁶ | R¹¹-3 |
| 1859 | CH₃ | NCH₃ | Ar⁶ | R¹¹-4 |
| 1860 | CH₃ | NCH₃ | Ar⁶ | R¹¹-5 |
| 1861 | CH₃ | NCH₃ | Ar⁶ | R¹¹-6 |
| 1862 | CH₃ | NCH₃ | Ar⁶ | R¹¹-7 |
| 1863 | CH₃ | NCH₃ | Ar⁶ | R¹¹-8 |
| 1864 | CH₃ | NCH₃ | Ar⁶ | R¹¹-9 |
| 1865 | CH₃ | NCH₃ | Ar⁶ | R¹¹-10 |
| 1866 | CH₃ | NCH₃ | Ar⁶ | R¹¹-11 |
| 1867 | CH₃ | NCH₃ | Ar⁶ | R¹¹-12 |
| 1868 | CH₃ | NCH₃ | Ar⁶ | R¹¹-13 |
| 1869 | CH₃ | NCH₃ | Ar⁶ | R¹¹-14 |
| 1870 | CH₃ | NCH₃ | Ar⁶ | R¹¹-15 |
| 1871 | CH₃ | NCH₃ | Ar⁶ | R¹¹-16 |
| 1872 | CH₃ | NCH₃ | Ar⁶ | R¹¹-17 |
| 1873 | CH₃ | NCH₃ | Ar⁶ | R¹¹-18 |
| 1874 | CH₃ | NCH₃ | Ar⁶ | R¹¹-19 |
| 1875 | CH₃ | NCH₃ | Ar⁶ | R¹¹-20 |
| 1876 | CH₃ | NCH₃ | Ar⁶ | R¹¹-21 |
| 1877 | CH₃ | NCH₃ | Ar⁶ | R¹¹-22 |
| 1878 | CH₃ | NCH₃ | Ar⁶ | R¹¹-23 |
| 1879 | CH₃ | NCH₃ | Ar⁶ | R¹¹-24 |
| 1880 | CH₃ | NCH₃ | Ar⁶ | R¹¹-25 |
| 1881 | CH₃ | NCH₃ | Ar⁶ | R¹¹-26 |
| 1882 | CH₃ | NCH₃ | Ar⁶ | R¹¹-27 |
| 1883 | CH₃ | NCH₃ | Ar⁶ | R¹¹-28 |
| 1884 | CH₃ | NCH₃ | Ar⁶ | R¹¹-29 |
| 1885 | CH₃ | NCH₃ | Ar⁶ | A¹¹-1a |
| 1886 | CH₃ | NCH₃ | Ar⁶ | A¹¹-1b |
| 1887 | CH₃ | NCH₃ | Ar⁶ | A¹¹-2a |
| 1888 | CH₃ | NCH₃ | Ar⁶ | A¹¹-2b |
| 1889 | CH₃ | NCH₃ | Ar⁶ | A¹¹-3a |
| 1890 | CH₃ | NCH₃ | Ar⁶ | A¹¹-3b |
| 1891 | CH₃ | NCH₃ | Ar⁷ | R¹¹-1 |
| 1892 | CH₃ | NCH₃ | Ar⁷ | R¹¹-2 |
| 1893 | CH₃ | NCH₃ | Ar⁷ | R¹¹-3 |
| 1894 | CH₃ | NCH₃ | Ar⁷ | R¹¹-4 |
| 1895 | CH₃ | NCH₃ | Ar⁷ | R¹¹-5 |
| 1896 | CH₃ | NCH₃ | Ar⁷ | R¹¹-6 |
| 1897 | CH₃ | NCH₃ | Ar⁷ | R¹¹-7 |
| 1898 | CH₃ | NCH₃ | Ar⁷ | R¹¹-8 |
| 1899 | CH₃ | NCH₃ | Ar⁷ | R¹¹-9 |
| 1900 | CH₃ | NCH₃ | Ar⁷ | R¹¹-10 |
| 1901 | CH₃ | NCH₃ | Ar⁷ | R¹¹-11 |
| 1902 | CH₃ | NCH₃ | Ar⁷ | R¹¹-12 |
| 1903 | CH₃ | NCH₃ | Ar⁷ | R¹¹-13 |
| 1904 | CH₃ | NCH₃ | Ar⁷ | R¹¹-14 |
| 1905 | CH₃ | NCH₃ | Ar⁷ | R¹¹-15 |
| 1906 | CH₃ | NCH₃ | Ar⁷ | R¹¹-16 |
| 1907 | CH₃ | NCH₃ | Ar⁷ | R¹¹-17 |
| 1908 | CH₃ | NCH₃ | Ar⁷ | R¹¹-18 |
| 1909 | CH₃ | NCH₃ | Ar⁷ | R¹¹-19 |
| 1910 | CH₃ | NCH₃ | Ar⁷ | R¹¹-20 |
| 1911 | CH₃ | NCH₃ | Ar⁷ | R¹¹-21 |
| 1912 | CH₃ | NCH₃ | Ar⁷ | R¹¹-22 |
| 1913 | CH₃ | NCH₃ | Ar⁷ | R¹¹-23 |
| 1914 | CH₃ | NCH₃ | Ar⁷ | R¹¹-24 |
| 1915 | CH₃ | NCH₃ | Ar⁷ | R¹¹-25 |
| 1916 | CH₃ | NCH₃ | Ar⁷ | R¹¹-26 |
| 1917 | CH₃ | NCH₃ | Ar⁷ | R¹¹-27 |
| 1918 | CH₃ | NCH₃ | Ar⁷ | R¹¹-28 |
| 1919 | CH₃ | NCH₃ | Ar⁷ | R¹¹-29 |
| 1920 | CH₃ | NCH₃ | Ar⁷ | A¹¹-1a |
| 1921 | CH₃ | NCH₃ | Ar⁷ | A¹¹-1b |
| 1922 | CH₃ | NCH₃ | Ar⁷ | A¹¹-2a |
| 1923 | CH₃ | NCH₃ | Ar⁷ | A¹¹-2b |
| 1924 | CH₃ | NCH₃ | Ar⁷ | A¹¹-3a |
| 1925 | CH₃ | NCH₃ | Ar⁷ | A¹¹-3b |
| 1926 | CH₃ | NCH₃ | Ar⁸ | R¹¹-1 |
| 1927 | CH₃ | NCH₃ | Ar⁸ | R¹¹-2 |
| 1928 | CH₃ | NCH₃ | Ar⁸ | R¹¹-3 |
| 1929 | CH₃ | NCH₃ | Ar⁸ | R¹¹-4 |
| 1930 | CH₃ | NCH₃ | Ar⁸ | R¹¹-5 |
| 1931 | CH₃ | NCH₃ | Ar⁸ | R¹¹-6 |
| 1932 | CH₃ | NCH₃ | Ar⁸ | R¹¹-7 |
| 1933 | CH₃ | NCH₃ | Ar⁸ | R¹¹-8 |
| 1934 | CH₃ | NCH₃ | Ar⁸ | R¹¹-9 |
| 1935 | CH₃ | NCH₃ | Ar⁸ | R¹¹-10 |
| 1936 | CH₃ | NCH₃ | Ar⁸ | R¹¹-11 |
| 1937 | CH₃ | NCH₃ | Ar⁸ | R¹¹-12 |
| 1938 | CH₃ | NCH₃ | Ar⁸ | R¹¹-13 |
| 1939 | CH₃ | NCH₃ | Ar⁸ | R¹¹-14 |
| 1940 | CH₃ | NCH₃ | Ar⁸ | R¹¹-15 |
| 1941 | CH₃ | NCH₃ | Ar⁸ | R¹¹-16 |
| 1942 | CH₃ | NCH₃ | Ar⁸ | R¹¹-17 |
| 1943 | CH₃ | NCH₃ | Ar⁸ | R¹¹-18 |
| 1944 | CH₃ | NCH₃ | Ar⁸ | R¹¹-19 |
| 1945 | CH₃ | NCH₃ | Ar⁸ | R¹¹-20 |
| 1946 | CH₃ | NCH₃ | Ar⁸ | R¹¹-21 |
| 1947 | CH₃ | NCH₃ | Ar⁸ | R¹¹-22 |
| 1948 | CH₃ | NCH₃ | Ar⁸ | R¹¹-23 |
| 1949 | CH₃ | NCH₃ | Ar⁸ | R¹¹-24 |
| 1950 | CH₃ | NCH₃ | Ar⁸ | R¹¹-25 |
| 1951 | CH₃ | NCH₃ | Ar⁸ | R¹¹-26 |
| 1952 | CH₃ | NCH₃ | Ar⁸ | R¹¹-27 |
| 1953 | CH₃ | NCH₃ | Ar⁸ | R¹¹-28 |
| 1954 | CH₃ | NCH₃ | Ar⁸ | R¹¹-29 |
| 1955 | CH₃ | NCH₃ | Ar⁸ | A¹¹-1a |
| 1956 | CH₃ | NCH₃ | Ar⁸ | A¹¹-1b |
| 1957 | CH₃ | NCH₃ | Ar⁸ | A¹¹-2a |
| 1958 | CH₃ | NCH₃ | Ar⁸ | A¹¹-2b |
| 1959 | CH₃ | NCH₃ | Ar⁸ | A¹¹-3a |
| 1960 | CH₃ | NCH₃ | Ar⁸ | A¹¹-3b |
| 1961 | CH₃ | NCH₃ | Ar⁹ | R¹¹-1 |
| 1962 | CH₃ | NCH₃ | Ar⁹ | R¹¹-2 |
| 1963 | CH₃ | NCH₃ | Ar⁹ | R¹¹-3 |
| 1964 | CH₃ | NCH₃ | Ar⁹ | R¹¹-4 |
| 1965 | CH₃ | NCH₃ | Ar⁹ | R¹¹-5 |
| 1966 | CH₃ | NCH₃ | Ar⁹ | R¹¹-6 |
| 1967 | CH₃ | NCH₃ | Ar⁹ | R¹¹-7 |
| 1968 | CH₃ | NCH₃ | Ar⁹ | R¹¹-8 |
| 1969 | CH₃ | NCH₃ | Ar⁹ | R¹¹-9 |
| 1970 | CH₃ | NCH₃ | Ar⁹ | R¹¹-10 |
| 1971 | CH₃ | NCH₃ | Ar⁹ | R¹¹-11 |
| 1972 | CH₃ | NCH₃ | Ar⁹ | R¹¹-12 |
| 1973 | CH₃ | NCH₃ | Ar⁹ | R¹¹-13 |
| 1974 | CH₃ | NCH₃ | Ar⁹ | R¹¹-14 |
| 1975 | CH₃ | NCH₃ | Ar⁹ | R¹¹-15 |
| 1976 | CH₃ | NCH₃ | Ar⁹ | R¹¹-16 |
| 1977 | CH₃ | NCH₃ | Ar⁹ | R¹¹-17 |
| 1978 | CH₃ | NCH₃ | Ar⁹ | R¹¹-18 |
| 1979 | CH₃ | NCH₃ | Ar⁹ | R¹¹-19 |
| 1980 | CH₃ | NCH₃ | Ar⁹ | R¹¹-20 |
| 1981 | CH₃ | NCH₃ | Ar⁹ | R¹¹-21 |
| 1982 | CH₃ | NCH₃ | Ar⁹ | R¹¹-22 |
| 1983 | CH₃ | NCH₃ | Ar⁹ | R¹¹-23 |
| 1984 | CH₃ | NCH₃ | Ar⁹ | R¹¹-24 |
| 1985 | CH₃ | NCH₃ | Ar⁹ | R¹¹-25 |
| 1986 | CH₃ | NCH₃ | Ar⁹ | R¹¹-26 |
| 1987 | CH₃ | NCH₃ | Ar⁹ | R¹¹-27 |
| 1988 | CH₃ | NCH₃ | Ar⁹ | R¹¹-28 |
| 1989 | CH₃ | NCH₃ | Ar⁹ | R¹¹-29 |
| 1990 | CH₃ | NCH₃ | Ar⁹ | A¹¹-1a |
| 1991 | CH₃ | NCH₃ | Ar⁹ | A¹¹-1b |
| 1992 | CH₃ | NCH₃ | Ar⁹ | A¹¹-2a |
| 1993 | CH₃ | NCH₃ | Ar⁹ | A¹¹-2b |
| 1994 | CH₃ | NCH₃ | Ar⁹ | A¹¹-3a |
| 1995 | CH₃ | NCH₃ | Ar⁹ | A¹¹-3b |
| 1996 | CH₃ | NCH₃ | Ar¹⁰ | R¹¹-1 |
| 1997 | CH₃ | NCH₃ | Ar¹⁰ | R¹¹-2 |
| 1998 | CH₃ | NCH₃ | Ar¹⁰ | R¹¹-3 |
| 1999 | CH₃ | NCH₃ | Ar¹⁰ | R¹¹-4 |
| 2000 | CH₃ | NCH₃ | Ar¹⁰ | R¹¹-5 |
| 2001 | CH₃ | NCH₃ | Ar¹⁰ | R¹¹-6 |
| 2002 | CH₃ | NCH₃ | Ar¹⁰ | R¹¹-7 |
| 2003 | CH₃ | NCH₃ | Ar¹⁰ | R¹¹-8 |
| 2004 | CH₃ | NCH₃ | Ar¹⁰ | R¹¹-9 |
| 2005 | CH₃ | NCH₃ | Ar¹⁰ | R¹¹-10 |
| 2006 | CH₃ | NCH₃ | Ar¹⁰ | R¹¹-11 |
| 2007 | CH₃ | NCH₃ | Ar¹⁰ | R¹¹-12 |
| 2008 | CH₃ | NCH₃ | Ar¹⁰ | R¹¹-13 |
| 2009 | CH₃ | NCH₃ | Ar¹⁰ | R¹¹-14 |
| 2010 | CH₃ | NCH₃ | Ar¹⁰ | R¹¹-15 |
| 2011 | CH₃ | NCH₃ | Ar¹⁰ | R¹¹-16 |
| 2012 | CH₃ | NCH₃ | Ar¹⁰ | R¹¹-17 |
| 2013 | CH₃ | NCH₃ | Ar¹⁰ | R¹¹-18 |
| 2014 | CH₃ | NCH₃ | Ar¹⁰ | R¹¹-19 |
| 2015 | CH₃ | NCH₃ | Ar¹⁰ | R¹¹-20 |
| 2016 | CH₃ | NCH₃ | Ar¹⁰ | R¹¹-21 |
| 2017 | CH₃ | NCH₃ | Ar¹⁰ | R¹¹-22 |
| 2018 | CH₃ | NCH₃ | Ar¹⁰ | R¹¹-23 |
| 2019 | CH₃ | NCH₃ | Ar¹⁰ | R¹¹-24 |
| 2020 | CH₃ | NCH₃ | Ar¹⁰ | R¹¹-25 |
| 2021 | CH₃ | NCH₃ | Ar¹⁰ | R¹¹-26 |
| 2022 | CH₃ | NCH₃ | Ar¹⁰ | R¹¹-27 |
| 2023 | CH₃ | NCH₃ | Ar¹⁰ | R¹¹-28 |
| 2024 | CH₃ | NCH₃ | Ar¹⁰ | R¹¹-29 |
| 2025 | CH₃ | NCH₃ | Ar¹⁰ | A¹¹-1a |
| 2026 | CH₃ | NCH₃ | Ar¹⁰ | A¹¹-1b |
| 2027 | CH₃ | NCH₃ | Ar¹⁰ | A¹¹-2a |
| 2028 | CH₃ | NCH₃ | Ar¹⁰ | A¹¹-2b |
| 2029 | CH₃ | NCH₃ | Ar¹⁰ | A¹¹-3a |
| 2030 | CH₃ | NCH₃ | Ar¹⁰ | A¹¹-3b |
| 2031 | CH₃ | NCH₃ | Ar¹¹ | R¹¹-1 |
| 2032 | CH₃ | NCH₃ | Ar¹¹ | R¹¹-2 |
| 2033 | CH₃ | NCH₃ | Ar¹¹ | R¹¹-3 |
| 2034 | CH₃ | NCH₃ | Ar¹¹ | R¹¹-4 |
| 2035 | CH₃ | NCH₃ | Ar¹¹ | R¹¹-5 |
| 2036 | CH₃ | NCH₃ | Ar¹¹ | R¹¹-6 |
| 2037 | CH₃ | NCH₃ | Ar¹¹ | R¹¹-7 |
| 2038 | CH₃ | NCH₃ | Ar¹¹ | R¹¹-8 |
| 2039 | CH₃ | NCH₃ | Ar¹¹ | R¹¹-9 |
| 2040 | CH₃ | NCH₃ | Ar¹¹ | R¹¹-10 |
| 2041 | CH₃ | NCH₃ | Ar¹¹ | R¹¹-11 |
| 2042 | CH₃ | NCH₃ | Ar¹¹ | R¹¹-12 |
| 2043 | CH₃ | NCH₃ | Ar¹¹ | R¹¹-13 |
| 2044 | CH₃ | NCH₃ | Ar¹¹ | R¹¹-14 |
| 2045 | CH₃ | NCH₃ | Ar¹¹ | R¹¹-15 |
| 2046 | CH₃ | NCH₃ | Ar¹¹ | R¹¹-16 |
| 2047 | CH₃ | NCH₃ | Ar¹¹ | R¹¹-17 |
| 2048 | CH₃ | NCH₃ | Ar¹¹ | R¹¹-18 |
| 2049 | CH₃ | NCH₃ | Ar¹¹ | R¹¹-19 |
| 2050 | CH₃ | NCH₃ | Ar¹¹ | R¹¹-20 |
| 2051 | CH₃ | NCH₃ | Ar¹¹ | R¹¹-21 |
| 2052 | CH₃ | NCH₃ | Ar¹¹ | R¹¹-22 |
| 2053 | CH₃ | NCH₃ | Ar¹¹ | R¹¹-23 |
| 2054 | CH₃ | NCH₃ | Ar¹¹ | R¹¹-24 |
| 2055 | CH₃ | NCH₃ | Ar¹¹ | R¹¹-25 |
| 2056 | CH₃ | NCH₃ | Ar¹¹ | R¹¹-26 |
| 2057 | CH₃ | NCH₃ | Ar¹¹ | R¹¹-27 |
| 2058 | CH₃ | NCH₃ | Ar¹¹ | R¹¹-28 |
| 2059 | CH₃ | NCH₃ | Ar¹¹ | R¹¹-29 |
| 2060 | CH₃ | NCH₃ | Ar¹¹ | A¹¹-1a |
| 2061 | CH₃ | NCH₃ | Ar¹¹ | A¹¹-1b |
| 2062 | CH₃ | NCH₃ | Ar¹¹ | A¹¹-2a |
| 2063 | CH₃ | NCH₃ | Ar¹¹ | A¹¹-2b |
| 2064 | CH₃ | NCH₃ | Ar¹¹ | A¹¹-3a |
| 2065 | CH₃ | NCH₃ | Ar¹¹ | A¹¹-3b |
| 2066 | CH₃ | NCH₃ | Ar¹² | R¹¹-1 |
| 2067 | CH₃ | NCH₃ | Ar¹² | R¹¹-2 |
| 2068 | CH₃ | NCH₃ | Ar¹² | R¹¹-3 |
| 2069 | CH₃ | NCH₃ | Ar¹² | R¹¹-4 |
| 2070 | CH₃ | NCH₃ | Ar¹² | R¹¹-5 |
| 2071 | CH₃ | NCH₃ | Ar¹² | R¹¹-6 |
| 2072 | CH₃ | NCH₃ | Ar¹² | R¹¹-7 |
| 2073 | CH₃ | NCH₃ | Ar¹² | R¹¹-8 |
| 2074 | CH₃ | NCH₃ | Ar¹² | R¹¹-9 |
| 2075 | CH₃ | NCH₃ | Ar¹² | R¹¹-10 |
| 2076 | CH₃ | NCH₃ | Ar¹² | R¹¹-11 |
| 2077 | CH₃ | NCH₃ | Ar¹² | R¹¹-12 |
| 2078 | CH₃ | NCH₃ | Ar¹² | R¹¹-13 |
| 2079 | CH₃ | NCH₃ | Ar¹² | R¹¹-14 |
| 2080 | CH₃ | NCH₃ | Ar¹² | R¹¹-15 |
| 2081 | CH₃ | NCH₃ | Ar¹² | R¹¹-16 |
| 2082 | CH₃ | NCH₃ | Ar¹² | R¹¹-17 |
| 2083 | CH₃ | NCH₃ | Ar¹² | R¹¹-18 |
| 2084 | CH₃ | NCH₃ | Ar¹² | R¹¹-19 |
| 2085 | CH₃ | NCH₃ | Ar¹² | R¹¹-20 |
| 2086 | CH₃ | NCH₃ | Ar¹² | R¹¹-21 |
| 2087 | CH₃ | NCH₃ | Ar¹² | R¹¹-22 |
| 2088 | CH₃ | NCH₃ | Ar¹² | R¹¹-23 |
| 2089 | CH₃ | NCH₃ | Ar¹² | R¹¹-24 |
| 2090 | CH₃ | NCH₃ | Ar¹² | R¹¹-25 |
| 2091 | CH₃ | NCH₃ | Ar¹² | R¹¹-26 |
| 2092 | CH₃ | NCH₃ | Ar¹² | R¹¹-27 |
| 2093 | CH₃ | NCH₃ | Ar¹² | R¹¹-28 |
| 2094 | CH₃ | NCH₃ | Ar¹² | R¹¹-29 |
| 2095 | CH₃ | NCH₃ | Ar¹² | A¹¹-1a |
| 2096 | CH₃ | NCH₃ | Ar¹² | A¹¹-1b |
| 2097 | CH₃ | NCH₃ | Ar¹² | A¹¹-2a |
| 2098 | CH₃ | NCH₃ | Ar¹² | A¹¹-2b |
| 2099 | CH₃ | NCH₃ | Ar¹² | A¹¹-3a |
| 2100 | CH₃ | NCH₃ | Ar¹² | A¹¹-3b |
| 2101 | CH₃ | O | Ar¹ | R¹¹-1 |
| 2102 | CH₃ | O | Ar¹ | R¹¹-2 |
| 2103 | CH₃ | O | Ar¹ | R¹¹-3 |
| 2104 | CH₃ | O | Ar¹ | R¹¹-4 |
| 2105 | CH₃ | O | Ar¹ | R¹¹-5 |
| 2106 | CH₃ | O | Ar¹ | R¹¹-6 |
| 2107 | CH₃ | O | Ar¹ | R¹¹-7 |
| 2108 | CH₃ | O | Ar¹ | R¹¹-8 |
| 2109 | CH₃ | O | Ar¹ | R¹¹-9 |
| 2110 | CH₃ | O | Ar¹ | R¹¹-10 |
| 2111 | CH₃ | O | Ar¹ | R¹¹-11 |
| 2112 | CH₃ | O | Ar¹ | R¹¹-12 |
| 2113 | CH₃ | O | Ar¹ | R¹¹-13 |
| 2114 | CH₃ | O | Ar¹ | R¹¹-14 |
| 2115 | CH₃ | O | Ar¹ | R¹¹-15 |
| 2116 | CH₃ | O | Ar¹ | R¹¹-16 |
| 2117 | CH₃ | O | Ar¹ | R¹¹-17 |
| 2118 | CH₃ | O | Ar¹ | R¹¹-18 |
| 2119 | CH₃ | O | Ar¹ | R¹¹-19 |
| 2120 | CH₃ | O | Ar¹ | R¹¹-20 |
| 2121 | CH₃ | O | Ar¹ | R¹¹-21 |
| 2122 | CH₃ | O | Ar¹ | R¹¹-22 |
| 2123 | CH₃ | O | Ar¹ | R¹¹-23 |
| 2124 | CH₃ | O | Ar¹ | R¹¹-24 |
| 2125 | CH₃ | O | Ar¹ | R¹¹-25 |
| 2126 | CH₃ | O | Ar¹ | R¹¹-26 |
| 2127 | CH₃ | O | Ar¹ | R¹¹-27 |
| 2128 | CH₃ | O | Ar¹ | R¹¹-28 |
| 2129 | CH₃ | O | Ar¹ | R¹¹-29 |
| 2130 | CH₃ | O | Ar¹ | A¹¹-1a |
| 2131 | CH₃ | O | Ar¹ | A¹¹-1b |
| 2132 | CH₃ | O | Ar¹ | A¹¹-2a |
| 2133 | CH₃ | O | Ar¹ | A¹¹-2b |
| 2134 | CH₃ | O | Ar¹ | A¹¹-3a |
| 2135 | CH₃ | O | Ar¹ | A¹¹-3b |
| 2136 | CH₃ | O | Ar² | R¹¹-1 |
| 2137 | CH₃ | O | Ar² | R¹¹-2 |
| 2138 | CH₃ | O | Ar² | R¹¹-3 |
| 2139 | CH₃ | O | Ar² | R¹¹-4 |
| 2140 | CH₃ | O | Ar² | R¹¹-5 |
| 2141 | CH₃ | O | Ar² | R¹¹-6 |
| 2142 | CH₃ | O | Ar² | R¹¹-7 |
| 2143 | CH₃ | O | Ar² | R¹¹-8 |
| 2144 | CH₃ | O | Ar² | R¹¹-9 |
| 2145 | CH₃ | O | Ar² | R¹¹-10 |
| 2146 | CH₃ | O | Ar² | R¹¹-11 |
| 2147 | CH₃ | O | Ar² | R¹¹-12 |
| 2148 | CH₃ | O | Ar² | R¹¹-13 |
| 2149 | CH₃ | O | Ar² | R¹¹-14 |
| 2150 | CH₃ | O | Ar² | R¹¹-15 |
| 2151 | CH₃ | O | Ar² | R¹¹-16 |
| 2152 | CH₃ | O | Ar² | R¹¹-17 |
| 2153 | CH₃ | O | Ar² | R¹¹-18 |
| 2154 | CH₃ | O | Ar² | R¹¹-19 |
| 2155 | CH₃ | O | Ar² | R¹¹-20 |
| 2156 | CH₃ | O | Ar² | R¹¹-21 |
| 2157 | CH₃ | O | Ar² | R¹¹-22 |
| 2158 | CH₃ | O | Ar² | R¹¹-23 |
| 2159 | CH₃ | O | Ar² | R¹¹-24 |
| 2160 | CH₃ | O | Ar² | R¹¹-25 |
| 2161 | CH₃ | O | Ar² | R¹¹-26 |
| 2162 | CH₃ | O | Ar² | R¹¹-27 |
| 2163 | CH₃ | O | Ar² | R¹¹-28 |
| 2164 | CH₃ | O | Ar² | R¹¹-29 |
| 2165 | CH₃ | O | Ar² | A¹¹-1a |
| 2166 | CH₃ | O | Ar² | A¹¹-1b |
| 2167 | CH₃ | O | Ar² | A¹¹-2a |
| 2168 | CH₃ | O | Ar² | A¹¹-2b |
| 2169 | CH₃ | O | Ar² | A¹¹-3a |
| 2170 | CH₃ | O | Ar² | A¹¹-3b |
| 2171 | CH₃ | O | Ar³ | R¹¹-1 |
| 2172 | CH₃ | O | Ar³ | R¹¹-2 |
| 2173 | CH₃ | O | Ar³ | R¹¹-3 |
| 2174 | CH₃ | O | Ar³ | R¹¹-4 |
| 2175 | CH₃ | O | Ar³ | R¹¹-5 |
| 2176 | CH₃ | O | Ar³ | R¹¹-6 |
| 2177 | CH₃ | O | Ar³ | R¹¹-7 |
| 2178 | CH₃ | O | Ar³ | R¹¹-8 |
| 2179 | CH₃ | O | Ar³ | R¹¹-9 |
| 2180 | CH₃ | O | Ar³ | R¹¹-10 |
| 2181 | CH₃ | O | Ar³ | R¹¹-11 |
| 2182 | CH₃ | O | Ar³ | R¹¹-12 |
| 2183 | CH₃ | O | Ar³ | R¹¹-13 |
| 2184 | CH₃ | O | Ar³ | R¹¹-14 |
| 2185 | CH₃ | O | Ar³ | R¹¹-15 |
| 2186 | CH₃ | O | Ar³ | R¹¹-16 |
| 2187 | CH₃ | O | Ar³ | R¹¹-17 |
| 2188 | CH₃ | O | Ar³ | R¹¹-18 |
| 2189 | CH₃ | O | Ar³ | R¹¹-19 |
| 2190 | CH₃ | O | Ar³ | R¹¹-20 |
| 2191 | CH₃ | O | Ar³ | R¹¹-21 |
| 2192 | CH₃ | O | Ar³ | R¹¹-22 |
| 2193 | CH₃ | O | Ar³ | R¹¹-23 |
| 2194 | CH₃ | O | Ar³ | R¹¹-24 |
| 2195 | CH₃ | O | Ar³ | R¹¹-25 |
| 2196 | CH₃ | O | Ar³ | R¹¹-26 |
| 2197 | CH₃ | O | Ar³ | R¹¹-27 |
| 2198 | CH₃ | O | Ar³ | R¹¹-28 |
| 2199 | CH₃ | O | Ar³ | R¹¹-29 |
| 2200 | CH₃ | O | Ar³ | A¹¹-1a |
| 2201 | CH₃ | O | Ar³ | A¹¹-1b |
| 2202 | CH₃ | O | Ar³ | A¹¹-2a |
| 2203 | CH₃ | O | Ar³ | A¹¹-2b |
| 2204 | CH₃ | O | Ar³ | A¹¹-3a |
| 2205 | CH₃ | O | Ar⁴ | A¹¹-3b |
| 2206 | CH₃ | O | Ar⁴ | R¹¹-1 |
| 2207 | CH₃ | O | Ar⁴ | R¹¹-2 |
| 2208 | CH₃ | O | Ar⁴ | R¹¹-3 |
| 2209 | CH₃ | O | Ar⁴ | R¹¹-4 |
| 2210 | CH₃ | O | Ar⁴ | R¹¹-5 |
| 2211 | CH₃ | O | Ar⁴ | R¹¹-6 |
| 2212 | CH₃ | O | Ar⁴ | R¹¹-7 |
| 2213 | CH₃ | O | Ar⁴ | R¹¹-8 |
| 2214 | CH₃ | O | Ar⁴ | R¹¹-9 |
| 2215 | CH₃ | O | Ar⁴ | R¹¹-10 |
| 2216 | CH₃ | O | Ar⁴ | R¹¹-11 |
| 2217 | CH₃ | O | Ar⁴ | R¹¹-12 |
| 2218 | CH₃ | O | Ar⁴ | R¹¹-13 |
| 2219 | CH₃ | O | Ar⁴ | R¹¹-14 |
| 2220 | CH₃ | O | Ar⁴ | R¹¹-15 |
| 2221 | CH₃ | O | Ar⁴ | R¹¹-16 |
| 2222 | CH₃ | O | Ar⁴ | R¹¹-17 |
| 2223 | CH₃ | O | Ar⁴ | R¹¹-18 |
| 2224 | CH₃ | O | Ar⁴ | R¹¹-19 |
| 2225 | CH₃ | O | Ar⁴ | R¹¹-20 |
| 2226 | CH₃ | O | Ar⁴ | R¹¹-21 |
| 2227 | CH₃ | O | Ar⁴ | R¹¹-22 |
| 2228 | CH₃ | O | Ar⁴ | R¹¹-23 |
| 2229 | CH₃ | O | Ar⁴ | R¹¹-24 |
| 2230 | CH₃ | O | Ar⁴ | R¹¹-25 |
| 2231 | CH₃ | O | Ar⁴ | R¹¹-26 |
| 2232 | CH₃ | O | Ar⁴ | R¹¹-27 |
| 2233 | CH₃ | O | Ar⁴ | R¹¹-28 |
| 2234 | CH₃ | O | Ar⁴ | R¹¹-29 |
| 2235 | CH₃ | O | Ar⁴ | A¹¹-1a |
| 2236 | CH₃ | O | Ar⁴ | A¹¹-1b |
| 2237 | CH₃ | O | Ar⁴ | A¹¹-2a |
| 2238 | CH₃ | O | Ar⁴ | A¹¹-2b |
| 2239 | CH₃ | O | Ar⁴ | A¹¹-3a |
| 2240 | CH₃ | O | Ar⁴ | A¹¹-3b |
| 2241 | CH₃ | O | Ar⁵ | R¹¹-1 |
| 2242 | CH₃ | O | Ar⁵ | R¹¹-2 |
| 2243 | CH₃ | O | Ar⁵ | R¹¹-3 |
| 2244 | CH₃ | O | Ar⁵ | R¹¹-4 |
| 2245 | CH₃ | O | Ar⁵ | R¹¹-5 |
| 2246 | CH₃ | O | Ar⁵ | R¹¹-6 |
| 2247 | CH₃ | O | Ar⁵ | R¹¹-7 |
| 2248 | CH₃ | O | Ar⁵ | R¹¹-8 |
| 2249 | CH₃ | O | Ar⁵ | R¹¹-9 |
| 2250 | CH₃ | O | Ar⁵ | R¹¹-10 |
| 2251 | CH₃ | O | Ar⁵ | R¹¹-11 |
| 2252 | CH₃ | O | Ar⁵ | R¹¹-12 |
| 2253 | CH₃ | O | Ar⁵ | R¹¹-13 |
| 2254 | CH₃ | O | Ar⁵ | R¹¹-14 |
| 2255 | CH₃ | O | Ar⁵ | R¹¹-15 |
| 2256 | CH₃ | O | Ar⁵ | R¹¹-16 |
| 2257 | CH₃ | O | Ar⁵ | R¹¹-17 |
| 2258 | CH₃ | O | Ar⁵ | R¹¹-18 |
| 2259 | CH₃ | O | Ar⁵ | R¹¹-19 |
| 2260 | CH₃ | O | Ar⁵ | R¹¹-20 |
| 2261 | CH₃ | O | Ar⁵ | R¹¹-21 |
| 2262 | CH₃ | O | Ar⁵ | R¹¹-22 |
| 2263 | CH₃ | O | Ar⁵ | R¹¹-23 |
| 2264 | CH₃ | O | Ar⁵ | R¹¹-24 |
| 2265 | CH₃ | O | Ar⁵ | R¹¹-25 |
| 2266 | CH₃ | O | Ar⁵ | R¹¹-26 |
| 2267 | CH₃ | O | Ar⁵ | R¹¹-27 |
| 2268 | CH₃ | O | Ar⁵ | R¹¹-28 |
| 2269 | CH₃ | O | Ar⁵ | R¹¹-29 |
| 2270 | CH₃ | O | Ar⁵ | A¹¹-1a |
| 2271 | CH₃ | O | Ar⁵ | A¹¹-1b |
| 2272 | CH₃ | O | Ar⁵ | A¹¹-2a |
| 2273 | CH₃ | O | Ar⁵ | A¹¹-2b |
| 2274 | CH₃ | O | Ar⁵ | A¹¹-3a |
| 2275 | CH₃ | O | Ar⁵ | A¹¹-3b |
| 2276 | CH₃ | O | Ar⁶ | R¹¹-1 |
| 2277 | CH₃ | O | Ar⁶ | R¹¹-2 |
| 2278 | CH₃ | O | Ar⁶ | R¹¹-3 |
| 2279 | CH₃ | O | Ar⁶ | R¹¹-4 |
| 2280 | CH₃ | O | Ar⁶ | R¹¹-5 |
| 2281 | CH₃ | O | Ar⁶ | R¹¹-6 |
| 2282 | CH₃ | O | Ar⁶ | R¹¹-7 |
| 2283 | CH₃ | O | Ar⁶ | R¹¹-8 |
| 2284 | CH₃ | O | Ar⁶ | R¹¹-9 |
| 2285 | CH₃ | O | Ar⁶ | R¹¹-10 |
| 2286 | CH₃ | O | Ar⁶ | R¹¹-11 |
| 2287 | CH₃ | O | Ar⁶ | R¹¹-12 |
| 2288 | CH₃ | O | Ar⁶ | R¹¹-13 |
| 2289 | CH₃ | O | Ar⁶ | R¹¹-14 |
| 2290 | CH₃ | O | Ar⁶ | R¹¹-15 |
| 2291 | CH₃ | O | Ar⁶ | R¹¹-16 |
| 2292 | CH₃ | O | Ar⁶ | R¹¹-17 |
| 2293 | CH₃ | O | Ar⁶ | R¹¹-18 |
| 2294 | CH₃ | O | Ar⁶ | R¹¹-19 |
| 2295 | CH₃ | O | Ar⁶ | R¹¹-20 |
| 2296 | CH₃ | O | Ar⁶ | R¹¹-21 |
| 2297 | CH₃ | O | Ar⁶ | R¹¹-22 |
| 2298 | CH₃ | O | Ar⁶ | R¹¹-23 |
| 2299 | CH₃ | O | Ar⁶ | R¹¹-24 |
| 2300 | CH₃ | O | Ar⁶ | R¹¹-25 |
| 2301 | CH₃ | O | Ar⁶ | R¹¹-26 |
| 2302 | CH₃ | O | Ar⁶ | R¹¹-27 |
| 2303 | CH₃ | O | Ar⁶ | R¹¹-28 |
| 2304 | CH₃ | O | Ar⁶ | R¹¹-29 |
| 2305 | CH₃ | O | Ar⁶ | A¹¹-1a |
| 2306 | CH₃ | O | Ar⁶ | A¹¹-1b |
| 2307 | CH₃ | O | Ar⁶ | A¹¹-2a |
| 2308 | CH₃ | O | Ar⁶ | A¹¹-2b |
| 2309 | CH₃ | O | Ar⁶ | A¹¹-3a |
| 2310 | CH₃ | O | Ar⁶ | A¹¹-3b |
| 2311 | CH₃ | O | Ar⁷ | R¹¹-1 |
| 2312 | CH₃ | O | Ar⁷ | R¹¹-2 |
| 2313 | CH₃ | O | Ar⁷ | R¹¹-3 |
| 2314 | CH₃ | O | Ar⁷ | R¹¹-4 |
| 2315 | CH₃ | O | Ar⁷ | R¹¹-5 |
| 2316 | CH₃ | O | Ar⁷ | R¹¹-6 |
| 2317 | CH₃ | O | Ar⁷ | R¹¹-7 |
| 2318 | CH₃ | O | Ar⁷ | R¹¹-8 |
| 2319 | CH₃ | O | Ar⁷ | R¹¹-9 |
| 2320 | CH₃ | O | Ar⁷ | R¹¹-10 |
| 2321 | CH₃ | O | Ar⁷ | R¹¹-11 |
| 2322 | CH₃ | O | Ar⁷ | R¹¹-12 |
| 2323 | CH₃ | O | Ar⁷ | R¹¹-13 |
| 2324 | CH₃ | O | Ar⁷ | R¹¹-14 |
| 2325 | CH₃ | O | Ar⁷ | R¹¹-15 |
| 2326 | CH₃ | O | Ar⁷ | R¹¹-16 |
| 2327 | CH₃ | O | Ar⁷ | R¹¹-17 |
| 2328 | CH₃ | O | Ar⁷ | R¹¹-18 |
| 2329 | CH₃ | O | Ar⁷ | R¹¹-19 |
| 2330 | CH₃ | O | Ar⁷ | R¹¹-20 |
| 2331 | CH₃ | O | Ar⁷ | R¹¹-21 |
| 2332 | CH₃ | O | Ar⁷ | R¹¹-22 |
| 2333 | CH₃ | O | Ar⁷ | R¹¹-23 |
| 2334 | CH₃ | O | Ar⁷ | R¹¹-24 |
| 2335 | CH₃ | O | Ar⁷ | R¹¹-25 |
| 2336 | CH₃ | O | Ar⁷ | R¹¹-26 |
| 2337 | CH₃ | O | Ar⁷ | R¹¹-27 |
| 2338 | CH₃ | O | Ar⁷ | R¹¹-28 |
| 2339 | CH₃ | O | Ar⁷ | R¹¹-29 |
| 2340 | CH₃ | O | Ar⁷ | A¹¹-1a |
| 2341 | CH₃ | O | Ar⁷ | A¹¹-1b |
| 2342 | CH₃ | O | Ar⁷ | A¹¹-2a |
| 2343 | CH₃ | O | Ar⁷ | A¹¹-2b |
| 2344 | CH₃ | O | Ar⁷ | A¹¹-3a |
| 2345 | CH₃ | O | Ar⁷ | A¹¹-3b |
| 2346 | CH₃ | O | Ar⁸ | R¹¹-1 |
| 2347 | CH₃ | O | Ar⁸ | R¹¹-2 |
| 2348 | CH₃ | O | Ar⁸ | R¹¹-3 |
| 2349 | CH₃ | O | Ar⁸ | R¹¹-4 |
| 2350 | CH₃ | O | Ar⁸ | R¹¹-5 |
| 2351 | CH₃ | O | Ar⁸ | R¹¹-6 |
| 2352 | CH₃ | O | Ar⁸ | R¹¹-7 |
| 2353 | CH₃ | O | Ar⁸ | R¹¹-8 |
| 2354 | CH₃ | O | Ar⁸ | R¹¹-9 |
| 2355 | CH₃ | O | Ar⁸ | R¹¹-10 |
| 2356 | CH₃ | O | Ar⁸ | R¹¹-11 |
| 2357 | CH₃ | O | Ar⁸ | R¹¹-12 |
| 2358 | CH₃ | O | Ar⁸ | R¹¹-13 |
| 2359 | CH₃ | O | Ar⁸ | R¹¹-14 |
| 2360 | CH₃ | O | Ar⁸ | R¹¹-15 |
| 2361 | CH₃ | O | Ar⁸ | R¹¹-16 |
| 2362 | CH₃ | O | Ar⁸ | R¹¹-17 |
| 2363 | CH₃ | O | Ar⁸ | R¹¹-18 |
| 2364 | CH₃ | O | Ar⁸ | R¹¹-19 |
| 2365 | CH₃ | O | Ar⁸ | R¹¹-20 |
| 2366 | CH₃ | O | Ar⁸ | R¹¹-21 |
| 2367 | CH₃ | O | Ar⁸ | R¹¹-22 |
| 2368 | CH₃ | O | Ar⁸ | R¹¹-23 |
| 2369 | CH₃ | O | Ar⁸ | R¹¹-24 |
| 2370 | CH₃ | O | Ar⁸ | R¹¹-25 |
| 2371 | CH₃ | O | Ar⁸ | R¹¹-26 |
| 2372 | CH₃ | O | Ar⁸ | R¹¹-27 |
| 2373 | CH₃ | O | Ar⁸ | R¹¹-28 |
| 2374 | CH₃ | O | Ar⁸ | R¹¹-29 |
| 2375 | CH₃ | O | Ar⁸ | A¹¹-1a |
| 2376 | CH₃ | O | Ar⁸ | A¹¹-1b |
| 2377 | CH₃ | O | Ar⁸ | A¹¹-2a |
| 2378 | CH₃ | O | Ar⁸ | A¹¹-2b |
| 2379 | CH₃ | O | Ar⁸ | A¹¹-3a |
| 2380 | CH₃ | O | Ar⁸ | A¹¹-3b |
| 2381 | CH₃ | O | Ar⁹ | R¹¹-1 |
| 2382 | CH₃ | O | Ar⁹ | R¹¹-2 |
| 2383 | CH₃ | O | Ar⁹ | R¹¹-3 |
| 2384 | CH₃ | O | Ar⁹ | R¹¹-4 |
| 2385 | CH₃ | O | Ar⁹ | R¹¹-5 |
| 2386 | CH₃ | O | Ar⁹ | R¹¹-6 |
| 2387 | CH₃ | O | Ar⁹ | R¹¹-7 |
| 2388 | CH₃ | O | Ar⁹ | R¹¹-8 |
| 2389 | CH₃ | O | Ar⁹ | R¹¹-9 |
| 2390 | CH₃ | O | Ar⁹ | R¹¹-10 |
| 2391 | CH₃ | O | Ar⁹ | R¹¹-11 |
| 2392 | CH₃ | O | Ar⁹ | R¹¹-12 |
| 2393 | CH₃ | O | Ar⁹ | R¹¹-13 |
| 2394 | CH₃ | O | Ar⁹ | R¹¹-14 |
| 2395 | CH₃ | O | Ar⁹ | R¹¹-15 |
| 2396 | CH₃ | O | Ar⁹ | R¹¹-16 |
| 2397 | CH₃ | O | Ar⁹ | R¹¹-17 |
| 2398 | CH₃ | O | Ar⁹ | R¹¹-18 |
| 2399 | CH₃ | O | Ar⁹ | R¹¹-19 |
| 2400 | CH₃ | O | Ar⁹ | R¹¹-20 |
| 2401 | CH₃ | O | Ar⁹ | R¹¹-21 |
| 2402 | CH₃ | O | Ar⁹ | R¹¹-22 |
| 2403 | CH₃ | O | Ar⁹ | R¹¹-23 |
| 2404 | CH₃ | O | Ar⁹ | R¹¹-24 |
| 2405 | CH₃ | O | Ar⁹ | R¹¹-25 |
| 2406 | CH₃ | O | Ar⁹ | R¹¹-26 |
| 2407 | CH₃ | O | Ar⁹ | R¹¹-27 |
| 2408 | CH₃ | O | Ar⁹ | R¹¹-28 |
| 2409 | CH₃ | O | Ar⁹ | R¹¹-29 |
| 2410 | CH₃ | O | Ar⁹ | A¹¹-1a |
| 2411 | CH₃ | O | Ar⁹ | A¹¹-1b |
| 2412 | CH₃ | O | Ar⁹ | A¹¹-2a |
| 2413 | CH₃ | O | Ar⁹ | A¹¹-2b |
| 2414 | CH₃ | O | Ar⁹ | A¹¹-3a |
| 2415 | CH₃ | O | Ar⁹ | A¹¹-3b |
| 2416 | CH₃ | O | Ar¹⁰ | R¹¹-1 |
| 2417 | CH₃ | O | Ar¹⁰ | R¹¹-2 |
| 2418 | CH₃ | O | Ar¹⁰ | R¹¹-3 |
| 2419 | CH₃ | O | Ar¹⁰ | R¹¹-4 |
| 2420 | CH₃ | O | Ar¹⁰ | R¹¹-5 |
| 2421 | CH₃ | O | Ar¹⁰ | R¹¹-6 |
| 2422 | CH₃ | O | Ar¹⁰ | R¹¹-7 |
| 2423 | CH₃ | O | Ar¹⁰ | R¹¹-8 |
| 2424 | CH₃ | O | Ar¹⁰ | R¹¹-9 |
| 2425 | CH₃ | O | Ar¹⁰ | R¹¹-10 |
| 2426 | CH₃ | O | Ar¹⁰ | R¹¹-11 |
| 2427 | CH₃ | O | Ar¹⁰ | R¹¹-12 |
| 2428 | CH₃ | O | Ar¹⁰ | R¹¹-13 |
| 2429 | CH₃ | O | Ar¹⁰ | R¹¹-14 |
| 2430 | CH₃ | O | Ar¹⁰ | R¹¹-15 |
| 2431 | CH₃ | O | Ar¹⁰ | R¹¹-16 |
| 2432 | CH₃ | O | Ar¹⁰ | R¹¹-17 |
| 2433 | CH₃ | O | Ar¹⁰ | R¹¹-18 |
| 2434 | CH₃ | O | Ar¹⁰ | R¹¹-19 |
| 2435 | CH₃ | O | Ar¹⁰ | R¹¹-20 |
| 2436 | CH₃ | O | Ar¹⁰ | R¹¹-21 |
| 2437 | CH₃ | O | Ar¹⁰ | R¹¹-22 |
| 2438 | CH₃ | O | Ar¹⁰ | R¹¹-23 |
| 2439 | CH₃ | O | Ar¹⁰ | R¹¹-24 |
| 2440 | CH₃ | O | Ar¹⁰ | R¹¹-25 |
| 2441 | CH₃ | O | Ar¹⁰ | R¹¹-26 |
| 2442 | CH₃ | O | Ar¹⁰ | R¹¹-27 |
| 2443 | CH₃ | O | Ar¹⁰ | R¹¹-28 |
| 2444 | CH₃ | O | Ar¹⁰ | R¹¹-29 |
| 2445 | CH₃ | O | Ar¹⁰ | A¹¹-1a |
| 2446 | CH₃ | O | Ar¹⁰ | A¹¹-1b |
| 2447 | CH₃ | O | Ar¹⁰ | A¹¹-2a |
| 2448 | CH₃ | O | Ar¹⁰ | A¹¹-2b |
| 2449 | CH₃ | O | Ar¹⁰ | A¹¹-3a |
| 2450 | CH₃ | O | Ar¹⁰ | A¹¹-3b |
| 2451 | CH₃ | O | Ar¹¹ | R¹¹-1 |
| 2452 | CH₃ | O | Ar¹¹ | R¹¹-2 |
| 2453 | CH₃ | O | Ar¹¹ | R¹¹-3 |
| 2454 | CH₃ | O | Ar¹¹ | R¹¹-4 |
| 2455 | CH₃ | O | Ar¹¹ | R¹¹-5 |
| 2456 | CH₃ | O | Ar¹¹ | R¹¹-6 |
| 2457 | CH₃ | O | Ar¹¹ | R¹¹-7 |
| 2458 | CH₃ | O | Ar¹¹ | R¹¹-8 |
| 2459 | CH₃ | O | Ar¹¹ | R¹¹-9 |
| 2460 | CH₃ | O | Ar¹¹ | R¹¹-10 |
| 2461 | CH₃ | O | Ar¹¹ | R¹¹-11 |
| 2462 | CH₃ | O | Ar¹¹ | R¹¹-12 |
| 2463 | CH₃ | O | Ar¹¹ | R¹¹-13 |
| 2464 | CH₃ | O | Ar¹¹ | R¹¹-14 |
| 2465 | CH₃ | O | Ar¹¹ | R¹¹-15 |
| 2466 | CH₃ | O | Ar¹¹ | R¹¹-16 |
| 2467 | CH₃ | O | Ar¹¹ | R¹¹-17 |
| 2468 | CH₃ | O | Ar¹¹ | R¹¹-18 |
| 2469 | CH₃ | O | Ar¹¹ | R¹¹-19 |
| 2470 | CH₃ | O | Ar¹¹ | R¹¹-20 |
| 2471 | CH₃ | O | Ar¹¹ | R¹¹-21 |
| 2472 | CH₃ | O | Ar¹¹ | R¹¹-22 |
| 2473 | CH₃ | O | Ar¹¹ | R¹¹-23 |
| 2474 | CH₃ | O | Ar¹¹ | R¹¹-24 |
| 2475 | CH₃ | O | Ar¹¹ | R¹¹-25 |
| 2476 | CH₃ | O | Ar¹¹ | R¹¹-26 |
| 2477 | CH₃ | O | Ar¹¹ | R¹¹-27 |
| 2478 | CH₃ | O | Ar¹¹ | R¹¹-28 |
| 2479 | CH₃ | O | Ar¹¹ | R¹¹-29 |
| 2480 | CH₃ | O | Ar¹¹ | A¹¹-1a |
| 2481 | CH₃ | O | Ar¹¹ | A¹¹-1b |
| 2482 | CH₃ | O | Ar¹¹ | A¹¹-2a |
| 2483 | CH₃ | O | Ar¹¹ | A¹¹-2b |
| 2484 | CH₃ | O | Ar¹¹ | A¹¹-3a |
| 2485 | CH₃ | O | Ar¹¹ | A¹¹-3b |
| 2486 | CH₃ | O | Ar¹² | R¹¹-1 |
| 2487 | CH₃ | O | Ar¹² | R¹¹-2 |
| 2488 | CH₃ | O | Ar¹² | R¹¹-3 |
| 2489 | CH₃ | O | Ar¹² | R¹¹-4 |
| 2490 | CH₃ | O | Ar¹² | R¹¹-5 |
| 2491 | CH₃ | O | Ar¹² | R¹¹-6 |
| 2492 | CH₃ | O | Ar¹² | R¹¹-7 |
| 2493 | CH₃ | O | Ar¹² | R¹¹-8 |
| 2494 | CH₃ | O | Ar¹² | R¹¹-9 |
| 2495 | CH₃ | O | Ar¹² | R¹¹-10 |
| 2496 | CH₃ | O | Ar¹² | R¹¹-11 |
| 2497 | CH₃ | O | Ar¹² | R¹¹-12 |
| 2498 | CH₃ | O | Ar¹² | R¹¹-13 |
| 2499 | CH₃ | O | Ar¹² | R¹¹-14 |
| 2500 | CH₃ | O | Ar¹² | R¹¹-15 |
| 2501 | CH₃ | O | Ar¹² | R¹¹-16 |
| 2502 | CH₃ | O | Ar¹² | R¹¹-17 |
| 2503 | CH₃ | O | Ar¹² | R¹¹-18 |
| 2504 | CH₃ | O | Ar¹² | R¹¹-19 |
| 2505 | CH₃ | O | Ar¹² | R¹¹-20 |
| 2506 | CH₃ | O | Ar¹² | R¹¹-21 |
| 2507 | CH₃ | O | Ar¹² | R¹¹-22 |
| 2508 | CH₃ | O | Ar¹² | R¹¹-23 |
| 2509 | CH₃ | O | Ar¹² | R¹¹-24 |
| 2510 | CH₃ | O | Ar¹² | R¹¹-25 |
| 2511 | CH₃ | O | Ar¹² | R¹¹-26 |
| 2512 | CH₃ | O | Ar¹² | R¹¹-27 |
| 2513 | CH₃ | O | Ar¹² | R¹¹-28 |
| 2514 | CH₃ | O | Ar¹² | R¹¹-29 |
| 2515 | CH₃ | O | Ar¹² | A¹¹-1a |
| 2516 | CH₃ | O | Ar¹² | A¹¹-1b |
| 2517 | CH₃ | O | Ar¹² | A¹¹-2a |
| 2518 | CH₃ | O | Ar¹² | A¹¹-2b |
| 2519 | CH₃ | O | Ar¹² | A¹¹-3a |
| 2520 | CH₃ | O | Ar¹² | A¹¹-3b |
| 2521 | Cl | NH | Ar¹ | R¹¹-1 |
| 2522 | Cl | NH | Ar¹ | R¹¹-2 |
| 2523 | Cl | NH | Ar¹ | R¹¹-3 |
| 2524 | Cl | NH | Ar¹ | R¹¹-4 |
| 2525 | Cl | NH | Ar¹ | R¹¹-5 |
| 2526 | Cl | NH | Ar¹ | R¹¹-6 |
| 2527 | Cl | NH | Ar¹ | R¹¹-7 |
| 2528 | Cl | NH | Ar¹ | R¹¹-8 |
| 2529 | Cl | NH | Ar¹ | R¹¹-9 |
| 2530 | Cl | NH | Ar¹ | R¹¹-10 |
| 2531 | Cl | NH | Ar¹ | R¹¹-11 |
| 2532 | Cl | NH | Ar¹ | R¹¹-12 |
| 2533 | Cl | NH | Ar¹ | R¹¹-13 |
| 2534 | Cl | NH | Ar¹ | R¹¹-14 |
| 2535 | Cl | NH | Ar¹ | R¹¹-15 |
| 2536 | Cl | NH | Ar¹ | R¹¹-16 |
| 2537 | Cl | NH | Ar¹ | R¹¹-17 |
| 2538 | Cl | NH | Ar¹ | R¹¹-18 |
| 2539 | Cl | NH | Ar¹ | R¹¹-19 |
| 2540 | Cl | NH | Ar¹ | R¹¹-20 |
| 2541 | Cl | NH | Ar¹ | R¹¹-21 |
| 2542 | Cl | NH | Ar¹ | R¹¹-22 |
| 2543 | Cl | NH | Ar¹ | R¹¹-23 |
| 2544 | Cl | NH | Ar¹ | R¹¹-24 |
| 2545 | Cl | NH | Ar¹ | R¹¹-25 |
| 2546 | Cl | NH | Ar¹ | R¹¹-26 |
| 2547 | Cl | NH | Ar¹ | R¹¹-27 |
| 2548 | Cl | NH | Ar¹ | R¹¹-28 |
| 2549 | Cl | NH | Ar¹ | R¹¹-29 |
| 2550 | Cl | NH | Ar¹ | A¹¹-1a |
| 2551 | Cl | NH | Ar¹ | A¹¹-1b |
| 2552 | Cl | NH | Ar¹ | A¹¹-2a |
| 2553 | Cl | NH | Ar¹ | A¹¹-2b |
| 2554 | Cl | NH | Ar¹ | A¹¹-3a |
| 2555 | Cl | NH | Ar¹ | A¹¹-3b |
| 2556 | Cl | NH | Ar² | R¹¹-1 |
| 2557 | Cl | NH | Ar² | R¹¹-2 |
| 2558 | Cl | NH | Ar² | R¹¹-3 |
| 2559 | Cl | NH | Ar² | R¹¹-4 |
| 2560 | Cl | NH | Ar² | R¹¹-5 |
| 2561 | Cl | NH | Ar² | R¹¹-6 |
| 2562 | Cl | NH | Ar² | R¹¹-7 |
| 2563 | Cl | NH | Ar² | R¹¹-8 |
| 2564 | Cl | NH | Ar² | R¹¹-9 |
| 2565 | Cl | NH | Ar² | R¹¹-10 |
| 2566 | Cl | NH | Ar² | R¹¹-11 |
| 2567 | Cl | NH | Ar² | R¹¹-12 |
| 2568 | Cl | NH | Ar² | R¹¹-13 |
| 2569 | Cl | NH | Ar² | R¹¹-14 |
| 2570 | Cl | NH | Ar² | R¹¹-15 |
| 2571 | Cl | NH | Ar² | R¹¹-16 |
| 2572 | Cl | NH | Ar² | R¹¹-17 |
| 2573 | Cl | NH | Ar² | R¹¹-18 |
| 2574 | Cl | NH | Ar² | R¹¹-19 |
| 2575 | Cl | NH | Ar² | R¹¹-20 |
| 2576 | Cl | NH | Ar² | R¹¹-21 |
| 2577 | Cl | NH | Ar² | R¹¹-22 |
| 2578 | Cl | NH | Ar² | R¹¹-23 |
| 2579 | Cl | NH | Ar² | R¹¹-24 |
| 2580 | Cl | NH | Ar² | R¹¹-25 |
| 2581 | Cl | NH | Ar² | R¹¹-26 |
| 2582 | Cl | NH | Ar² | R¹¹-27 |
| 2583 | Cl | NH | Ar² | R¹¹-28 |
| 2584 | Cl | NH | Ar² | R¹¹-29 |
| 2585 | Cl | NH | Ar² | A¹¹-1a |
| 2586 | Cl | NH | Ar² | A¹¹-1b |
| 2587 | Cl | NH | Ar² | A¹¹-2a |
| 2588 | Cl | NH | Ar² | A¹¹-2b |
| 2589 | Cl | NH | Ar² | A¹¹-3a |
| 2590 | Cl | NH | Ar² | A¹¹-3b |
| 2591 | Cl | NH | Ar³ | R¹¹-1 |
| 2592 | Cl | NH | Ar³ | R¹¹-2 |
| 2593 | Cl | NH | Ar³ | R¹¹-3 |
| 2594 | Cl | NH | Ar³ | R¹¹-4 |
| 2595 | Cl | NH | Ar³ | R¹¹-5 |
| 2596 | Cl | NH | Ar³ | R¹¹-6 |
| 2597 | Cl | NH | Ar³ | R¹¹-7 |
| 2598 | Cl | NH | Ar³ | R¹¹-8 |
| 2599 | Cl | NH | Ar³ | R¹¹-9 |
| 2600 | Cl | NH | Ar³ | R¹¹-10 |
| 2601 | Cl | NH | Ar³ | R¹¹-11 |
| 2602 | Cl | NH | Ar³ | R¹¹-12 |
| 2603 | Cl | NH | Ar³ | R¹¹-13 |
| 2604 | Cl | NH | Ar³ | R¹¹-14 |
| 2605 | Cl | NH | Ar³ | R¹¹-15 |
| 2606 | Cl | NH | Ar³ | R¹¹-16 |
| 2607 | Cl | NH | Ar³ | R¹¹-17 |
| 2608 | Cl | NH | Ar³ | R¹¹-18 |
| 2609 | Cl | NH | Ar³ | R¹¹-19 |
| 2610 | Cl | NH | Ar³ | R¹¹-20 |
| 2611 | Cl | NH | Ar³ | R¹¹-21 |
| 2612 | Cl | NH | Ar³ | R¹¹-22 |
| 2613 | Cl | NH | Ar³ | R¹¹-23 |
| 2614 | Cl | NH | Ar³ | R¹¹-24 |
| 2615 | Cl | NH | Ar³ | R¹¹-25 |
| 2616 | Cl | NH | Ar³ | R¹¹-26 |
| 2617 | Cl | NH | Ar³ | R¹¹-27 |
| 2618 | Cl | NH | Ar³ | R¹¹-28 |
| 2619 | Cl | NH | Ar³ | R¹¹-29 |
| 2620 | Cl | NH | Ar³ | A¹¹-1a |
| 2621 | Cl | NH | Ar³ | A¹¹-1b |
| 2622 | Cl | NH | Ar³ | A¹¹-2a |
| 2623 | Cl | NH | Ar³ | A¹¹-2b |
| 2624 | Cl | NH | Ar³ | A¹¹-3a |
| 2625 | Cl | NH | Ar⁴ | A¹¹-3b |
| 2626 | Cl | NH | Ar⁴ | R¹¹-1 |
| 2627 | Cl | NH | Ar⁴ | R¹¹-2 |
| 2628 | Cl | NH | Ar⁴ | R¹¹-3 |
| 2629 | Cl | NH | Ar⁴ | R¹¹-4 |
| 2630 | Cl | NH | Ar⁴ | R¹¹-5 |
| 2631 | Cl | NH | Ar⁴ | R¹¹-6 |
| 2632 | Cl | NH | Ar⁴ | R¹¹-7 |
| 2633 | Cl | NH | Ar⁴ | R¹¹-8 |
| 2634 | Cl | NH | Ar⁴ | R¹¹-9 |
| 2635 | Cl | NH | Ar⁴ | R¹¹-10 |
| 2636 | Cl | NH | Ar⁴ | R¹¹-11 |
| 2637 | Cl | NH | Ar⁴ | R¹¹-12 |
| 2638 | Cl | NH | Ar⁴ | R¹¹-13 |
| 2639 | Cl | NH | Ar⁴ | R¹¹-14 |
| 2640 | Cl | NH | Ar⁴ | R¹¹-15 |
| 2641 | Cl | NH | Ar⁴ | R¹¹-16 |
| 2642 | Cl | NH | Ar⁴ | R¹¹-17 |
| 2643 | Cl | NH | Ar⁴ | R¹¹-18 |
| 2644 | Cl | NH | Ar⁴ | R¹¹-19 |
| 2645 | Cl | NH | Ar⁴ | R¹¹-20 |
| 2646 | Cl | NH | Ar⁴ | R¹¹-21 |
| 2647 | Cl | NH | Ar⁴ | R¹¹-22 |
| 2648 | Cl | NH | Ar⁴ | R¹¹-23 |
| 2649 | Cl | NH | Ar⁴ | R¹¹-24 |
| 2650 | Cl | NH | Ar⁴ | R¹¹-25 |
| 2651 | Cl | NH | Ar⁴ | R¹¹-26 |
| 2652 | Cl | NH | Ar⁴ | R¹¹-27 |
| 2653 | Cl | NH | Ar⁴ | R¹¹-28 |
| 2654 | Cl | NH | Ar⁴ | R¹¹-29 |
| 2655 | Cl | NH | Ar⁴ | A¹¹-1a |
| 2656 | Cl | NH | Ar⁴ | A¹¹-1b |
| 2657 | Cl | NH | Ar⁴ | A¹¹-2a |
| 2658 | Cl | NH | Ar⁴ | A¹¹-2b |
| 2659 | Cl | NH | Ar⁴ | A¹¹-3a |
| 2660 | Cl | NH | Ar⁴ | A¹¹-3b |
| 2661 | Cl | NH | Ar⁵ | R¹¹-1 |
| 2662 | Cl | NH | Ar⁵ | R¹¹-2 |
| 2663 | Cl | NH | Ar⁵ | R¹¹-3 |
| 2664 | Cl | NH | Ar⁵ | R¹¹-4 |
| 2665 | Cl | NH | Ar⁵ | R¹¹-5 |
| 2666 | Cl | NH | Ar⁵ | R¹¹-6 |
| 2667 | Cl | NH | Ar⁵ | R¹¹-7 |
| 2668 | Cl | NH | Ar⁵ | R¹¹-8 |
| 2669 | Cl | NH | Ar⁵ | R¹¹-9 |
| 2670 | Cl | NH | Ar⁵ | R¹¹-10 |
| 2671 | Cl | NH | Ar⁵ | R¹¹-11 |
| 2672 | Cl | NH | Ar⁵ | R¹¹-12 |
| 2673 | Cl | NH | Ar⁵ | R¹¹-13 |
| 2674 | Cl | NH | Ar⁵ | R¹¹-14 |
| 2675 | Cl | NH | Ar⁵ | R¹¹-15 |
| 2676 | Cl | NH | Ar⁵ | R¹¹-16 |
| 2677 | Cl | NH | Ar⁵ | R¹¹-17 |
| 2678 | Cl | NH | Ar⁵ | R¹¹-18 |
| 2679 | Cl | NH | Ar⁵ | R¹¹-19 |
| 2680 | Cl | NH | Ar⁵ | R¹¹-20 |
| 2681 | Cl | NH | Ar⁵ | R¹¹-21 |
| 2682 | Cl | NH | Ar⁵ | R¹¹-22 |
| 2683 | Cl | NH | Ar⁵ | R¹¹-23 |
| 2684 | Cl | NH | Ar⁵ | R¹¹-24 |
| 2685 | Cl | NH | Ar⁵ | R¹¹-25 |
| 2686 | Cl | NH | Ar⁵ | R¹¹-26 |
| 2687 | Cl | NH | Ar⁵ | R¹¹-27 |
| 2688 | Cl | NH | Ar⁵ | R¹¹-28 |
| 2689 | Cl | NH | Ar⁵ | R¹¹-29 |
| 2690 | Cl | NH | Ar⁵ | A¹¹-1a |
| 2691 | Cl | NH | Ar⁵ | A¹¹-1b |
| 2692 | Cl | NH | Ar⁵ | A¹¹-2a |
| 2693 | Cl | NH | Ar⁵ | A¹¹-2b |
| 2694 | Cl | NH | Ar⁵ | A¹¹-3a |
| 2695 | Cl | NH | Ar⁵ | A¹¹-3b |
| 2696 | Cl | NH | Ar⁶ | R¹¹-1 |
| 2697 | Cl | NH | Ar⁶ | R¹¹-2 |
| 2698 | Cl | NH | Ar⁶ | R¹¹-3 |
| 2699 | Cl | NH | Ar⁶ | R¹¹-4 |
| 2700 | Cl | NH | Ar⁶ | R¹¹-5 |
| 2701 | Cl | NH | Ar⁶ | R¹¹-6 |
| 2702 | Cl | NH | Ar⁶ | R¹¹-7 |
| 2703 | Cl | NH | Ar⁶ | R¹¹-8 |
| 2704 | Cl | NH | Ar⁶ | R¹¹-9 |
| 2705 | Cl | NH | Ar⁶ | R¹¹-10 |
| 2706 | Cl | NH | Ar⁶ | R¹¹-11 |
| 2707 | Cl | NH | Ar⁶ | R¹¹-12 |
| 2708 | Cl | NH | Ar⁶ | R¹¹-13 |
| 2709 | Cl | NH | Ar⁶ | R¹¹-14 |
| 2710 | Cl | NH | Ar⁶ | R¹¹-15 |
| 2711 | Cl | NH | Ar⁶ | R¹¹-16 |
| 2712 | Cl | NH | Ar⁶ | R¹¹-17 |
| 2713 | Cl | NH | Ar⁶ | R¹¹-18 |
| 2714 | Cl | NH | Ar⁶ | R¹¹-19 |
| 2715 | Cl | NH | Ar⁶ | R¹¹-20 |
| 2716 | Cl | NH | Ar⁶ | R¹¹-21 |
| 2717 | Cl | NH | Ar⁶ | R¹¹-22 |
| 2718 | Cl | NH | Ar⁶ | R¹¹-23 |
| 2719 | Cl | NH | Ar⁶ | R¹¹-24 |
| 2720 | Cl | NH | Ar⁶ | R¹¹-25 |
| 2721 | Cl | NH | Ar⁶ | R¹¹-26 |
| 2722 | Cl | NH | Ar⁶ | R¹¹-27 |
| 2723 | Cl | NH | Ar⁶ | R¹¹-28 |
| 2724 | Cl | NH | Ar⁶ | R¹¹-29 |
| 2725 | Cl | NH | Ar⁶ | A¹¹-1a |
| 2726 | Cl | NH | Ar⁶ | A¹¹-1b |
| 2727 | Cl | NH | Ar⁶ | A¹¹-2a |
| 2728 | Cl | NH | Ar⁶ | A¹¹-2b |
| 2729 | Cl | NH | Ar⁶ | A¹¹-3a |
| 2730 | Cl | NH | Ar⁶ | A¹¹-3b |
| 2731 | Cl | NH | Ar⁷ | R¹¹-1 |
| 2732 | Cl | NH | Ar⁷ | R¹¹-2 |
| 2733 | Cl | NH | Ar⁷ | R¹¹-3 |
| 2734 | Cl | NH | Ar⁷ | R¹¹-4 |
| 2735 | Cl | NH | Ar⁷ | R¹¹-5 |
| 2736 | Cl | NH | Ar⁷ | R¹¹-6 |
| 2737 | Cl | NH | Ar⁷ | R¹¹-7 |
| 2738 | Cl | NH | Ar⁷ | R¹¹-8 |
| 2739 | Cl | NH | Ar⁷ | R¹¹-9 |
| 2740 | Cl | NH | Ar⁷ | R¹¹-10 |
| 2741 | Cl | NH | Ar⁷ | R¹¹-11 |
| 2742 | Cl | NH | Ar⁷ | R¹¹-12 |
| 2743 | Cl | NH | Ar⁷ | R¹¹-13 |
| 2744 | Cl | NH | Ar⁷ | R¹¹-14 |
| 2745 | Cl | NH | Ar⁷ | R¹¹-15 |
| 2746 | Cl | NH | Ar⁷ | R¹¹-16 |
| 2747 | Cl | NH | Ar⁷ | R¹¹-17 |
| 2748 | Cl | NH | Ar⁷ | R¹¹-18 |
| 2749 | Cl | NH | Ar⁷ | R¹¹-19 |
| 2750 | Cl | NH | Ar⁷ | R¹¹-20 |
| 2751 | Cl | NH | Ar⁷ | R¹¹-21 |
| 2752 | Cl | NH | Ar⁷ | R¹¹-22 |
| 2753 | Cl | NH | Ar⁷ | R¹¹-23 |
| 2754 | Cl | NH | Ar⁷ | R¹¹-24 |
| 2755 | Cl | NH | Ar⁷ | R¹¹-25 |
| 2756 | Cl | NH | Ar⁷ | R¹¹-26 |
| 2757 | Cl | NH | Ar⁷ | R¹¹-27 |
| 2758 | Cl | NH | Ar⁷ | R¹¹-28 |
| 2759 | Cl | NH | Ar⁷ | R¹¹-29 |
| 2760 | Cl | NH | Ar⁷ | A¹¹-1a |
| 2761 | Cl | NH | Ar⁷ | A¹¹-1b |
| 2762 | Cl | NH | Ar⁷ | A¹¹-2a |
| 2763 | Cl | NH | Ar⁷ | A¹¹-2b |
| 2764 | Cl | NH | Ar⁷ | A¹¹-3a |
| 2765 | Cl | NH | Ar⁷ | A¹¹-3b |
| 2766 | Cl | NH | Ar⁸ | R¹¹-1 |
| 2767 | Cl | NH | Ar⁸ | R¹¹-2 |
| 2768 | Cl | NH | Ar⁸ | R¹¹-3 |
| 2769 | Cl | NH | Ar⁸ | R¹¹-4 |
| 2770 | Cl | NH | Ar⁸ | R¹¹-5 |
| 2771 | Cl | NH | Ar⁸ | R¹¹-6 |
| 2772 | Cl | NH | Ar⁸ | R¹¹-7 |
| 2773 | Cl | NH | Ar⁸ | R¹¹-8 |
| 2774 | Cl | NH | Ar⁸ | R¹¹-9 |
| 2775 | Cl | NH | Ar⁸ | R¹¹-10 |
| 2776 | Cl | NH | Ar⁸ | R¹¹-11 |
| 2777 | Cl | NH | Ar⁸ | R¹¹-12 |
| 2778 | Cl | NH | Ar⁸ | R¹¹-13 |
| 2779 | Cl | NH | Ar⁸ | R¹¹-14 |
| 2780 | Cl | NH | Ar⁸ | R¹¹-15 |
| 2781 | Cl | NH | Ar⁸ | R¹¹-16 |
| 2782 | Cl | NH | Ar⁸ | R¹¹-17 |
| 2783 | Cl | NH | Ar⁸ | R¹¹-18 |
| 2784 | Cl | NH | Ar⁸ | R¹¹-19 |
| 2785 | Cl | NH | Ar⁸ | R¹¹-20 |
| 2786 | Cl | NH | Ar⁸ | R¹¹-21 |
| 2787 | Cl | NH | Ar⁸ | R¹¹-22 |
| 2788 | Cl | NH | Ar⁸ | R¹¹-23 |
| 2789 | Cl | NH | Ar⁸ | R¹¹-24 |
| 2790 | Cl | NH | Ar⁸ | R¹¹-25 |
| 2791 | Cl | NH | Ar⁸ | R¹¹-26 |
| 2792 | Cl | NH | Ar⁸ | R¹¹-27 |
| 2793 | Cl | NH | Ar⁸ | R¹¹-28 |
| 2794 | Cl | NH | Ar⁸ | R¹¹-29 |
| 2795 | Cl | NH | Ar⁸ | A¹¹-1a |
| 2796 | Cl | NH | Ar⁸ | A¹¹-1b |
| 2797 | Cl | NH | Ar⁸ | A¹¹-2a |
| 2798 | Cl | NH | Ar⁸ | A¹¹-2b |
| 2799 | Cl | NH | Ar⁸ | A¹¹-3a |
| 2800 | Cl | NH | Ar⁸ | A¹¹-3b |
| 2801 | Cl | NH | Ar⁹ | R¹¹-1 |
| 2802 | Cl | NH | Ar⁹ | R¹¹-2 |
| 2803 | Cl | NH | Ar⁹ | R¹¹-3 |
| 2804 | Cl | NH | Ar⁹ | R¹¹-4 |
| 2805 | Cl | NH | Ar⁹ | R¹¹-5 |
| 2806 | Cl | NH | Ar⁹ | R¹¹-6 |
| 2807 | Cl | NH | Ar⁹ | R¹¹-7 |
| 2808 | Cl | NH | Ar⁹ | R¹¹-8 |
| 2809 | Cl | NH | Ar⁹ | R¹¹-9 |
| 2810 | Cl | NH | Ar⁹ | R¹¹-10 |
| 2811 | Cl | NH | Ar⁹ | R¹¹-11 |
| 2812 | Cl | NH | Ar⁹ | R¹¹-12 |
| 2813 | Cl | NH | Ar⁹ | R¹¹-13 |
| 2814 | Cl | NH | Ar⁹ | R¹¹-14 |
| 2815 | Cl | NH | Ar⁹ | R¹¹-15 |
| 2816 | Cl | NH | Ar⁹ | R¹¹-16 |
| 2817 | Cl | NH | Ar⁹ | R¹¹-17 |
| 2818 | Cl | NH | Ar⁹ | R¹¹-18 |
| 2819 | Cl | NH | Ar⁹ | R¹¹-19 |
| 2820 | Cl | NH | Ar⁹ | R¹¹-20 |
| 2821 | Cl | NH | Ar⁹ | R¹¹-21 |
| 2822 | Cl | NH | Ar⁹ | R¹¹-22 |
| 2823 | Cl | NH | Ar⁹ | R¹¹-23 |
| 2824 | Cl | NH | Ar⁹ | R¹¹-24 |
| 2825 | Cl | NH | Ar⁹ | R¹¹-25 |
| 2826 | Cl | NH | Ar⁹ | R¹¹-26 |
| 2827 | Cl | NH | Ar⁹ | R¹¹-27 |
| 2828 | Cl | NH | Ar⁹ | R¹¹-28 |
| 2829 | Cl | NH | Ar⁹ | R¹¹-29 |
| 2830 | Cl | NH | Ar⁹ | A¹¹-1a |
| 2831 | Cl | NH | Ar⁹ | A¹¹-1b |
| 2832 | Cl | NH | Ar⁹ | A¹¹-2a |
| 2833 | Cl | NH | Ar⁹ | A¹¹-2b |
| 2834 | Cl | NH | Ar⁹ | A¹¹-3a |
| 2835 | Cl | NH | Ar⁹ | A¹¹-3b |
| 2836 | Cl | NH | Ar¹⁰ | R¹¹-1 |
| 2837 | Cl | NH | Ar¹⁰ | R¹¹-2 |
| 2838 | Cl | NH | Ar¹⁰ | R¹¹-3 |
| 2839 | Cl | NH | Ar¹⁰ | R¹¹-4 |
| 2840 | Cl | NH | Ar¹⁰ | R¹¹-5 |
| 2841 | Cl | NH | Ar¹⁰ | R¹¹-6 |
| 2842 | Cl | NH | Ar¹⁰ | R¹¹-7 |
| 2843 | Cl | NH | Ar¹⁰ | R¹¹-8 |
| 2844 | Cl | NH | Ar¹⁰ | R¹¹-9 |
| 2845 | Cl | NH | Ar¹⁰ | R¹¹-10 |
| 2846 | Cl | NH | Ar¹⁰ | R¹¹-11 |
| 2847 | Cl | NH | Ar¹⁰ | R¹¹-12 |
| 2848 | Cl | NH | Ar¹⁰ | R¹¹-13 |
| 2849 | Cl | NH | Ar¹⁰ | R¹¹-14 |
| 2850 | Cl | NH | Ar¹⁰ | R¹¹-15 |
| 2851 | Cl | NH | Ar¹⁰ | R¹¹-16 |
| 2852 | Cl | NH | Ar¹⁰ | R¹¹-17 |
| 2853 | Cl | NH | Ar¹⁰ | R¹¹-18 |
| 2854 | Cl | NH | Ar¹⁰ | R¹¹-19 |
| 2855 | Cl | NH | Ar¹⁰ | R¹¹-20 |
| 2856 | Cl | NH | Ar¹⁰ | R¹¹-21 |
| 2857 | Cl | NH | Ar¹⁰ | R¹¹-22 |
| 2858 | Cl | NH | Ar¹⁰ | R¹¹-23 |
| 2859 | Cl | NH | Ar¹⁰ | R¹¹-24 |
| 2860 | Cl | NH | Ar¹⁰ | R¹¹-25 |
| 2861 | Cl | NH | Ar¹⁰ | R¹¹-26 |
| 2862 | Cl | NH | Ar¹⁰ | R¹¹-27 |
| 2863 | Cl | NH | Ar¹⁰ | R¹¹-28 |
| 2864 | Cl | NH | Ar¹⁰ | R¹¹-29 |
| 2865 | Cl | NH | Ar¹⁰ | A¹¹-1a |
| 2866 | Cl | NH | Ar¹⁰ | A¹¹-1b |
| 2867 | Cl | NH | Ar¹⁰ | A¹¹-2a |
| 2868 | Cl | NH | Ar¹⁰ | A¹¹-2b |
| 2869 | Cl | NH | Ar¹⁰ | A¹¹-3a |
| 2870 | Cl | NH | Ar¹⁰ | A¹¹-3b |
| 2871 | Cl | NH | Ar¹¹ | R¹¹-1 |
| 2872 | Cl | NH | Ar¹¹ | R¹¹-2 |
| 2873 | Cl | NH | Ar¹¹ | R¹¹-3 |
| 2874 | Cl | NH | Ar¹¹ | R¹¹-4 |
| 2875 | Cl | NH | Ar¹¹ | R¹¹-5 |
| 2876 | Cl | NH | Ar¹¹ | R¹¹-6 |
| 2877 | Cl | NH | Ar¹¹ | R¹¹-7 |
| 2878 | Cl | NH | Ar¹¹ | R¹¹-8 |
| 2879 | Cl | NH | Ar¹¹ | R¹¹-9 |
| 2880 | Cl | NH | Ar¹¹ | R¹¹-10 |
| 2881 | Cl | NH | Ar¹¹ | R¹¹-11 |
| 2882 | Cl | NH | Ar¹¹ | R¹¹-12 |
| 2883 | Cl | NH | Ar¹¹ | R¹¹-13 |
| 2884 | Cl | NH | Ar¹¹ | R¹¹-14 |
| 2885 | Cl | NH | Ar¹¹ | R¹¹-15 |
| 2886 | Cl | NH | Ar¹¹ | R¹¹-16 |
| 2887 | Cl | NH | Ar¹¹ | R¹¹-17 |
| 2888 | Cl | NH | Ar¹¹ | R¹¹-18 |
| 2889 | Cl | NH | Ar¹¹ | R¹¹-19 |
| 2890 | Cl | NH | Ar¹¹ | R¹¹-20 |
| 2891 | Cl | NH | Ar¹¹ | R¹¹-21 |
| 2892 | Cl | NH | Ar¹¹ | R¹¹-22 |
| 2893 | Cl | NH | Ar¹¹ | R¹¹-23 |
| 2894 | Cl | NH | Ar¹¹ | R¹¹-24 |
| 2895 | Cl | NH | Ar¹¹ | R¹¹-25 |
| 2896 | Cl | NH | Ar¹¹ | R¹¹-26 |
| 2897 | Cl | NH | Ar¹¹ | R¹¹-27 |
| 2898 | Cl | NH | Ar¹¹ | R¹¹-28 |
| 2899 | Cl | NH | Ar¹¹ | R¹¹-29 |
| 2900 | Cl | NH | Ar¹¹ | A¹¹-1a |
| 2901 | Cl | NH | Ar¹¹ | A¹¹-1b |
| 2902 | Cl | NH | Ar¹¹ | A¹¹-2a |
| 2903 | Cl | NH | Ar¹¹ | A¹¹-2b |
| 2904 | Cl | NH | Ar¹¹ | A¹¹-3a |
| 2905 | Cl | NH | Ar¹¹ | A¹¹-3b |
| 2906 | Cl | NH | Ar¹² | R¹¹-1 |
| 2907 | Cl | NH | Ar¹² | R¹¹-2 |
| 2908 | Cl | NH | Ar¹² | R¹¹-3 |
| 2909 | Cl | NH | Ar¹² | R¹¹-4 |
| 2910 | Cl | NH | Ar¹² | R¹¹-5 |
| 2911 | Cl | NH | Ar¹² | R¹¹-6 |
| 2912 | Cl | NH | Ar¹² | R¹¹-7 |
| 2913 | Cl | NH | Ar¹² | R¹¹-8 |
| 2914 | Cl | NH | Ar¹² | R¹¹-9 |
| 2915 | Cl | NH | Ar¹² | R¹¹-10 |
| 2916 | Cl | NH | Ar¹² | R¹¹-11 |
| 2917 | Cl | NH | Ar¹² | R¹¹-12 |
| 2918 | Cl | NH | Ar¹² | R¹¹-13 |
| 2919 | Cl | NH | Ar¹² | R¹¹-14 |
| 2920 | Cl | NH | Ar¹² | R¹¹-15 |
| 2921 | Cl | NH | Ar¹² | R¹¹-16 |
| 2922 | Cl | NH | Ar¹² | R¹¹-17 |
| 2923 | Cl | NH | Ar¹² | R¹¹-18 |
| 2924 | Cl | NH | Ar¹² | R¹¹-19 |
| 2925 | Cl | NH | Ar¹² | R¹¹-20 |
| 2926 | Cl | NH | Ar¹² | R¹¹-21 |
| 2927 | Cl | NH | Ar¹² | R¹¹-22 |
| 2928 | Cl | NH | Ar¹² | R¹¹-23 |
| 2929 | Cl | NH | Ar¹² | R¹¹-24 |
| 2930 | Cl | NH | Ar¹² | R¹¹-25 |
| 2931 | Cl | NH | Ar¹² | R¹¹-26 |
| 2932 | Cl | NH | Ar¹² | R¹¹-27 |
| 2933 | Cl | NH | Ar¹² | R¹¹-28 |
| 2934 | Cl | NH | Ar¹² | R¹¹-29 |
| 2935 | Cl | NH | Ar¹² | A¹¹-1a |
| 2936 | Cl | NH | Ar¹² | A¹¹-1b |
| 2937 | Cl | NH | Ar¹² | A¹¹-2a |
| 2938 | Cl | NH | Ar¹² | A¹¹-2b |
| 2939 | Cl | NH | Ar¹² | A¹¹-3a |
| 2940 | Cl | NH | Ar¹² | A¹¹-3b |
| 2941 | Cl | NCH₃ | Ar¹ | R¹¹-1 |
| 2942 | Cl | NCH₃ | Ar¹ | R¹¹-2 |
| 2943 | Cl | NCH₃ | Ar¹ | R¹¹-3 |
| 2944 | Cl | NCH₃ | Ar¹ | R¹¹-4 |
| 2945 | Cl | NCH₃ | Ar¹ | R¹¹-5 |
| 2946 | Cl | NCH₃ | Ar¹ | R¹¹-6 |
| 2947 | Cl | NCH₃ | Ar¹ | R¹¹-7 |
| 2948 | Cl | NCH₃ | Ar¹ | R¹¹-8 |
| 2949 | Cl | NCH₃ | Ar¹ | R¹¹-9 |
| 2950 | Cl | NCH₃ | Ar¹ | R¹¹-10 |
| 2951 | Cl | NCH₃ | Ar¹ | R¹¹-11 |
| 2952 | Cl | NCH₃ | Ar¹ | R¹¹-12 |
| 2953 | Cl | NCH₃ | Ar¹ | R¹¹-13 |
| 2954 | Cl | NCH₃ | Ar¹ | R¹¹-14 |
| 2955 | Cl | NCH₃ | Ar¹ | R¹¹-15 |
| 2956 | Cl | NCH₃ | Ar¹ | R¹¹-16 |
| 2957 | Cl | NCH₃ | Ar¹ | R¹¹-17 |
| 2958 | Cl | NCH₃ | Ar¹ | R¹¹-18 |
| 2959 | Cl | NCH₃ | Ar¹ | R¹¹-19 |
| 2960 | Cl | NCH₃ | Ar¹ | R¹¹-20 |
| 2961 | Cl | NCH₃ | Ar¹ | R¹¹-21 |
| 2962 | Cl | NCH₃ | Ar¹ | R¹¹-22 |
| 2963 | Cl | NCH₃ | Ar¹ | R¹¹-23 |
| 2964 | Cl | NCH₃ | Ar¹ | R¹¹-24 |
| 2965 | Cl | NCH₃ | Ar¹ | R¹¹-25 |
| 2966 | Cl | NCH₃ | Ar¹ | R¹¹-26 |
| 2967 | Cl | NCH₃ | Ar¹ | R¹¹-27 |
| 2968 | Cl | NCH₃ | Ar¹ | R¹¹-28 |
| 2969 | Cl | NCH₃ | Ar¹ | R¹¹-29 |
| 2970 | Cl | NCH₃ | Ar¹ | A¹¹-1a |
| 2971 | Cl | NCH₃ | Ar¹ | A¹¹-1b |
| 2972 | Cl | NCH₃ | Ar¹ | A¹¹-2a |
| 2973 | Cl | NCH₃ | Ar¹ | A¹¹-2b |
| 2974 | Cl | NCH₃ | Ar¹ | A¹¹-3a |
| 2975 | Cl | NCH₃ | Ar¹ | A¹¹-3b |
| 2976 | Cl | NCH₃ | Ar² | R¹¹-1 |
| 2977 | Cl | NCH₃ | Ar² | R¹¹-2 |
| 2978 | Cl | NCH₃ | Ar² | R¹¹-3 |
| 2979 | Cl | NCH₃ | Ar² | R¹¹-4 |
| 2980 | Cl | NCH₃ | Ar² | R¹¹-5 |
| 2981 | Cl | NCH₃ | Ar² | R¹¹-6 |
| 2982 | Cl | NCH₃ | Ar² | R¹¹-7 |
| 2983 | Cl | NCH₃ | Ar² | R¹¹-8 |
| 2984 | Cl | NCH₃ | Ar² | R¹¹-9 |
| 2985 | Cl | NCH₃ | Ar² | R¹¹-10 |
| 2986 | Cl | NCH₃ | Ar² | R¹¹-11 |
| 2987 | Cl | NCH₃ | Ar² | R¹¹-12 |
| 2988 | Cl | NCH₃ | Ar² | R¹¹-13 |
| 2989 | Cl | NCH₃ | Ar² | R¹¹-14 |
| 2990 | Cl | NCH₃ | Ar² | R¹¹-15 |
| 2991 | Cl | NCH₃ | Ar² | R¹¹-16 |
| 2992 | Cl | NCH₃ | Ar² | R¹¹-17 |
| 2993 | Cl | NCH₃ | Ar² | R¹¹-18 |
| 2994 | Cl | NCH₃ | Ar² | R¹¹-19 |
| 2995 | Cl | NCH₃ | Ar² | R¹¹-20 |
| 2996 | Cl | NCH₃ | Ar² | R¹¹-21 |
| 2997 | Cl | NCH₃ | Ar² | R¹¹-22 |
| 2998 | Cl | NCH₃ | Ar² | R¹¹-23 |
| 2999 | Cl | NCH₃ | Ar² | R¹¹-24 |
| 3000 | Cl | NCH₃ | Ar² | R¹¹-25 |
| 3001 | Cl | NCH₃ | Ar² | R¹¹-26 |
| 3002 | Cl | NCH₃ | Ar² | R¹¹-27 |
| 3003 | Cl | NCH₃ | Ar² | R¹¹-28 |
| 3004 | Cl | NCH₃ | Ar² | R¹¹-29 |
| 3005 | Cl | NCH₃ | Ar² | A¹¹-1a |
| 3006 | Cl | NCH₃ | Ar² | A¹¹-1b |
| 3007 | Cl | NCH₃ | Ar² | A¹¹-2a |
| 3008 | Cl | NCH₃ | Ar² | A¹¹-2b |
| 3009 | Cl | NCH₃ | Ar² | A¹¹-3a |
| 3010 | Cl | NCH₃ | Ar² | A¹¹-3b |
| 3011 | Cl | NCH₃ | Ar³ | R¹¹-1 |
| 3012 | Cl | NCH₃ | Ar³ | R¹¹-2 |
| 3013 | Cl | NCH₃ | Ar³ | R¹¹-3 |
| 3014 | Cl | NCH₃ | Ar³ | R¹¹-4 |
| 3015 | Cl | NCH₃ | Ar³ | R¹¹-5 |
| 3016 | Cl | NCH₃ | Ar³ | R¹¹-6 |
| 3017 | Cl | NCH₃ | Ar³ | R¹¹-7 |
| 3018 | Cl | NCH₃ | Ar³ | R¹¹-8 |
| 3019 | Cl | NCH₃ | Ar³ | R¹¹-9 |
| 3020 | Cl | NCH₃ | Ar³ | R¹¹-10 |
| 3021 | Cl | NCH₃ | Ar³ | R¹¹-11 |
| 3022 | Cl | NCH₃ | Ar³ | R¹¹-12 |
| 3023 | Cl | NCH₃ | Ar³ | R¹¹-13 |
| 3024 | Cl | NCH₃ | Ar³ | R¹¹-14 |
| 3025 | Cl | NCH₃ | Ar³ | R¹¹-15 |
| 3026 | Cl | NCH₃ | Ar³ | R¹¹-16 |
| 3027 | Cl | NCH₃ | Ar³ | R¹¹-17 |
| 3028 | Cl | NCH₃ | Ar³ | R¹¹-18 |
| 3029 | Cl | NCH₃ | Ar³ | R¹¹-19 |
| 3030 | Cl | NCH₃ | Ar³ | R¹¹-20 |
| 3031 | Cl | NCH₃ | Ar³ | R¹¹-21 |
| 3032 | Cl | NCH₃ | Ar³ | R¹¹-22 |
| 3033 | Cl | NCH₃ | Ar³ | R¹¹-23 |
| 3034 | Cl | NCH₃ | Ar³ | R¹¹-24 |
| 3035 | Cl | NCH₃ | Ar³ | R¹¹-25 |
| 3036 | Cl | NCH₃ | Ar³ | R¹¹-26 |
| 3037 | Cl | NCH₃ | Ar³ | R¹¹-27 |
| 3038 | Cl | NCH₃ | Ar³ | R¹¹-28 |
| 3039 | Cl | NCH₃ | Ar³ | R¹¹-29 |
| 3040 | Cl | NCH₃ | Ar³ | A¹¹-1a |
| 3041 | Cl | NCH₃ | Ar³ | A¹¹-1b |
| 3042 | Cl | NCH₃ | Ar³ | A¹¹-2a |
| 3043 | Cl | NCH₃ | Ar³ | A¹¹-2b |
| 3044 | Cl | NCH₃ | Ar³ | A¹¹-3a |
| 3045 | Cl | NCH₃ | Ar⁴ | A¹¹-3b |
| 3046 | Cl | NCH₃ | Ar⁴ | R¹¹-1 |
| 3047 | Cl | NCH₃ | Ar⁴ | R¹¹-2 |
| 3048 | Cl | NCH₃ | Ar⁴ | R¹¹-3 |
| 3049 | Cl | NCH₃ | Ar⁴ | R¹¹-4 |
| 3050 | Cl | NCH₃ | Ar⁴ | R¹¹-5 |
| 3051 | Cl | NCH₃ | Ar⁴ | R¹¹-6 |
| 3052 | Cl | NCH₃ | Ar⁴ | R¹¹-7 |
| 3053 | Cl | NCH₃ | Ar⁴ | R¹¹-8 |
| 3054 | Cl | NCH₃ | Ar⁴ | R¹¹-9 |
| 3055 | Cl | NCH₃ | Ar⁴ | R¹¹-10 |
| 3056 | Cl | NCH₃ | Ar⁴ | R¹¹-11 |
| 3057 | Cl | NCH₃ | Ar⁴ | R¹¹-12 |
| 3058 | Cl | NCH₃ | Ar⁴ | R¹¹-13 |
| 3059 | Cl | NCH₃ | Ar⁴ | R¹¹-14 |
| 3060 | Cl | NCH₃ | Ar⁴ | R¹¹-15 |
| 3061 | Cl | NCH₃ | Ar⁴ | R¹¹-16 |
| 3062 | Cl | NCH₃ | Ar⁴ | R¹¹-17 |
| 3063 | Cl | NCH₃ | Ar⁴ | R¹¹-18 |
| 3064 | Cl | NCH₃ | Ar⁴ | R¹¹-19 |
| 3065 | Cl | NCH₃ | Ar⁴ | R¹¹-20 |
| 3066 | Cl | NCH₃ | Ar⁴ | R¹¹-21 |
| 3067 | Cl | NCH₃ | Ar⁴ | R¹¹-22 |
| 3068 | Cl | NCH₃ | Ar⁴ | R¹¹-23 |
| 3069 | Cl | NCH₃ | Ar⁴ | R¹¹-24 |
| 3070 | Cl | NCH₃ | Ar⁴ | R¹¹-25 |
| 3071 | Cl | NCH₃ | Ar⁴ | R¹¹-26 |
| 3072 | Cl | NCH₃ | Ar⁴ | R¹¹-27 |
| 3073 | Cl | NCH₃ | Ar⁴ | R¹¹-28 |
| 3074 | Cl | NCH₃ | Ar⁴ | R¹¹-29 |
| 3075 | Cl | NCH₃ | Ar⁴ | A¹¹-1a |
| 3076 | Cl | NCH₃ | Ar⁴ | A¹¹-1b |
| 3077 | Cl | NCH₃ | Ar⁴ | A¹¹-2a |
| 3078 | Cl | NCH₃ | Ar⁴ | A¹¹-2b |
| 3079 | Cl | NCH₃ | Ar⁴ | A¹¹-3a |
| 3080 | Cl | NCH₃ | Ar⁴ | A¹¹-3b |
| 3081 | Cl | NCH₃ | Ar⁵ | R¹¹-1 |
| 3082 | Cl | NCH₃ | Ar⁵ | R¹¹-2 |
| 3083 | Cl | NCH₃ | Ar⁵ | R¹¹-3 |
| 3084 | Cl | NCH₃ | Ar⁵ | R¹¹-4 |
| 3085 | Cl | NCH₃ | Ar⁵ | R¹¹-5 |
| 3086 | Cl | NCH₃ | Ar⁵ | R¹¹-6 |
| 3087 | Cl | NCH₃ | Ar⁵ | R¹¹-7 |
| 3088 | Cl | NCH₃ | Ar⁵ | R¹¹-8 |
| 3089 | Cl | NCH₃ | Ar⁵ | R¹¹-9 |
| 3090 | Cl | NCH₃ | Ar⁵ | R¹¹-10 |
| 3091 | Cl | NCH₃ | Ar⁵ | R¹¹-11 |
| 3092 | Cl | NCH₃ | Ar⁵ | R¹¹-12 |
| 3093 | Cl | NCH₃ | Ar⁵ | R¹¹-13 |
| 3094 | Cl | NCH₃ | Ar⁵ | R¹¹-14 |
| 3095 | Cl | NCH₃ | Ar⁵ | R¹¹-15 |
| 3096 | Cl | NCH₃ | Ar⁵ | R¹¹-16 |
| 3097 | Cl | NCH₃ | Ar⁵ | R¹¹-17 |
| 3098 | Cl | NCH₃ | Ar⁵ | R¹¹-18 |
| 3099 | Cl | NCH₃ | Ar⁵ | R¹¹-19 |
| 3100 | Cl | NCH₃ | Ar⁵ | R¹¹-20 |
| 3101 | Cl | NCH₃ | Ar⁵ | R¹¹-21 |
| 3102 | Cl | NCH₃ | Ar⁵ | R¹¹-22 |
| 3103 | Cl | NCH₃ | Ar⁵ | R¹¹-23 |
| 3104 | Cl | NCH₃ | Ar⁵ | R¹¹-24 |
| 3105 | Cl | NCH₃ | Ar⁵ | R¹¹-25 |
| 3106 | Cl | NCH₃ | Ar⁵ | R¹¹-26 |
| 3107 | Cl | NCH₃ | Ar⁵ | R¹¹-27 |
| 3108 | Cl | NCH₃ | Ar⁵ | R¹¹-28 |
| 3109 | Cl | NCH₃ | Ar⁵ | R¹¹-29 |
| 3110 | Cl | NCH₃ | Ar⁵ | A¹¹-1a |
| 3111 | Cl | NCH₃ | Ar⁵ | A¹¹-1b |
| 3112 | Cl | NCH₃ | Ar⁵ | A¹¹-2a |
| 3113 | Cl | NCH₃ | Ar⁵ | A¹¹-2b |
| 3114 | Cl | NCH₃ | Ar⁵ | A¹¹-3a |
| 3115 | Cl | NCH₃ | Ar⁵ | A¹¹-3b |
| 3116 | Cl | NCH₃ | Ar⁶ | R¹¹-1 |
| 3117 | Cl | NCH₃ | Ar⁶ | R¹¹-2 |
| 3118 | Cl | NCH₃ | Ar⁶ | R¹¹-3 |
| 3119 | Cl | NCH₃ | Ar⁶ | R¹¹-4 |
| 3120 | Cl | NCH₃ | Ar⁶ | R¹¹-5 |
| 3121 | Cl | NCH₃ | Ar⁶ | R¹¹-6 |
| 3122 | Cl | NCH₃ | Ar⁶ | R¹¹-7 |
| 3123 | Cl | NCH₃ | Ar⁶ | R¹¹-8 |
| 3124 | Cl | NCH₃ | Ar⁶ | R¹¹-9 |
| 3125 | Cl | NCH₃ | Ar⁶ | R¹¹-10 |
| 3126 | Cl | NCH₃ | Ar⁶ | R¹¹-11 |
| 3127 | Cl | NCH₃ | Ar⁶ | R¹¹-12 |
| 3128 | Cl | NCH₃ | Ar⁶ | R¹¹-13 |
| 3129 | Cl | NCH₃ | Ar⁶ | R¹¹-14 |
| 3130 | Cl | NCH₃ | Ar⁶ | R¹¹-15 |
| 3131 | Cl | NCH₃ | Ar⁶ | R¹¹-16 |
| 3132 | Cl | NCH₃ | Ar⁶ | R¹¹-17 |
| 3133 | Cl | NCH₃ | Ar⁶ | R¹¹-18 |
| 3134 | Cl | NCH₃ | Ar⁶ | R¹¹-19 |
| 3135 | Cl | NCH₃ | Ar⁶ | R¹¹-20 |
| 3136 | Cl | NCH₃ | Ar⁶ | R¹¹-21 |
| 3137 | Cl | NCH₃ | Ar⁶ | R¹¹-22 |
| 3138 | Cl | NCH₃ | Ar⁶ | R¹¹-23 |
| 3139 | Cl | NCH₃ | Ar⁶ | R¹¹-24 |
| 3140 | Cl | NCH₃ | Ar⁶ | R¹¹-25 |
| 3141 | Cl | NCH₃ | Ar⁶ | R¹¹-26 |
| 3142 | Cl | NCH₃ | Ar⁶ | R¹¹-27 |
| 3143 | Cl | NCH₃ | Ar⁶ | R¹¹-28 |
| 3144 | Cl | NCH₃ | Ar⁶ | R¹¹-29 |
| 3145 | Cl | NCH₃ | Ar⁶ | A¹¹-1a |
| 3146 | Cl | NCH₃ | Ar⁶ | A¹¹-1b |
| 3147 | Cl | NCH₃ | Ar⁶ | A¹¹-2a |
| 3148 | Cl | NCH₃ | Ar⁶ | A¹¹-2b |
| 3149 | Cl | NCH₃ | Ar⁶ | A¹¹-3a |
| 3150 | Cl | NCH₃ | Ar⁶ | A¹¹-3b |
| 3151 | Cl | NCH₃ | Ar⁷ | R¹¹-1 |
| 3152 | Cl | NCH₃ | Ar⁷ | R¹¹-2 |
| 3153 | Cl | NCH₃ | Ar⁷ | R¹¹-3 |
| 3154 | Cl | NCH₃ | Ar⁷ | R¹¹-4 |
| 3155 | Cl | NCH₃ | Ar⁷ | R¹¹-5 |
| 3156 | Cl | NCH₃ | Ar⁷ | R¹¹-6 |
| 3157 | Cl | NCH₃ | Ar⁷ | R¹¹-7 |
| 3158 | Cl | NCH₃ | Ar⁷ | R¹¹-8 |
| 3159 | Cl | NCH₃ | Ar⁷ | R¹¹-9 |
| 3160 | Cl | NCH₃ | Ar⁷ | R¹¹-10 |
| 3161 | Cl | NCH₃ | Ar⁷ | R¹¹-11 |
| 3162 | Cl | NCH₃ | Ar⁷ | R¹¹-12 |
| 3163 | Cl | NCH₃ | Ar⁷ | R¹¹-13 |
| 3164 | Cl | NCH₃ | Ar⁷ | R¹¹-14 |
| 3165 | Cl | NCH₃ | Ar⁷ | R¹¹-15 |
| 3166 | Cl | NCH₃ | Ar⁷ | R¹¹-16 |
| 3167 | Cl | NCH₃ | Ar⁷ | R¹¹-17 |
| 3168 | Cl | NCH₃ | Ar⁷ | R¹¹-18 |
| 3169 | Cl | NCH₃ | Ar⁷ | R¹¹-19 |
| 3170 | Cl | NCH₃ | Ar⁷ | R¹¹-20 |
| 3171 | Cl | NCH₃ | Ar⁷ | R¹¹-21 |
| 3172 | Cl | NCH₃ | Ar⁷ | R¹¹-22 |
| 3173 | Cl | NCH₃ | Ar⁷ | R¹¹-23 |
| 3174 | Cl | NCH₃ | Ar⁷ | R¹¹-24 |
| 3175 | Cl | NCH₃ | Ar⁷ | R¹¹-25 |
| 3176 | Cl | NCH₃ | Ar⁷ | R¹¹-26 |
| 3177 | Cl | NCH₃ | Ar⁷ | R¹¹-27 |
| 3178 | Cl | NCH₃ | Ar⁷ | R¹¹-28 |
| 3179 | Cl | NCH₃ | Ar⁷ | R¹¹-29 |
| 3180 | Cl | NCH₃ | Ar⁷ | A¹¹-1a |
| 3181 | Cl | NCH₃ | Ar⁷ | A¹¹-1b |
| 3182 | Cl | NCH₃ | Ar⁷ | A¹¹-2a |
| 3183 | Cl | NCH₃ | Ar⁷ | A¹¹-2b |
| 3184 | Cl | NCH₃ | Ar⁷ | A¹¹-3a |
| 3185 | Cl | NCH₃ | Ar⁷ | A¹¹-3b |
| 3186 | Cl | NCH₃ | Ar⁸ | R¹¹-1 |
| 3187 | Cl | NCH₃ | Ar⁸ | R¹¹-2 |
| 3188 | Cl | NCH₃ | Ar⁸ | R¹¹-3 |
| 3189 | Cl | NCH₃ | Ar⁸ | R¹¹-4 |
| 3190 | Cl | NCH₃ | Ar⁸ | R¹¹-5 |
| 3191 | Cl | NCH₃ | Ar⁸ | R¹¹-6 |
| 3192 | Cl | NCH₃ | Ar⁸ | R¹¹-7 |
| 3193 | Cl | NCH₃ | Ar⁸ | R¹¹-8 |
| 3194 | Cl | NCH₃ | Ar⁸ | R¹¹-9 |
| 3195 | Cl | NCH₃ | Ar⁸ | R¹¹-10 |
| 3196 | Cl | NCH₃ | Ar⁸ | R¹¹-11 |
| 3197 | Cl | NCH₃ | Ar⁸ | R¹¹-12 |
| 3198 | Cl | NCH₃ | Ar⁸ | R¹¹-13 |
| 3199 | Cl | NCH₃ | Ar⁸ | R¹¹-14 |
| 3200 | Cl | NCH₃ | Ar⁸ | R¹¹-15 |
| 3201 | Cl | NCH₃ | Ar⁸ | R¹¹-16 |
| 3202 | Cl | NCH₃ | Ar⁸ | R¹¹-17 |
| 3203 | Cl | NCH₃ | Ar⁸ | R¹¹-18 |
| 3204 | Cl | NCH₃ | Ar⁸ | R¹¹-19 |
| 3205 | Cl | NCH₃ | Ar⁸ | R¹¹-20 |
| 3206 | Cl | NCH₃ | Ar⁸ | R¹¹-21 |
| 3207 | Cl | NCH₃ | Ar⁸ | R¹¹-22 |
| 3208 | Cl | NCH₃ | Ar⁸ | R¹¹-23 |
| 3209 | Cl | NCH₃ | Ar⁸ | R¹¹-24 |
| 3210 | Cl | NCH₃ | Ar⁸ | R¹¹-25 |
| 3211 | Cl | NCH₃ | Ar⁸ | R¹¹-26 |
| 3212 | Cl | NCH₃ | Ar⁸ | R¹¹-27 |
| 3213 | Cl | NCH₃ | Ar⁸ | R¹¹-28 |
| 3214 | Cl | NCH₃ | Ar⁸ | R¹¹-29 |
| 3215 | Cl | NCH₃ | Ar⁸ | A¹¹-1a |
| 3216 | Cl | NCH₃ | Ar⁸ | A¹¹-1b |
| 3217 | Cl | NCH₃ | Ar⁸ | A¹¹-2a |
| 3218 | Cl | NCH₃ | Ar⁸ | A¹¹-2b |
| 3219 | Cl | NCH₃ | Ar⁸ | A¹¹-3a |
| 3220 | Cl | NCH₃ | Ar⁸ | A¹¹-3b |
| 3221 | Cl | NCH₃ | Ar⁹ | R¹¹-1 |
| 3222 | Cl | NCH₃ | Ar⁹ | R¹¹-2 |
| 3223 | Cl | NCH₃ | Ar⁹ | R¹¹-3 |
| 3224 | Cl | NCH₃ | Ar⁹ | R¹¹-4 |
| 3225 | Cl | NCH₃ | Ar⁹ | R¹¹-5 |
| 3226 | Cl | NCH₃ | Ar⁹ | R¹¹-6 |
| 3227 | Cl | NCH₃ | Ar⁹ | R¹¹-7 |
| 3228 | Cl | NCH₃ | Ar⁹ | R¹¹-8 |
| 3229 | Cl | NCH₃ | Ar⁹ | R¹¹-9 |
| 3230 | Cl | NCH₃ | Ar⁹ | R¹¹-10 |
| 3231 | Cl | NCH₃ | Ar⁹ | R¹¹-11 |
| 3232 | Cl | NCH₃ | Ar⁹ | R¹¹-12 |
| 3233 | Cl | NCH₃ | Ar⁹ | R¹¹-13 |
| 3234 | Cl | NCH₃ | Ar⁹ | R¹¹-14 |
| 3235 | Cl | NCH₃ | Ar⁹ | R¹¹-15 |
| 3236 | Cl | NCH₃ | Ar⁹ | R¹¹-16 |
| 3237 | Cl | NCH₃ | Ar⁹ | R¹¹-17 |
| 3238 | Cl | NCH₃ | Ar⁹ | R¹¹-18 |
| 3239 | Cl | NCH₃ | Ar⁹ | R¹¹-19 |
| 3240 | Cl | NCH₃ | Ar⁹ | R¹¹-20 |
| 3241 | Cl | NCH₃ | Ar⁹ | R¹¹-21 |
| 3242 | Cl | NCH₃ | Ar⁹ | R¹¹-22 |
| 3243 | Cl | NCH₃ | Ar⁹ | R¹¹-23 |
| 3244 | Cl | NCH₃ | Ar⁹ | R¹¹-24 |
| 3245 | Cl | NCH₃ | Ar⁹ | R¹¹-25 |
| 3246 | Cl | NCH₃ | Ar⁹ | R¹¹-26 |
| 3247 | Cl | NCH₃ | Ar⁹ | R¹¹-27 |
| 3248 | Cl | NCH₃ | Ar⁹ | R¹¹-28 |
| 3249 | Cl | NCH₃ | Ar⁹ | R¹¹-29 |
| 3250 | Cl | NCH₃ | Ar⁹ | A¹¹-1a |
| 3251 | Cl | NCH₃ | Ar⁹ | A¹¹-1b |
| 3252 | Cl | NCH₃ | Ar⁹ | A¹¹-2a |
| 3253 | Cl | NCH₃ | Ar⁹ | A¹¹-2b |
| 3254 | Cl | NCH₃ | Ar⁹ | A¹¹-3a |
| 3255 | Cl | NCH₃ | Ar⁹ | A¹¹-3b |
| 3256 | Cl | NCH₃ | Ar¹⁰ | R¹¹-1 |
| 3257 | Cl | NCH₃ | Ar¹⁰ | R¹¹-2 |
| 3258 | Cl | NCH₃ | Ar¹⁰ | R¹¹-3 |
| 3259 | Cl | NCH₃ | Ar¹⁰ | R¹¹-4 |
| 3260 | Cl | NCH₃ | Ar¹⁰ | R¹¹-5 |
| 3261 | Cl | NCH₃ | Ar¹⁰ | R¹¹-6 |
| 3262 | Cl | NCH₃ | Ar¹⁰ | R¹¹-7 |
| 3263 | Cl | NCH₃ | Ar¹⁰ | R¹¹-8 |
| 3264 | Cl | NCH₃ | Ar¹⁰ | R¹¹-9 |
| 3265 | Cl | NCH₃ | Ar¹⁰ | R¹¹-10 |
| 3266 | Cl | NCH₃ | Ar¹⁰ | R¹¹-11 |
| 3267 | Cl | NCH₃ | Ar¹⁰ | R¹¹-12 |
| 3268 | Cl | NCH₃ | Ar¹⁰ | R¹¹-13 |
| 3269 | Cl | NCH₃ | Ar¹⁰ | R¹¹-14 |
| 3270 | Cl | NCH₃ | Ar¹⁰ | R¹¹-15 |
| 3271 | Cl | NCH₃ | Ar¹⁰ | R¹¹-16 |
| 3272 | Cl | NCH₃ | Ar¹⁰ | R¹¹-17 |
| 3273 | Cl | NCH₃ | Ar¹⁰ | R¹¹-18 |
| 3274 | Cl | NCH₃ | Ar¹⁰ | R¹¹-19 |
| 3275 | Cl | NCH₃ | Ar¹⁰ | R¹¹-20 |
| 3276 | Cl | NCH₃ | Ar¹⁰ | R¹¹-21 |
| 3277 | Cl | NCH₃ | Ar¹⁰ | R¹¹-22 |
| 3278 | Cl | NCH₃ | Ar¹⁰ | R¹¹-23 |
| 3279 | Cl | NCH₃ | Ar¹⁰ | R¹¹-24 |
| 3280 | Cl | NCH₃ | Ar¹⁰ | R¹¹-25 |
| 3281 | Cl | NCH₃ | Ar¹⁰ | R¹¹-26 |
| 3282 | Cl | NCH₃ | Ar¹⁰ | R¹¹-27 |
| 3283 | Cl | NCH₃ | Ar¹⁰ | R¹¹-28 |
| 3284 | Cl | NCH₃ | Ar¹⁰ | R¹¹-29 |
| 3285 | Cl | NCH₃ | Ar¹⁰ | A¹¹-1a |
| 3286 | Cl | NCH₃ | Ar¹⁰ | A¹¹-1b |
| 3287 | Cl | NCH₃ | Ar¹⁰ | A¹¹-2a |
| 3288 | Cl | NCH₃ | Ar¹⁰ | A¹¹-2b |
| 3289 | Cl | NCH₃ | Ar¹⁰ | A¹¹-3a |
| 3290 | Cl | NCH₃ | Ar¹⁰ | A¹¹-3b |
| 3291 | Cl | NCH₃ | Ar¹¹ | R¹¹-1 |
| 3292 | Cl | NCH₃ | Ar¹¹ | R¹¹-2 |
| 3293 | Cl | NCH₃ | Ar¹¹ | R¹¹-3 |
| 3294 | Cl | NCH₃ | Ar¹¹ | R¹¹-4 |
| 3295 | Cl | NCH₃ | Ar¹¹ | R¹¹-5 |
| 3296 | Cl | NCH₃ | Ar¹¹ | R¹¹-6 |
| 3297 | Cl | NCH₃ | Ar¹¹ | R¹¹-7 |
| 3298 | Cl | NCH₃ | Ar¹¹ | R¹¹-8 |
| 3299 | Cl | NCH₃ | Ar¹¹ | R¹¹-9 |
| 3300 | Cl | NCH₃ | Ar¹¹ | R¹¹-10 |
| 3301 | Cl | NCH₃ | Ar¹¹ | R¹¹-11 |
| 3302 | Cl | NCH₃ | Ar¹¹ | R¹¹-12 |
| 3303 | Cl | NCH₃ | Ar¹¹ | R¹¹-13 |
| 3304 | Cl | NCH₃ | Ar¹¹ | R¹¹-14 |
| 3305 | Cl | NCH₃ | Ar¹¹ | R¹¹-15 |
| 3306 | Cl | NCH₃ | Ar¹¹ | R¹¹-16 |
| 3307 | Cl | NCH₃ | Ar¹¹ | R¹¹-17 |
| 3308 | Cl | NCH₃ | Ar¹¹ | R¹¹-18 |
| 3309 | Cl | NCH₃ | Ar¹¹ | R¹¹-19 |
| 3310 | Cl | NCH₃ | Ar¹¹ | R¹¹-20 |
| 3311 | Cl | NCH₃ | Ar¹¹ | R¹¹-21 |
| 3312 | Cl | NCH₃ | Ar¹¹ | R¹¹-22 |
| 3313 | Cl | NCH₃ | Ar¹¹ | R¹¹-23 |
| 3314 | Cl | NCH₃ | Ar¹¹ | R¹¹-24 |
| 3315 | Cl | NCH₃ | Ar¹¹ | R¹¹-25 |
| 3316 | Cl | NCH₃ | Ar¹¹ | R¹¹-26 |
| 3317 | Cl | NCH₃ | Ar¹¹ | R¹¹-27 |
| 3318 | Cl | NCH₃ | Ar¹¹ | R¹¹-28 |
| 3319 | Cl | NCH₃ | Ar¹¹ | R¹¹-29 |
| 3320 | Cl | NCH₃ | Ar¹¹ | A¹¹-1a |
| 3321 | Cl | NCH₃ | Ar¹¹ | A¹¹-1b |
| 3322 | Cl | NCH₃ | Ar¹¹ | A¹¹-2a |
| 3323 | Cl | NCH₃ | Ar¹¹ | A¹¹-2b |
| 3324 | Cl | NCH₃ | Ar¹¹ | A¹¹-3a |
| 3325 | Cl | NCH₃ | Ar¹¹ | A¹¹-3b |
| 3326 | Cl | NCH₃ | Ar¹² | R¹¹-1 |
| 3327 | Cl | NCH₃ | Ar¹² | R¹¹-2 |
| 3328 | Cl | NCH₃ | Ar¹² | R¹¹-3 |
| 3329 | Cl | NCH₃ | Ar¹² | R¹¹-4 |
| 3330 | Cl | NCH₃ | Ar¹² | R¹¹-5 |
| 3331 | Cl | NCH₃ | Ar¹² | R¹¹-6 |
| 3332 | Cl | NCH₃ | Ar¹² | R¹¹-7 |
| 3333 | Cl | NCH₃ | Ar¹² | R¹¹-8 |
| 3334 | Cl | NCH₃ | Ar¹² | R¹¹-9 |
| 3335 | Cl | NCH₃ | Ar¹² | R¹¹-10 |
| 3336 | Cl | NCH₃ | Ar¹² | R¹¹-11 |
| 3337 | Cl | NCH₃ | Ar¹² | R¹¹-12 |
| 3338 | Cl | NCH₃ | Ar¹² | R¹¹-13 |
| 3339 | Cl | NCH₃ | Ar¹² | R¹¹-14 |
| 3340 | Cl | NCH₃ | Ar¹² | R¹¹-15 |
| 3341 | Cl | NCH₃ | Ar¹² | R¹¹-16 |
| 3342 | Cl | NCH₃ | Ar¹² | R¹¹-17 |
| 3343 | Cl | NCH₃ | Ar¹² | R¹¹-18 |
| 3344 | Cl | NCH₃ | Ar¹² | R¹¹-19 |
| 3345 | Cl | NCH₃ | Ar¹² | R¹¹-20 |
| 3346 | Cl | NCH₃ | Ar¹² | R¹¹-21 |
| 3347 | Cl | NCH₃ | Ar¹² | R¹¹-22 |
| 3348 | Cl | NCH₃ | Ar¹² | R¹¹-23 |
| 3349 | Cl | NCH₃ | Ar¹² | R¹¹-24 |
| 3350 | Cl | NCH₃ | Ar¹² | R¹¹-25 |
| 3351 | Cl | NCH₃ | Ar¹² | R¹¹-26 |
| 3352 | Cl | NCH₃ | Ar¹² | R¹¹-27 |
| 3353 | Cl | NCH₃ | Ar¹² | R¹¹-28 |
| 3354 | Cl | NCH₃ | Ar¹² | R¹¹-29 |
| 3355 | Cl | NCH₃ | Ar¹² | A¹¹-1a |
| 3356 | Cl | NCH₃ | Ar¹² | A¹¹-1b |
| 3357 | Cl | NCH₃ | Ar¹² | A¹¹-2a |
| 3358 | Cl | NCH₃ | Ar¹² | A¹¹-2b |
| 3359 | Cl | NCH₃ | Ar¹² | A¹¹-3a |
| 3360 | Cl | NCH₃ | Ar¹² | A¹¹-3b |
| 3361 | Cl | O | Ar¹ | R¹¹-1 |
| 3362 | Cl | O | Ar¹ | R¹¹-2 |
| 3363 | Cl | O | Ar¹ | R¹¹-3 |
| 3364 | Cl | O | Ar¹ | R¹¹-4 |
| 3365 | Cl | O | Ar¹ | R¹¹-5 |
| 3366 | Cl | O | Ar¹ | R¹¹-6 |
| 3367 | Cl | O | Ar¹ | R¹¹-7 |
| 3368 | Cl | O | Ar¹ | R¹¹-8 |
| 3369 | Cl | O | Ar¹ | R¹¹-9 |
| 3370 | Cl | O | Ar¹ | R¹¹-10 |
| 3371 | Cl | O | Ar¹ | R¹¹-11 |
| 3372 | Cl | O | Ar¹ | R¹¹-12 |
| 3373 | Cl | O | Ar¹ | R¹¹-13 |
| 3374 | Cl | O | Ar¹ | R¹¹-14 |
| 3375 | Cl | O | Ar¹ | R¹¹-15 |
| 3376 | Cl | O | Ar¹ | R¹¹-16 |
| 3377 | Cl | O | Ar¹ | R¹¹-17 |
| 3378 | Cl | O | Ar¹ | R¹¹-18 |
| 3379 | Cl | O | Ar¹ | R¹¹-19 |
| 3380 | Cl | O | Ar¹ | R¹¹-20 |
| 3381 | Cl | O | Ar¹ | R¹¹-21 |
| 3382 | Cl | O | Ar¹ | R¹¹-22 |
| 3383 | Cl | O | Ar¹ | R¹¹-23 |
| 3384 | Cl | O | Ar¹ | R¹¹-24 |
| 3385 | Cl | O | Ar¹ | R¹¹-25 |
| 3386 | Cl | O | Ar¹ | R¹¹-26 |
| 3387 | Cl | O | Ar¹ | R¹¹-27 |
| 3388 | Cl | O | Ar¹ | R¹¹-28 |
| 3389 | Cl | O | Ar¹ | R¹¹-29 |
| 3390 | Cl | O | Ar¹ | A¹¹-1a |
| 3391 | Cl | O | Ar¹ | A¹¹-1b |
| 3392 | Cl | O | Ar¹ | A¹¹-2a |
| 3393 | Cl | O | Ar¹ | A¹¹-2b |
| 3394 | Cl | O | Ar¹ | A¹¹-3a |
| 3395 | Cl | O | Ar¹ | A¹¹-3b |
| 3396 | Cl | O | Ar² | R¹¹-1 |
| 3397 | Cl | O | Ar² | R¹¹-2 |
| 3398 | Cl | O | Ar² | R¹¹-3 |
| 3399 | Cl | O | Ar² | R¹¹-4 |
| 3400 | Cl | O | Ar² | R¹¹-5 |
| 3401 | Cl | O | Ar² | R¹¹-6 |
| 3402 | Cl | O | Ar² | R¹¹-7 |
| 3403 | Cl | O | Ar² | R¹¹-8 |
| 3404 | Cl | O | Ar² | R¹¹-9 |
| 3405 | Cl | O | Ar² | R¹¹-10 |
| 3406 | Cl | O | Ar² | R¹¹-11 |
| 3407 | Cl | O | Ar² | R¹¹-12 |
| 3408 | Cl | O | Ar² | R¹¹-13 |
| 3409 | Cl | O | Ar² | R¹¹-14 |
| 3410 | Cl | O | Ar² | R¹¹-15 |
| 3411 | Cl | O | Ar² | R¹¹-16 |
| 3412 | Cl | O | Ar² | R¹¹-17 |
| 3413 | Cl | O | Ar² | R¹¹-18 |
| 3414 | Cl | O | Ar² | R¹¹-19 |
| 3415 | Cl | O | Ar² | R¹¹-20 |
| 3416 | Cl | O | Ar² | R¹¹-21 |
| 3417 | Cl | O | Ar² | R¹¹-22 |
| 3418 | Cl | O | Ar² | R¹¹-23 |
| 3419 | Cl | O | Ar² | R¹¹-24 |
| 3420 | Cl | O | Ar² | R¹¹-25 |
| 3421 | Cl | O | Ar² | R¹¹-26 |
| 3422 | Cl | O | Ar² | R¹¹-27 |
| 3423 | Cl | O | Ar² | R¹¹-28 |
| 3424 | Cl | O | Ar² | R¹¹-29 |
| 3425 | Cl | O | Ar² | A¹¹-1a |
| 3426 | Cl | O | Ar² | A¹¹-1b |
| 3427 | Cl | O | Ar² | A¹¹-2a |
| 3428 | Cl | O | Ar² | A¹¹-2b |
| 3429 | Cl | O | Ar² | A¹¹-3a |
| 3430 | Cl | O | Ar² | A¹¹-3b |
| 3431 | Cl | O | Ar³ | R¹¹-1 |
| 3432 | Cl | O | Ar³ | R¹¹-2 |
| 3433 | Cl | O | Ar³ | R¹¹-3 |
| 3434 | Cl | O | Ar³ | R¹¹-4 |
| 3435 | Cl | O | Ar³ | R¹¹-5 |
| 3436 | Cl | O | Ar³ | R¹¹-6 |
| 3437 | Cl | O | Ar³ | R¹¹-7 |
| 3438 | Cl | O | Ar³ | R¹¹-8 |
| 3439 | Cl | O | Ar³ | R¹¹-9 |
| 3440 | Cl | O | Ar³ | R¹¹-10 |
| 3441 | Cl | O | Ar³ | R¹¹-11 |
| 3442 | Cl | O | Ar³ | R¹¹-12 |
| 3443 | Cl | O | Ar³ | R¹¹-13 |
| 3444 | Cl | O | Ar³ | R¹¹-14 |
| 3445 | Cl | O | Ar³ | R¹¹-15 |
| 3446 | Cl | O | Ar³ | R¹¹-16 |
| 3447 | Cl | O | Ar³ | R¹¹-17 |
| 3448 | Cl | O | Ar³ | R¹¹-18 |
| 3449 | Cl | O | Ar³ | R¹¹-19 |
| 3450 | Cl | O | Ar³ | R¹¹-20 |
| 3451 | Cl | O | Ar³ | R¹¹-21 |
| 3452 | Cl | O | Ar³ | R¹¹-22 |
| 3453 | Cl | O | Ar³ | R¹¹-23 |
| 3454 | Cl | O | Ar³ | R¹¹-24 |
| 3455 | Cl | O | Ar³ | R¹¹-25 |
| 3456 | Cl | O | Ar³ | R¹¹-26 |
| 3457 | Cl | O | Ar³ | R¹¹-27 |
| 3458 | Cl | O | Ar³ | R¹¹-28 |
| 3459 | Cl | O | Ar³ | R¹¹-29 |
| 3460 | Cl | O | Ar³ | A¹¹-1a |
| 3461 | Cl | O | Ar³ | A¹¹-1b |
| 3462 | Cl | O | Ar³ | A¹¹-2a |
| 3463 | Cl | O | Ar³ | A¹¹-2b |
| 3464 | Cl | O | Ar³ | A¹¹-3a |
| 3465 | Cl | O | Ar⁴ | A¹¹-3b |
| 3466 | Cl | O | Ar⁴ | R¹¹-1 |
| 3467 | Cl | O | Ar⁴ | R¹¹-2 |
| 3468 | Cl | O | Ar⁴ | R¹¹-3 |
| 3469 | Cl | O | Ar⁴ | R¹¹-4 |
| 3470 | Cl | O | Ar⁴ | R¹¹-5 |
| 3471 | Cl | O | Ar⁴ | R¹¹-6 |
| 3472 | Cl | O | Ar⁴ | R¹¹-7 |
| 3473 | Cl | O | Ar⁴ | R¹¹-8 |
| 3474 | Cl | O | Ar⁴ | R¹¹-9 |
| 3475 | Cl | O | Ar⁴ | R¹¹-10 |
| 3476 | Cl | O | Ar⁴ | R¹¹-11 |
| 3477 | Cl | O | Ar⁴ | R¹¹-12 |
| 3478 | Cl | O | Ar⁴ | R¹¹-13 |
| 3479 | Cl | O | Ar⁴ | R¹¹-14 |
| 3480 | Cl | O | Ar⁴ | R¹¹-15 |
| 3481 | Cl | O | Ar⁴ | R¹¹-16 |
| 3482 | Cl | O | Ar⁴ | R¹¹-17 |
| 3483 | Cl | O | Ar⁴ | R¹¹-18 |
| 3484 | Cl | O | Ar⁴ | R¹¹-19 |
| 3485 | Cl | O | Ar⁴ | R¹¹-20 |
| 3486 | Cl | O | Ar⁴ | R¹¹-21 |
| 3487 | Cl | O | Ar⁴ | R¹¹-22 |
| 3488 | Cl | O | Ar⁴ | R¹¹-23 |
| 3489 | Cl | O | Ar⁴ | R¹¹-24 |
| 3490 | Cl | O | Ar⁴ | R¹¹-25 |
| 3491 | Cl | O | Ar⁴ | R¹¹-26 |
| 3492 | Cl | O | Ar⁴ | R¹¹-27 |
| 3493 | Cl | O | Ar⁴ | R¹¹-28 |
| 3494 | Cl | O | Ar⁴ | R¹¹-29 |
| 3495 | Cl | O | Ar⁴ | A¹¹-1a |
| 3496 | Cl | O | Ar⁴ | A¹¹-1b |
| 3497 | Cl | O | Ar⁴ | A¹¹-2a |
| 3498 | Cl | O | Ar⁴ | A¹¹-2b |
| 3499 | Cl | O | Ar⁴ | A¹¹-3a |
| 3500 | Cl | O | Ar⁴ | A¹¹-3b |
| 3501 | Cl | O | Ar⁵ | R¹¹-1 |
| 3502 | Cl | O | Ar⁵ | R¹¹-2 |
| 3503 | Cl | O | Ar⁵ | R¹¹-3 |
| 3504 | Cl | O | Ar⁵ | R¹¹-4 |
| 3505 | Cl | O | Ar⁵ | R¹¹-5 |
| 3506 | Cl | O | Ar⁵ | R¹¹-6 |
| 3507 | Cl | O | Ar⁵ | R¹¹-7 |
| 3508 | Cl | O | Ar⁵ | R¹¹-8 |
| 3509 | Cl | O | Ar⁵ | R¹¹-9 |
| 3510 | Cl | O | Ar⁵ | R¹¹-10 |
| 3511 | Cl | O | Ar⁵ | R¹¹-11 |
| 3512 | Cl | O | Ar⁵ | R¹¹-12 |
| 3513 | Cl | O | Ar⁵ | R¹¹-13 |
| 3514 | Cl | O | Ar⁵ | R¹¹-14 |
| 3515 | Cl | O | Ar⁵ | R¹¹-15 |
| 3516 | Cl | O | Ar⁵ | R¹¹-16 |
| 3517 | Cl | O | Ar⁵ | R¹¹-17 |
| 3518 | Cl | O | Ar⁵ | R¹¹-18 |
| 3519 | Cl | O | Ar⁵ | R¹¹-19 |
| 3520 | Cl | O | Ar⁵ | R¹¹-20 |
| 3521 | Cl | O | Ar⁵ | R¹¹-21 |
| 3522 | Cl | O | Ar⁵ | R¹¹-22 |
| 3523 | Cl | O | Ar⁵ | R¹¹-23 |
| 3524 | Cl | O | Ar⁵ | R¹¹-24 |
| 3525 | Cl | O | Ar⁵ | R¹¹-25 |
| 3526 | Cl | O | Ar⁵ | R¹¹-26 |
| 3527 | Cl | O | Ar⁵ | R¹¹-27 |
| 3528 | Cl | O | Ar⁵ | R¹¹-28 |
| 3529 | Cl | O | Ar⁵ | R¹¹-29 |
| 3530 | Cl | O | Ar⁵ | A¹¹-1a |
| 3531 | Cl | O | Ar⁵ | A¹¹-1b |
| 3532 | Cl | O | Ar⁵ | A¹¹-2a |
| 3533 | Cl | O | Ar⁵ | A¹¹-2b |
| 3534 | Cl | O | Ar⁵ | A¹¹-3a |
| 3535 | Cl | O | Ar⁵ | A¹¹-3b |
| 3536 | Cl | O | Ar⁶ | R¹¹-1 |
| 3537 | Cl | O | Ar⁶ | R¹¹-2 |
| 3538 | Cl | O | Ar⁶ | R¹¹-3 |
| 3539 | Cl | O | Ar⁶ | R¹¹-4 |
| 3540 | Cl | O | Ar⁶ | R¹¹-5 |
| 3541 | Cl | O | Ar⁶ | R¹¹-6 |
| 3542 | Cl | O | Ar⁶ | R¹¹-7 |
| 3543 | Cl | O | Ar⁶ | R¹¹-8 |
| 3544 | Cl | O | Ar⁶ | R¹¹-9 |
| 3545 | Cl | O | Ar⁶ | R¹¹-10 |
| 3546 | Cl | O | Ar⁶ | R¹¹-11 |
| 3547 | Cl | O | Ar⁶ | R¹¹-12 |
| 3548 | Cl | O | Ar⁶ | R¹¹-13 |
| 3549 | Cl | O | Ar⁶ | R¹¹-14 |
| 3550 | Cl | O | Ar⁶ | R¹¹-15 |
| 3551 | Cl | O | Ar⁶ | R¹¹-16 |
| 3552 | Cl | O | Ar⁶ | R¹¹-17 |
| 3553 | Cl | O | Ar⁶ | R¹¹-18 |
| 3554 | Cl | O | Ar⁶ | R¹¹-19 |
| 3555 | Cl | O | Ar⁶ | R¹¹-20 |
| 3556 | Cl | O | Ar⁶ | R¹¹-21 |
| 3557 | Cl | O | Ar⁶ | R¹¹-22 |
| 3558 | Cl | O | Ar⁶ | R¹¹-23 |
| 3559 | Cl | O | Ar⁶ | R¹¹-24 |
| 3560 | Cl | O | Ar⁶ | R¹¹-25 |
| 3561 | Cl | O | Ar⁶ | R¹¹-26 |
| 3562 | Cl | O | Ar⁶ | R¹¹-27 |
| 3563 | Cl | O | Ar⁶ | R¹¹-28 |
| 3564 | Cl | O | Ar⁶ | R¹¹-29 |
| 3565 | Cl | O | Ar⁶ | A¹¹-1a |
| 3566 | Cl | O | Ar⁶ | A¹¹-1b |
| 3567 | Cl | O | Ar⁶ | A¹¹-2a |
| 3568 | Cl | O | Ar⁶ | A¹¹-2b |
| 3569 | Cl | O | Ar⁶ | A¹¹-3a |
| 3570 | Cl | O | Ar⁶ | A¹¹-3b |
| 3571 | Cl | O | Ar⁷ | R¹¹-1 |
| 3572 | Cl | O | Ar⁷ | R¹¹-2 |
| 3573 | Cl | O | Ar⁷ | R¹¹-3 |
| 3574 | Cl | O | Ar⁷ | R¹¹-4 |
| 3575 | Cl | O | Ar⁷ | R¹¹-5 |
| 3576 | Cl | O | Ar⁷ | R¹¹-6 |
| 3577 | Cl | O | Ar⁷ | R¹¹-7 |
| 3578 | Cl | O | Ar⁷ | R¹¹-8 |
| 3579 | Cl | O | Ar⁷ | R¹¹-9 |
| 3580 | Cl | O | Ar⁷ | R¹¹-10 |
| 3581 | Cl | O | Ar⁷ | R¹¹-11 |
| 3582 | Cl | O | Ar⁷ | R¹¹-12 |
| 3583 | Cl | O | Ar⁷ | R¹¹-13 |
| 3584 | Cl | O | Ar⁷ | R¹¹-14 |
| 3585 | Cl | O | Ar⁷ | R¹¹-15 |
| 3586 | Cl | O | Ar⁷ | R¹¹-16 |
| 3587 | Cl | O | Ar⁷ | R¹¹-17 |
| 3588 | Cl | O | Ar⁷ | R¹¹-18 |
| 3589 | Cl | O | Ar⁷ | R¹¹-19 |
| 3590 | Cl | O | Ar⁷ | R¹¹-20 |
| 3591 | Cl | O | Ar⁷ | R¹¹-21 |
| 3592 | Cl | O | Ar⁷ | R¹¹-22 |
| 3593 | Cl | O | Ar⁷ | R¹¹-23 |
| 3594 | Cl | O | Ar⁷ | R¹¹-24 |
| 3595 | Cl | O | Ar⁷ | R¹¹-25 |
| 3596 | Cl | O | Ar⁷ | R¹¹-26 |
| 3597 | Cl | O | Ar⁷ | R¹¹-27 |
| 3598 | Cl | O | Ar⁷ | R¹¹-28 |
| 3599 | Cl | O | Ar⁷ | R¹¹-29 |
| 3600 | Cl | O | Ar⁷ | A¹¹-1a |
| 3601 | Cl | O | Ar⁷ | A¹¹-1b |
| 3602 | Cl | O | Ar⁷ | A¹¹-2a |
| 3603 | Cl | O | Ar⁷ | A¹¹-2b |
| 3604 | Cl | O | Ar⁷ | A¹¹-3a |
| 3605 | Cl | O | Ar⁷ | A¹¹-3b |
| 3606 | Cl | O | Ar⁸ | R¹¹-1 |
| 3607 | Cl | O | Ar⁸ | R¹¹-2 |
| 3608 | Cl | O | Ar⁸ | R¹¹-3 |
| 3609 | Cl | O | Ar⁸ | R¹¹-4 |
| 3610 | Cl | O | Ar⁸ | R¹¹-5 |
| 3611 | Cl | O | Ar⁸ | R¹¹-6 |
| 3612 | Cl | O | Ar⁸ | R¹¹-7 |
| 3613 | Cl | O | Ar⁸ | R¹¹-8 |
| 3614 | Cl | O | Ar⁸ | R¹¹-9 |
| 3615 | Cl | O | Ar⁸ | R¹¹-10 |
| 3616 | Cl | O | Ar⁸ | R¹¹-11 |
| 3617 | Cl | O | Ar⁸ | R¹¹-12 |
| 3618 | Cl | O | Ar⁸ | R¹¹-13 |
| 3619 | Cl | O | Ar⁸ | R¹¹-14 |
| 3620 | Cl | O | Ar⁸ | R¹¹-15 |
| 3621 | Cl | O | Ar⁸ | R¹¹-16 |
| 3622 | Cl | O | Ar⁸ | R¹¹-17 |
| 3623 | Cl | O | Ar⁸ | R¹¹-18 |
| 3624 | Cl | O | Ar⁸ | R¹¹-19 |
| 3625 | Cl | O | Ar⁸ | R¹¹-20 |
| 3626 | Cl | O | Ar⁸ | R¹¹-21 |
| 3627 | Cl | O | Ar⁸ | R¹¹-22 |
| 3628 | Cl | O | Ar⁸ | R¹¹-23 |
| 3629 | Cl | O | Ar⁸ | R¹¹-24 |
| 3630 | Cl | O | Ar⁸ | R¹¹-25 |
| 3631 | Cl | O | Ar⁸ | R¹¹-26 |
| 3632 | Cl | O | Ar⁸ | R¹¹-27 |
| 3633 | Cl | O | Ar⁸ | R¹¹-28 |
| 3634 | Cl | O | Ar⁸ | R¹¹-29 |
| 3635 | Cl | O | Ar⁸ | A¹¹-1a |
| 3636 | Cl | O | Ar⁸ | A¹¹-1b |
| 3637 | Cl | O | Ar⁸ | A¹¹-2a |
| 3638 | Cl | O | Ar⁸ | A¹¹-2b |
| 3639 | Cl | O | Ar⁸ | A¹¹-3a |
| 3640 | Cl | O | Ar⁸ | A¹¹-3b |
| 3641 | Cl | O | Ar⁹ | R¹¹-1 |
| 3642 | Cl | O | Ar⁹ | R¹¹-2 |
| 3643 | Cl | O | Ar⁹ | R¹¹-3 |
| 3644 | Cl | O | Ar⁹ | R¹¹-4 |
| 3645 | Cl | O | Ar⁹ | R¹¹-5 |
| 3646 | Cl | O | Ar⁹ | R¹¹-6 |
| 3647 | Cl | O | Ar⁹ | R¹¹-7 |
| 3648 | Cl | O | Ar⁹ | R¹¹-8 |
| 3649 | Cl | O | Ar⁹ | R¹¹-9 |
| 3650 | Cl | O | Ar⁹ | R¹¹-10 |
| 3651 | Cl | O | Ar⁹ | R¹¹-11 |
| 3652 | Cl | O | Ar⁹ | R¹¹-12 |
| 3653 | Cl | O | Ar⁹ | R¹¹-13 |
| 3654 | Cl | O | Ar⁹ | R¹¹-14 |
| 3655 | Cl | O | Ar⁹ | R¹¹-15 |
| 3656 | Cl | O | Ar⁹ | R¹¹-16 |
| 3657 | Cl | O | Ar⁹ | R¹¹-17 |
| 3658 | Cl | O | Ar⁹ | R¹¹-18 |
| 3659 | Cl | O | Ar⁹ | R¹¹-19 |
| 3660 | Cl | O | Ar⁹ | R¹¹-20 |
| 3661 | Cl | O | Ar⁹ | R¹¹-21 |
| 3662 | Cl | O | Ar⁹ | R¹¹-22 |
| 3663 | Cl | O | Ar⁹ | R¹¹-23 |
| 3664 | Cl | O | Ar⁹ | R¹¹-24 |
| 3665 | Cl | O | Ar⁹ | R¹¹-25 |
| 3666 | Cl | O | Ar⁹ | R¹¹-26 |
| 3667 | Cl | O | Ar⁹ | R¹¹-27 |
| 3668 | Cl | O | Ar⁹ | R¹¹-28 |
| 3669 | Cl | O | Ar⁹ | R¹¹-29 |
| 3670 | Cl | O | Ar⁹ | A¹¹-1a |
| 3671 | Cl | O | Ar⁹ | A¹¹-1b |
| 3672 | Cl | O | Ar⁹ | A¹¹-2a |
| 3673 | Cl | O | Ar⁹ | A¹¹-2b |
| 3674 | Cl | O | Ar⁹ | A¹¹-3a |
| 3675 | Cl | O | Ar⁹ | A¹¹-3b |
| 3676 | Cl | O | Ar¹⁰ | R¹¹-1 |
| 3677 | Cl | O | Ar¹⁰ | R¹¹-2 |
| 3678 | Cl | O | Ar¹⁰ | R¹¹-3 |
| 3679 | Cl | O | Ar¹⁰ | R¹¹-4 |
| 3680 | Cl | O | Ar¹⁰ | R¹¹-5 |
| 3681 | Cl | O | Ar¹⁰ | R¹¹-6 |
| 3682 | Cl | O | Ar¹⁰ | R¹¹-7 |
| 3683 | Cl | O | Ar¹⁰ | R¹¹-8 |
| 3684 | Cl | O | Ar¹⁰ | R¹¹-9 |
| 3685 | Cl | O | Ar¹⁰ | R¹¹-10 |
| 3686 | Cl | O | Ar¹⁰ | R¹¹-11 |
| 3687 | Cl | O | Ar¹⁰ | R¹¹-12 |
| 3688 | Cl | O | Ar¹⁰ | R¹¹-13 |
| 3689 | Cl | O | Ar¹⁰ | R¹¹-14 |
| 3690 | Cl | O | Ar¹⁰ | R¹¹-15 |
| 3691 | Cl | O | Ar¹⁰ | R¹¹-16 |
| 3692 | Cl | O | Ar¹⁰ | R¹¹-17 |
| 3693 | Cl | O | Ar¹⁰ | R¹¹-18 |
| 3694 | Cl | O | Ar¹⁰ | R¹¹-19 |
| 3695 | Cl | O | Ar¹⁰ | R¹¹-20 |
| 3696 | Cl | O | Ar¹⁰ | R¹¹-21 |
| 3697 | Cl | O | Ar¹⁰ | R¹¹-22 |
| 3698 | Cl | O | Ar¹⁰ | R¹¹-23 |
| 3699 | Cl | O | Ar¹⁰ | R¹¹-24 |
| 3700 | Cl | O | Ar¹⁰ | R¹¹-25 |
| 3701 | Cl | O | Ar¹⁰ | R¹¹-26 |
| 3702 | Cl | O | Ar¹⁰ | R¹¹-27 |
| 3703 | Cl | O | Ar¹⁰ | R¹¹-28 |
| 3704 | Cl | O | Ar¹⁰ | R¹¹-29 |
| 3705 | Cl | O | Ar¹⁰ | A¹¹-1a |
| 3706 | Cl | O | Ar¹⁰ | A¹¹-1b |
| 3707 | Cl | O | Ar¹⁰ | A¹¹-2a |
| 3708 | Cl | O | Ar¹⁰ | A¹¹-2b |
| 3709 | Cl | O | Ar¹⁰ | A¹¹-3a |
| 3710 | Cl | O | Ar¹⁰ | A¹¹-3b |
| 3711 | Cl | O | Ar¹¹ | R¹¹-1 |
| 3712 | Cl | O | Ar¹¹ | R¹¹-2 |
| 3713 | Cl | O | Ar¹¹ | R¹¹-3 |
| 3714 | Cl | O | Ar¹¹ | R¹¹-4 |
| 3715 | Cl | O | Ar¹¹ | R¹¹-5 |
| 3716 | Cl | O | Ar¹¹ | R¹¹-6 |
| 3717 | Cl | O | Ar¹¹ | R¹¹-7 |
| 3718 | Cl | O | Ar¹¹ | R¹¹-8 |
| 3719 | Cl | O | Ar¹¹ | R¹¹-9 |
| 3720 | Cl | O | Ar¹¹ | R¹¹-10 |
| 3721 | Cl | O | Ar¹¹ | R¹¹-11 |
| 3722 | Cl | O | Ar¹¹ | R¹¹-12 |
| 3723 | Cl | O | Ar¹¹ | R¹¹-13 |
| 3724 | Cl | O | Ar¹¹ | R¹¹-14 |
| 3725 | Cl | O | Ar¹¹ | R¹¹-15 |
| 3726 | Cl | O | Ar¹¹ | R¹¹-16 |
| 3727 | Cl | O | Ar¹¹ | R¹¹-17 |
| 3728 | Cl | O | Ar¹¹ | R¹¹-18 |
| 3729 | Cl | O | Ar¹¹ | R¹¹-19 |
| 3730 | Cl | O | Ar¹¹ | R¹¹-20 |
| 3731 | Cl | O | Ar¹¹ | R¹¹-21 |
| 3732 | Cl | O | Ar¹¹ | R¹¹-22 |
| 3733 | Cl | O | Ar¹¹ | R¹¹-23 |
| 3734 | Cl | O | Ar¹¹ | R¹¹-24 |
| 3735 | Cl | O | Ar¹¹ | R¹¹-25 |
| 3736 | Cl | O | Ar¹¹ | R¹¹-26 |
| 3737 | Cl | O | Ar¹¹ | R¹¹-27 |
| 3738 | Cl | O | Ar¹¹ | R¹¹-28 |
| 3739 | Cl | O | Ar¹¹ | R¹¹-29 |
| 3740 | Cl | O | Ar¹¹ | A¹¹-1a |
| 3741 | Cl | O | Ar¹¹ | A¹¹-1b |
| 3742 | Cl | O | Ar¹¹ | A¹¹-2a |
| 3743 | Cl | O | Ar¹¹ | A¹¹-2b |
| 3744 | Cl | O | Ar¹¹ | A¹¹-3a |
| 3745 | Cl | O | Ar¹¹ | A¹¹-3b |
| 3746 | Cl | O | Ar¹² | R¹¹-1 |
| 3747 | Cl | O | Ar¹² | R¹¹-2 |
| 3748 | Cl | O | Ar¹² | R¹¹-3 |
| 3749 | Cl | O | Ar¹² | R¹¹-4 |
| 3750 | Cl | O | Ar¹² | R¹¹-5 |
| 3751 | Cl | O | Ar¹² | R¹¹-6 |
| 3752 | Cl | O | Ar¹² | R¹¹-7 |
| 3753 | Cl | O | Ar¹² | R¹¹-8 |
| 3754 | Cl | O | Ar¹² | R¹¹-9 |
| 3755 | Cl | O | Ar¹² | R¹¹-10 |
| 3756 | Cl | O | Ar¹² | R¹¹-11 |
| 3757 | Cl | O | Ar¹² | R¹¹-12 |
| 3758 | Cl | O | Ar¹² | R¹¹-13 |
| 3759 | Cl | O | Ar¹² | R¹¹-14 |
| 3760 | Cl | O | Ar¹² | R¹¹-15 |
| 3761 | Cl | O | Ar¹² | R¹¹-16 |
| 3762 | Cl | O | Ar¹² | R¹¹-17 |
| 3763 | Cl | O | Ar¹² | R¹¹-18 |
| 3764 | Cl | O | Ar¹² | R¹¹-19 |
| 3765 | Cl | O | Ar¹² | R¹¹-20 |
| 3766 | Cl | O | Ar¹² | R¹¹-21 |
| 3767 | Cl | O | Ar¹² | R¹¹-22 |
| 3768 | Cl | O | Ar¹² | R¹¹-23 |
| 3769 | Cl | O | Ar¹² | R¹¹-24 |
| 3770 | Cl | O | Ar¹² | R¹¹-25 |
| 3771 | Cl | O | Ar¹² | R¹¹-26 |
| 3772 | Cl | O | Ar¹² | R¹¹-27 |
| 3773 | Cl | O | Ar¹² | R¹¹-28 |
| 3774 | Cl | O | Ar¹² | R¹¹-29 |
| 3775 | Cl | O | Ar¹² | A¹¹-1a |
| 3776 | Cl | O | Ar¹² | A¹¹-1b |
| 3777 | Cl | O | Ar¹² | A¹¹-2a |
| 3778 | Cl | O | Ar¹² | A¹¹-2b |
| 3779 | Cl | O | Ar¹² | A¹¹-3a |
| 3780 | Cl | O | Ar¹² | A¹¹-3b |

As used herein, the term “compound(s) of the present invention” or “compound(s) according to the invention” refers to the compound(s) of formula (I) as defined above, which are also referred to as “compound(s) of formula I” or “compound(s) I” or “formula I compound(s)”, and includes their salts, tautomers, stereoisomers, and N-oxides.

The present invention also relates to a mixture of at least one compound of the present invention with at least one mixing partner as defined herein after. Preferred are binary mixtures of one compound of the present invention as component I with one mixing partner as defined herein after as component II. Preferred weight ratios for such binary mixtures are from 5000:1 to 1:5000, preferably from 1000:1 to 1:1000, more preferably from 100:1 to 1:100, particularly preferably from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers, and the like. Preferred mixing partners are insecticides, nematicides and fungicides.

The following list M of pesticides, grouped and numbered according the Mode of Action Classification of the Insecticide Resistance Action Committee (IRAC), together with which the compounds of the present invention can be used and with which potential synergistic effects might be produced, is intended to illustrate the possible combinations, but not to impose any limitation:

M.1 Acetylcholine esterase (AChE) inhibitors from the class of: M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of M.1B organophosphates, for example acephate, azamethiphos, azinphos-ethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothio-phosphoryl) salicylate, isoxathion, malathion, mecarbam, methamidophos, methidathion, mevinphos, monocrotophos, naled, omethoate, oxydemetonmethyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;

M.2. GABA-gated chloride channel antagonists such as: M.2A cyclodiene organochlorine compounds, as for example endosulfan or chlordane; or M.2B fiproles (phenylpyrazoles), as for exampie ethiprole, fipronil, flufiprole, pyrafluprole and pyriprole;

M.3 Sodium channel modulators from the class of M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gammacyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, heptafluthrin, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or M.3B sodium channel modulators such as DDT or methoxychlor;

M.4 Nicotinic acetylcholine receptor agonists (nAChR) from the class of M.4A neonicotinoids, for example acetamiprid, clothianidin, cycloxaprid, dinotefuran, imidacloprid, nitenpyram, thiacloprid and thiamethoxam; or the compounds M.4A.2: (2E-)-1-[(6-Chloropyridin-3-yl)methyl]-N′-nitro-2-pentylidenehydrazinecarboximidamide; or M4.A.3: 1-[(6-Chloropyridin-3-yl)methyl]-7-methyl-8-nitro-5-propoxy-1,2,3,5,6,7-hexahydroimidazo[1,2-a]pyridine; or from the class M.4B nicotine;

M.5 Nicotinic acetylcholine receptor allosteric activators from the class of spinosyns, for example spinosad or spinetoram;

M.6 Chloride channel activators from the class of avermectins and milbemycins, for example abamectin, emamectin benzoate, ivermectin, lepimectin or milbemectin;

M.7 Juvenile hormone mimics, such as M.7A juvenile hormone analogues as hydroprene, kinoprene and methoprene; or others as M.7B fenoxycarb or M.7C pyriproxyfen;

M.8 miscellaneous non-specific (multi-site) inhibitors, for example M.8A alkyl halides as methyl bromide and other alkyl halides, or M.8B chloropicrin, or M.8C sulfuryl fluoride, or M.8D borax, or M.8E tartar emetic;

M.9 Selective homopteran feeding blockers, for example M.9B pymetrozine, or M.9C flonicamid;

M.10 Mite growth inhibitors, for example M.10A clofentezine, hexythiazox and diflovidazin, or M.10B etoxazole;

M.11 Microbial disruptors of insect midgut membranes, for example Bacillus thuringiensis or Bacillus sphaericus and the insecticdal proteins they produce such as Bacillus thuringiensis subsp. israelensis, Bacillus sphaericus, Bacillus thuringiensis subsp. aizawai, Bacillus thuringiensis subsp. kurstaki and Bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;

M.12 Inhibitors of mitochondrial ATP synthase, for example M.12A diafenthiuron, or M.12B organotin miticides such as azocyclotin, cyhexatin or fenbutatin oxide, or M.12C propargite, or M.12D tetradifon;

M.13 Uncouplers of oxidative phosphorylation via disruption of the proton gradient, for example chlorfenapyr, DNOC or sulfluramid;

M.14 Nicotinic acetylcholine receptor (nAChR) channel blockers, for example nereistoxin analogues as bensultap, cartap hydrochloride, thiocyclam or thiosultap sodium;

M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;

M.16 Inhibitors of the chitin biosynthesis type 1, as for example buprofezin;

M.17 Moulting disruptors, Dipteran, as for example cyromazine;

M.18 Ecdyson receptor agonists such as diacylhydrazines, for example methoxyfenozide, tebufenozide, halofenozide, fufenozide or chromafenozide;

M.19 Octopamin receptor agonists, as for example amitraz;

M.20 Mitochondrial complex III electron transport inhibitors, for example M.20A hydramethylnon, or M.20B acequinocyl, or M.20C fluacrypyrim;

M.21 Mitochondrial complex I electron transport inhibitors, for example M.21A METI acaricides and insecticides such as fenazaquin, fenpyroximate, pyrimidifen, pyridaben, tebufenpyrad or tolfenpyrad, or M.21 B rotenone;

M.22 Voltage-dependent sodium channel blockers, for example M.22A indoxacarb, or M.22B metaflumizone, or M.22B.1: 2-[2-(4-Cyanophenyl)-1-[3-(trifluoromethyl)phenyl]ethyl idene]-N-[4-(difluoromethoxy)phenyl]-hydrazinecarboxamide or M.22B.2: N-(3-Chloro-2-methylphenyl)-2-[(4-chlorophenyl)[4-[methyl(methylsulfonyl)amino]phenyl]methylene]-hydrazinecarboxamide;

M.23 Inhibitors of the of acetyl CoA carboxylase, such as Tetronic and Tetramic acid derivatives, for example spirodiclofen, spiromesifen or spirotetramat;

M.24 Mitochondrial complex IV electron transport inhibitors, for example M.24A phosphine such as aluminium phosphide, calcium phosphide, phosphine or zinc phosphide, or M.24B cyanide;

M.25 Mitochondrial complex II electron transport inhibitors, such as beta-ketonitrile derivatives, for example cyenopyrafen or cyflumetofen;

M.28 Ryanodine receptor-modulators from the class of diamides, as for example flubendiamide, chlorantraniliprole (Rynaxypyr®), cyantraniliprole (Cyazypyr®), tetraniliprole, or the phthalamide compounds M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound M.28.4: methyl-2-[3,5-dibromo-2-({[3-bromo-1-(3-chlorpyridin-2-yl)-1H-pyrazol-5-yl]carbonyl}-amino)benzoyl]-1,2-dimethylhydrazinecarboxylate; or a compound selected from M.28.5a) to M.28.5d) and M.28.5h) to M.28.5l): M.28.5a) N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide; M.28.5b) N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide; M.28.5c) N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide; M.28.5d) N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide; M.28.5h) N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide; M.28.5i) N-[2-(5-Amino-1,3,4-thiadiazol-2-yl)-4-chloro-6-methylphenyl]-3-bromo-1-(3-chloro-2-pyridinyl)-1H-pyrazole-5-carboxamide; M.28.5j) 3-Chloro-1-(3-chloro-2-pyridinyl)-N-[2,4-dichloro-6-[[(1-cyano-1-methylethyl)amino]carbonyl]phenyl]-1H-pyrazole-5-carboxamide; M.28.5k) 3-Bromo-N[2,4-dichloro-6-(methylcarbamoyl)phenyl]-1-(3,5-dichloro-2-pyridyl)-1H-pyrazole-5-carboxamide; M.28.5l) N-[4-Chloro-2-[[(1,1-dimethylethyl)amino]carbonyl]-6-methylphenyl]-1-(3-chloro-2-pyridinyl)-3-(fluoromethoxy)-1H-pyrazole-5-carboxamide; or

M.28.6: cyhalodiamide; or;

M.29. insecticidal active compounds of unknown or uncertain mode of action, as for example afidopyropen, afoxolaner, azadirachtin, amidoflumet, benzoximate, bifenazate, broflanilide, bromopropylate, chinomethionat, cryolite, dicloromezotiaz, dicofol, flufenerim, flometoquin, fluensulfone, fluhexafon, fluopyram, flupyradifurone, fluralaner, metoxadiazone, piperonyl butoxide, pyflubumide, pyridalyl, pyrifluquinazon, sulfoxaflor, tioxazafen, triflumezopyrim, or the compounds

M.29.3: 11-(4-chloro-2,6-dimethylphenyl)-12-hydroxy-1,4-dioxa-9-azadispiro[4.2.4.2]-tetradec-11-en-10-one, or the compound

M.29.4: 3-(4′-fluoro-2,4-dimethylbiphenyl-3-yl)-4-hydroxy-8-oxa-1-azaspiro[4.5]dec-3-en-2-one, or the compound

M.29.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of Bacillus firmus (Votivo, 1-1582); or

a compound selected from the of M.29.6, wherein the compound M.29.6a) to M.29.6k): M.29.6a) (E/Z)—N-[1-[(6-chloro-3-pyridyl)methyl]-2-pyridylidene]-2,2,2-trifluoro-acetamide; M.29.6b) (E/Z)—N-[1-[(6-chloro-5-fluoro-3-pyridyl)methyl]-2-pyridylidene]-2,2,2-trifluoro-acetamide; M.29.6c) (E/Z)-2,2,2-trifluoro-N-[1-[(6-fluoro-3-pyridyl)methyl]-2-pyridylidene]acetamide; M.29.6d) (E/Z)—N-[1-[(6-bromo-3-pyridyl)methyl]-2-pyridylidene]-2,2,2-trifluoro-acetamide; M.29.6e) (E/Z)—N-[1-[1-(6-chloro-3-pyridyl)ethyl]-2-pyridylidene]-2,2,2-trifluoro-acetamide; M.29.6f) (E/Z)—N-[1-[(6-chloro-3-pyridyl)methyl]-2-pyridylidene]-2,2-difluoro-acetamide; M.29.6g) (E/Z)-2-chloro-N-[1-[(6-chloro-3-pyridyl)methyl]-2-pyridylidene]-2,2-difluoro-acetamide; M.29.6h) (E/Z)—N-[1-[(2-chloropyrimidin-5-yl)methyl]-2-pyridylidene]-2,2,2-trifluoro-acetamide; M.29.6i) (E/Z)—N-[1-[(6-chloro-3-pyridyl)methyl]-2-pyridylidene]-2,2,3,3,3-pentafluoro-propanamide.); M.29.6j) N-[1-[(6-chloro-3-pyridyl)methyl]-2-pyridylidene]-2,2,2-trifluoro-thioacetamide; or M.29.6k) N-[1-[(6-chloro-3-pyridyl)methyl]-2-pyridylidene]-2,2,2-trifluoro-N′-isopropyl-acetamidine; or the compounds

M.29.8: fluazaindolizine; or the compounds

M.29.9.a): 4-[5-(3,5-dichlorophenyl)-5-(trifluoromethyl)-4H-isoxazol-3-yl]-2-methyl-N-(1-oxothietan-3-yl)benzamide; or M.29.9.b): fluxametamide; or

M.29.10: 5-[3-[2,6-dichloro-4-(3,3-dichloroallyloxy)phenoxy]propoxy]-1H-pyrazole; or

a compound selected from the of M.29.11, wherein the compound M.29.11b) to M.29.11p): M.29.11.b) 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide; M.29.11.c) 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide; M.29.11.d) N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.e) N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide; M.29.11.f) 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.g) 3-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide; M.29.11.h) 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide; M.29.11.i) 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide; M.29.11.j) 4-cyano-3-[(4-cyano-2-methyl-benzoyl)amino]-N[2,6-dichloro-4-[1,2,2,3,3,3-hexafluoro-1-(trifluoromethyl)propyl]phenyl]-2-fluoro-benzamide; M.29.11.k) N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide; M.29.11.l) N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methylbenzamide; M.29.11.m) N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide; M.29.11.n) 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide; M.29.11.o) 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide; M.29.11.p) N-[5-[[2-bromo-6-chloro-4-[1,2,2,2-tetrafluoro-1-(trifluoromethyl)ethyl]phenyl]carbamoyl]-2-cyano-phenyl]-4-cyano-2-methylbenzamide; or

a compound selected from the of M.29.12, wherein the compound M.29.12a) to M.29.12m): M.29.12.a) 2-(1,3-Dioxan-2-yl)-6-[2-(3-pyridinyl)-5-thiazolyl]-pyridine; M.29.12.b) 2-[6-[2-(5-Fluoro-3-pyridinyl)-5-thiazolyl]-2-pyridinyl]-pyrimidine; M.29.12.c) 2-[6-[2-(3-Pyridinyl)-5-thiazolyl]-2-pyridinyl]-pyrimidine; M.29.12.d) N-Methylsulfonyl-6-[2-(3-pyridyl)thiazol-5-yl]pyridine-2-carboxamide; M.29.12.e) N-Methylsulfonyl-6-[2-(3-pyridyl)thiazol-5-yl]pyridine-2-carboxamide; M.29.12.f) N-Ethyl-N-[4-methyl-2-(3-pyridyl)thiazol-5-yl]-3-methylthio-propanamide; M.29.12.g) N-Methyl-N-[4-methyl-2-(3-pyridyl)thiazol-5-yl]-3-methylthio-propanamide; M.29.12.h) N,2-Dimethyl-N-[4-methyl-2-(3-pyridyl)thiazol-5-yl]-3-methylthio-propanamide; M.29.12.i) N-Ethyl-2-methyl-N-[4-methyl-2-(3-pyridyl)thiazol-5-yl]-3-methylthio-propanamide; M.29.12.j) N-[4-Chloro-2-(3-pyridyl)thiazol-5-yl]-N-ethyl-2-methyl-3-methylthio-propanamide; M.29.12.k) N-[4-Chloro-2-(3-pyridyl)thiazol-5-yl]-N,2-dimethyl-3-methylthio-propanamide; M.29.12.l) N-[4-Chloro-2-(3-pyridyl)thiazol-5-yl]-N-methyl-3-methylthio-propanamide; M.29.12.m) N-[4-Chloro-2-(3-pyridyl)thiazol-5-yl]-N-ethyl-3-methylthio-propanamide; or the compounds

M.29.14a) 1-[(6-Chloro-3-pyridinyl)methyl]-1,2,3,5,6,7-hexahydro-5-methoxy-7-methyl-8-nitro-imidazo[1,2-a]pyridine; or M.29.14b) 1-[(6-Chloropyridin-3-yl)methyl]-7-methyl-8-nitro-1,2,3,5,6,7-hexahydroimidazo[1,2-a]pyridin-5-ol; or the compounds

M.29.16a) 1-isopropyl-N,5-dimethyl-N-pyridazin-4-yl-pyrazole-4-carboxamide; or M.29.16b) 1-(1,2-dimethylpropyl)-N-ethyl-5-methyl-N-pyridazin-4-yl-pyrazole-4-carboxamide; M.29.16c) N,5-dimethyl-N-pyridazin-4-yl-1-(2,2,2-trifluoro-1-methyl-ethyl)pyrazole-4-carboxamide; M.29.16d) 1-[1-(1-cyanocyclopropyl)ethyl]-N-ethyl-5-methyl-N-pyridazin-4-yl-pyrazole-4-carboxamide; M.29.16e) N-ethyl-1-(2-fluoro-1-methyl-propyl)-5-methyl-N-pyridazin-4-yl-pyrazole-4-carboxamide; M.29.16f) 1-(1,2-dimethylpropyl)-N,5-dimethyl-N-pyridazin-4-yl-pyrazole-4-carboxamide; M.29.16g) 1-[1-(1-cyanocyclopropyl)ethyl]-N,5-dimethyl-N-pyridazin-4-yl-pyrazole-4-carboxamide; M.29.16h) N-methyl-1-(2-fluoro-1-methyl-propyl]-5-methyl-N-pyridazin-4-yl-pyrazole-4-carboxamide; M.29.16i) 1-(4,4-difluorocyclohexyl)-N-ethyl-5-methyl-N-pyridazin-4-yl-pyrazole-4-carboxamide; or M.29.16j) 1-(4,4-difluorocyclohexyl)-N,5-dimethyl-N-pyridazin-4-yl-pyrazole-4-carboxamide, or

M.29.17 a compound selected from the compounds M.29.17a) to M.29.17j): M.29.17a) N-(1-methylethyl)-2-(3-pyridinyl)-2H-indazole-4-carboxamide; M.29.17b) N-cyclopropyl-2-(3-pyridinyl)-2H-indazole-4-carboxamide; M.29.17c) N-cyclohexyl-2-(3-pyridinyl)-2H-indazole-4-carboxamide; M.29.17d) 2-(3-pyridinyl)-N-(2,2,2-trifluoroethyl)-2H-indazole-4-carboxamide; M.29.17e) 2-(3-pyridinyl)-N-[(tetrahydro-2-furanyl)methyl]-2H-indazole-5-carboxamide; M.29.17f) methyl 2-[[2-(3-pyridinyl)-2H-indazol-5-yl]carbonyl]hydrazinecarboxylate; M.29.17g) N-[(2,2-difluorocyclopropyl)methyl]-2-(3-pyridinyl)-2H-indazole-5-carboxamide; M.29.17h) N-(2,2-difluoropropyl)-2-(3-pyridinyl)-2H-indazole-5-carboxamide; M.29.17i) 2-(3-pyridinyl)-N-(2-pyrimidinylmethyl)-2H-indazole-5-carboxamide; M.29.17j) N-[(5-methyl-2-pyrazinyl)methyl]-2-(3-pyridinyl)-2H-indazole-5-carboxamide, or

M.29.18 a compound selected from the compounds M.29.18a) to M.29.18d): M.29.18a) N-[3-chloro-1-(3-pyridyl)pyrazol-4-yl]-N-ethyl-3-(3,3,3-trifluoropropylsulfanyl)propanamide; M.29.18b) N[3-chloro-1-(3-pyridyl)pyrazol-4-yl]-N-ethyl-3-(3,3,3-trifluoropropylsulfinyl)propanamide; M.29.18c) N-[3-chloro-1-(3-pyridyl)pyrazol-4-yl]-3-[(2,2-difluorocyclopropyl)methylsulfanyl]-N-ethyl-propanamide;

M.29.18d) N-[3-chloro-1-(3-pyridyl)pyrazol-4-yl]-3-[(2,2-difluorocyclopropyl)methylsulfinyl]-N-ethyl-propanamide; or the compound

M.29.19 sarolaner, or the compound

M.29.20 lotilaner.

The commercially available compounds of the M listed above may be found in The Pesticide Manual, 16th Edition, C. MacBean, British Crop Protection Council (2013) among other publications. The online Pesticide Manual is updated regularly and is accessible through http://bcpcdata.com/pesticide-manual.html.

Another online data base for pesticides providing the ISO common names is http://www.alanwood. net/pesticides.

The M.4 neonicotinoid cycloxaprid is known from WO2010/069266 and WO2011/069456, the neonicotinoid M.4A.2, sometimes also to be named as guadipyr, is known from WO2013/003977, and the neonicotinoid M.4A.3 (approved as paichongding in China) is known from WO2007/101369. The metaflumizone analogue M.22B.1 is described in CN10171577 and the analogue M.22B.2 in CN102126994. The phthalamides M.28.1 and M.28.2 are both known from WO2007/101540. The anthranilamide M.28.3 is described in WO2005/077934. The hydrazide compound M.28.4 is described in WO2007/043677. The anthranilamides M.28.5a) to M.28.5d) and M.28.5h) are described in WO 2007/006670, WO2013/024009 and WO2013/024010, the anthranilamide M.28.5i) is described in WO2011/085575, M.28.5j) in WO2008/134969, M.28.5k) in US2011/046186 and M.28.51) in WO2012/034403. The diamide compound M.28.6 can be found in WO2012/034472. The spiroketal-substituted cyclic ketoenol derivative M.29.3 is known from WO2006/089633 and the biphenyl-substituted spirocyclic ketoenol derivative M.29.4 from WO2008/067911. The triazoylphenylsulfide M.29.5 is described in WO2006/043635, and biological control agents on the basis of Bacillus firmus are described in WO2009/124707. The compounds M.29.6a) to M.29.6i) listed under M.29.6 are described in WO2012/029672, and M.29.6j) and M.29.6k) in WO2013/129688. The nematicide M.29.8 is known from WO2013/055584. The isoxazoline M.29.9.a) is described in WO2013/050317. The isoxazoline M.29.9.b) is described in WO2014/126208. The pyridalyl-type analogue M.29.10 is known from WO2010/060379. The carboxamides broflanilide and M.29.11.b) to M.29.11.h) are described in WO2010/018714, and the carboxamides M.29.11i) to M.29.11.p) in WO2010/127926. The pyridylthiazoles M.29.12.a) to M.29.12.c) are known from WO2010/006713, M.29.12.d) and M.29.12.e) are known from WO2012/000896, and M.29.12.f) to M.29.12.m) from WO2010/129497. The compounds M.29.14a) and M.29.14b) are known from WO2007/101369. The pyrazoles M.29.16.a) to M.29.16h) are described in WO2010/034737, WO2012/084670, and WO2012/143317, respectively, and the pyrazoles M.29.16i) and M.29.16j) are described in U.S. 61/891,437. The pyridinylindazoles M.29.17a) to M.29.17.j) are described in WO2015/038503. The pyridylpyrazoles M.29.18a) to M.29.18d) are described in US2014/0213448. The isoxazoline M.29.19 is described in WO2014/036056. The isoxazoline M.29.20 is known from WO2014/090918.

The following list of fungicides, in conjunction with which the compounds of the present invention can be used, is intended to illustrate the possible combinations but does not limit them:

A) Respiration Inhibitors
Inhibitors of complex III at Qₒ site (e. g. strobilurins): azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxy.strobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21), methyl-N-[2-[(1,4-dimethyl-5-phenyl-pyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyltetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methyl pyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), (Z,2E)5-[1-(4-chloro-2-fluoro-phenyl)pyrazol-3-yl]oxy-2-methoxyimino-N,3-dimethyl-pent-3-enamide (A.1.36),
inhibitors of complex III at Qᵢ site: cyazofamid (A.2.1), amisulbrom (A.2.2), [(3S,6S,7R,8R)8-benzyl-3-[(3-acetoxy-4-methoxy-pyridine-2-carbonyl)amino]-6-methyl-4,9-dioxo-1,5-dioxonan-7-yl] 2-methylpropanoate (A.2.3), [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl] 2-methylpropanoate (A.2.4), [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl] 2-methylpropanoate (A.2.5), [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl] 2-methylpropanoate (A.2.6); (3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate (A.2.7), (3S,6S,7R,8R)-8-benzyl-3-[3-[(isobutyryloxy)methoxy]-4-methoxypicolinamido]-6-methyl-4,9-dioxo-1,5-dioxonan-7-yl isobutyrate (A.2.8);
inhibitors of complex II (e. g. carboxamides): benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.14), penthiopyrad (A.3.15), sedaxane (A.3.16), tecloftalam (A.3.17), thifluzamide (A.3.18), N-(4′-trifluoromethylthiobiphenyl-2-yl)-3-difluoromethyl-1-methyl-1i H-pyrazole-4-carboxamide (A.3.19), N-(2-(1,3,3-trimethyl-butyl)-phenyl)-1,3-dimethyl-5-fluoro-1H-pyrazole-4-carboxamide (A.3.20), 3-(difluoromethyl)-1-methyl-N-(1,1,3-trimethylindan-4-yl)pyrazole-4-carboxamide (A.3.21), 3-(trifluoromethyl)-1-methyl-N-(1,1,3-trimethylindan-4-yl)pyrazole-4-carboxamide (A.3.22), 1,3-dimethyl-N-(1,1,3-trimethylindan-4-yl)pyrazole-4-carboxamide (A.3.23), 3-(trifluoromethyl)-1,5-dimethyl-N-(1,1,3-trimethylindan-4-yl)pyrazole-4-carboxamide (A.3.24), 1,3,5-trimethyl-N-(1,1,3-trimethylindan-4-yl)pyrazole-4-carboxamide (A.3.25), N-(7-fluoro-1,1,3-trimethyl-indan-4-yl)-1,3-dimethyl-pyrazole-4-carboxamide (A.3.26), N-[2-(2,4-dichlorophenyl)-2-methoxy-1-methyl-ethyl]-3-(difluoromethyl)-1-methyl-pyrazole-4-carboxamide (A.3.27);
other respiration inhibitors (e. g. complex I, uncouplers): diflumetorim (A.4.1), (5,8-difluoroquinazolin-4-yl)-{2-[2-fluoro-4-(4-trifluoromethylpyridin-2-yloxy)-phenyl]-ethyl}-amine (A.4.2); nitrophenyl derivates: binapacryl (A.4.3), dinobuton (A.4.4), dinocap (A.4.5), fluazinam (A.4.6); ferimzone (A.4.7); organometal compounds: fentin salts, such as fentin-acetate (A.4.8), fentin chloride (A.4.9) or fentin hydroxide (A.4.10); ametoctradin (A.4.11); and silthiofam (A.4.12);

B) Sterol Biosynthesis Inhibitors (SBI Fungicides)
C14 demethylase inhibitors (DMI fungicides): triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel(2S;3R)-3-(2-chlorophenyl)-2-(2,4-difluorophenyl)-oxiranylmethyl]-5-thiocyanato-1H-[1,2,4]triazolo (B.1.31), 2-[rel-(2S,3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), 2-[4-(4-chlorophenoxy)-2-(trifluoromethyl)phenyl]-3-methyl-1-(1,2,4-triazol-1-yl)butan-2-ol (B.1.37), 2-[4-(4-chlorophenoxy)-2-(trifluoromethyl)phenyl]-1-(1,2,4-triazol-1-yl)propan-2-ol (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.51); imidazoles: imazalil (B.1.42), pefurazoate (B.1.43), prochloraz (B.1.44), triflumizol (B.1.45); pyrimidines, pyridines and piperazines: fenarimol (B.1.46), nuarimol (B.1.47), pyrifenox (B.1.48), triforine (B.1.49), [3-(4-chloro-2-fluoro-phenyl)-5-(2,4-difluorophenyl)isoxazol-4-yl]-(3-pyridyl)methanol (B.1.50);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);

C) Nucleic Acid Synthesis Inhibitors
phenylamides or acyl amino acid fungicides: benalaxyl (C.1.1), benalaxyl-M (C.1.2), kiralaxyl (C.1.3), metalaxyl (C.1.4), metalaxyl-M (mefenoxam, C.1.5), ofurace (C.1.6), oxadixyl (C.1.7);
others: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7);

D) Inhibitors of Cell Division and Cytoskeleton
tubulin inhibitors, such as benzimidazoles, thiophanates: benomyl (D1.1), carbendazim (D1.2), fuberidazole (D1.3), thiabendazole (D1.4), thiophanate-methyl (D1.5); triazolopyrimidines: 5-chloro-7-(4-methylpiperidin-1-yl)-6-(2,4,6-trifluorophenyl)-[1,2,4]triazolo[1,5-a]pyrimidine (D1.6);
other cell division inhibitors: diethofencarb (D2.1), ethaboxam (D2.2), pencycuron (D2.3), fluopicolide (D2.4), zoxamide (D2.5), metrafenone (D2.6), pyriofenone (D2.7);

E) Inhibitors of Amino Acid and Protein Synthesis
methionine synthesis inhibitors (anilino-pyrimidines): cyprodinil (E.1.1), mepanipyrim (E.1.2), pyrimethanil (E.1.3);
protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6), polyoxine (E.2.7), validamycin A (E.2.8);

F) Signal Transduction Inhibitors
MAP/histidine kinase inhibitors: fluoroimid (F.1.1), iprodione (F.1.2), procymidone (F.1.3), vinclozolin (F.1.4), fenpiclonil (F.1.5), fludioxonil (F.1.6);
G protein inhibitors: quinoxyfen (F.2.1);

G) Lipid and Membrane Synthesis Inhibitors
Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7) and N-(1-(1-(4-cyano-phenyl)ethanesulfonyl)-but-2-yl) carbamic acid-(4-fluorophenyl) ester (G.3.8);
compounds affecting cell membrane permeability and fatty acides: propamocarb (G.4.1);
fatty acid amide hydrolase inhibitors: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3);

H) Inhibitors with Multi Site Action
inorganic active substances: Bordeaux mixture (H.1.1), copper acetate (H.1.2), copper hydroxide (H.1.3), copper oxychloride (H.1.4), basic copper sulfate (H.1.5), sulfur (H.1.6);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds (e. g. phthalimides, sulfamides, chloronitriles): anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11), N-(4-chloro-2-nitro-phenyl)-N-ethyl-4-methyl-benzenesulfonamide (H.3.12);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

I) Cell Wall Synthesis Inhibitors
inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

J) Plant Defence Inducers
acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9);

K) Unknown Mode of Action
bronopol (K.1.1), chinomethionat (K.1.2), cyflufenamid (K.1.3), cymoxanil (K.1.4), dazomet (K.1.5), debacarb (K.1.6), diclomezine (K.1.7), difenzoquat (K.1.8), difenzoquat-methylsulfate (K.1.9), diphenylamin (K.1.10), fenpyrazamine (K.1.11), flumetover (K.1.12), flusulfamide (K.1.13), flutianil (K.1.14), methasulfocarb (K.1.15), nitrapyrin (K.1.16), nitrothal-isopropyl (K.1.18), oxathiapiprolin (K.1.19), tolprocarb (K.1.20), oxin-copper (K.1.21), proquinazid (K.1.22), tebufloquin (K.1.23), tecloftalam (K.1.24), triazoxide (K.1.25), 2-butoxy-6-iodo-3-propylchromen-4-one (K.1.26), 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone (K.1.27), 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone (K.1.28), 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone (K.1.29), N-(cyclopropylmethoxyimino-(6-difluoro-methoxy-2,3-difluoro-phenyl)-methyl)-2-phenyl acetamide (K.1.30), N′-(4-(4-chloro-3-trifluoromethyl-phenoxy)-2,5-dimethyl-phenyl)-N-ethyl-N-methyl formamidine (K.1.31), N′-(4-(4-fluoro-3-trifluoromethyl-phenoxy)-2,5-dimethyl-phenyl)-N-ethyl-N-methyl formamidine (K.1.32), N′-(2-methyl-5-trifluoromethyl-4-(3-trimethylsilanyl-propoxy)-phenyl)-N-ethyl-N-methyl formamidine (K.1.33), N′-(5-difluoromethyl-2-methyl-4-(3-trimethylsilanylpropoxy)-phenyl)-N-ethyl-N-methyl formamidine (K.1.34), methoxy-acetic acid 6-tert-butyl-8-fluoro-2,3-dimethyl-quinolin-4-yl ester (K.1.35), 3-[5-(4-methylphenyl)-2,3-dimethyl-isoxazolidin-3-yl]-pyridine (K.1.36), 3-[5-(4-chloro-phenyl)-2,3-dimethyl-isoxazolidin-3-yl]-pyridine (pyrisoxazole) (K.1.37), N-(6-methoxy-pyridin-3-yl) cyclopropanecarboxylic acid amide (K.1.38), 5-chloro-1-(4,6-dimethoxypyrimidin-2-yl)-2-methyl-1H-benzoimidazole (K.1.39), 2-(4-chloro-phenyl)-N-[4-(3,4-dimethoxy-phenyl)-isoxazol-5-yl]-2-prop-2-ynyloxy-acetamide, ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), 2-[2-[(7,8-difluoro-2-methyl-3-quinolyl)oxy]-6-fluorophenyl]propan-2-ol (K.1.43), 2-[2-fluoro-6-[(8-fluoro-2-methyl-3-quinolyl)oxy]phen-yl]propan-2-ol (K.1.44), 3-(5-fluoro-3,3,4,4-tetramethyl-3,4-dihydroisoquinolin-1-yl)quinoline (K.1.45), 3-(4,4-difluoro-3,3-dimethyl-3,4-dihydroisoquinolin-1-yl)quinoline (K.1.46), 3-(4,4,5-trifluoro-3,3-dimethyl-3,4-dihydroisoquinolin-1-yl)quinoline (K.1.47), 9-fluoro-2,2-dimethyl-5-(3-quinolyl)-3H-1,4-benzoxazepine (K.1.48).

The fungicides described by common names, their preparation and their activity e.g. against harmful fungi is known (cf.: http://www.alanwood.net/pesticides/); these substances are commercially available.

The fungicides described by IUPAC nomenclature, their preparation and their pesticidal activity is also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657, WO2012/168188, WO 2007/006670, WO 2011/77514; WO13/047749, WO 10/069882, WO 13/047441, WO 03/16303, WO 09/90181, WO 13/007767, WO 13/010862, WO 13/127704, WO 13/024009, WO 13/024010 and WO 13/047441, WO 13/162072, WO 13/092224, WO 11/135833).

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Mono-graph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfac-tants, dispersants, emulsifiers, wetters, adjuvants, solubilizers, penetration enhancers, protec-tive colloids, adhesion agents, thickeners, humectants, repellents, attractants, feeding stimu-lants, compatibilizers, bactericides, anti-freezing agents, anti-foaming agents, colorants, tackifi-ers and binders.

Suitable solvents and liquid carriers are water and organic solvents, such as mineral oil frac-tions of medium to high boiling point, e.g. kerosene, diesel oil; oils of vegetable or animal origin; aliphatic, cyclic and aromatic hydrocarbons, e. g. toluene, paraffin, tetrahydronaphthalene, al-kylated naphthalenes; alcohols, e.g. ethanol, propanol, butanol, benzylalcohol, cyclo-hexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sul-fates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl-naphthalenes, sulfosuccinates or sulfosuccinamates. Examples of sulfates are sulfates of fatty acids and oils, of ethoxylated alkylphenols, of alcohols, of ethox-ylated alcohols, or of fatty acid esters. Examples of phosphates are phosphate esters. Exam-ples of carboxylates are alkyl carboxylates, and carboxylated alcohol or alkylphenol eth-oxylates.

Suitable nonionic surfactants are alkoxylates, N-subsituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Exam-ples of N-subsititued fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxilaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazoli-nones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanofer-rate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

ii) Dispersible Concentrates (DC)

5-25 wt % of a compound I according to the invention and 1-10 wt % dispersant (e. g. polyvi-nylpyrrolidone) are dissolved in up to 100 wt % organic solvent (e.g. cyclohexanone). Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active sub-stance. For FS type composition up to 40 wt % binder (e.g. polyvinylalcohol) is added.

vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with ad-dition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dis-persion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active sub-stance. Dilution with water gives a stable suspension of the active substance.

ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water up to 100%. This mixture is stirred for 1 h to produce spontaneously a thermodynamically stable microemulsion.
x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radi-cal initiator results in the formation of poly(meth)acrylate microcapsules. Alternatively, an oil phase comprising 5-50 wt % of a compound I according to the invention, 0-40 wt % water insolu-ble organic solvent (e.g. aromatic hydrocarbon), and an isocyanate monomer (e.g. diphenylme-thene-4,4′-diisocyanatae) are dispersed into an aqueous solution of a protective colloid (e.g. polyvinyl alcohol). The addition of a polyamine (e.g. hexamethylenediamine) results in the for-mation of a polyurea microcapsule. The monomers amount to 1-10 wt %. The wt % relate to the total CS composition.

xi) Dustable Powders (DP, DS)

1-10 wt % of a compound I according to the invention are ground finely and mixed intimately with up to 100 wt % solid carrier, e.g. finely divided kaolin.

xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably be-tween 0.1 and 90%, and most preferably between 0.5 and 75%, by weight of active sub-stance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions com-prising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the in-vention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the in-vention or partially premixed components, e. g. components comprising compounds of the present invention and/or mixing partners as defined above, can be applied jointly (e.g. after tank mix) or consecutively.

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds/compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including wild type plants and plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, cry1Ab, bar, cry1Fa2, cry1Ac, cry34Ab1, cry35AB1, cry3A, cryF, cry1F, mcry3a, cry2Ab2, cry3Bbl, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

It has surprisingly been found that the pesticidal activity of the compounds of the present invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the present invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/I Surfactant, 0 to 200 g/I antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/I) of the active ingredient, from 0.1 to 20% by weight (1 to 200 g/I) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

The compounds of the present invention may also be used for improving the health of a plant. Therefore, the present invention also relates to a method for improving plant health by treating a plant, plant propagation material and/or the locus where the plant is growing or is to grow with an effective and non-phytotoxic amount of a compound of the present invention.

As used herein “an effective and non-phytotoxic amount” means that the compound is used in a quantity which allows to obtain the desired effect but which does not give rise to any phytotoxic symptom on the treated plant or on the plant grown from the treated propagule or treated soil.

The terms “plant” and “plant propagation material” are defined above.

“Plant health” is defined as a condition of the plant and/or its products which is determined by several aspects alone or in combination with each other such as yield (for example increased biomass and/or increased content of valuable ingredients), quality (for example improved content or composition of certain ingredients or shelf life), plant vigour (for example improved plant growth and/or greener leaves (“greening effect”), tolerance to abiotic (for example drought) and/or biotic stress (for example disease) and production efficiency (for example, harvesting efficiency, processability).

The above identified indicators for the health condition of a plant may be interdependent and may result from each other. Each indicator is defined in the art and can be determined by methods known to a skilled person.

The compounds of the invention are also suitable for use against non-crop insect pests. For use against said non-crop pests, compounds of the present invention can be used as bait composition, gel, general insect spray, aerosol, as ultra-low volume application and bed net (impregnated or surface applied). Furthermore, drenching and rodding methods can be used.

As used herein, the term “non-crop insect pest” refers to pests, which are particularly relevant for non-crop targets, such as ants, termites, wasps, flies, ticks, mosquitos, crickets, or cockroaches.

The bait can be a liquid, a solid or a semisolid preparation (e.g. a gel). The bait employed in the composition is a product, which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature (e.g. http://www.pherobase.com), and are known to those skilled in the art.

For use in bait compositions, the typical content of active ingredient is from 0.001 weight % to 15 weight %, desirably from 0.001 weight % to 5% weight % of active compound.

Formulations of the compounds of the present invention as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents, furthermore auxiliaries such as emulsifiers, perfume oils, if appropriate stabilizers, and, if required, propellants.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder.

The compounds of the present invention and its compositions can be used for protecting wooden materials such as trees, board fences, sleepers, frames, artistic artifacts, etc. and buildings, but also construction materials, furniture, leathers, fibers, vinyl articles, electric wires and cables etc. from ants and/or termites, and for controlling ants and termites from doing harm to crops or human being (e.g. when the pests invade into houses and public facilities).

Customary application rates in the protection of materials are, for example, from 0.001 g to 2000 g or from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

The compounds of the the present invention are especially suitable for efficiently combating animal pests such as arthropods, gastropods and nematodes including but not limited to:

insects from the order of Lepidoptera, for example Achroia grisella, Acleris spp. such as A. fimbriana, A. gloverana, A. variiana; Acrolepiopsis assectella, Acronicta major, Adoxophyes spp. such as A. cyrtosema, A. orana; Aedia leucomelas, Agrotis spp. such as A. exclamationis, A. fucosa, A. ipsion, A. orthogoma, A. segetum, A. subterranea; Alabama argillacea, Aleurodicus dispersus, Alsophilla pometaria, Ampelophaga rubiginosa, Amyelois transitella, Anacampsis sarcitella, Anagasta kuehniella, Anarsia lineatella, Anisota senatorla, Antheraea pernyi, Anticarsia (=Thermesia) spp. such as A. gemmatalls; Apamea spp., Aproaerema modicella, Archips spp. such as A. argyrosplla, A. fuscocupreanus, A. rosana, A. xyloseanus; Argyresthia conjugella, Argyroploce spp., Argyrotaenia spp. such as A. velutinana; Athetis mindara, Austroasca viridigrisea, Autographa gamma, Autographa nigrisigna, Barathra brassicae, Bedellia spp., Bonagota salubricola, Borbo cinnara, Bucculatrix thurberlella, Bupalus pinlarlus, Busseola spp., Cacoecia spp. such as C. murinana, C. podana; Cactoblastis cactorum, Cadra cautella, Calingo brazilliensis, Caloptllis theivora, Capua reticulana, Carposina spp. such as C. niponensis, C. sasakii, Cephus spp., Chaetocnema aridula, Cheimatobia brumata, Chilo spp. such as C. Indicus, C. suppressalis, C. partellus; Choreutis parlana, Choristoneura spp. such as C. conflctana, C. fumiferana, C. longicellana, C. murinana, C. occidentals, C. rosaceana; Chrysodeixis (=Pseudoplusia) spp. such as C. eriosoma, C. includens; Cilrphis unipuncta, Clysia ambiguella, Cnaphalocerus spp., Cnaphalocrocis medinalis, Cnephasia spp., Cochylis hospes, Coleophora spp., Collas eurytheme, Conopomorpha spp., Conotrachelus spp., Copitarsia spp., Corcyra cephalonica, Crambus caliginosellus, Crambus teterrellus, Crocidosema (=Epinotia) aporema, Cydalima (=Diaphania) perspectalis, Cydia (=Carpocapsa) spp. such as C. pomonella, C. latiferreana; Dalaca noctuides, Datana integerrima, Dasychira pinlcola, Dendrolimus spp. such as D. pini, D. spectabilis, D. sibiricus; Desmia funeralis, Diaphania spp. such as D. nitidalis, D. hyalinata; Diatraea grandiosella, Diatraea saccharalis, Diphthera festiva, Earias spp. such as E. insulana, E. vittella, Ecdytolopha aurantianu, Egira (=Xylomyges) curials, Elasmopalpus lignosellus, Eldana saccharina, Endopiza viteana, Ennomos subsignaria, Eoreuma loftini, Ephestia spp. such as E. cautella, E. elutella, E. kuehniella, Epinotia aporema, Epiphyas postvittana, Erannis tillaria, Erionota thrax, Etiella spp., Eulia spp., Eupoecilia ambiguella, Euproctis chrysorrhoea, Euxoa spp., Evetria bouliana, Faronta albilinea, Feltia spp. such as F. subterranean; Galleria mellonella, Gracillaria spp., Graphollta spp. such as G. funebrana, G. molesta, G. inopinata; Halysidota spp., Harrisina americana, Hedylepta spp., Hellcoverpa spp. such as H. armigera (=Hellothis armigera), H. zea (=Hellothis zea); Hellothis spp. such as H. assulta, H. subflexa, H. virescens; Hellula spp. such as H. undalis, H. rogatalis; Helocoverpa gelotopoeon, Hemlleuca ollviae, Herpetogramma licarsisalis, Hibernia defollaria, Hofmannophilla pseudospretella, Homoeosoma electellum, Homona magnanima, Hypena scabra, Hyphantria cunea, Hyponomeuta padella, Hyponomeuta malinellus, Kakivoria flavofasciata, Keiferia lycopersicella, Lambdina fiscellaria fiscellaria, Lambdina fiscellaria lugubrosa, Lamprosema indicata, Laspeyresia molesta, Leguminivora glycinivorella, Lerodea eufala, Leucinodes orbonalis, Leucoma sallcis, Leucoptera spp. such as L. coffeella, L. scitella; Leuminivora lycinivorella, Lithocolletis blancardella, Lithophane antennata, Llattlia octo (=Amyna axis), Lobesia botrana, Lophocampa spp., Loxagrotis alblcosta, Loxostege spp. such as L. sticticals, L. cereralis Lymantria spp. such as L. dispar, L. monacha; Lyonetia clerkella, Lyonetia prunifoliella, Malacosoma spp. such as M. americanum, M. californicum, M. constrictum, M. neustria; Mamestra spp. such as M. brassicae, M. configurata; Mamstra brassicae, Manduca spp. such as M. quinquemaculata, M. sexta; Marasmia spp, Marmara spp., Maruca testulalis, Megalopyge lanata, Melanchra picta, Melanitis leda, Mocis spp. such as M. lapites, M. repanda; Mocis latipes, Monochroa fragariae, Mythimna separata, Nemapogon cloacella, Neoleucinodes elegantalis, Nepytia spp., Nymphula spp., Oiketicus spp., Omiodes indicata, Omphisa anastomosals, Operophtera brumata, Orgyia pseudotsugata, Oria spp., Orthaga thyrisalis, Ostrinia spp. such as O. nubilalis; Oulema oryzae, Paleacrita vernata, Panolis flammea, Parnara spp., Papaipema nebris, Papilio cresphontes, Paramyelois transitella, Paranthrene regalls, Paysandisia archon, Pectinophora spp. such as P. gossyplella; Peridroma saucia, Perlileucoptera spp., such as P. coffeella; Phalera bucephala, Phryganidia calfornica, Phthorimaea spp. such as P. operculella; Phyllocnistis citrella, Phyllonorycterspp. such as P. blancardella, P. crataegella, P. issiki, P. ringoniella; Pieris spp. such as P. brassicae, P. rapae, P. napi; Plilocrocis tripunctata, Plathypena scabra, Platynota spp. such as P. flavedana, P. idaeusalis, P. stultana; Platyptilila carduidactyla, Plebejus argus, Plodia interpunctella, Plusia spp, Plutella maculipennis, Plutella xylostella, Pontia protodica, Prays spp., Prodenia spp., Proxenusiepigone, Pseudaletia spp. such as P. sequax, P. unipuncta; Pyrausta nubilalis, Rachiplusia nu, Richia albicosta, Rhizobius ventralis, Rhyacionia frustrana, Sabulodes aegrotata, Schizura concinna, Schoenobius spp., Schreckensteinia festalella, Scirpophaga spp. such as S. incertulas, S. innotata; Scotia segetum, Sesamia spp. such as S. inferens, Seudyra subflava, Sitotroga cerealella, Sparganothis pleriana, Spilonota lechriaspis, S. ocellana, Spodoptera (=Lamphygma) spp. such as S. cosmoides, S. eridania, S. exigua, S. frugiperda, S. latisfascia, S. llttoralis, S. lltura, S. omithogali, Stigmella spp., Stomopteryx subsecivella, Strymon bazochilsylepta derogata, Synanthedon spp. such as S. exitiosa, Tecia sollanivora, Telehin llcus, Thaumatopoea pityocampa, Thaumatotibia (=Cryptophlebia) leucotreta, Thaumetopoea pityocampa, Thecla spp., Theresimima ampellophaga, Thyrintelna spp, Tildenia inconspicuella, Tinea spp. such as T. cloacella, T. pellillonella; Tineola bissellella, Tortrix spp. such as T. viridana; Trichophaga tapetzella, Trichoplusia spp. such as T. ni; Tuta (=Scrobipalpula) absoluta, Udea spp. such as U. rubigalis, U. rubigalis; Virachola spp., Yponomeuta padella, and Zeiraphera canadensis;

insects from the order of Coleoptera, for example Acalymma vittatum, Acanthoscehdes obtectus, Adoretus spp., Agelastica alni, Agrilus spp. such as A. anxlus, A. planipennis, A. sinuatus; Agriotes spp. such as A. fuscicollis, A. lineatus, A. obscurus; Alphitobius diaperinus, Amphimallus sollstitialis, Anisandrus dispar, Anisoplila austriaca, Anobium punctatum, Anomala corpulenta, Anomala rufocuprea, Anopllophora spp. such as A. glabripennis; Anthonomus spp. such as A. eugenil, A. grandis, A. pomorum; Anthrenus spp., Aphthona euphoridae, Apion spp., Apogonia spp., Athous haemorrhoidalis, Atomaria spp. such as A. linearis; Attagenus spp., Aulacophora femoralis, Blastophagus piniperda, Bliltophaga undata, Bruchidius obtectus, Bruchus spp. such as B. lentis, B. pisorum, B. rufimanus, Byctiscus betulae, Callidiellum rufipenne, Callopistria floridensis, Callosobruchus chinensis, Cameraria ohridella, Cassida nebulosa, Cerotoma trifurcata, Cetonia aurata, Ceuthorhynchus spp. such as C. assimilis, C. napi; Chaetocnema tibialis, Cleonus mendicus, Conoderus spp. such as C. vespertinus; Conotrachelus nenuphar, Cosmopolites spp., Costelytra zealandica, Crioceris asparagi, Cryptolestes ferrugineus, Cryptorhynchus lapathi, Ctenicera spp. such as C. destructor; Curculio spp., Cylindrocopturus spp., Cyclocephala spp., Dactylispa balyi, Dectes texanus, Dermestes spp., Diabrotica spp. such as D. undecimpunctata, D. speciosa, D. longicornis, D. semi punctata, D. virgifera; Diaprepes abbreviates, Dichocrocis spp., Dicladispa armigera, Diloboderus abderus, Diocalandra frumenti (Diocalandra stigmaticollis), Enaphalodes rufulus, Epilachna spp. such as E. varivestis, E. vigintioctomaculata; Epitrix spp. such as E. hirtipennis, E. similaris; Eutheola humiils, Eutinobothrus brasiliensis, Faustinus cubae, Gibbium psylloides, Gnathocerus cornutus, Hellula undalls, Heteronychus arator, Hylamorpha elegans, Hylobius abietis, Hylotrupes bajulus, Hypera spp. such as H. brunneipennis, H. postica; Hypomeces squamosus, Hypothenemus spp., lps typographus, Lachnosterna consanguinea, Lasioderma serricorne, Latheticus oryzae, Lathridius spp., Lema spp. such as L. billineata, L. melanopus; Leptinotarsa spp. such as L. decemlineata; Leptispa pygmaea, Limonius callifornicus, Lissorhoptrus oryzophilus, Lixus spp., Luperodes spp., Lyctus spp. such as L. bruneus; Liogenys fuscus, Macrodactylus spp. such as M. subspinosus; Maladera matrida, Megaplatypus mutates, Megascelis spp., Melanotus communis, Meligethes spp. such as M. aeneus; Melolontha spp. such as M. hippocastani, M. melolontha; Metamasius hemipterus, Microtheca spp., Migdolus spp. such as M. fryanus, Monochamus spp. such as M. alternatus; Naupactus xanthographus, Niptus hololeucus, Oberia brevis, Oemona hirta, Oryctes rhinoceros, Oryzaephilus surinamensis, Oryzaphagus oryzae, Otiorrhynchus sulcatus, Otiorrhynchus ovatus, Otiorrhynchus sulcatus, Oulema melanopus, Oulema oryzae, Oxycetonia jucunda, Phaedon spp. such as P. brassicae, P. cochleariae; Phoracantha recurva, Phyllobius pyri, Phyllopertha horticola, Phyllophaga spp. such as P. helleri; Phyllotreta spp. such as P. chrysocephala, P. nemorum, P. striolata, P. vittula; Phyllopertha horticola, Poplilla japonica, Premnotrypes spp., Psacothea hilaris, Psylliodes chrysocephala, Prostephanus truncates, Psylliodes spp., Ptinus spp., Pulga saltona, Rhizopertha dominica, Rhynchophorus spp. such as R. biillineatus, R. ferrugineus, R. palmarum, R. phoenicis, R. vulneratus; Saperda candida, Scolytus schevyrewi, Scyphophorus acupunctatus, Sitona lineatus, Sitophilus spp. such as S. granaria, S. oryzae, S. zeamals; Sphenophorus spp. such as S. levis; Stegobium paniceum, Sternechus spp. such as S. subsignatus; Strophomorphus ctenotus, Symphyletes spp., Tanymecus spp., Tenebrio moliltor, Tenebrioides mauretanicus, Tribollum spp. such as T. castaneum; Trogoderma spp., Tychius spp., Xylotrechus spp. such as X. pyrrhoderus; and, Zabrus spp. such as Z. tenebrioides;

insects from the order of Diptera for example Aedes spp. such as A. aegypti, A. albopictus, A. vexans; Anastrepha ludens, Anopheles spp. such as A. albimanus, A. crucians, A. freeborni, A. gambiae, A. leucosphyrus, A. maculipennis, A. minimus, A. quadrimaculatus, A. sinensis; Bactrocera invadens, Bibio hortulanus, Calliphora erythrocephala, Calliphora vicina, Ceratitis capitata, Chrysomyia spp. such as C. bezziana, C. hominivorax, C. macellaria, Chrysops atlanticus, Chrysops discalis, Chrysops silacea, Cochllomya spp. such as C. hominivorax; Contarinia spp. such as C. sorghicola; Cordylobia anthropophaga, Culex spp. such as C. nigripalpus, C. pipiens, C. quinquefasciatus, C. tarsalis, C. tritaeniorhynchus, Cullcoides furens, Culiseta inornata, Cullseta melanura, Cuterebra spp., Dacus cucurbitae, Dacus oleae, Dasineura brassicae, Dasineura oxycoccana, Delia spp. such as D. antique, D. coarctata, D. platura, D. radicum; Dermatobia hominis, Drosophila spp. such as D. suzuki, Fannia spp. such as F. canicularis; Gastraphilus spp. such as G. intestinalis; Geomyza tipunctata, Glossina spp. such as G. fuscipes, G. morsitans, G. palpalis, G. tachinoides; Haematobia irritans, Haplodiplosis equestris, Hippelates spp., Hylemyia spp. such as H. platura; Hypoderma spp. such as H. lineata; Hyppobosca spp., Hydrellia philippina, Leptoconops torrens, Liriomyza spp. such as L. sativae, L. trifolii; Lucilia spp. such as L. caprina, L. cuprina, L. sericata; Lycoria pectorallis, Mansonia titllanus, Mayetiola spp. such as M. destructor; Musca spp. such as M. autumnalis, M. domestica; Muscina stabulans, Oestrus spp. such as O. ovis; Opomyza florum, Oscinella spp. such as O. frit; Orseolia oryzae, Pegomya hysocyam, Phlebotomus argentipes, Phorbia spp. such as P. antiqua, P. brassicae, P. coarctata; Phytomyza gymnostoma, Prosimulum mixtum, Psila rosae, Psorophora columbiae, Psorophora discolor, Rhagoletis spp. such as R. cerasi, R. cingulate, R. indifferens, R. mendax, R. pomonella; Rivellia quadrifasciata, Sarcophaga spp. such as S. haemorrhoidalls; Simullum vittatum, Sitodiplosis mosellana, Stomoxys spp. such as S. calcitrans; Tabanus spp. such as T. atratus, T. bovinus, T. lineola, T. similis; Tannia spp., Thecodiplosis japonensis, Tipula oleracea, Tipula paludosa, and Wohlfahrtia spp;

insects from the order of Thysanoptera for example, Ballothrips biformis, Dichromothrips corbetti, Dichromothrips ssp., Echinothrips americanus, Enneothrips flavens, Franklniella spp. such as F. fusca, F. occidentallis, F. tritici; Hellothrips spp., Hercinothrips femoralis, Kakothrips spp., Microcephalothrips abdominalis, Neohydatothrips samayunkur, Pezothrips kellyanus, Rhipiphorothrips cruentatus, Scirtothrips spp. such as S. citr S. dorsalis, S. perseae; Stenchaetothrips spp, Taeniothrips cardamoni, Taeniothrips inconsequens, Thrips spp. such as T. imagines, T. hawaiiensis, T. oryzae, T. palmi, T. parvispinus, T. tabaci;

insects from the order of Hemiptera for example, Acizzia jamatonica, Acrosternum spp. such as A. hilare; Acyrthosipon spp. such as A. onobrychis, A. pisum; Adelges laricis, Adelges tsugae, Adelphocoris spp., such as A. rapidus, A. superbus; Aeneolamia spp., Agonoscena spp., Aulacorthum solani. Aleurocanthus woglumli, Aleurodes spp., Aleurodicus disperses, Aleurolobus barodensis, Aleurothrixus spp., Amrasca spp., Anasa tristis, Antestiopsis spp., Anuraphis cardui, Aonidiella spp., Aphanostigma piri, Aphidula nasturti Aphis spp. such as A. craccivora, A. fabae, A. forbesi, A. gossypil, A. grossulariae, A. maidlradicis, A. pomi, A. sambuci, A. schneider A. spiraecola; Arboridia apicalis, Arlilus critatus, Aspidlella spp., Aspidiotus spp., Atanus spp., Aulacaspis yasumatsui, Aulacorthum solani, Bactericera cockerelli, (Paratrioza cockerelli), Bemisia spp. such as B. argentifolil, B. tabaci (Aleurodes tabaci); Blissus spp. such as B. leucopterus; Brachycaudus spp. such as B. cardui, B. helilchrysi, B. persicae, B. prunicola; Brachycolus spp., Brachycorynella asparagi, Brevicoryne brassicae, Cacopsylla spp. such as C. fulguralis, C. pyricola (Psylla piri); Calligypona marginata, Calocoris spp., Campylomma lilvida, Capitophorus horni, Carneocephala fulgida, Cavelerius spp., Ceraplastes spp., Ceratovacuna lanigera, Ceroplastes ceriferus, Cerosipha gossypii, Chaetosiphon fragaefolili, Chionaspis tegalensis, Chlorita onukii, Chromaphis juglandicola, Chrysomphalus ficus, Cicadulina mbila, Cimex spp. such as C. hemipterus, C. lectularius; Coccomytilus halli, Coccus spp. such as C. hesperidum, C. pseudomagnolarum; Corythucha arcuata, Creontiades diilutus, Cryptomnyzus ribis, Chrysomphalus aonidum, Cryptomnyzus ribis, Ctenarytaina spatulata, Cyrtopeltis notatus, Dalbulus spp., Dasynus piperis, Dialeurodes spp. such as D. citrifolii; Dalbulus maidis, Diaphorina spp. such as D. citi; Diaspis spp. such as D. bromeliae; Dichelops fur catus, Diconocoris hewetti, Doralis spp., Dreyfusia nordmannianae, Dreyfusia piceae, Drosicha spp., Dysaphis spp. such as D. plantaginea, D. pyri, D. radicola; Dysaulacorthum pseudosolani, Dysdercus spp. such as D. cingulatus, D. intermedius; Dysmicoccus spp., Edessa spp., Geocoris spp., Empoasca spp. such as E. fabae, E. solana; Epidiaspis leperii Eriosoma spp. such as E. lanigerum, E. pyricola; Erythroneura spp., Eurygaster spp. such as E. integriceps; Euscelis bilobatus, Euschistus spp. such as E. heros, E. impictiventris, E. servus; Fiorinia theae, Geococcus coffeae, Glycaspis brimblecombei, Halyomorpha spp. such as H. halys; Helliopeltis spp., Homalodisca vitripennis (=H. coagulata), Horcias nobilellus, Hyalopterus pruni, Hyperomyzus lactucae, lcerya spp. such as I. purchase; Idiocerus spp., Idioscopus spp., Laodelphax striatellus, Lecanium spp., Lecanoideus floccissimus, Lepidosaphes spp. such as L. ulmi; Leptocorisa spp., Leptoglossus phyllopus, Lipaphis erysimi Lygus spp. such as L. hesperus, L. lineolaris, L. pratensis; Maconellicoccus hirsutus, Marchalina hellenica, Macropes excavatus, Macrosiphum spp. such as M. rosae, M. avenae, M. euphorbiae; Macrosteles quadrillneatus, Mahanarva fimbriolata, Megacopta cribraria, Megoura viciae, Melanaphis pyrarius, Melanaphis sacchar Melanocallis (=Tinocallis) caryaefoliae, Metcafiella spp., Metopolophium dirhodum, Monellia costalis, Monelliopsis pecanis, Myzocallis coryi, Murgantia spp., Myzus spp. such as M. ascalonicus, M. cerasi, M. nicotianae, M. persicae, M. varians; Nasonovia ribis-nigr Neotoxoptera formosana, Neomegalotomus spp, Nephotettix spp. such as N. malayanus, N. nigropictus, N. parvus, N. virescens; Nezara spp. such as N. viridula; Nilaparvata lugens, Nysius huttoni, Oebalus spp. such as O. pugnax; Oncometopia spp., Orthezia praeonga, Oxycaraenus hyalinipennis, Parabemisia myricae, Parlatoria spp., Parthenolecanium spp. such as P. corn, P. persicae; Pemphigus spp. such as P. bursarius, P. populivenae; Peregrinus maidis, Perkinsiella saccharicida, Phenacoccus spp. such as P. aceris, P. gossypii; Phloeomyzus passerinil Phorodon humuli, Phylloxera spp. such as P. devastatrix, Piesma quadrata, Piezodorus spp. such as P. guildinii; Pinnaspis aspidistrae, Planococcus spp. such as P. citri, P. ficus; Prosapia bicincta, Protopulvinaria pyriformis, Psallus seriatus, Pseudacysta persea, Pseudaulacaspis pentagona, Pseudococcus spp. such as P. comstocki Psyilla spp. such as P. mali; Pteromalus spp., Pulvinaria amygdali Pyrilla spp., Quadraspidiotus spp., such as Q. perniciosus; Quesada gigas, Rastrococcus spp., Reduvius senillis, Rhizoecus americanus, Rhodnius spp., Rhopalomyzus ascalonicus, Rhopalosiphum spp. such as R. pseudobrassicas, R. insertum, R. maidis, R. padl; Sagatodes spp., Sahibergeilla singularis, Saissetia spp., Sappaphis mala, Sappaphis mali; Scaptocoris spp., Scaphoides titanus, Schizaphis graminum, Schizoneura lanuginosa, Scotinophora spp., Selenaspidus articulatus, Sitobion avenae, Sogata spp., Sogatella furcifera, Solubea insularis, Spissistllus festinus (=Stictocephala festina), Stephanitis nashi, Stephanitis pyrioides, Stephanitis takeyai, Tenalaphara malayensis, Tetraleurodes perseae, Therioaphis maculate, Thyanta spp. such as T. accerra, T. perditor; Tibraca spp., Tomaspis spp., Toxoptera spp. such as T. auranti; Trialeurodes spp. such as T. abutlonea, T. ricini, T. vaporariorum; Triatoma spp., Trioza spp., Typhlocyba spp., Unaspis spp. such as U. citr U. yanonensis; and Viteus vitifoii,

Insects from the order Hymenoptera for example Acanthomyops interjectus, Athaia rosae, Atta spp. such as A. capiguara, A. cephalotes, A. cephalotes, A. laevigata, A. robusta, A. sexdens, A. texana, Bombus spp., Brachymyrmex spp., Camponotus spp. such as C. floridanus, C. pennsylvanicus, C. modoc, Cardiocondyla nuda, Chalibion sp, Crematogasterspp., Dasymutilla occidentals, Diprion spp., Dolichovespula maculata, Dorymyrmex spp., Dryocosmus kuriphilus, Formica spp., Hoplocampa spp. such as H. minuta, H. testudinea; Iridomyrmex humllis, Lasius spp. such as L. niger, Linepithema humile, Liometopum spp., Leptocybe invasa, Monomorium spp. such as M. pharaonis, Monomorium, Nylandria fulva, Pachycondyla chinensis, Paratrechina longicornis, Para vespula spp., such as P. germanica, P. pennsylvanica, P. vulgaris; Pheidole spp. such as P. megacephala; Pogonomyrmex spp. such as P. barbatus, P. calfornicus, Polistes rubiginosa, Prenolepis impairs, Pseudomyrmex graclilis, Schelipron spp., Sirex cyaneus, Solenopsis spp. such as S. geminata, S. invicta, S. molesta, S. richteri, S. xyloni, Sphecius speciosus, Sphex spp., Tapinoma spp. such as T. melanocephalum, T. sessile; Tetramorium spp. such as T. caespitum, T. bicarinatum, Vespa spp. such as V. crabro; Vespula spp. such as V. squamosal; Wasmannia auropunctata, Xylocopa sp;

Insects from the order Orthoptera for example Acheta domesticus, Calliptamus italilcus, Chortoicetes terminifera, Ceuthophilus spp., Diastrammena asynamora, Dociostaurus maroccanus, Gryllotalpa spp. such as G. africana, G. gryllotalpa; Gryllus spp., Hieroglyphus daganensis, Kraussaria angullfera, Locusta spp. such as L. migratoria, L. pardalina; Melanoplus spp. such as M. bivittatus, M. femurrubrum, M. mexicanus, M. sanguinipes, M. spretus; Nomadacris septemfasciata, Oedaleus senegalensis, Scapteriscus spp., Schistocerca spp. such as S. americana, S. gregaria, Stemopelmatus spp., Tachycines asynamorus, and Zonozerus variegatus;

Pests from the Class Arachnida for example Acari, e.g. of the families Argasidae, Ixodidae and Sarcoptidae, such as Amblyomma spp. (e.g. A. americanum, A. variegatum, A. maculatum), Argas spp. such as A. persicu), Boophilus spp. such as B. annulatus, B. decoloratus, B. microplus, Dermacentorspp. such as D. silvarum, D. andersoni, D. variabilis, Hyalomma spp. such as H. truncatum, Ixodes spp. such as I. ricinus, I. rubicundus, I. scapularis, I. holocyclus, I. pacificus, Rhiplcephalus sanguineus, Ornithodorus spp. such as O. moubata, O. hermsi, O. turicata, Ornithonyssus bacoti, Otobius megnini, Dermanyssus gaillnae, Psoroptes spp. such as P. ovis, Rhipicephalus spp. such as R. sanguineus, R. appendiculatus, Rhipicephalus evertsi, Rhizoglyphus spp., Sarcoptes spp. such as S. Scabie and Family Eriophyidae including Aceria spp. such as A. sheldonl A. anthocoptes, Acailltus spp., Aculops spp. such as A. lycopersici, A. pelekassl Aculus spp. such as A. schlechtendali; Colomerus vitis, Epitrimerus pyri, Phyllocoptruta oleivora; Eriophytes ribis and Eriophyes spp. such as Eriophyes sheldon; Family Tarsonemidae including Hemitarsonemus spp., Phytonemus pallidus and Polyphagotarsonemus latus, Stenotarsonemus spp. Steneotarsonemus spinkl Family Tenuipalpidae including Brevipalpus spp. such as B. phoenicis; Family Tetranychidae including Eotetranychus spp., Eutetranychus spp., Oligonychus spp., Petrobia latens, Tetranychus spp. such as T. cinnabarinus, T. evansi, T. kanzawali, pacificus, T. phaseulus, T. telarius and T. urticae; Bryobia praetlosa; Panonychus spp. such as P. ulmi, P. citr; Metatetranychus spp. and Oligonychus spp. such as O. pratensis, O. perseae, Vasates lycopersic; Raoiella indica, FamilyCarpoglyphidae including Carpoglyphus spp.; Penthaleidae spp. such as Halotydeus destructor, Family Demodicidae with species such as Demodex spp.; Family Trombicidea including Trombicula spp.; Family Macronyssidae including Ornothonyssus spp.; Family Pyemotidae including Pyemotes tritic; Tyrophagus putrescentiae; Family Acaridae including Acarus siro; Family Araneida including Latrodectus mactans, Tegenaria agrestis, Chiracanthium sp, Lycosa sp Achaearanea tepidariorum and Loxosceles reclusa;

Pests from the Phylum Nematoda, for example, plant parasitic nematodes such as root-knot nematodes, Meloidogyne spp. such as M. hapla, M. incognita, M. javanica; cyst-forming nematodes, Globodera spp. such as G. rostochiensis; Heterodera spp. such as H. avenae, H. glycines, H. schachtii, H. trifolii; Seed gall nematodes, Anguina spp.; Stem and foliar nematodes, Aphelenchoides spp. such as A. besseyi; Sting nematodes, Belonolaimus spp. such as B. longicaudatus; Pine nematodes, Bursaphelenchus spp. such as B. lignicolus, B. xylophilus; Ring nematodes, Criconema spp., Criconemella spp. such as C. xenoplax and C. ornata; and, Criconemoides spp. such as Criconemoides informis; Mesocriconema spp.; Stem and bulb nematodes, Ditylenchus spp. such as D. destructor, D. dipsaci; Awl nematodes, Dolichodorus spp.; Spiral nematodes, Helliocotylenchus multicinctus; Sheath and sheathoid nematodes, Hemicycliophora spp. and Hemicriconemoides spp.; Hirshmanniella spp.; Lance nematodes, Hoploaimus spp.; False rootknot nematodes, Nacobbus spp.; Needle nematodes, Longidorus spp. such as L. elongatus; Lesion nematodes, Pratylenchus spp. such as P. brachyurus, P. neglectus, P. penetrans, P. curvitatus, P. goodeyi; Burrowing nematodes, Radopholus spp. such as R. similis; Rhadopholus spp.; Rhodopholus spp.; Reniform nematodes, Rotylenchus spp. such as R. robustus, R. reniformis; Scutellonema spp.; Stubby-root nematode, Trichodorus spp. such as T. obtusus, T. primitivus; Paratrichodorus spp. such as P. minor; Stunt nematodes, Tylenchorhynchus spp. such as T. clayton, T. dublus; Citrus nematodes, Tylenchulus spp. such as T. semipenetrans; Dagger nematodes, Xiphinema spp.; and other plant parasitic nematode species;

Insects from the order Isoptera for example Calotermes flavicollis, Coptotermes spp. such as C. formosanus, C. gestroi, C. acinaciformis; Cornitermes cumulans, Cryptotermes spp. such as C. brevis, C. cavifrons; Globitermes sulfureus, Heterotermes spp. such as H. aureus, H. longiceps, H. tenuis; Leucotermes flavipes, Odontotermes spp., Incisltermes spp. such as I. minor, I. Snyder Marginitermes hubbard Mastotermes spp. such as M. darwiniensis Neocapritermes spp. such as N. opacus, N. parvus; Neotermes spp., Procornitermes spp., Zootermopsis spp. such as Z angusticollis, Z nevadensis, Reticulltermes spp. such as R. hesperus, R. tibialis, R. speratus, R. flavipes, R. grassei R. lucifugus, R. santonensis, R. virginicus; Termes natalensis,

Insects from the order Blattaria for example Blatta spp. such as B. orientalis, B. lateralis; Blattella spp. such as B. asahinae, B. germanica; Leucophaea maderae, Panchiora nivea, Periplaneta spp. such as P. americana, P. australasiae, P. brunnea, P. fuligginosa, P. japonica; Supella longipalpa, Parcoblatta pennsylvanica, Eurycotis floridana, Pycnoscelus surinamensis,

Insects from the order Siphonoptera for example Cediopsylla simples, Ceratophyllus spp., Ctenocephallides spp. such as C. felis, C. canis, Xenopsylla cheopis, Pulex irritans, Trichodectes canis, Tunga penetrans, and Nosopsyllus fasciatus,

Insects from the order Thysanura for example Lepisma saccharina, Ctenolepisma urbana, and Thermobia domestica,

Pests from the class Chilopoda for example Geophilus spp., Scutigera spp. such as Scutigera coleoptrata;

Pests from the class Diplopoda for example Blaniulus guttulatus, Julus spp., Narceus spp.,

Pests from the class Symphyla for example Scutigerella immaculata,

Insects from the order Dermaptera, for example Forficula auricularia,

Insects from the order Collembola, for example Onychiurus spp., such as Onychiurus armatus,

Pests from the order Isopoda for example, Armadillidium vulgare, Oniscus asellus, Porcellio scaber,

Insects from the order Phthiraptera, for example Damalinia spp., Pediculus spp. such as Pediculus humanus capitis, Pediculus humanus corporis, Pediculus humanus humanus; Pthirus pubis, Haematopinus spp. such as Haematopinus eurysternus, Haematopinus suis; Linognathus spp. such as Linognathus vituti, Bovicola bovis, Menopon gallinae, Menacanthus stramineus and Solenopotes capillatus, Trichodectes spp.,

Examples of further pest species which may be controlled by compounds of formula (I) include: from the Phylum Mollusca, class Bivalvia, for example, Dreissena spp.; class Gastropoda, for example, Arion spp., Biomphalaria spp., Bulinus spp., Deroceras spp., Galba spp., Lymnaea spp., Oncomelania spp., Pomacea canaliclata, Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malay Brugia timor Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multllocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora is, Stronyloides spp., Taenia saginata, Taenia sollum, Trichinella spiralis, Trichinella nativa, Trichinella britov Trichinella nelsonl, Trichinella pseudopsiralis, Trichostrongulus spp., Trichuris trichiura, Wuchereria bancrofti.

The compounds of the present invention are suitable for use in treating or protecting animals against infestation or infection by parasites. Therefore, the present invention also relates to the use of a compound of the present invention for the manufacture of a medicament for the treatment or protection of animals against infestation or infection by parasites. Furthermore, the present invention relates to a method of treating or protecting animals against infestation and infection by parasites, which comprises orally, topically or parenterally administering or applying to the animals a parasiticidally effective amount of a compound of the present invention.

The present invention also relates to the non-therapeutic use of compounds of the present invention for treating or protecting animals against infestation and infection by parasites. Moreover, the present invention relates to a non-therapeutic method of treating or protecting animals against infestation and infection by parasites, which comprises applying to a locus a parasiticidally effective amount of a compound of the present invention.

The compounds of the present invention are further suitable for use in combating or controlling parasites in and on animals. Furthermore, the present invention relates to a method of combating or controlling parasites in and on animals, which comprises contacting the parasites with a parasitically effective amount of a compound of the present invention.

The present invention also relates to the non-therapeutic use of compounds of the present invention for controlling or combating parasites. Moreover, the present invention relates to a non-therapeutic method of combating or controlling parasites, which comprises applying to a locus a parasiticidally effective amount of a compound of the present invention.

The compounds of the present invention can be effective through both contact (via soil, glass, wall, bed net, carpet, blankets or animal parts) and ingestion (e.g. baits). Furthermore, the compounds of the present invention can be applied to any and all developmental stages.

The compounds of the present invention can be applied as such or in form of compositions comprising the compounds of the present invention.

The compounds of the present invention can also be applied together with a mixing partner, which acts against pathogenic parasites, e.g. with synthetic coccidiosis compounds, polyetherantibiotics such as Amprolium, Robenidin, Toltrazuril, Monensin, Salinomycin, Maduramicin, Lasalocid, Narasin or Semduramicin, or with other mixing partners as defined above, or in form of compositions comprising said mixtures.

The compounds of the present invention and compositions comprising them can be applied orally, parenterally or topically, e.g. dermally. The compounds of the present invention can be systemically or non-systemically effective.

The application can be carried out prophylactically, therapeutically or non-therapeutically. Furthermore, the application can be carried out preventively to places at which occurrence of the parasites is expected.

As used herein, the term “contacting” includes both direct contact (applying the compounds/compositions directly on the parasite, including the application directly on the animal or excluding the application directly on the animal, e.g. at it's locus for the latter) and indirect contact (applying the compounds/compositions to the locus of the parasite). The contact of the parasite through application to its locus is an example of a non-therapeutic use of the compounds of the present invention.

The term “locus” means the habitat, food supply, breeding ground, area, material or environment in which a parasite is growing or may grow outside of the animal.

As used herein, the term “parasites” includes endo- and ectoparasites. In some embodiments of the present invention, endoparasites can be preferred. In other embodiments, ectoparasites can be preferred. Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus; cockroaches (Blattaria-Blattodea), e.g. Battella germanica, Battella asahinae, Periplaneta americana, Periplaneta japonica, Periplaneta brunnea, Periplaneta fuligginosa, Periplaneta australasiae, and Blatta orientalis; flies, mosquitoes (Diptera), e.g. Aedes aegypt Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discallis, Chrysops silacea, Chrysops atlanticus, Cochliomya hominivorax, Cordylobia anthropophaga, Culicoldes furens, Culex pipiens, Culex nigrpalpus, Culex quinquefasciatus, Culex tarsalis, Culiseta inornata, Culiseta melanura, Dermatobia hominis, Fannia canicularis, Gasterophilus intestinals, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Mus cina stabulans, Oestrus ovis, Phlebotomus argentipes, Psorophora columbiae, Psorophora discolor, Prosimullum mixtum, Sarcophaga haemorrhoidalis, Sarcophaga sp., Simullum vittatum, Stomoxys calcitrans, Tabanus bovinus, Tabanus atratus, Tabanus lineola, and Tabanus similis; lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus; ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Omithodorus hermsi, Omithodorus turicata and parasitic mites (Mesostigmata), e.g. Omithonyssus bacoti and Dermanyssus galinae; Actinedida (Prostigmata) und Acaridida (Astigmata), e.g. Acarapis spp., Cheyletiella spp., Omithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp; Bugs (Heteropterida): Cimex lectularlus, Cimex hemipterus, Reduvius senilis, Triatoma spp., Rhodnius ssp., Panstrongylus ssp., and Arilus critatus, Anoplurida, e.g. Haematopinus spp., Linognathus spp., Pediculus spp., Phtirus spp., and Solenopotes spp.; Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp.; Roundworms Nematoda: Wipeworms and Trichinosis (Trichosyringida), e.g. Trichinellidae (Trichinella spp.), (Trichuridae) Trichuris spp., Capillaria spp.; Rhabditida, e.g. Rhabditis spp., Strongyloides spp., Helicephalobus spp.; Strongylida, e.g. Strongylus spp., Ancylostoma spp., Necatoramericanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capllaris, Protostrongylus spp., Angiostrongylus spp., Parelaphostrongylus spp., Aleurostrongylus abstrusus, and Dioctophyma renale; Intestinal roundworms (Ascaridida), e.g. Ascaris lumbricoldes, Ascaris suum, Ascaridia gali, Parascaris equorum, Enterobius vermicularis (Threadworm), Toxocara canis, Toxascaris leonine, Skrjabinema spp., and Oxyuris equi; Camallanida, e.g. Dracunculus medinensis (guinea worm); Spirurida, e.g. Theiazia spp., Wuchereria spp., Brugia spp., Onchocerca spp., Dirofilari spp.a, Dipetalonema spp., Setaria spp., Elaeophora spp., Spirocerca lupi, and Habronema spp.; Thorny headed worms (Acanthocephala), e.g. Acanthocephalus spp., Macracanthorhynchus hirudinaceus and Oncicola spp.; Planarians (Plathelminthes): Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp.; Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

As used herein, the term “animal” includes warm-blooded animals (including humans) and fish. Preferred are mammals, such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels. Particularly preferred are domestic animals, such as dogs or cats.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

Generally, it is favorable to apply the compounds of the present invention in total amounts of 0.5 mg/kg to 100 mg/kg per day, preferably 1 mg/kg to 50 mg/kg per day.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further auxiliaries such as acids, bases, buffer salts, preservatives, and solubilizers. Suitable auxiliaries for injection solutions are known in the art. The solutions are filtered and filled sterile.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on. Solutions for use on the skin are prepared according to the state of the art and according to what is described above for injection solutions, sterile procedures not being necessary.

Gels are applied to or spread on the skin or introduced into body cavities. Gels are prepared by treating solutions which have been prepared as described in the case of the injection solutions with sufficient thickener that a clear material having an ointment-like consistency results. Suitable thickeners are known in the art.

Pour-on formulations are poured or sprayed onto limited areas of the skin, the active compound penetrating the skin and acting systemically. Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added. Suitable such auxiliaries are known in the art.

Emulsions can be administered orally, dermally or as injections. Emulsions are either of the water-in-oil type or of the oil-in-water type. They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances. Suitable hydrophobic phases (oils), suitable hydrophilic phases, suitable emulsifiers, and suitable further auxiliaries for emulsions are known in the art.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers. Suitable suspending agents, and suitable other auxiliaries for suspensions including wetting agents are known in the art.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form. Suitable auxiliaries for this purpose are known in the art.

The compositions which can be used in the invention can comprise generally from about 0.001 to 95% of the compound of the present invention.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

Topical application may be conducted with compound-containing shaped articles such as collars, medallions, ear tags, bands for fixing at body parts, and adhesive strips and foils.

Generally it is favorable to apply solid formulations which release compounds of the present invention in total amounts of 10 mg/kg to 300 mg/kg, preferably 20 mg/kg to 200 mg/kg, most preferably 25 mg/kg to 160 mg/kg body weight of the treated animal in the course of three weeks.

### PREPARATION EXAMPLES

With appropriate modification of the starting materials, the procedures as described in the preparation examples below were used to obtain further compounds of formula I. The compounds obtained in this manner are listed in the table C that follows, together with physical data.

Compounds can be characterized e.g. by coupled High Performance Liquid Chromatography/mass spectrometry (HPLC/MS), by ¹H-NMR and/or by their melting points.

Analytical HPLC-Method: Agilent Eclipse Plus C18, 50×4.6 mm, ID 5 μm; Elution: A=10 mM Amm. Formate (0.1% Formic Acid), B=Acetonitrile (0.1% Formic Acid), Flow=1.2 ml/min. at 30° C.; Gradient:=10% B to 100% B—3 min, hold for 1 min, 1 min—10% B. Run Time=5.01 min.

¹H-NMR: The signals are characterized by chemical shift (ppm, 8 [delta]) vs. tetramethylsilane respectively, CDCl₃ for ¹³C-NMR, by their multiplicity and by their integral (relative number of hydrogen atoms given). The following abbreviations are used to characterize the multiplicity of the signals: m=multiplet, q=quartet, t=triplet, d=doublet and s=singlet.

Abbreviations used are: d for day(s), h for hour(s), min for minute(s), r.t./room temperature for 20-25° C., Rt for retention time; DMSO for dimethyl sulfoxide, OAc for acetate, EtOAc for ethyl acetate, TH F for tetrahydrofuran, and t-BuOH for tert-butanol.

### Example1: 1-(2-isopropylphenyl)-3-[(E)-[(E)-2-methyl-3-[2-[4-(trifluoromethoxy)anilino] pyrimidin-5-yl]prop-2-enylidene]amino]thiourea (C-1)

### Step 1: 5-bromo-N-[4-(trifluoromethoxy)phenyl]pyrimidin-2-amine

To a solution of 5-bromo-2-chloro-pyrimidine (0.1 g) in N,N-Dimethyl formamide (3 mL), was added Potassium carbonate (0.142 g), Copper (I) iodide (0.01 g), 8-hydroxy quinoline (0.08 g) and 4-(trifluoromethoxy) aniline (0.11 g). The mixture was heated at 95° C. for 24 h. The mixture was diluted with water (15 mL) and extracted with Ethyl acetate. The organic extracts were dried over anhydrous Sodium sulfate and evaporated under reduced pressure and the resulting residue was subjected to flash silica gel column chromatography using a gradient of Ethyl acetate and Heptane as eluent to afford the titled compound as a off-white solid (0.05 g). LC/MS: Rₜ: 1.86 min; MS: m/z=334 (M+1)⁺; ¹H NMR (300 MHz, DMSO-d₆) δ 10.12-9.94 (m, 1H), 8.74-8.56 (m, 2H), 7.89-7.72 (m, 2H), 7.38-7.24 (m, 2H).

### Step 2: (E)-2-methyl-3-[2-[4-(trifluoromethoxy)anilino]pyrimidin-5-yl]prop-2-enal

A solution of 5-bromo-N-[4-(trifluoromethoxy)phenyl]pyrimidin-2-amine (0.1 g) was taken up in 1,4-Dioxane (4 mL) and water (1 mL) and the mixture degassed with nitrogen for 15 min. [1,1′-Bis(diphenylphosphino)ferrocene]dichloro palladium(II) (0.01 g), Cesium carbonate (0.2 g) and 2-[(E)-3,3-diethoxy-2-methyl-prop-1-enyl]-4,4,5,5-tetramethyl-1,3,2-dioxaborolane (0.11 g) were added and the mixture degassed with nitrogen for an additional 10 min. The mixture was heated at 95° C. for 4 h and subsequently cooled to ambient temperature. A solution of Hydrochloric acid (1 N) was added and the mixture stirred for 30 min. The mixture was neutralized with solid Sodium bicarbonate and extracted with Ethyl acetate. The organic extracts were dried over anhydrous sodium sulfate and evaporated under reduced pressure and the residue obtained was purified by flash column chromatography eluting with a gradient of Ehyl acetate and Heptane to afford (E)-2-methyl-3-[2-[4-(trifluoromethoxy)anilino]pyrimidin-5-yl]prop-2-enal as a solid (0.03 g). LC/MS: Rₜ: 1.65 min; MS: m/z=324 (M+1)⁺; ¹H NMR (300 MHz, DMSO-d₆) δ 10.33 (s, 1H), 9.54 (s, 1H), 8.80 (s, 2H), 8.01-7.80 (m, 2H), 7.48-7.34 (m, 2H), 7.32 (s, 1H), 2.02 (s, 3H).

### Step 3: 1-(2-isopropylphenyl)-3-[(E)-[(E)-2-methyl-3-[2-[4-(trifluoromethoxy)anilino] pyrimidin-5-yl]prop-2-enylidene]amino]thiourea

A mixture of (E)-2-methyl-3-[2-[4-(trifluoromethoxy)anilino]pyrimidin-5-yl]prop-2-enal (0.2 g) and 1-amino-3-(2-isopropylphenyl)thiourea (0.130 g) in Ethanol (3 mL) was heated at 80° C. for 3 h. The mixture was cooled to ambient temperature and the precipitated solid was filtered and washed with cold Ethanol and n-pentane to afford the titled compound (0.2 g). LC/MS: Rₜ: 1.96 min; MS: m/z=515 (M+1)⁺; ¹H NMR (300 MHz, DMSO-d₆) δ 11.74 (s, 1H), 10.10 (s, 1H), 9.72 (s, 1H), 8.69 (s, 2H), 7.96 (s, 1H), 7.91-7.88 (m, 2H), 7.36-7.19 (m, 6H), 6.69 (s, 1H), 3.14-3.05 (m, 1H), 2.14 (s, 3H), 1.19-1.17 (m, 6H).

### Example 2: Synthesis of 1-(2,6-dimethylphenyl)-3-[(E)-[(E)-2-methyl-3-[2-[4-(trifluoromethoxy) anilino]pyrimidin-5-yl]prop-2-enylidene]amino]thiourea (C-2)

A mixture of (E)-2-methyl-3-[2-[4-(trifluoromethoxy)anilino]pyrimidin-5-yl]prop-2-enal (0.2 g) and 1-amino-3-(2,6-dimethylphenyl)thiourea (0.12 g) in Ethanol (3 mL) was heated at 80° C. for 3 h. The mixture was subsequently cooled to ambient temperature, the suspended solids filtered, washed sequentially with cold ethanol, pentane and dried to afford the title compound (0.2 g). LC/MS: Rₜ: 1.89 min; MS: m/z=501 (M+1)⁺; ¹H NMR (300 MHz, DMSO-d₆) δ 11.67 (s, 1H), 10.09 (s, 1H), 9.61 (s, 1H), 8.69 (s, 2H), 7.94 (s, 1H), 7.91-7.80 (m, 2H), 7.42-7.22 (m, 2H), 7.21-7.02 (m, 3H), 6.67 (s, 1H), 2.21 (s, 3H), 2.18 (s, 6H).

### Example 3: Synthesis of (2Z)-3-(2-isopropylphenyl)-2-[(E)-[(E)-2-methyl-3-[2-[4-(trifluoromethoxy) anilino]pyrimidin-5-yl]prop-2-enylidene]hydrazono]thiazolidin-4-one (C-3)

To a stirred solution of 1-(2-isopropylphenyl)-3-[(2-methyl-3-[2-[4-(trifluoromethoxy) anilino]pyrimidin-5-yl]prop-2-enylidene]amino]thiourea (0.170 g) in Ethanol (3 mL) was added Sodium acetate (0.082 g) and Methyl bromoacetate (0.25 g). The mixture was stirred at room temperature for 30 h and subsequently diluted with water and extracted with Ethyl acetate. The organic extracts were separated, dried over anhydrous Sodium sulfate and evaporated under reduced pressure. The residue obtained was subjected to flash silica gel column chromatography eluting with a gradient of Ethylacetate-Heptane to afford the title compound as a solid (0.16 g). LC/MS: Rₜ: 1.99 min; MS: m/z=555 (M+1)⁺; ¹H NMR (300 MHz, DMSO-d₆) δ 10.14 (s, 1H), 8.61 (s, 2H), 8.02 (s, 1H), 7.96-7.78 (m, 2H), 7.57-7.40 (m, 2H), 7.40-7.27 (m, 3H), 7.27-7.19 (m, 1H), 6.78 (s, 1H), 4.33-3.99 (m, 2H), 2.85-2.67 (m, 1H), 2.01 (s, 3H), 1.14-1.12 (m, 6H).

### Example 4: Synthesis of (2Z)-3-(2,6-dimethylphenyl)-2-[(2-methyl-3-[2-[4-(trifluoromethoxy) anilino]pyrimidin-5-yl] prop-2-enylidene]hydrazono]thiazolidin-4-one (C-4)

A mixture of 1-(2,6-dimethylphenyl)-3-[2-methyl-3-[2-[4(trifluoromethoxy)anilino] pyrimidin-5-yl]prop-2-enylidene]amino]thiourea (0.19 g), Sodium acetate (0.094 g) and Methyl bromoacetate (0.29 g) in Ethanol (4 mL) was stirred at r.t. for 24 h. The mixture was subsequently diluted with water and extracted with Ethyl acetate. The organic extracts were dried over anhydrous Sodium sulfate and evaporated under reduced pressure and the residue obtained was subjected to silica gel flash column chromatography, eluting with a gradient of Ethyl acetate-Heptane to obtain the title compound (0.170 g). LC/MS: Rₜ: 1.95 min; MS: m/z=541 (M+1)⁺; ¹H NMR (300 MHz, DMSO-d₆) δ 10.11 (s, 1H), 8.61 (s, 2H), 8.02 (s, 1H), 7.95-7.75 (m, 2H), 7.38-7.28 (m, 2H), 7.28-7.24 (m, 1H), 7.24-7.16 (m, 2H), 6.78 (s, 1H), 4.22 (s, 2H), 2.14 (s, 3H), 2.08 (s, 6H).

### Example 5: Synthesis of (2Z)-3-(2-isopropylphenyl)-2-[(E)-[(E)-3-[2-[N-methyl-4-(trifluoromethoxy)anilino]pyrimidin-5-yl]prop-2-enylidene]hydrazono]thiazolidin-4-one (C-5)

### Step 1: 5-bromo-N-methyl-N-[4-(trifluoromethoxy)phenyl]pyrimidin-2-amine

To a stirred solution of 5-bromo-N-[4-(trifluoromethoxy)phenyl]pyrimidin-2-amine (0.1 g) in N, N-Dimethylformamide (3 mL) at 0° C. was added sodium hydride (0.01 g). Methyl iodide (0.064 g) was added and the mixture stirred at r.t. for 12 h. The mixture was diluted with saturated Ammonium chloride solution, extracted with Ethyl acetate, the organic extracts dried over anhydrous Sodium sulfate and concentrated under reduced pressure. The residue obtained was subjected to silica gel flash column chromatography eluting with a gradient of Ethyl acetate and Heptane to afford the desired product (0.05 g). LC/MS: Rₜ: 2.262 min; MS: m/z=348 (M+1)⁺; ¹H NMR (300 MHz, DMSO-d₆) δ 8.53 (s, 2H), 7.54-7.43 (m, 2H), 7.43-7.32 (m, 2H), 3.44 (s, 3H), 1.20 (d, J=19.4 Hz, 2H).

### Step 2: (E)-3-[2-[N-methyl-4-(trifluoromethoxy)anilino]pyrimidin-5-yl]prop-2-enal

A mixture of 5-bromo-N-methyl-N-[4-(trifluoromethoxy)phenyl] pyrimidin-2-amine (0.5 g) in 1,4-Dioxane (8 mL) and water (2 mL) was degassed with nitrogen gas for 15 min. [1,1′-Bis(diphenylphosphino)ferrocene]dichloro palladium(II) (0.055 g), Cesium carbonate (1 g) and 2-[(E)-3,3-diethoxyprop-1-enyl]-4, 4, 5, 5-tetramethyl-1, 3, 2-dioxaborolane (0.7 g) were added and the mixture heated at 95° C. for 3 h. The reaction mixture was cooled to ambient temperature, acidified with 1N HCl solution and stirred at r.t. for 30 min. The mixture was neutralized with solid Sodium bicarbonate, extracted with ethyl acetate, the organic layers dried over Sodium sulfate and evaporated under reduced pressure. The resulting residue was subjected to silica gel flash column chromatography eluting with a gradient of Ethyl acetate and heptane to obtain the title compound as a solid (150 mg). LC/MS: Rₜ: 1.98 min; MS: m/z=324.2 (M+1)⁺; ¹H NMR (300 MHz, DMSO-d₆) δ 9.59 (d, J=7.8 Hz, 1H), 8.79 (s, 2H), 7.59 (d, J=16.0 Hz, 1H), 7.56-7.48 (m, 2H), 7.42 (d, J=8.6 Hz, 2H), 6.84 (dd, J=16.0, 7.8 Hz, 1H), 3.53 (s, 3H).

### Step 3: 1-(2-isopropylphenyl)-3-[[3-[2-[N-methyl-4-(trifluoromethoxy)anilino] pyrimidin-5-yl]prop-2-enylidene]amino]thiourea

A mixture of (E)-3-[2-[N-methyl-4-(trifluoromethoxy)anilino]pyrimidin-5-yl]prop-2-enal (0.09 g) and 1-amino-3-(2-isopropylphenyl)thiourea (0.058 g) in Ethanol (3 mL) was heated at 80° C. for 2 h. The mixture was subsequently cooled to ambient temperature, the suspended solids filtered and washed with cold Ethanol to afford the title compound (0.08 g). LC/MS: Rₜ: 1.90 min; MS: m/z=515 (M+1)⁺; ¹H NMR (300 MHz, DMSO-d₆) δ 8.64 (s, 2H), 8.00-7.87 (m, 1H), 7.57-7.43 (m, 2H), 7.45-7.35 (m, 2H), 7.35-7.29 (m, 1H), 7.29-7.22 (m, 1H), 7.22-7.12 (m, 2H), 6.99-6.82 (m, 2H), 3.50 (s, 3H), 3.15-2.94 (m, 1H), 1.15 (d, J=6.9 Hz, 6H).

### Step 4: (2Z)-3-(2-isopropylphenyl)-2-[[3-[2-[N-methyl-4-(trifluoromethoxy)anilino] pyrimidin-5-yl]prop-2-enylidene]hydrazono]thiazolidin-4-one

A mixture of 1-(2-isopropylphenyl)-3-[(E)-[(E)-3-[2-[N-methyl-4-(trifluoromethoxy)aniline]pyrimidin-5-yl]prop-2-enylidene]amino]thiourea (0.08 g), Sodium acetate (0.039 g) and Methyl bromo acetate (0.120 g) in Ethanol (3 mL) was stirred at r.t. for 12 h. The reaction mixture was subsequently diluted with water and extracted with Ethyl acetate. The organic extracts were dried over anhydrous Sodium sulfate and evaporated under reduced pressure and the residue obtained subjected to silica gel flash column chromatograph eluting with a gradient of Ethyl acetate-Heptane to afford the title compound (0.04 g). LC/MS: Rₜ: 1.97 min; MS: m/z=555 (M+1)⁺; ¹H NMR (300 MHz, DMSO-d₆) δ 8.65 (s, 2H), 8.00 (d, J=9.3 Hz, 1H), 7.55-7.44 (m, 4H), 7.44-7.35 (m, 2H), 7.35-7.27 (m, 1H), 7.26-7.19 (m, 1H), 7.12-6.99 (m, 1H), 6.99-6.85 (m, 1H), 4.29-3.99 (m, 2H), 3.50 (s, 3H), 2.82-2.64 (m, 1H), 1.21-1.00 (m, 6H).

### Example 6: Synthesis of 1-(2-isopropylphenyl)-3-[(E)-[(E)-2-methyl-3-[2-[N-methyl-4-(trifluoro methoxy)anilino]pyrimidin-5-yl]prop-2-enylidene]amino]thiourea (C-6)

### Step 1: (E)-2-methyl-3-[2-[N-methyl-4-(trifluoromethoxy)anilino]pyrimidin-5-yl]prop-2-enal

A mixture of 5-bromo-N-methyl-N-[4-(trifluoromethoxy) phenyl] pyrimidin-2-amine (0.4 g) in 1,4 Dioxane (6 mL) and water (1.5 mL) was degassed with nitrogen gas for 15 min. [1,1′-Bis(diphenylphosphino)ferrocene]dichloro palladium(II) (0.042 g), Cesium carbonate (0.751 g) and 2-[(E)-3, 3-diethoxyprop-1-enyl]-4, 4, 5, 5-tetramethyl-1, 3, 2-dioxaborolane (0.590 g) were added and the mixture heated at 95° C. for 4 h. The reaction mixture was cooled to ambient temperature, acidified with 1N HCl solution and stirred at r.t. for 30 min. The mixture was neutralized with solid Sodium bicarbonate, extracted with ethyl acetate, the organic layers dried over Sodium sulfate and evaporated under reduced pressure. The resulting residue was subjected to silica gel flash column chromatography eluting with a gradient of Ethyl acetate and heptane to obtain the title compound as a solid (0.2 g). LC/MS: Rₜ: 2.15 min; MS: m/z=338.2 (M+1)⁺; ¹H NMR (300 MHz, DMSO-d₆) δ 9.51 (s, 1H), 8.71 (s, 2H), 7.60-7.47 (m, 2H), 7.47-7.37 (m, 2H), 7.33 (s, 1H), 3.54 (s, 3H), 1.97 (s, 3H).

### Step 2: 1-(2-isopropylphenyl)-3-[(E)-[(E)-2-methyl-3-[2-[N-methyl-4-(trifluoromethoxy) anilino]pyrimidin-5-yl]prop-2-enylidene]amino]thiourea

A mixture of (E)-2-methyl-3-[2-[N-methyl-4-(trifluoromethoxy)anilino]pyrimidin-5-yl] prop-2-enal (0.83 g) and 1-amino-3-(2-isopropylphenyl)thiourea (0.51 g) in Ethanol (6 mL) was heated at 85° C. for 3 h. The mixture was cooled to ambient temperature and the precipitated solid was filtered and washed with cold ethanol and n-pentane and dried to afford the desired product (0.850 g). LC/MS: Rₜ: 2.37 min; MS: m/z=529.3 (M+1)⁺; ¹H NMR (300 MHz, DMSO-d₆) δ 11.71 (s, 1H), 9.69 (s, 1H), 8.59 (s, 2H), 7.94 (s, 1H), 7.53-7.50 (m, 2H), 7.41-7.38 (m, 2H), 7.35-7.15 (m, 4H), 6.64 (s, 1H), 3.52 (s, 3H), 3.14-3.01 (m, 1H), 2.16 (s, 3H), 1.17 (d, J=6.9 Hz, 6H).

### Example 7: Synthesis of (2Z)-3-(2-isopropylphenyl)-2-[(E)-[(E)-2-methyl-3-[2-[N-methyl-4-(trifluoromethoxy)anilino]pyrimidin-5-yl]prop-2-enylidene]hydrazono]thiazolidin-4-one (C-7)

1-(2-isopropylphenyl)-3-[(E)-[(E)-2-methyl-3-[2-[N-methyl-4-(trifluoromethoxy)anilino] pyrimidin-5-yl]prop-2-enylidene]amino]thiourea (0.67 g) was taken up in Ethanol (15 mL). Ethyl-2-bromo acetate (0.97 g) and Sodium acetate (0.31 g) added and the mixture stirred a r.t. for 24 h. The reaction mixture was diluted with water and extracted with Ethyl acetate, the organic extracts dried over anhydrous Sodium sulfate and evaporated under reduced pressure. The residue obtained was purified by silica gel flash column chromatography using a gradient of Ethyl acetate and Heptane to afford the desired product. (0.59 g). LC/MS: Rₜ: 2.49 min; MS: m/z=569.4 (M+1)⁺; ¹H NMR (300 MHz, DMSO-d₆) δ 8.51 (s, 2H), 7.99 (s, 1H), 7.62-7.47 (m, 3H), 7.44 (dd, J=8.1, 1.5 Hz, 1H), 7.42-7.36 (m, 2H), 7.32 (ddd, J=8.6, 6.8, 2.0 Hz, 1H), 7.23 (dd, J=7.9, 1.3 Hz, 1H), 6.74 (s, 1H), 4.29-3.95 (m, 2H), 3.51 (s, 3H), 2.84-2.66 (m, 1H), 2.10 (s, 3H), 1.13 (t, J=6.3 Hz, 6H).

### Example 8: Synthesis of 1-[(E)-[(E)-3-[5-(dimethylamino)-6-[N-methyl-4-(trifluoromethoxy)anilino]-3-pyridyl]-2-methyl-prop-2-enylidene]amino]-3-(2-isopropylphenyl)thiourea (C-8)

### Step 1: Synthesis of 5-bromo-3-nitro-N-[4-(trifluoromethoxy)phenyl]pyridin-2-amine

To a solution of 5-bromo-2-chloro-3-nitro pyridine (12 g, 0.050 mol) in n-butanol (100 mL) were added Triethyl amine (6.13 g, 0.060 mol) and 4-Trifluoromethoxy aniline (10.74 g, 0.060 mol). The mixture was heated at 125° C. for 2 h. The mixture was subsequently cooled to ambient temperature and the precipitated solids were filtered and dried under vacuum to obtain the desired product as a brown solid (12.1 g, 63.3% yield. LC/MS: Rt: 1.938 min; MS: m/z=378 (M+1)⁺; ¹H NMR (300 MHz, DMSO-d6); δ 8.69 (d, J=2.3 Hz, OH), 8.60 (d, J=2.3 Hz, OH), 7.71 (d, J=9.0 Hz, 1H), 7.37 (d, J=8.6 Hz, 1H).

### Step 2: Synthesis of 5-bromo-N2-[4-(trifluoromethoxy)phenyl]pyridine-2,3-diamine

To a stirred solution of 5-bromo-3-nitro-N-[4-(trifluoromethoxy)phenyl]pyridin-2-amine (1.33 g) in Ethyl acetate (15 mL) was added Tin chloride dihydrate (3.1 g) and the mixture was heated at 80° C. for 2 h. The mixture was subsequently cooled to ambient temperature and Sodium bicarbonate solution was added and the resultant mixture was filtered through a Celite bed. The organic layer was separated, washed with saturated Sodium chloride solution and water and dried over anhydrous Sodium sulphate. The organic layer was then evaporated under reduced pressure to get the desired product as a light brown solid (0.63 g, 53% yield). LC/MS: Rt: 1.723 min; MS: m/z=348.3 (M+1)⁺; ¹H NMR (300 MHz, DMSO-d6); δ 8.08 (s, 1H), 7.68 (d, J=9.0 Hz, 2H), 7.52 (d, J=2.2 Hz, 1H), 7.24 (d, J=8.6 Hz, 2H), 7.07 (d, J=2.2 Hz, 1H), 5.45 (s, 2H).

### Step 3: Synthesis of 5-bromo-N2,N3,N3-trimethyl-N2-[4-(trifluoromethoxy)phenyl]pyridine-2,3-diamine

To a solution of 5-bromo-N2-[4-(trifluoromethoxy)phenyl]pyridine-2,3-diamine (3.8 g) in N, N-Dimethyl formamide (30 mL) was added Sodium hydride, 60% suspension in mineral oil, (1 g) at 0° C. and stirred for 15 minutes. Methyl iodide (7 g) was added drop-wise. The mixture was stirred at ambient temperature for 12 h and subsequently a saturated solution of Ammonium chloride was added and the mixture extracted with Ethyl acetate. The Ethyl acetate extracts were dried over anhydrous Sodium sulfate and evaporated under reduced pressure and the resultant solids subjected to flash column chromatography on Silica gel using a gradient of Ethyl acetate/Heptane as eluent to afford the desired product as a brown solid (1.33 g, 31%). LC/MS: Rt: 2.441 min; MS: m/z=390.4 (M+1)⁺; ¹H NMR (300 MHz, DMSO-d6); δ 8.04 (d, J=2.2 Hz, 1H), 7.51 (d, J=2.2 Hz, 1H), 7.21 (d, J=8.1 Hz, 1H), 6.84 (d, J=9.1 Hz, 2H), 3.29 (s, 3H), 2.65 (s, 6H).

### Step 4: Synthesis of (E)-3-[5-(dimethylamino)-6-[N-methyl-4-(trifluoromethoxy)anilino]-3-pyridyl]-2-methyl-prop-2-enal

A mixture of 5-bromo-N2,N3,N3-trimethyl-N2-[4-(trifluoromethoxy)phenyl]pyridine-2,3-diamine (0.130 g) in 1,4 Dioxane (4 mL) and water (1 mL) was degassed with Nitrogen gas for 15 minutes. [1,1 ′-Bis(diphenylphosphino)ferrocene]palladium(II) dichloride (0.012 g), Cesium carbonate (0.217 g,) and 2-[(E)-3,3-diethoxy-2-methyl-prop-1-enyl]-4,4,5,5-tetramethyl-1,3,2-dioxaborolane (0.171 g) were added and the mixture heated at 95° C. for 4 h. 1N Hydrochloric acid solution was subsequently added and the mixture was neutralized with solid Sodium bicarbonate and extracted with Ethyl acetate. The Ethyl acetate extracts were separated and filtered through Celite, dried over anhydrous Sodium sulphate and evaporated under reduced pressure. The residue obtained was subjected to Silica gel flash column chromatography using a gradient of Ethyl acetate/Heptane as eluent to afford the desired product as a pale yellow solid (0.090 g, 71%, yield). LC/MS: Rt: 2.302 min; MS: m/z=380.3 (M+1)⁺; ¹H NMR (300 MHz, DMSO-d6); δ 9.57 (s, 1H), 8.22 (d, J=2.0 Hz, 1H), 7.69-7.39 (m, 2H), 7.29-7.10 (m, 2H), 6.91 (d, J=9.1 Hz, 2H), 3.37 (s, 3H), 2.57 (s, 6H).

### Step 5: Synthesis of 1-[(E)-[(E)-3-[5-(dimethylamino)-6-[N-methyl-4-(trifluoromethoxy)anilino]-3-pyridyl]-2-methyl-prop-2-enyl idene]amino]-3-(2-isopropylphenyl)thiourea

To a solution of (E)-3-[5-(dimethylamino)-6-[N-methyl-4-(trifluoromethoxy)anilino]-3-pyridyl]-2-methyl-prop-2-enal (0.420 g) in Ethanol (5 mL) was added 1-amino-3-(2-isopropyl phenyl)thiourea (0.231 g) and the mixture heated at 85° C. for 2 h. The reaction mixture was cooled to ambient temperature and the resulting precipitate was filtered, washed with cold Ethanol and n-Pentane to get the desired product as yellow solid (0.5 g, 78.3%, yield). LC/MS: Rt: 2.49 min; MS: m/z=571.4 (M+1)⁺; ¹H NMR (300 MHz, DMSO-d6); δ 11.75 (s, 1H), 9.71 (s, 1H), 8.11 (d, J=1.9 Hz, 1H), 7.98 (d, J=1.2 Hz, 1H), 7.44 (d, J=2.0 Hz, 1H), 7.38-7.20 (m, 4H), 7.20-7.13 (m, 2H), 6.83 (d, J=9.3 Hz, 3H), 3.31 (s, 3H), 3.19-2.95 (m, 1H), 2.60 (s, 6H), 2.29-2.17 (m, 3H), 1.19 (d, J=6.9 Hz, 6H).

### Example 9: Synthesis of (2Z)-2-[(E)-[(E)-3-[5-(dimethylamino)-6-[N-methyl-4-(trifluoromethoxy) anilino]-3-pyridyl]-2-methyl-prop-2-enylidene]hydrazono]-3-(2-isopropylphenyl)thiazolidin-4-one (C-9

To a stirred solution of 1-[(E)-[(E)-3-[5-(dimethylamino)-6-[N-methyl-4-(trifluoromethoxy)anilino]-3-pyridyl]-2-methyl-prop-2-enylidene]amino]-3-(2-isopropylphenyl)thiourea (0.370 g) in Ethanol (5 mL) was added Sodium acetate (0.160 g) and Methyl-2-bromo acetate (0.496 g). The mixture was stirred for 12 h, Water was added and the mixture was subsequently extracted with Ethyl acetate, the organic extracts dried over anhydrous Sodium sulfate and evaporated under reduced pressure. The residue obtained was subjected to flash column chromatography using a gradient of Ethyl acetate/Heptane as eluent to obtain the desired product as a yellow solid (0.170 g, 42%). LC/MS: Rt: 2.550 min; MS: m/z=611.4 (M+1)⁺; ¹H NMR (300 MHz, DMSO-d6); δ 8.04 (d, J=2.1 Hz, 2H), 7.59-7.41 (m, 2H), 7.39-7.27 (m, 2H), 7.24 (dd, J=7.9, 1.3 Hz, 1H), 7.16 (d, J=8.7 Hz, 2H), 6.91 (s, 1H), 6.83 (d, J=9.1 Hz, 2H), 4.56-3.86 (m, 2H), 3.33 (s, 4H), 2.76 (dd, J=13.4, 6.5 Hz, 1H), 2.58 (s, 6H), 2.17 (d, J=1.2 Hz, 3H), 1.14 (t, J=6.9 Hz, 6H).

### Example 10: Synthesis of (2E)-2-[(Z)-[3-(2-isopropylphenyl)-4-oxo-thiazolidin-2-ylidene]hydrazono]-N-methyl-N-[2-[N-methyl-4-(trifluoromethoxy)anilino]pyrimidin-5-yl]acetamide (C-10)

### Step 1: 5-bromo-N-[4-(trifluoromethoxy)phenyl]pyrimidin-2-amine

To a stirred solution of 5-bromo-2-chloro-pyrimidine (0.1 g) in N,N-Dimethylformamide (3 mL) were added Potassium carbonate (0.142 g), Copper(I)iodide (0.01 g), 8-hydroxy quinoline (0.08 g) and 4-(trifluoromethoxy) aniline (0.11 g). The mixture was heated at 95° C. for 24 h, cooled to ambient temperature, diluted with Water and extracted with Ethyl acetate. The Ethyl acetate extracts were dried over anhydrous Sodium sulfate and concentrated under reduced pressure. The residue obtained was purified by silica gel flash column chromatography using a gradient of Ethyl acetate and Heptane as eluent to afford the desired compound as yellow solid (0.05 g, 27% yield). LC/MS: Rt: 1.80 min; MS: m/z=336 (M+1)+.

### Step 2: 5-bromo-N-methyl-N-[4-(trifluoromethoxy)phenyl]pyrimidin-2-amine

To a solution of 5-bromo-N-[4-(trifluoromethoxy)phenyl]pyrimidin-2-amine (2.5 g) in N, N-Dimethylformamide (10 mL) at 0° C. was added Sodium Hydride (60% dispersion in mineral oil) (0.449g) portion wise. Methyl iodide (0.7 mL) was added and the mixture stirred at ambient temperature for 12 h. The mixture was poured into ice and the precipitated solids were filtered and dried to get the desired product (2.5 g, 96%). LC/MS: Rt: 2.25 min; MS: m/z=348.15 (M+1)+.

### Step 3: N2,N5-dimethyl-N2-[4-(trifluoromethoxy)phenyl]pyrimidine-2,5-diamine

To a solution of 5-bromo-N-methyl-N-[4-(trifluoromethoxy)phenyl]pyrimidin-2-amine (0.5 g) in N-Methyl Pyrrolidone (6 mL) in a sealed tube was added Cu (I) oxide (0.021g) and a 40% solution of Methylamine in water (6 mL). The mixture was heated at 80° C. for 12 h and water (20 mL) followed by Ethyl acetate (20 mL) were added. The mixture was filtered through a Celite bed, the organic layer separated, dried over anhydrous Sodium sulphate and evaporated to dryness under reduced pressure. The reside was purified by Silica gel flash column chromatography eluting with a gradient of Ethyl acetate and Heptane to afford the desired product (0.3 g, 70%) as a beige solid. LC/MS: Rt: 1.88 min; MS: m/z=288.3 (M-1); ¹H NMR (300 MHz, DMSO-d6) δ 7.91 (s, 2H), 7.45-7.33 (m, 2H), 7.33-7.24 (m, 2H), 5.41 (q, J=5.3 Hz, 1H), 3.42 (s, 3H), 2.67 (d, J=5.3 Hz, 3H).

### Step 4: (2E)-2-[(2-isopropylphenyl)carbamothioylhydrazono]acetic acid

To a solution of 1-amino-3-(2-isopropylphenyl)thiourea (1 g) in Methanol (20 mL) was added Glyoxylic acid monohydrate (0.44 g) and the mixture stirred at ambient temperature for 2 h. The mixture was evaporated under reduced pressure and the residue was washed with n-Pentane to get the desired product (1.2g, 95%) as a off white solid. LC/MS: Rt: 1.439 min; MS: m/z=264 (M-1); ¹H NMR (300 MHz, DMSO-d6) δ 12.34 (s, 1H), 10.32 (s, 1H), 7.44-7.37 (m, 2H), 7.33 (td, J=7.8, 7.4, 1.7 Hz, 1H), 7.24 (td, J=7.4, 1.8 Hz, 1H), 7.17 (dd, J=7.8, 1.6 Hz, 1H), 3.05 (p, J=6.9 Hz, 1H), 1.17 (d, J=6.9 Hz, 6H).

### Step 5: (2E)-2-[(2-isopropylphenyl)carbamothioylhydrazono]-N-methyl-N-[2-[N-methyl-4-(trifluoromethoxy)anilino]pyrimidin-5-yl]acetamide

To a solution of N2,N5-dimethyl-N2-[4-(trifluoromethoxy)phenyl]pyrimidine-2,5-diamine (0.2 g, 0.67 mmol) and (2E)-2-[(2-isopropylphenyl)carbamothioylhydrazono]acetic acid (0.196 g) in Dichloromethane (20 mL) was added Diisopropylethylamine (0.25 mL) and a 50% solution of Propylphosphonic anhydride in Ethyl acetate (0.835g). The mixture was stirred for 12 h and subsequently poured into Water (30 mL) and extracted with Ethyl acetate (2×20 mL). The organic extracts were dried over anhydrous Sodium suphate and evaporated under reduced pressure and the residue obtained was subjected to Silica gel flash column chromatography eluting with a gradient of Ethyl acetate and Heptane to get the desired product and a yellow solid (0.32 g, 87% yield). LC/MS: Rt: 1.178 min; MS: m/z=546 (M+1)⁺; ¹H NMR (300 MHz, DMSO-d6) δ 11.77 (s, 1H), 9.43 (s, 1H), 8.48 (s, 2H), 7.60 (s, 1H), 7.48-7.27 (m, 8H), 7.27-7.07 (m, 4H), 3.45 (s, 5H), 3.24 (s, 5H), 2.58-2.50 (m, 120H), 1.13 (t, J=6.1 Hz, 11H).

### Step 6: (2E)-2-[(Z)-[3-(2-isopropylphenyl)-4-oxo-thiazolidin-2-ylidene]hydrazono]-N-methyl-N-[2-[N-methyl-4-(trifluoromethoxy)anilino]pyrimidin-5-yl]acetamide

A mixture of (2E)-2-[(2-isopropylphenyl)carbamothioylhydrazono]-N-methyl-N-[2-[N-methyl-4-(trifluoromethoxy)anilino]pyrimidin-5-yl]acetamide (0.15 g), Sodium acetate (0.26 g, 2.75 mmol) and Methyl bromoacetate (0.11 mL) in Ethanol (20 mL) was stirred at 40° C. for 12 h. The mixture was cooled to ambient temperature and Water (50 mL) was added and the mixture extracted with Ethyl acetate (2×50 mL). The combined organic extracts were dried over anhydrous Sodium sulphate and evaporated invacuo to a residue which was subjected to Silica gel flash column chromatography eluting with a gradient of Ethyl acetate and Heptane to afford the desired product (0.1 g, 62%). LC/MS: Rt: 2.10 min; MS: m/z=586.3 (M+1)⁺; ¹H NMR (300 MHz, DMSO-d6) δ 8.34 (s, 2H), 7.66-7.09 (m, 9H), 4.40-4.00 (m, 2H), 3.47 (s, 4H), 2.62 (d, J=6.9 Hz, 1H), 1.03 (dd, J=22.9, 6.8 Hz, 6H).

### Example 11: Synthesis of (2E)-2-[(Z)-[3-(2-isopropylphenyl)-4-oxo-thiazolidin-2-ylidene]hydrazono]-N-[2-[N-methyl-4-(trifluoromethoxy)anilino]pyrimidin-5-yl]acetamide (C-11)

A mixture of (2E)-2-[(2-isopropylphenyl)carbamothioylhydrazono]-N-[2-[N-methyl-4-(trifluoromethoxy)anilino]pyrimidin-5-yl]acetamide (0.1 g, 0.188 mmol), Sodium acetate (0.154 g) and Methyl bromoacetate (0.076 mL) was taken up in Ethanol (20 mL) and stirred at 40° C. for 12 h. The mixture was cooled to ambient temperature and Water (50 mL) was added and extracted with Ethyl acetate (2×50 mL). The combined Ethyl acetate extracts were dried over anhydrous Sodium sulphate and evaporated invacuo and the resultant residue was subjected to Silica gel flash column chromatography eluting with a gradient of Ethyl acetate and Heptane to afford the desired product (0.05 g, 46%). LC/MS: Rt: 2.149 min; MS: m/z=572.3 (M+1)⁺; ¹H NMR (300 MHz, DMSO-d6) δ 10.36 (s, 1H), 8.66 (s, 2H), 7.67 (s, 1H), 7.49 (d, J=9.0 Hz, 3H), 7.37 (d, J=8.4 Hz, 3H), 7.26 (dd, J=7.9, 1.4 Hz, 1H), 4.51-3.98 (m, 2H), 3.48 (s, 3H), 1.13 (dd, J=6.9, 4.4 Hz, 6H).

### Example 12: Synthesis of (2Z)-3-(2-isopropylphenyl)-2-[(E)-[(E)-2-methyl-3-[6-[N-methyl-4-(trifluoromethoxy)anilino]-3-pyridyl]prop-2-enylidene]hydrazono]thiazolidin-4-one (C-12)

### Step 1: Synthesis of 5-bromo-N-[4-(trifluoromethoxy)phenyl]pyridin-2-amine

A mixture of 2-amino-5-bromo pyridine (4.4 g), Copper (II) acetate (10.85 g) and Potassium phosphate (6.128 g) in Dimethylsulfoxide (70 mL) was heated at 100° C. for 24 h. The mixture was subsequently cooled to ambient temperature, Ethyl acetate was added and the mixture filtered through Celite. The organic layer was separated, washed with saturated Sodium chloride solution, Water and subsequently dried over anhydrous Sodium sulphate. The organic layer was then evaporated invacuo and the residue subjected to Silica gel flash column chromatography eluting with a gradient of Ethyl acetate and heptane to afford the desired product as a brown solid. (2.3 g, 27%). LC/MS: Rt: 2.139 min; MS: m/z=335.30 (M+1)⁺; ¹H NMR (300 MHz, DMSO-d6) δ 8.29 (d, J=2.5 Hz, 1H), 7.79 (d, J=9.1 Hz, 2H), 7.48-7.14 (m, 2H), 6.88 (dd, J=8.9, 1.4 Hz, 2H).

### Step 2: Synthesis of 5-bromo-N-methyl-N-[4-(trifluoromethoxy)phenyl]pyridin-2-amine

To a 0° C. solution of 5-bromo-N-[4-(trifluoromethoxy)phenyl]pyridin-2-amine (2.3 g) in N, N-Dimethyl formamide (15 mL) was added Sodium hydride (0.2 g). Methyl iodide (1.47 g) was added drop-wise and the mixture stirred at ambient temperature for 12 h. Saturated Ammonium chloride solution was added and the mixture extracted with Ethyl acetate and the extract was dried over anhydrous Sodium sulphate and evaporated invacuo and the residue obtained was subjected to silica gel flash column chromatography eluting with a gradient of Ethyl acetate and heptane to get the desired product as a off-white solid (1.75 g, 73%). LC/MS: Rt: 2.209 min; MS: m/z=349.3 (M+1)⁺; ¹H NMR (300 MHz, DMSO-d6) δ 8.23 (d, J=2.5 Hz, 1H), 7.65 (dt, J=9.1, 2.0 Hz, 1H), 7.43 (s, 4H), 6.58 (d, J=9.1 Hz, 1H), 3.37 (s, 3H).

### Step 3: Synthesis of (E)-2-methyl-3-[6-[N-methyl-4-(trifluoromethoxy)anilino]-3-pyridyl]prop-2-enal

A mixture of 5-bromo-N-methyl-N-[4-(trifluoromethoxy)phenyl]pyridin-2-amine (1.75 g, 5.05 mmol), 1,4 Dioxane (20 mL) and Water (5 mL) was degassed with nitrogen gas. [1,1′-Bis(diphenyl phosphino)ferrocene]palladium(II) dichloride (0.370 g), Cesium carbonate (3.3 g) and 2-[(E)-3,3-diethoxy-2-methyl-prop-1-enyl]-4,4,5,5-tetramethyl-1,3,2-dioxaborolane (2.59 g) were added and the degassing continued for a further 10 min. The mixture was heated at 90° C. for 3 h and cooled to ambient temperature. 1N Hydrochloric acid solution was added and the mixture was neutralized with solid Sodium bicarbonate. The mixture was extracted with Ethyl acetate and the extracts were dried over anhydrous Sodium sulphate and evaporated invacuo and the resultant residue was subjected to Silica gel flash chromatography eluting with a Ethyl acetate/Heptane gradient to get the desired compound as a white solid solid (1.2 g, 71%). LC/MS: Rt: 2.170 min; MS: m/z=337.2 (M+¹); ¹H NMR (300 MHz, DMSO-d6) δ 9.49 (s, 1H), 8.47 (d, J=2.4 Hz, 1H), 7.84 (dd, J=9.1, 2.5 Hz, 1H), 7.61-7.41 (m, 4H), 7.37 (s, 1H), 6.66 (d, J=9.0 Hz, 1H), 3.47 (s, 3H), 1.97 (d, J=1.1 Hz, 3H).

### Step 4: Synthesis of 1-(2-isopropylphenyl)-3-[(E)-[(E)-2-methyl-3-[6-[N-methyl-4-(trifluoromethoxy) anilino]-3-pyridyl]prop-2-enylidene]amino]thiourea

To a stirred solution of (E)-2-methyl-3-[6-[N-methyl-4-(trifluoromethoxy)anilino]-3-pyridyl]prop-2-enal (0.350 g) in Ethanol (4 mL) was added 1-amino-3-(2-isopropylphenyl)thiourea (0.217 g) and the mixture heated at 85° C. for 3 h. Water was added and the mixture was extracted with ethyl acetate, the extracts dried over anhydrous Sodium sulphate, evaporated and the residue was flash chromatographed over Silica gel to get the desired product as a yellow solid (0.22 g, 40%). LC/MS: Rt: 2.423 min; MS: m/z=528.4 (M+1)⁺; ¹H NMR (300 MHz, DMSO-d6) δ 11.68 (s, 1H), 9.66 (s, 1H), 8.37-8.25 (m, 1H), 7.94 (s, 1H), 7.77-7.62 (m, 1H), 7.52-7.38 (m, 5H), 7.44-7.26 (m, 2H), 7.27 (s, 1H), 7.26-7.10 (m, 1H), 6.72-6.61 (m, 2H), 4.03 (q, J=7.1 Hz, 1H), 3.44 (s, 4H), 3.09 (p, J=6.8 Hz, 1H), 2.16 (s, 3H), 1.99 (s, 1H), 1.18 (d, J=6.9 Hz, 6H).

### Step 5: Synthesis of (2Z)-3-(2-isopropylphenyl)-2-[(E)-[(E)-2-methyl-3-[6-[N-methyl-4-(trifluoromethoxy)anilino]-3-pyridyl]prop-2-enylidene]hydrazono]thiazolidin-4-one

To a stirred solution of 1-(2-isopropylphenyl)-3-[(E)-[(E)-2-methyl-3-[6-[N-methyl-4-(trifluoromethoxy) anilino]-3-pyridyl]prop-2-enylidene]amino]thiourea (0.175 g) in Ethanol (4 mL) were added sodium acetate (0.082 g), and Methyl-2-bromo acetate (0.25 g) and the mixture stirred for 16 h. The mixture was subsequently diluted with Water and extracted with Ethyl acetate, the extracts were dried over anhydrous Sodium sulfate and concentrated under reduced pressure to get a residue which was flash chromatographed to get the desired product as yellow solid (0.08 g, 40%, yield). LC/MS: Rt: 2.465 min; MS: m/z=568.4 (M+1)⁺; 1H NMR (300 MHz, DMSO-d6) δ 8.26 (d, J=2.3 Hz, 1H), 7.99 (s, 1H), 7.64 (dd, J=9.0, 2.4 Hz, 1H), 7.55-7.38 (m, 6H), 7.32 (ddd, J=8.6, 6.8, 2.0 Hz, 1H), 7.23 (dd, J=7.9, 1.4 Hz, 1H), 6.80 (s, 1H), 6.64 (d, J=8.9 Hz, 1H), 4.20 (d, J=17.4 Hz, 1H), 4.09 (d, J=17.4 Hz, 1H), 3.43 (s, 3H), 2.76 (p, J=6.6 Hz, 1H), 2.14-2.07 (m, 3H), 1.13 (t, J=6.4 Hz, 6H).

### Example 13: Synthesis of (2E)-2-[(2-isopropylphenyl)carbamothioylhydrazono]-N-[2-[N-methyl-4-(trifluoromethoxy)anilino]pyrimidin-5-yl]acetamide (C-13)

### Step 1: N2-methyl-N2-[4-(trifluoromethoxy)phenyl]pyrimidine-2,5-diamine

To a solution of 5-bromo-N-methyl-N-[4-(trifluoromethoxy)phenyl]pyrimidin-2-amine (1g) in N-Methylpyrrolidone (10 mL) in a sealed tube was added Copper (I) oxide (0.041 g) and a 30% solution of Methyl amine in water (10 mL). The mixture was heated at 80° C. for 12 h. Water (20 mL) and Ethyl acetate (20 mL) were added and the mixture filtered through Celite. The organic layer was separated, washed with a saturated solution of Sodium chloride, dried over anhydrous Sodium sulphate and evaporated invacuo. The residue obtained was subjected to Silica gel flash column chromatography using a Ethyl acetate/Heptane gradient to obtain the desired product (0.7 g, 85%) as a beige solid. LC/MS: Rt: 1.715 min; MS: m/z=285.2 (M-1).

### Step-2: (2E)-2-[(2-isopropylphenyl)carbamothioylhydrazono]-N-[2-[N-methyl-4-(trifluoromethoxy) anilino]pyrimidin-5-yl]acetamide

To a solution of N2-methyl-N2-[4-(trifluoromethoxy)phenyl]pyrimidine-2,5-diamine (0.1 g) and (2E)-2-[(2-isopropylphenyl)carbamothioylhydrazono]acetic acid (0.10 g) in Dichloromethane (10 mL) was added Diisopropylethylamine (0.125 mL) and a 50% solution of Propylphosphonic anhydride solution (0.45 g). The mixture was stirred at ambient temperature for 12 h. Water (30 mL) was added and the mixture extracted with Ethyl acetate (2×20 mL). The organic extracts were dried over anhydrous Sodium sulphate and evaporated under reduced pressure and the resultant residue was subjected to Silica gel flash column chromatography to get the desired product (0.32g, 87%) as a yellow solid. LC/MS: Rt: 2.134 min; MS: m/z=532 (M+1)⁺; ¹H NMR (300 MHz, DMSO-d6) δ 12.29 (s, 1H), 10.36 (s, 1H), 10.13 (s, 1H), 8.55 (s, 2H), 7.56-7.45 (m, 3H), 7.45-7.34 (m, 4H), 7.26 (dtd, J=22.5, 8.5, 7.8, 1.7 Hz, 2H), 3.48 (s, 3H), 3.07 (p, J=6.8 Hz, 1H), 1.19 (d, J=6.9 Hz, 6H).

### Example 14: Synthesis of 1-(2-isopropylphenyl)-3-[(E)-[(E)-3-[2-[N-methyl-4-(trifluoromethoxy) anilino]pyrimidin-5-yl]prop-2-enylidene]amino]imidazolidine-2,4-dione (C-14)

A mixture of (E)-3-[2-[N-methyl-4-(trifluoromethoxy)anilino]pyrimidin-5-yl]prop-2-enal (0.135 g), 3-amino-1-(2-isopropylphenyl)imidazolidine-2,4-dione (0.097g) and Concentrated Hydrochloric acid (2 drops) in Ethanol was heated at 80° C. for 3 h. The mixture was cooled to ambient temperature and the precipitated solids were filtered and washed with cold ethanol and dried under vacuum to afford the desired product as a beige colored solid. (0.064 g, 28%). LC/MS: Rt: 2.180 min; MS: m/z=539 (M+1)⁺.

### Example 15: Synthesis of 1-(2-isopropylphenyl)-3-[(E)-[(E)-2-methyl-3-[6-[N-methyl-4-(trifluoromethoxy)anilino]-3-pyridyl]prop-2-enylidene]amino]thiourea (C-15)

A mixture of (E)-2-methyl-3-[6-[N-methyl-4-(trifluoromethoxy)anilino]-3-pyridyl]prop-2-enal (0.32 g) and 1-amino-3-(2-isopropylphenyl)thiourea (0.217 g) in Ethanol (5 mL) was heated at 85° C. for 3 h. The mixture was cooled to ambient temperature, diluted with water, extracted with Ethyl acetate and the extracts were dried over anhydrous Sodium sulphate and evaporated invacuo. The residue obtained was subjected to flash column chromatography using a gradient of Ethyl acetate and heptane as eluent to afford the desired product as a yellow solid (0.26 g, 46%). LC/MS: Rt: 2.39 min; MS: m/z=528.7 (M+1)⁺; ¹H NMR (300 MHz, DMSO-d6) δ 11.70 (s, 1H), 9.68 (s, 1H), 8.31 (d, J=2.3 Hz, 2H), 7.94 (s, 1H), 7.81 (dd, J=9.2, 2.4 Hz, 2H), 7.59-7.14 (m, 12H), 6.72 (d, J=8.5 Hz, 3H), 3.09 (p, J=6.8 Hz, 1H), 2.16 (s, 3H), 1.18 (d, J=6.8 Hz, 7H).

### Example 16: Synthesis of 1-(2-isopropylphenyl)-3-[(E)-[(E)-2-methyl-3-[4-methyl-2-[N-methyl-4-(trifluoromethoxy)anilino]pyrimidin-5-yl]prop-2-enylidene]amino]thiourea (C-16)

### Step 1: 5-bromo-2-chloro-4-methyl-pyrimidine

A mixture of 5-Bromo 2,4-dichloropyrimidine (0.4 g) and Iron(III)acetylacetonate (0.062 g) in Tetrahydrofuran (10 mL) was cooled to 0° C. Methyl magnesium bromide (3 M solution in Diethyl ether) (0.76 mL) was added dropwise and the mixture stirred for 2 h. Saturated Ammonium Chloride solution was added and the mixture extracted with Ethyl acetate (2×20 mL). The organic extracts were dried over anhydrous Sodium sulphate and evaporated under reduced pressure to get a residue which was purified by Silica gel flash column chromatography eluting with a gradient of Ethyl acetate and n-heptane to afford the desired product (0.220 g, 60%) as a white solid. 1H NMR (300 MHz, CDCl3) δ 8.52 (s, 1H), 2.57 (s, 3H).

### Step 2: 5-bromo-4-methyl-N-[4-(trifluoromethoxy)phenyl]pyrimidin-2-amine

A mixture of 5-bromo-2-chloro-4-methyl-pyrimidine (2 g), 4-Trifluoromethoxyaniline (2.04 g), concentrated Hydrochloric acid solution (0.2 mL) in in 2-propanol (20 mL) was heated at 100° C. for 4 h. The mixture was subsequently cooled to room temperature, poured into ice and basified with a saturated solution Sodium bicarbonate solution and precipitated solids were filtered. The filtered solid was subjected to Silica gel flash column chromatography eluting with a gradient of Ethyl acetate and n-Heptane to afford the desired product (1.75 g, 52%) as an off-white solid. LC/MS: Rt: 2.20 min; MS: m/z=346.1 (M-1); 1H NMR (300 MHz, CDCl3) δ 8.32 (s, 1H), 7.88 (s, 1H), 7.63-7.51 (m, 2H), 7.19-7.07 (m, 2H), 2.50 (s, 3H).

### Step 3: 5-bromo-N,4-dimethyl-N-[4-(trifluoromethoxy)phenyl]pyrimidin-2-amine

To the solution of 5-bromo-4-methyl-N-[4-(trifluoromethoxy)phenyl]pyrimidin-2-amine (1.5 g) in N, N-Dimethylformamide (30 mL) at 0° C. was added Sodium hydride (60% dispersion in mineral oil) (0.207g) portion wise. Methyl iodide (0.35 mL) was added and the mixture stirred at 0° C. for 1 h. The reaction mixture was poured into ice and extracted with Ethyl acetate (2×20 mL). The combined organic layer was dried over anhydrous Sodium sulphate and evaporated under reduced pressure and the residue subjected to Silica gel flash column chromatography using a gradient of Ethyl acetate and n-heptane as eluent to get the desired product to get the desired product (1.4 g, 89% yield) as a beige solid. LC/MS: Rt: 2.388 min; MS: m/z=364.3 (M+1)⁺; ¹H NMR (300 MHz, CDCl3) δ 8.20 (s, 1H), 7.27 (d, J=9.0 Hz, 2H), 7.17 (d, J=8.1 Hz, 2H), 3.47 (d, J=1.3 Hz, 3H), 2.40 (d, J=7.0 Hz, 3H).

### Step 4: (E)-2-methyl-3-[4-methyl-2-[N-methyl-4-(trifluoromethoxy)anilino]pyrimidin-5-yl]prop-2-enal

A mixture of 5-bromo-N,4-dimethyl-N-[4-(trifluoromethoxy)phenyl]pyrimidin-2-amine (0.25 g), Cesium carbonate (0.45 g) and 2-[(Z)-3,3-diethoxy-1-methyl-prop-1-enyl]-4,4,5,5-tetramethyl-1,3,2-dioxaborolane (0.28 g) in 1,4-Dioxane (12 mL) and water (3 mL) was degassed for 10 min, followed by the addition of [1,1′-Bis(diphenylphosphino)ferrocene] palladium(II) dichloride (0.05 g) and the mixture heated at 90° C. for 2 h. The mixture was subsequently cooled to ambient temperature and water (10 mL) was added and the mixture extracted with Ethyl acetate (20 mL). The Ethyl acetate extracts were dried over anhydrous Sodium sulphate and evaporated under reduced pressure and the residue obtained was subjected to Silica gel flash column chromatography eluting with a gradient of Ethyl acetate/n-Heptane to afford the desired product (0.2 4g, 99%) as an off white solid. Rt: 2.173 min; MS: m/z=352.4 (M+1)⁺; ¹H NMR (300 MHz, CDCl₃) δ 9.52 (s, 1H), 8.33 (s, 1H), 7.29 (d, 2H), 7.17 (d, 2H), 3.52 (s, 3H), 2.39 (s, 3H).

### Step 5: 1-(2-isopropylphenyl)-3-[(E)-[(E)-2-methyl-3-[4-methyl-2-[N-methyl-4-(trifluoromethoxy) anilino] pyrimidin-5-yl]prop-2-enylidene]amino]thiourea

A mixture of (E)-2-methyl-3-[4-methyl-2-[N-methyl-4-(trifluoromethoxy)anilino]pyrimidin-5-yl]prop-2-enal (0.2 g) and 1-amino-3-(2-isopropylphenyl)thiourea (0.12 g) in Ethanol (10 mL) was heated at 80° C. for 2 h. The mixture was cooled to ambient temperature and the precipitated solids were collected by filtration, washed with cold Ethanol and dried under vacuum to afford the title compound 0.2 g (65%) as a yellow solid. LC/MS: Rt: 2.411 min; MS: m/z=541.7 (M)⁻; ¹H NMR (DMSO-d6): δ 11.71 (s, 1H), 9.67 (s, 1H), 8.32 (s, 1H), 7.98 (s, 1H), 7.56-7.46 (m, 2H), 7.43-7.30 (m, 3H), 7.30-7.14 (m, 3H), 6.74 (s, 1H), 3.51 (s, 3H), 3.08 (p, J=6.9 Hz, 2H), 2.34 (s, 3H), 2.06-1.98 (m, 3H), 1.18 (d, J=6.9 Hz, 6H).

### Example 17: Synthesis of (2Z)-3-(2-isopropylphenyl)-2-[(E)-[(E)-2-methyl-3-[4-methyl-2-[N-methyl-4-(trifluoromethoxy)anilino]pyrimidin-5-yl]prop-2-enylidene]hydrazono]thiazolidin-4-one (C-17)

A mixture of 1-(2-isopropylphenyl)-3-[(E)-[(E)-2-methyl-3-[4-methyl-2-[N-methyl-4-(trifluoromethoxy) anilino] pyrimidin-5-yl]prop-2-enylidene]amino]thiourea (0.1g), Sodium acetate (0.091 g), Methyl bromoacetate (0.075 mL) was taken up in Ethanol (20 mL) and the mixture stirred at 40° C. for 12 h. The mixture was diluted with water (50 mL) at ambient temperature and extracted with Ethyl acetate (2×50 mL). The combined organic layers were dried over anhydrous Sodium sulphate and evaporated invacuo and the residue obtained was subjected to Silica gel Flash column chromatography using a gradient of Ethylacetate/Heptane to afford the title compound (0.08 g, 75%) as a yellow solid. LC/MS: Rt: 2.498 min; MS: m/z=583.4 (M+1)⁺; ¹H NMR (300 MHz, DMSO-d6) δ 8.33 (s, 1H), 8.10 (s, 1H), 7.55-7.46 (m, 4H), 7.41 (dd, J=19.5, 8.0 Hz, 3H), 7.36-7.19 (m, 2H), 6.91 (s, 1H), 4.28-4.03 (m, 2H), 3.50 (s, 3H), 2.28 (s, 3H), 1.98 (d, J=1.2 Hz, 3H), 1.19-1.08 (m, 6H).

### Example 18: Synthesis of 1-(2-isopropylphenyl)-3-[(E)-2-[2-[N-methyl-4-(trifluoromethoxy)anilino]pyrimidin-5-yl]oxyethylideneamino]thiourea (C-18)

### Step 1: 2-[N-methyl-4-(trifluoromethoxy)anilino]pyrimidin-5-ol

A mixture of 5-bromo-N-methyl-N-[4-(trifluoromethoxy)phenyl]pyrimidin-2-amine (1.8 g) and Triisopropylborate (1.82 mL) was taken up in Tetrahydrofuran (30 mL) and cooled to −78° C. A solution of n-BuLi (1.6 M in n-hexane, 4.827 mL) was added and the mixture allowed to warm upto −20° C. for 20 min. Acetic acid (1.5 mL) followed by Methanol (8 mL) was added and the mixture evaporated under reduced pressure. To the resultant residue, Methanol (2 mL), Water (12 mL) and a solution of Hydrogen Peroxide (20% in water, 1.5 mL, 10.34 mmol) was added and the mixture stirred at ambient temperature for 12 h. The mixture was subsequently diluted with water (50 mL) and extracted with Ethyl acetate (2×50 mL). The combined organic layer was dried over anhydrous Sodium sulphate and evaporated invacuo and the resultant solid was purified by Silica gel flash column chromatography with a gradient of Ethyl acetate/Heptane as eluent to afford the desired product (1 g, 68% yield) as a white solid. LC/MS: Rt: 1.788 min; MS: m/z=286.3 (M+1)⁺; ¹H NMR (300 MHz, DMSO-d6) δ 9.47 (s, 1H), 8.05 (s, 2H), 7.48-7.37 (m, 2H), 7.32 (d, J=8.6 Hz, 2H), 3.43 (s, 3H).

### Step 2: 5-(2,2-diethoxyethoxy)-N-methyl-N-[4-(trifluoromethoxy)phenyl]pyrimidin-2-amine

To a solution of 2-[N-methyl-4-(trifluoromethoxy)anilino]pyrimidin-5-ol (0.25 g) in N,N-Dimethylacetamide (2 mL), was added KOH (0.098 g) followed by Bromoacetaldehyde diethylacetal (0.2 mL). The mixture was heated to 100° C. for 2 h and subsequently cooled to room temperature. The mixture was diluted with water (20 mL) and extracted with Ethyl acetate EtOAc (2×20 mL). The combined organic extracts were washed with a saturated solution of Sodium chloride, dried over anhydrous Sodium sulphate and evaporated under reduced pressure to afford the title compound as a pale yellow oil (0.25g, 71%). LC/MS: Rt: 2.23 min; MS: m/z=402.9 (M+1)⁺; ¹H NMR (300 MHz, CDCl3) δ 8.10 (s, 1H), 7.32-7.21 (m, 1H), 7.16 (d, J=8.5 Hz, 3H), 4.72 (t, J=5.1 Hz, 1H), 3.91 (d, J=5.1 Hz, 1H), 3.77-3.63 (m, 2H), 3.63-3.48 (m, 3H), 3.46 (s, 2H), 3.30 (d, J=5.5 Hz, 1H), 1.17 (td, J=7.1, 1.8 Hz, 6H).

### Step 3: 2-[2-[N-methyl-4-(trifluoromethoxy)anilino]pyrimidin-5-yl]oxyacetaldehyde

To a solution of 5-(2,2-diethoxyethoxy)-N-methyl-N-[4-(trifluoromethoxy)phenyl]pyrimidin-2-amine (0.2 g) in Acetone (4 mL) was added a solution of Hydrochloric acid (1 N, 1 ml) and the mixture heated at 70° C. for 5 h. The reaction was diluted with water (4 mL) and extracted with Ethyl acetate (2×10 mL). The combined organic layer was dried over anhydrous Sodium sulphate and evaporated under reduced pressure and the resultant residue was subjected to flash column chromatography using a gradient of Ethyl acetate/Heptane to afford the title compound as a colorless oil (0.14 g, 86%). LC/MS: Rt: 1.612 min; MS: m/z=327 (M+1)⁺; ¹H NMR (300 MHz, CDCl3) δ 9.85 (s, 1H), 8.17 (s, 2H), 7.36 (d, J=9.0 Hz, 2H), 7.25 (d, J=8.4 Hz, 2H), 4.61 (s, 2H), 3.54 (s, 3H).

### Step 4: 1-(2-isopropylphenyl)-3-[(E)-2-[2-[N-methyl-4-(trifluoromethoxy)anilino]pyrimidin-5-yl]oxyethylideneamino]thiourea

A mixture of 2-[2-[N-methyl-4-(trifluoromethoxy)anilino]pyrimidin-5-yl]oxyacetaldehyde (0.15 g) and 1-amino-3-(2-isopropylphenyl)thiourea (0.10 g) in Ethanol (5 mL) was heated at 80° C. for 1 h. Subsequently, the mixture was evaporated under reduced pressure to get the title compound as a white solid (0.16 g, 67%). LC/MS: Rt: 2.187 min; MS: m/z=519.65 (M+1)⁺; ¹H NMR (300 MHz, DMSO-d6) δ 11.73 (s, 1H), 9.82 (s, 1H), 8.34 (s, 2H), 7.57 (t, J=5.2 Hz, 1H), 7.51-7.39 (m, 2H), 7.36 (s, 2H), 7.30 (dd, J=11.3, 5.3 Hz, 2H), 7.27-7.11 (m, 3H), 4.77 (d, J=5.2 Hz, 2H), 3.45 (s, 3H), 3.02 (p, J=6.9 Hz, 2H), 1.12 (d, J=6.9 Hz, 6H).

### Example 19: Synthesis of (2Z)-3-(2-isopropylphenyl)-2-[(E)-2-[2-[N-methyl-4-(trifluoromethoxy)anilino]pyrimidin-5-yl]oxyethylidenehydrazono]thiazolidin-4-one (C-19)

A mixture of 1-(2-isopropylphenyl)-3-[(E)-2-[2-[N-methyl-4-(trifluoromethoxy)anilino]pyrimidin-5-yl]oxyethylideneamino]thiourea (0.1 g), Sodium acetate (0.095 g), Methyl bromoacetate (0.078 mL) in Ethanol (20 mL) was stirred at 40° C. for 12 h. The mixture was subsequently diluted with Water (50 mL) and extracted with Ethyl acetate (2×20 mL). The combined extracts were dried over anhydrous Sodium sulphate and evaporated invacuo and the residue obtained was purified by Silica gel flash column chromatography using a gradient of Ethyl acetate and heptane as eluent to afford the title compound (0.06 g, 56%) as a white solid. LC/MS: Rt: 2.25 min; MS: m/z=559.3 (M+1)+. ¹H NMR (300 MHz, DMSO-d6) δ 8.256 (s, 2H), 7.672 (m, 1H), 7.431-7.462 (m, 4H), 7.327-7.356 (m, 3H), 7.195-7.251 (m, 1H), 4.79 (d, J=4.5 Hz, 2H), 4.112 (m, 2H), 3.443 (s, 3H), 2.702-2.790 (m, 1H), 1.096-1.127 (m, 6H).

### Example 20: Synthesis of 1-(2-isopropylphenyl)-3-[(E)-2-[2-[N-methyl-4-(trifluoromethoxy)anilino]pyrimidin-5-yl]oxypropylideneamino]thiourea (C-20)

### Step 1: 5-(2,2-dimethoxy-1-methyl-ethoxy)-N-methyl-N-[4-(trifluoromethoxy)phenyl]pyrimidin-2-amine

A mixture of 2-[N-methyl-4-(trifluoromethoxy)anilino]pyrimidin-5-ol (0.4 g), Potassium hydroxide (0.158 g) and 2-bromo-1,1-dimethoxy-propane (0.4 mL) in N,N-Dimethylacetamide (4 mL) was heated at 100° C. for 24 h. The mixture was cooled to ambient temperature and water (50 mL) was added and the mixture extracted with Ethyl acetate (2×30 mL). The combined organic layer was washed with a saturated solution of Sodium chloride and dried over anhydrous Sodium sulphate and evaporated under reduced pressure and the crude obtained was purified by Silica gel flash column chromatography using a gradient of Ethyl acetate/Hepatane to obtain the title compound (0.1 g, 18%). LC/MS: Rt: 2.144 min; MS: m/z=388.4 (M+1)⁺; ¹H NMR (300 MHz, Chloroform-d) δ 8.14 (s, 2H), 7.36 (s, 3H), 7.25 (s, 2H), 7.16 (s, 1H), 3.51 (s, 2H), 3.43 (d, J=5.7 Hz, 4H), 3.39 (t, J=2.8 Hz, 5H).

### Step 2: 2-[2-[N-methyl-4-(trifluoromethoxy)anilino]pyrimidin-5-yl]oxypropanal

A solution of 5-(2,2-dimethoxy-1-methyl-ethoxy)-N-methyl-N-[4-(trifluoromethoxy)phenyl]pyrimidin-2-amine (0.1 g) and a solution of Hydrochloric acid (1 N, 1 mL) was taken up in in Acetone (4 mL) and heated at 70° C. for 12 h. The reaction was diluted with water (20 mL) and extracted with Ethyl acetate (2×20 mL). The combined organic layer was dried over anhydrous Sodium sulphate and evaporated under reduced pressure to get the title compound. (0.07 g, 79.4%) LC/MS: Rt: 1.685 min; MS: m/z=342.2 (M+1)⁺; ¹H NMR (300 MHz, CDCl3) δ 9.67 (s, 1H), 8.08 (s, 2H), 7.26 (dd, J=7.5, 5.1 Hz, 2H), 7.16 (d, J=7.9 Hz, 2H), 4.417-4.444 (m, 1H), 3.43 (s, 3H), 1.59 (d, J=7.5 Hz, 3H).

### Step 3: 1-(2-isopropylphenyl)-3-[(E)-2-[2-[N-methyl-4-(trifluoromethoxy)anilino]pyrimidin-5-yl]oxypropylideneamino]thiourea

The mixture of 2-[2-[N-methyl-4-(trifluoromethoxy)anilino]pyrimidin-5-yl]oxypropanal (0.15 g) and 1-amino-3-(2-isopropylphenyl)thiourea (0.09 g) in Ethanol (2 mL) was heated at 80° C. for 1 h. The mixture was evaporated under reduced pressure and the residue subjected to Silica gel column chromatography to get the title compound (0.16 g, 68%). LC/MS: Rt: 2.275 min; MS: m/z=533.3 (M+1)⁺; ¹H NMR (300 MHz, DMSO-d6) δ 11.63 (s, 1H), 9.81 (s, 1H), 8.34 (s, 2H), 7.51-7.40 (m, 2H), 7.38 (d, J=6.3 Hz, 1H), 7.35-7.22 (m, 4H), 7.22-7.11 (m, 2H), 4.97 (p, J=6.4 Hz, 2H), 3.44 (s, 3H), 3.01 (p, J=6.9 Hz, 2H), 1.49 (d, J=6.3 Hz, 3H), 1.11 (dd, J=6.9, 2.4 Hz, 7H).

### Example 21: Synthesis of (2Z)-3-(2-isopropylphenyl)-2-[(E)-2-[2-[N-methyl-4-(trifluoromethoxy) anilino] pyrimidin-5-yl]oxypropylidenehydrazono]thiazolidin-4-one (C-21)

A mixture of 1-(2-isopropylphenyl)-3-[(E)-2-[2-[N-methyl-4-(trifluoromethoxy)anilino]pyrimidin-5-yl]oxypropylideneamino]thiourea (0.1 g), Sodium acetate (0.092 g) and Methyl bromoacetate (0.076 mL) in Ethanol (20 mL) was heated at 40° C. for 12 h. The mixture was cooled to ambient temperature diluted with Water (50 mL) and extracted with Ethyl acetate (2×15 mL). The combined Ethyl acetate extracts were dried over anhydrous Sodium sulphate and the were evaporated invacuo and the resultant residue was subjected to flash column chromatography with Silica gel, eluting with a Ethyl acetate/Heptane gradient wo get the title compound as a yellow solid 0.108 g (70%). LC/MS: Rt: 2.309 min; MS: m/z=573.3 (M+1)⁺; ¹H NMR (300 MHz, DMSO-d6) δ 8.23 (d, J=2.0 Hz, 2H), 7.57 (t, J=5.4 Hz, 1H), 7.52-7.39 (m, 4H), 7.39-7.28 (m, 3H), 7.20 (d, J=7.9 Hz, 1H), 5.03 (q, J=6.1 Hz, 1H), 4.40-3.93 (m, 2H), 3.44 (s, 3H), 1.42 (dd, J=6.4, 1.7 Hz, 3H), 1.16-1.02 (m, 6H).

### Example 22: Synthesis of 1-(2-isopropylphenyl)-3-[(E)-[2-methyl-3-[4-methyl-2-[N-methyl-4-(trifluoromethoxy)anilino]pyrimidin-5-yl]propylidene]amino]thiourea (C-22)

### Step 1

To the solution of (E)-2-methyl-3-[4-methyl-2-[N-methyl-4-(trifluoromethoxy)anilino]pyrimidin-5-yl]prop-2-enal (0.2 g) in Ethyl acetate (5 mL) was added 10% Palladium on charcoal (0.02 g) and the mixture stirred under a Hydrogen atmosphere via a gas bladder for 12 h. The mixture was filtered through a Celite bed and evaporated under reduced pressure, the residue obtained subjected to flash column chromatography to afford the desired product (0.11 g). LC/MS Rt: 2.166 min; MS: m/z=354.4 (M+1)+.

### Step 2: 1-(2-isopropylphenyl)-3-[(E)-[2-methyl-3-[4-methyl-2-[N-methyl-4-(trifluoromethoxy) anilino] pyrimidin-5-yl]propylidene]amino]thiourea

A mixture of 2-methyl-3-[4-methyl-2-[N-methyl-4-(trifluoromethoxy)anilino]pyrimidin-5-yl]propanal (0.1 g) and 1-amino-3-(2-isopropylphenyl)thiourea (0.06 g) in Ethanol (2 mL) was heated at 80° C. for 2 h. The mixture was evaporated invacuo and the residue obtained was subjected to flash column chromatography to afford the desired product (0.13 g, 84%) as a yellow solid. LC/MS Rt: 2.374 min; MS: m/z=545.6 (M+1)⁺; ¹H NMR (300 MHz, DMSO-d6) δ 11.42 (s, 1H), 9.49 (s, 1H), 8.13 (s, 1H), 7.48-7.42 (m, 3H), 7.38-7.14 (m, 6H), 3.46 (s, 3H), 2.91 (ddd, J=47.9, 13.5, 7.0 Hz, 2H), 2.33 (s, 3H).

### Example 23: Synthesis of N-methyl-5-[(E,3E)-2-methyl-3-[(3R,4R,5S,6S)-3,4,5-trimethoxy-6-methyl-tetrahydropyran-2-yl]oxyimino-prop-1-enyl]-N-[4-(trifluoromethoxy)phenyl]pyrimidin-2-amine (C-23)

(E)-2-methyl-3-[2-[N-methyl-4-(trifluoromethoxy)anilino]pyrimidin-5-yl]prop-2-enal (0.17 g), O[(3R,4R,5S,6S)-3,4,5-trimethoxy-6-methyl-tetrahydropyran-2-yl]hydroxylamine (0.123 g) and Concentrated hydrochloric acid solution (1.5 mL) was taken in Ethanol (15 mL) and the mixture heated to 80° C. for 48 h. The mixture was diluted with Ethyl acetate (20 mL) and the organic layer separated, dried over anhydrous Sodium sulphate, evaporated invacuo and the residue subjected to flash column chromatography on Silica gel eluting with a gradient of Ethyl acetate and n-heptane to afford the title compound (0.055 g, 18%). LC/MS: Rt: 2.278 min; MS: m/z=541 (M+1)⁺; ¹H NMR (300 MHz, DMSO-d6) δ 8.51 (s, 1H), 8.09 (s, OH), 7.68-7.21 (m, 2H), 6.71 (s, 1H), 5.37 (dd, J=27.0, 2.0 Hz, 1H), 3.52 (s, 2H), 3.44-3.36 (m, 6H), 2.03 (d, J=1.2 Hz, 2H), 1.83 (d, J=1.4 Hz, 1H), 1.16 (d, J=5.9 Hz, 2H).

### Example 24: Synthesis of (2Z)-3-(2-isopropylphenyl)-2-[(E)-[2-methyl-3-[4-methyl-2-[N-methyl-4-(trifluoromethoxy)anilino]pyrimidin-5-yl]propylidene]hydrazono]thiazolidin-4-one (C-24)

A mixture of 1-(2-isopropylphenyl)-3-[(E)-[2-methyl-3-[4-methyl-2-[N-methyl-4-(trifluoromethoxy) anilino] pyrimidin-5-yl]propylidene]amino]thiourea (0.07 g), Sodium acetate (0.063 g), Methyl bromoacetate (0.04 mL) in Ethanol (10 mL) was stirred at 40° C. for 12 h. The reaction mixture was cooled to ambient temperature, diluted with Water (50 mL), Sodium hydroxide solution (1 N, 2 mL) and extracted with Ethyl acetate (2×50 mL). The combined organic extracts were dried over anhydrous Sodium sulphate, evaporated invacuo and the residue subjected to flash column chromatography on Silic gel, eluting with a gradient of Ethyl acetate and Heptane to obtain the title compound as a white solid 0.05 g (67%). LC/MS: Rt: 2.409 min; MS: m/z=585.4 (M+1)+. 1H NMR (300 MHz, DMSO-d6) δ 8.05 (d, J=8.2 Hz, 1H), 7.54 (dd, J=7.6, 5.3 Hz, 1H), 7.46 (dt, J=7.7, 3.0 Hz, 4H), 7.40-7.23 (m, 3H), 7.27-7.11 (m, 1H), 4.28-3.86 (m, 2H), 3.45 (s, 3H), 2.77-2.61 (m, 1H), 2.27 (d, J=2.5 Hz, 3H), 1.16-0.92 (m, 10H).

### Example 25: Synthesis of 1-(2-isopropylphenyl)-3-[(E)-[(E)-2-methyl-3-[2-[N-methyl-4-(trifluoromethoxy)anilino]pyrimidin-5-yl]prop-2-enylidene]amino]imidazolidine-2,4-dione (C-25)

(E)-2-methyl-3-[2-[N-methyl-4-(trifluoromethoxy)anilino]pyrimidin-5-yl]prop-2-enal (0.227 g) and 3-amino-1-(2-isopropylphenyl)imidazolidine-2,4-dione (0.157 g) were taken up in Ethanol (10 mL. 2 drops of conc. Hydrochloric acid solution was added and the mixture heated to 80° C. for 3 h. The mixture was evaporated invacuo and the residue taken up in Ethyl acetate and washed with a saturated solution of Sodium bicarbonate solution. The organic layer was separated, dried and evaporated to obtain a residue which was subjected to preparative HPLC to get the title compound (0.058 g, 13%), LC/MS: Rt: 2.26 min; MS: m/z=553.8 (M)+.

### Example 26: Synthesis of 1-(2-isopropylphenyl)-3-[(E)-[(E)-2-methyl-3-[6-[N-methyl-4-(trifluoromethoxy)anilino]pyridazin-3-yl]prop-2-enylidene]amino]thiourea (C-26)

### Step 1: Synthesis of 6-chloro-N-[4-(trifluoromethoxy)phenyl]pyridazin-3-amine

A mixture of 3,6 Dichloro pyridazine (0.2 g) and 4-(trifluoromethoxy) aniline (0.180 g) was taken up in in Acetic acid (3 mL) and heated at 90° C. for 4 h. The mixture was cooled to ambient temperature, neutralized with Sodium bicarbonate solution and extracted with Ethyl acetate. The organic layer was dried over anhydrous Sodium sulfate and concentrated under reduced pressure and the residue obtained, was purified by column chromatography using Ethyl acetate and heptane as eluent to offer the desired compound as off-white solid (0.150 g, 51%). LC/MS: Rt: 1.841 min; MS: m/z=290.25 (M+1)⁺; ¹H NMR (300 MHz, DMSO-d6) δ 9.69 (s, 1H), 7.89-7.74 (m, 2H), 7.62 (d, J=9.3 Hz, 1H), 7.35 (d, J=8.6 Hz, 2H), 7.22 (d, J=9.3 Hz, 1H).

### Step 2: Synthesis of 6-chloro-N-methyl-N-[4-(trifluoromethoxy)phenyl]pyridazin-3-amine

To a stirred solution of 6-chloro-N-[4-(trifluoromethoxy)phenyl]pyridazin-3-amine (2.6 g) in dry DMF (35 mL) was added Sodium hydride (0.323 g) at 0° C. and stirred for 10 min. Methyl iodide (2.55 g) was added and the mixture stirred at ambient temperature for 12 h. Saturated ammonium chloride solution was added and the mixture extracted with Ethyl acetate. The Ethyl acetate extracts were dried over anhydrous Sodium sulfate and concentrated under reduced pressure and the residue obtained was subjected to flash column chromatography to get the title compound as a light brown solid. (1.9 g, 70%). LC/MS: Rt: 1.996 min; MS: m/z=304.1 (M+1)⁺; ¹H NMR (300 MHz, DMSO-d6) δ 7.55-7.41 (m, 5H), 6.98 (d, J=9.5 Hz, 1H), 3.47 (s, 3H).

### Step 3: Synthesis of (E)-2-methyl-3-[6-[N-methyl-4-(trifluoromethoxy)anilino]pyridazin-3-yl]prop-2-enal

A mixture of 6-bromo-N-methyl-N-[4-(trifluoromethoxy)phenyl]pyridazin-3-amine (1.8 g), [1,1′-Bis(diphenylphosphino)ferrocene]palladium(II) dichloride (0.434 g), Cesium carbonate (3.9 g), and 2-[(E)-3,3-diethoxy-2-methyl-prop-1-enyl]-4,4,5,5-tetramethyl-1,3,2-dioxaborolane (3 g) was taken up in a mixture of Dioxan (4 mL) and water (16 mL) and degassed with Nitrogen gas for 10 min and subsequently heated at 95° C. for 2 h. The mixture was diluted with 1N HCl solution, neutralized with Sodium bicarbonate solution and filtered through Celite. The filtrate was extracted with Ethyl acetate and the extracts dried over anhydrous Sodium sulfate and evaporated invacuo to obtain a residue which was subjected to Silica gel flash column chromatography to obtain the desired compound as an off-white solid (1 g, 57%). LC/MS: Rt: 2.00 min; MS: m/z=338.5 (M+1)⁺; 1H NMR (300 MHz, DMSO-d6) δ 9.63 (s, 1H), 7.63 (d, J=9.5 Hz, 1H), 7.65-7.23 (m, 5H), 6.97 (d, J=9.5 Hz, 1H), 3.57 (s, 3H), 2.13 (d, J=1.2 Hz, 3H).

### Step 4: Synthesis of 1-(2-isopropylphenyl)-3-[(E)-[(E)-2-methyl-3-[6-[N-methyl-4-(trifluoromethoxy) anilino]pyridazin-3-yl]prop-2-enylidene]amino]thiourea

A mixture of (E)-2-methyl-3-[6-[N-methyl-4-(trifluoromethoxy)anilino]pyridazin-3-yl]prop-2-enal (0.25 g) and 1-amino-3-(2-isopropylphenyl)thiourea (0.150 g) in Methanol (4 mL) was heated at 80° C. for 3 h. The mixture was evaporated, Water and Ethyl acetate added and the ethyl acetate layer separated, dried evaporated invacuo to obtain a residue which was subjected to flash column chromatography to obtain the title compound as a brown solid (0.190 g, 47%). LC/MS: Rt: 2.256 min; MS: m/z=529.3 (M+1,)+; 1H NMR (300 MHz, DMSO-d6) δ 11.77 (s, 1H), 9.74 (s, 1H), 7.99 (s, 1H), 7.79-7.44 (m, 5H), 7.37-7.12 (m, 2H), 6.94 (d, J=9.5 Hz, 1H), 6.79 (s, 1H), 3.54 (s, 3H), 3.19-3.02 (m, 1H), 2.44-2.23 (m, 3H), 1.19 (d, J=6.9 Hz, 6H).

### Example 27: Synthesis of (2Z)-3-(2-isopropylphenyl)-2-[(E)-[(E)-2-methyl-3-[6-[N-methyl-4-(trifluoromethoxy)anilino]pyridazin-3-yl]prop-2-enylidene]hydrazono]thiazolidin-4-one (C-27)

A mixture of 1-(2-isopropylphenyl)-3-[(E)-[(E)-2-methyl-3-[6-[N-methyl-4-(trifluoromethoxy) anilino]pyridazin-3-yl]prop-2-enylidene]amino]thiourea (0.158 g), Sodium acetate (0.049 g), and Methylbromo acetate (0.137 g) in Methanol (4 mL) was stirred for 12 h. The mixture was evaporated invacuo and the residue was subjected to Silica gel column chromatography eluting with a gradient of Dichloromethane and methanol to obtain the title compound as a brown solid (0.09 g, 50%). LC/MS: Rt: 2.261 min; MS: m/z=569.90 (M+1)⁺; ¹H NMR (300 MHz, DMSO-d6) δ 8.08 (s, 1H), 7.55-7.38 (m, 7H), 7.38-7.20 (m, 2H), 6.97-6.87 (m, 2H), 4.29-4.04 (m, 2H), 3.54 (s, 3H), 2.90-2.67 (m, 1H), 2.30 (d, J=1.2 Hz, 3H), 1.14 (t, J=6.7 Hz, 6H).

### Example 28: Synthesis of (2Z)-3-(2-isopropylphenyl)-2-[(E)-2-[6-[N-methyl-4-(trifluoromethoxy)anilino]pyridazin-3-yl]oxypropylidenehydrazono]thiazolidin-4-one (C-28)

### Step 1: Synthesis of N-methyl-6-(1-methylallyloxy)-N-[4-(trifluoromethoxy)phenyl]pyridazin-3-amine

To a stirred solution of 6-chloro-N-methyl-N-[4-(trifluoromethoxy)phenyl]pyridazin-3-amine (1 g) and allyl alcohol (0.475 g) in N, N-Dimethylformamide (15 mL) at 0° C. was added Sodium hydride (0.160 g) and the mixture stirred at ambient temperature for 12 h. Saturated Ammonium chloride solution was subsequently added and the mixture extracted with Ethy acetate. The Ethyl acetate extracts were separated, dried over anhydrous Sodium sulphate and evaporated invacuo, and the residue obtained was subjected to flash column chromatography eluting with a gradient of Ethyl acetate and Heptane to get the desired product as a brown solid (0.56 g, 50%). LC/MS: Rt: 2.193 min; MS: m/z=340.5 (M+1)⁺; ¹H NMR (300 MHz, DMSO-d6) δ 7.38 (s, 4H), 7.06 (d, J=9.6 Hz, 1H), 6.96 (d, J=9.6 Hz, 1H), 6.00 (ddd, J=17.3, 10.6, 5.5 Hz, 1H), 5.69 (dtd, J=7.8, 6.4, 5.1 Hz, 1H), 5.29 (dt, J=17.3, 1.5 Hz, 1H), 5.15 (dt, J=10.6, 1.4 Hz, 1H), 3.42 (s, 3H), 1.40 (d, J=6.5 Hz, 3H).

### Step 2: 2-[6-[N-methyl-4-(trifluoromethoxy)anilino]pyridazin-3-yl]oxypropanal

A mixture of N-methyl-6-(1-methylallyloxy)-N-[4-(trifluoromethoxy)phenyl]pyridazin-3-amine (0.580 g), Osmium tetroxide (0.022 g) in Water (2 mL) and Sodium periodate (1 g) was taken up in a mixture of 1,4 Dioxane (16 mL) and water (2 mL) and the mixture stirred at ambient temperature for 4 h. 2% Sodium sulfite solution was added and the mixture extracted with Ethyl acetate. The Ethyl acetate extracts were dried over anhydrous Sodium sulfate and concentrated under reduced pressure to get a solid residue which was purified by Silica gel flash column chromatography using Ethyl acetate/heptane mixture as eluent to afford the title compound. (0.32 g, 55%). LC/MS: Rt: 1.420 min; MS: m/z=342.4 (M+1)⁺; ¹H NMR (300 MHz, DMSO-d6) δ 9.65 (d, J=1.3 Hz, 1H), 7.39 (d, J=6.7 Hz, 5H), 7.11 (d, J=1.7 Hz, 1H), 3.42 (s, 3H), 3.28 (d, J=2.8 Hz, 1H), 1.44 (d, J=7.1 Hz, 3H).

### Step 3:1-(2-isopropylphenyl)-3-[(E)-2-[6-[N-methyl-4-(trifluoromethoxy)anilino]pyridazin-3-yl]oxypropylideneamino]thiourea

A mixture of (E)-2-methyl-3-[6-[N-methyl-4-(trifluoromethoxy)anilino]-3-pyridyl]prop-2-enal (0.220 g) and 1-amino-3-(2-isopropylphenyl)thiourea (0.150 g) was taken up in Tetrahydrofuran (5 mL) and heated at 50° C. for 4 h. The mixture was diluted with Water and extracted with Ethyl acetate, the Ethyl acetate extracts dried over anhydrous Sodium sulphate and evaporated invacuo and the residue obtained was subjected to Silica gel flash column chromatography to obtain the title compound as a viscous liquid. (0.130 g, 38%). LC/MS: Rt: 2.233 min; MS: m/z=533.3 (M+1)⁺; ¹H NMR (300 MHz, DMSO-d6) δ 11.62 (s, 1H), 9.62 (s, 1H), 7.66 (d, J=4.4 Hz, 1H), 7.47-6.86 (m, 11H), 5.76 (dd, J=6.6, 4.5 Hz, 1H), 3.43 (s, 3H), 3.07-2.93 (m, 1H), 1.56 (d, J=6.5 Hz, 3H), 1.15 (d, J=6.9 Hz, 7H).

### Step 4: Synthesis of (2Z)-3-(2-isopropylphenyl)-2-[(E)-2-[6-[N-methyl-4-(trifluoromethoxy)anilino]pyridazin-3-yl]oxypropylidenehydrazono]thiazolidin-4-one

A mixture of 1-(2-isopropylphenyl)-3-[(E)-2-[6-[N-methyl-4-(trifluoromethoxy)anilino]Pyridazine-3-yl]oxypropylideneamino]thiourea (0.160 g), Sodium acetate (0.050 g), and Methyl bromo acetate (0.138 g) in Methanol (4 mL) was stirred at ambient temperature for 12 h. The reaction mixture was subsequently diluted with Water and extracted with Ethyl acetate, the Ethyl acetate extracts dried over anhydrous Sodium sulphte and evaporated invacuo and the residue subjected to Silica gel flash column chromatography to afford the title compound (0.06 g, 37%). LC/MS: Rt: 2.264 min; MS: m/z=573.4 (M+1)⁺; ¹H NMR (300 MHz, DMSO-d6) δ 7.55 (dd, J=12.8, 4.1 Hz, 1H), 7.47-7.13 (m, 9H), 7.08 (dd, J=7.8, 1.4 Hz, 1H), 7.02-6.77 (m, 2H), 5.62 (ddd, J=6.4, 4.3, 2.0 Hz, 1H), 4.36-3.82 (m, 2H), 3.29 (s, 3H), 2.72-2.55 (m, 1H), 1.36 (d, J=6.6 Hz, 3H), 0.99 (dd, J=6.9, 1.9 Hz, 8H).

### Example 29: Synthesis of (2Z)-3-(2-isopropylphenyl)-2-[(E)-2-[6-[N-methyl-4-(trifluoromethoxy) anilino]pyridazin-3-yl]oxyethylidenehydrazono]thiazolidin-4-one (C-29)

### Step 1: Synthesis of 6-allyloxy-N-methyl-N-[4-(trifluoromethoxy)phenyl]pyridazin-3-amine

To a stirred solution of Allyl alcohol (1 g) in N, N-Dimethylformamide (15 mL) at 0° C. was added Sodium hydride (0.320 g) and the mixture stirred for 20 min. A solution of 6-chloro-N-methyl-N-[4-(trifluoromethoxy)phenyl]pyridazin-3-amine (2.7 g) in N,N-Dimethylformamide (5 mL) was added drop-wise and the mixture stirred for a further 3 h. Saturated Ammonium chloride solution was subsequently added, the mixture extracted with Ethyl acetate, the Ethyl acetate extracts dried over anhydrous Sodium sulphate and evaporated invacuo. The residue obtained was subjected to Silica gel flash column chromatography to obtain the title compound as a white solid (2.5 g, 86%). LC/MS: Rt: 2.086 min; MS: m/z=326.25 (M+1)⁺; ¹H NMR (300 MHz, DMSO-d6) δ 7.38 (d, J=1.5 Hz, 4H), 7.13-6.96 (m, 2H), 6.10 (ddt, J=17.3, 10.7, 5.5 Hz, 1H), 5.40 (dq, J=17.3, 1.7 Hz, 1H), 5.26 (dq, J=10.5, 1.5 Hz, 1H), 4.87 (dt, J=5.5, 1.5 Hz, 2H), 3.43 (s, 3H).

### Step 2: 2-[6-[N-methyl-4-(trifluoromethoxy)anilino]pyridazin-3-yl]oxyacetaldehyde

A mixture of 6-allyloxy-N-methyl-N-[4-(trifluoromethoxy)phenyl]pyridazin-3-amine (0.22 g), Sodium periodate (0.434 g) and Osmium tetraoxide (catalytic) was taken up in 1,4-Dioxane (6 mL) and Water (1 mL) and the mixture stirred at ambient temperature for 12 h. The mixture was subsequently quenched with Sodium sulfite solution and extracted with Ethyl acetate. The Ethyl acetate extracts were dried over anhydrous Sodium sulphate and evaporated invacuo and the residue obtained was subjected to Silica gel flash column chromatography eluting with a gradient of Ethyl acetate and n-Heptane to afford the desired compound as an off-white solid. (0.150 g, 68%). LC/MS: Rt: 1.382 min; MS: m/z=328.15 (M+1)⁺; ¹H NMR (300 MHz, DMSO-d6) δ 7.45-7.31 (m, 4H), 7.03 (q, J=9.6 Hz, 2H), 6.40 (d, J=7.8 Hz, 1H), 4.82 (dt, J=7.8, 5.1 Hz, 1H), 4.21 (h, J=5.7 Hz, 2H), 3.43 (s, 3H), 3.34 (s, 4H), 1.39-1.25 (m, 1H), 1.24 (s, 3H).

### Step 3: Synthesis of 1-(2-isopropylphenyl)-3-[(E)-2-[6-[N-methyl-4-(trifluoromethoxy) anilino]pyridazin-3-yl]oxyethylideneamino]thiourea

A mixture of 2-[6-[N-methyl-4-(trifluoromethoxy)anilino]pyridazin-3-yl]oxyacetaldehyde (0.530 g) and 1-amino-3-(2-isopropylphenyl)thiourea (0.338 g) in Tetrahydrofuran (5 mL) was heated at 50° C. for 3 h. The mixture was diluted with Water and extracted with Ethyl acetate, the Ethyl acetate extracts dried over anhydrous Sodium sulphate and evaporated under reduced pressure. The residue obtained was subjected to Silica gel flash column chromatography using a gradient of Ethyl acetate and Heptane to obtain the title compound as a white solid (0.2 g, 24%). LC/MS: Rt: 2.175 min; MS: m/z=519.3 (M+1)⁺; ¹H NMR (300 MHz, DMSO-d6) δ 11.71 (s, 1H), 9.73 (s, 1H), 7.72 (t, J=5.0 Hz, 1H), 7.39 (s, 4H), 7.37-7.21 (m, 3H), 7.18 (dd, J=3.7, 2.3 Hz, 2H), 7.14-7.03 (m, 2H), 5.04 (d, J=4.9 Hz, 2H), 3.44 (s, 3H), 3.04 (p, J=6.9 Hz, 1H), 1.15 (d, J=6.8 Hz, 6H).

### Step 4: Synthesis of (2Z)-3-(2-isopropylphenyl)-2-[(E)-2-[6-[N-methyl-4-(trifluoromethoxy)anilino]pyridazin-3-yl]oxyethylidenehydrazono]thiazolidin-4-one

A mixture of 1-(2-isopropylphenyl)-3-[(E)-2-[6-[N-methyl-4-(trifluoromethoxy)anilino]pyridazin-3-yl]oxyethylideneamino]thiourea (0.090 g), Sodium acetate (0.029 g) and Methyl bromoacetate (0.080 g) in Tetrahydrofuran (2 mL) was stirred at ambient temperature for 12 h. The mixture was subsequently diluted with Water, extracted with Ethyl acetate, the Ethyl acetate extracts dried over anhydrous Sodium sulphate and evaporated invacuo. The resultant solid was subjected to Silica gel flash column chromatography using Ethyl acetate/Heptane gradient to obtain the title compound as a white solid (0.070 g, 57%). LC/MS Rt: 2.189 min; MS: m/z=559.55 (M+1)⁺; ¹H NMR (300 MHz, DMSO-d6); 1H NMR (300 MHz, DMSO-d6) δ 7.47 (td, J=7.9, 1.7 Hz, 3H), 7.43-7.24 (m, 7H), 7.21 (d, J=7.5 Hz, 1H), 7.05 (q, J=9.3 Hz, 3H), 5.05 (d, J=4.4 Hz, 2H), 4.31-4.02 (m, 3H), 3.41 (d, J=5.3 Hz, 12H), 2.75 (h, J=6.8 Hz, 2H), 1.27-1.07 (m, 9H).

### Example 30: Synthesis of 1-[(E)-2-[[2,4-dimethyl-6-[N-methyl-4-(trifluoromethoxy)anilino]-3-pyridyl]oxy]ethylideneamino]-3-(2-isopropylphenyl)thiourea (C-30)

### Step 1: 5-Brom-4,6-dimethyl-pyridin-2-amine

A mixture of 4,6-dimethylpyridin-2-amine (3 g) and Bromine (1.39 mL) taken up in Acetonitrile (30 mL) was stirred at ambient temperature for 1 h. The mixture was diluted with Water (100 mL) and the precipitate was collected by filteration and dried to afford the title product as a off white solid (3.8 g, 77%). LC/MS: Rt: 1.2 min; MS: m/z=203 (M+1)⁺; ¹H NMR (300 MHz, Chloroform-d) δ 7.28 (s, 1H), 6.37 (s, 2H), 2.43-2.12 (m, 6H).

### Step 2: 5-bromo-4,6-dimethyl-N-[4-(trifluoromethoxy)phenyl]pyridin-2-amine

A mixture of 5-Bromo-4,6-dimethyl-pyridin-2-amine (2 g), Cesium carbonate (6.46 g), 4-Trifluoromethoxy iodobenzene (4.29 g), Palladium (II) acetate (0.22 g, 0.99 mmol) and 4,5-Bis(diphenylphosphino)-9,9-dimethylxanthene (0.57 g) was taken up in Toleune (40 mL) and degassed with nitrogen gas and heated at 120° C. for 5 h. The reaction was diluted with Water (50 mL) and extracted with Ethyl acetate (2×30 mL) and the combined organic extracts were dried over anhydrous Sodium sulphate and evaporated under reduced pressure. The residue was subjected to Silica gel flash column chromatography eluting with a gradient of Ethyl acetate and Heptane to obtain the title compound as an off white solid (2.7 g, 75%). LC/MS: Rt: 2.44 min; MS: m/z=363.2 (M+2)+; ¹H NMR (300 MHz, CDCl3) δ 7.36 (d, J=9.0 Hz, 2H), 7.24 (d, J=8.8 Hz, 2H), 2.66 (s, 3H), 2.39 (s, 3H).

### Step 3: 5-bromo-N,4,6-trimethyl-N-[4-(trifluoromethoxy)phenyl]pyridin-2-amine

To the solution of 5-bromo-4,6-dimethyl-N-[4-(trifluoromethoxy)phenyl]pyridin-2-amine (2.5 g) in N, N-Dimethylformamide (30 mL) at 0° C. was added Sodium hydride (60% dispersion in mineral oil, 0.415 g) portion wise. Methyl iodide (0.7 mL) was added and the mixture stirred at 0° C. for 1 h. The mixture was poured into ice water and extracted with Ethyl acetate (2×30 mL) and the extracts dried over anhydrous Sodium sulphate. The extracts were evaporated invacuo and the residue subjected to Silica gel flash column chromatography eluting with a gradient of Ethyl acetate and Heptane to afford the title compound as an off white solid (2.5 g, 96%). LC/MS: Rt: 2.628 min; MS: m/z=375.2 (M+2)+; ¹H NMR (300 MHz, CDCl3) δ 7.18 (d, J=5.3 Hz, 5H), 6.20 (s, 1H), 3.41 (s, 3H), 2.55 (d, J=2.6 Hz, 3H), 2.16 (s, 3H).

### Step 4: 2,4-dimethyl-6-[N-methyl-4-(trifluoromethoxy)anilino]pyridin-3-ol

A mixture of 5-bromo-N,4,6-trimethyl-N-[4-(trifluoromethoxy)phenyl]pyridin-2-amine (0.5 g), Tris(dibenzylideneacetone)dipalladium(0) (0.12 g), 2-Di-tert-butylphosphino-2′,4′,6′-triisopropylbiphenyl (0.057 g) and Potassium hydroxide (0.15 g) was taken up in 1,4-Dioxan (4 mL) and Water (4 mL) and heated at 90° C. for 2 h. The mixture was cooled to ambient temperature and diluted with Water (50 mL) and extracted with Ethyl acetate (2×30 mL).The combined organic layer was dried over anhydrous Sodium sulphate evaporated invacuo and the residue was subjected to Silica gel flash column chromatography to get the title compound as an off white solid (0.4 g, 96%). LC/MS: Rt: 1.614 min; MS: m/z=313.25 (M+1)⁺; ¹H NMR (300 MHz, DMSO-d6) δ 8.25 (s, 1H), 7.22 (s, 1H), 7.10 (d, J=9.1 Hz, 1H), 6.59 (s, 1H), 3.28 (s, 3H), 2.29 (s, 3H), 2.11 (s, 3H).

### Step 5: 5-(2, 2-diethoxyethoxy)-N,4,6-trimethyl-N-[4-(trifluoromethoxy)phenyl]pyridin-2-amine

A mixture of 2,4-dimethyl-6-[N-methyl-4-(trifluoromethoxy)anilino]pyridin-3-ol (0.3 g), Potassium hydroxide (0.27 g) and Bromoacetaldehyde diethylacetal (0.217 mL) was taken up in N,N-Dimethylacetamide (4 mL) and heated at 100° C. for 1 h. The mixture was subsequently cooled to ambient temperature and Water (50 mL) was added and extracted with Ethyl acetate (2×20 mL). The combined Ethyl acetate extracts were washed with brine, dried over anhydrous Sodium sulphate and evaporated invacuo to get the title compound as a pale yellow oil (0.27 g, 66%). LC/MS: Rt: 2.45 min; MS: m/z=429.3 (M)+; ¹H NMR (DMSO-d6): 1H NMR (300 MHz, DMSO-d6) δ 7.32 (d, J=2.0 Hz, 4H), 6.42 (s, 1H), 4.78 (t, J=5.1 Hz, 1H), 3.79-3.46 (m, 5H), 2.32 (s, 4H), 2.13 (s, 3H), 1.15 (t, J=7.0 Hz, 6H).

### Step 6: 2-[[2,4-dimethyl-6-[N-methyl-4-(trifluoromethoxy)anilino]-3-pyridyl]oxy]acetaldehyde

To the solution of 5-(2,2-diethoxyethoxy)-N,4,6-trimethyl-N-[4-(trifluoromethoxy)phenyl] pyridin-2-amine (0.25 g) in Acetone (5 mL) was added a solution of Hydrochloric acid (1 mL). The mixture was heated at 70° C. for 2 h. The mixture was subsequently basified with sat. Sodium bicarbonate solution and extracted with Ethyl acetate (2×20 mL). The combined Ethyl acetate extracts were dried over anhydrous Sodium sulphate and evaporated invacuo to get the title compound as a pale yellow oil (0.2 g, 96%). LC/MS: Rt: 1.56 min; MS: m/z=355 (M)+; 1H NMR (DMSO-d6): ¹H NMR (300 MHz, DMSO-d6) δ 9.70 (s, OH), 7.32 (d, J=5.0 Hz, 8H), 6.43 (d, J=5.5 Hz, 1H), 5.18 (s, OH), 4.55 (s, 1H), 2.30 (d, J=6.2 Hz, 3H), 2.17-1.91 (m, 3H).

### Step 7: 1-[(E)-2-[[2,4-dimethyl-6-[N-methyl-4-(trifluoromethoxy)anilino]-3-pyridyl]oxy]ethylideneamino]-3-(2-isopropylphenyl)thiourea

A mixture of 2-[[2,4-dimethyl-6-[N-methyl-4-(trifluoromethoxy)anilino]-3-pyridyl]oxy]acetaldehyde (0.15 g) and 1-amino-3-(2-isopropylphenyl)thiourea (0.09 g) in Ethanol (4 mL) was heated at 80° C. for 2 h. The mixture was evaporated under reduced pressure and the residue obtained was subjected to Silica gel flash column chromatography eluting with a gradient of Ethyl acetate and Heptane to obtain the title compound as a white solid. (0.16 g, 69%). LC/MS: Rt: 2.37 min; MS: m/z=546.3 (M)+; ¹H NMR (DMSO-d6) δ 11.69 (s, 1H), 9.75 (s, 1H), 7.70 (t, J=5.4 Hz, 1H), 7.39-7.07 (m, 7H), 6.44 (s, 1H), 4.48 (d, J=5.4 Hz, 2H), 2.34 (s, 3H), 2.15 (s, 3H), 1.14 (d, J=6.8 Hz, 6H).

### Example 31: Synthesis of (2Z)-2-[(E)-2-[[2,4-dimethyl-6-[N-methyl-4-(trifluoromethoxy)anilino]-3-pyridyl]oxy]ethylidenehydrazono]-3-(2-isopropylphenyl)thiazolidin-4-one (C-31)

A mixture of 1-[(E)-2-[[2,4-dimethyl-6-[N-methyl-4-(trifluoromethoxy)anilino]-3-pyridyl]oxy]ethylideneamino]-3-(2-isopropylphenyl)thiourea (0.1 g), Sodium acetate (0.09 g) and Methyl bromoacetate (0.056 mL) in Ethanol (4 mL) was stirred at 40° C. for 12 h. The mixture was cooled to ambient temperature and diluted with Water (50 mL) and a solution of Sodium hydroxide (1 N, 1 mL) and extracted with Ethyl acetate (2×50 mL). The combined Ethyl acetate extracts were dried over anhydrous Sodium sulphate, evaporated invacuo and the residue subjected to Silica gel flash column chromatography to get the title compound as a yellow solid (0.042 g, 39%). LC/MS: Rt: 2.41 min; MS: m/z=586.4 (M+1)⁺; ¹H NMR (300 MHz, DMSO-d6) δ 7.75 (s, 1H), 7.48 (s, 1H), 7.32 (s, 7H), 7.24 (s, 1H), 6.42 (s, 2H), 4.67-4.37 (m, 4H), 4.16 (d, J=20.4 Hz, 3H), 2.28 (s, 8H), 2.10 (s, 6H), 1.12 (dd, J=6.9, 5.0 Hz, 12H).

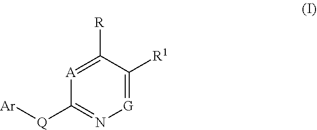

TABLE C

| No. | Ar | Q | A | G | R | R¹ | M/z | Rt [min |
|---|---|---|---|---|---|---|---|---|
| C-1 | 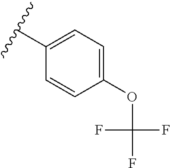 [SMILES: *c1ccc(OC(F)(F)F)cc1] | NH | N | CH | H | 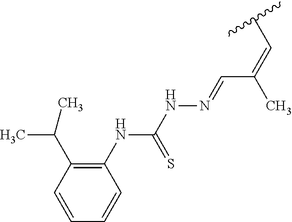 [SMILES: */C=C(C)\C=N\NC(=S)Nc1ccccc1C(C)C] | 515 | 1.95 |
| C-2 | 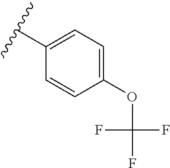 [SMILES: *c1ccc(OC(F)(F)F)cc1] | NH | N | CH | H | 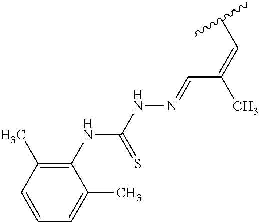 [SMILES: */C=C(C)\C=N\NC(=S)Nc1c(C)cccc1C] | 501 | 1.89 |
| C-3 | 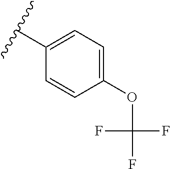 [SMILES: *c1ccc(OC(F)(F)F)cc1] | NH | N | CH | H | 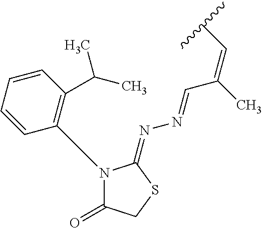 [SMILES: *\C=C(C)/C=N/N=C1\SCC(=O)N1Cc1ccccc1C(C)C] | 555 | 1.99 |
| C-4 | 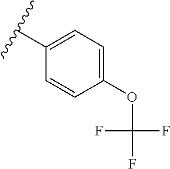 [SMILES: *c1ccc(OC(F)(F)F)cc1] | NH | N | CH | H | 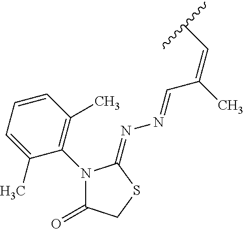 [SMILES: *\C=C(C)/C=N/N=C1\SCC(=O)N1c1c(C)cccc1C] | 541 | 1.95 |
| C-5 | 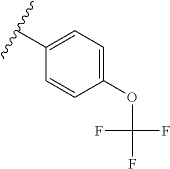 [SMILES: *c1ccc(OC(F)(F)F)cc1] | NCH₃ | N | CH | H | 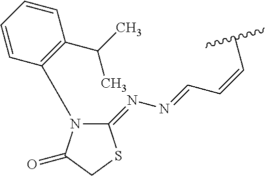 [SMILES: *\C=C/C=N/N=C1\SCC(=O)N1Cc1ccccc1C(C)C] | 555 | 1.97 |
| C-6 | 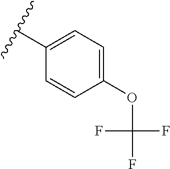 [SMILES: *c1ccc(OC(F)(F)F)cc1] | NCH₃ | N | CH | H | 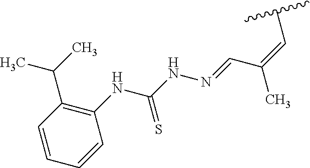 [SMILES: */C=C(C)\C=N\NC(=S)Nc1ccccc1C(C)C] | 529.3 | 2.37 |
| C-7 | 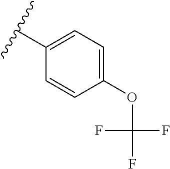 [SMILES: *c1ccc(OC(F)(F)F)cc1] | NCH₃ | N | CH | H | 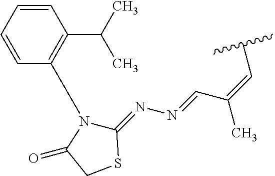 [SMILES: *\C=C(C)/C=N/N=C1\SCC(=O)N1Cc1ccccc1C(C)C] | 569.4 | 2.49 |
| C-8 | 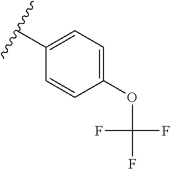 [SMILES: *c1ccc(OC(F)(F)F)cc1] | NCH₃ | C—N(CH₃)₂ | CH | H | 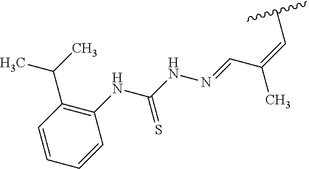 [SMILES: */C=C(C)\C=N\NC(=S)Nc1ccccc1C(C)C] | 571.4 | 2.498 |
| C-9 | 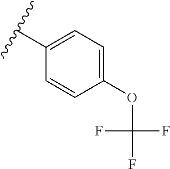 [SMILES: *c1ccc(OC(F)(F)F)cc1] | NCH₃ | C—N(CH₃)₂ | CH | H | 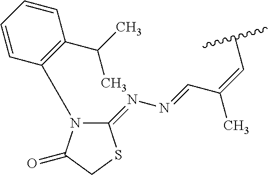 [SMILES: *\C=C(C)/C=N/N=C1\SCC(=O)N1Cc1ccccc1C(C)C] | 611.9 | 2.576 |
| C-10 | 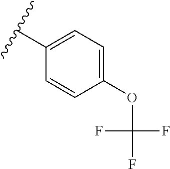 [SMILES: *c1ccc(OC(F)(F)F)cc1] | NCH₃ | N | CH | H | 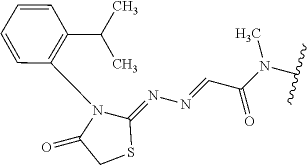 [SMILES: *N(C)C(=O)/C=N/N=C1\SCC(=O)N1Cc1ccccc1C(C)C] | 586.3 | 2.102 |
| C-11 | 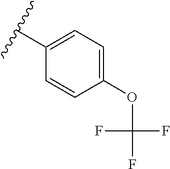 [SMILES: *c1ccc(OC(F)(F)F)cc1] | NCH₃ | N | CH | H | 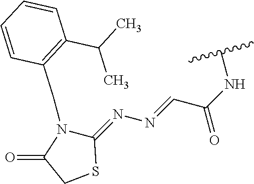 [SMILES: *NC(=O)/C=N/N=C1\SCC(=O)N1Cc1ccccc1C(C)C] | 572.3 | 2.149 |
| C-12 | 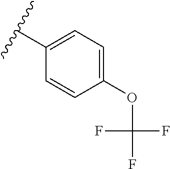 [SMILES: *c1ccc(OC(F)(F)F)cc1] | NCH₃ | CH | CH | H | 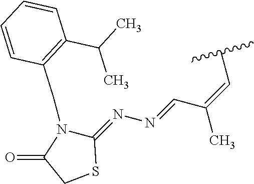 [SMILES: *\C=C(C)/C=N/N=C1\SCC(=O)N1Cc1ccccc1C(C)C] | 568.4 | 2.465 |
| C-13 | 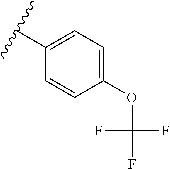 [SMILES: *c1ccc(OC(F)(F)F)cc1] | NCH₃ | N | CH | H | 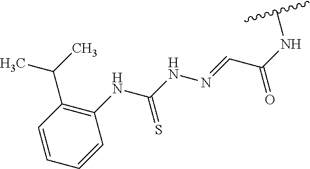 [SMILES: *NC(=O)/C=N/NC(=S)Nc1ccccc1C(C)C] | 532.3 | 2.134 |
| C-14 | 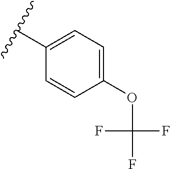 [SMILES: *c1ccc(OC(F)(F)F)cc1] | NCH₃ | N | CH | H | 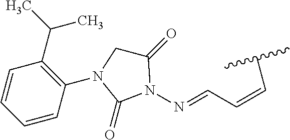 [SMILES: */C=C\C=N\N1C(=O)CN(c2ccccc2C(C)C)C1=O] | 539 | 2.18 |
| C-15 | 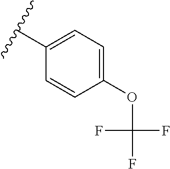 [SMILES: *c1ccc(OC(F)(F)F)cc1] | NCH₃ | CH | CH | H | 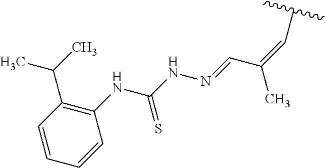 [SMILES: */C=C(C)\C=N\NC(=S)Nc1ccccc1C(C)C] | 528.75 | 2.396 |
| C-16 | 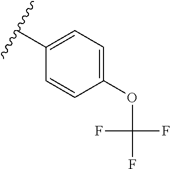 [SMILES: *c1ccc(OC(F)(F)F)cc1] | NCH₃ | N | CH | CH₃ | 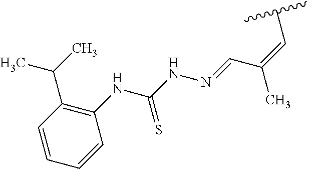 [SMILES: */C=C(C)\C=N\NC(=S)Nc1ccccc1C(C)C] | 543.7 | 2.411 |
| C-17 | 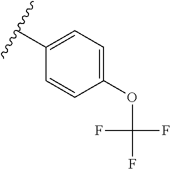 [SMILES: *c1ccc(OC(F)(F)F)cc1] | NCH₃ | N | CH | CH₃ | 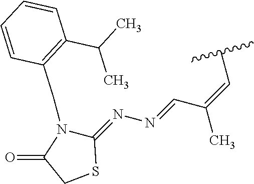 [SMILES: *\C=C(C)/C=N/N=C1\SCC(=O)N1Cc1ccccc1C(C)C] | 583.4 | 2.498 |
| C-18 | 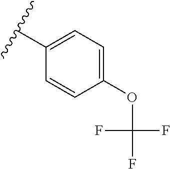 [SMILES: *c1ccc(OC(F)(F)F)cc1] | NCH₃ | N | CH | H | 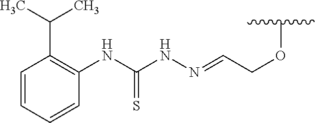 [SMILES: *OC/C=N/NC(=S)Nc1ccccc1C(C)C] | 519.65 | 2.187 |
| C-19 | 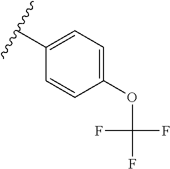 [SMILES: *c1ccc(OC(F)(F)F)cc1] | NCH₃ | N | CH | H | 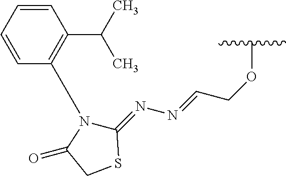 [SMILES: *OC/C=N/N=C1\SCC(=O)N1Cc1ccccc1C(C)C] | 559.3 | 2.25 |
| C-20 | 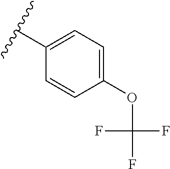 [SMILES: *c1ccc(OC(F)(F)F)cc1] | NCH₃ | N | CH | H | 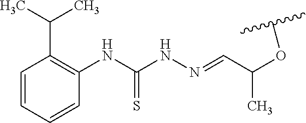 [SMILES: *OC(C)/C=N/NC(=S)Nc1ccccc1C(C)C] | 533.3 | 2.275 |
| C-21 | 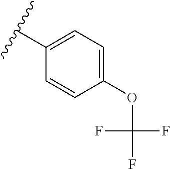 [SMILES: *c1ccc(OC(F)(F)F)cc1] | NCH₃ | N | CH | H | 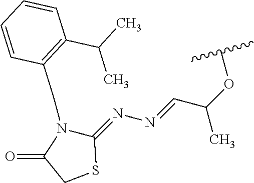 [SMILES: *OC(C)/C=N/N=C1\SCC(=O)N1Cc1ccccc1C(C)C] | 573.3 | 2.309 |
| C-22 | 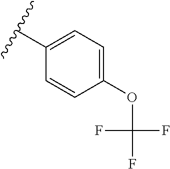 [SMILES: *c1ccc(OC(F)(F)F)cc1] | NCH₃ | N | CH | CH₃ | 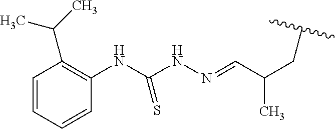 [SMILES: *CC(C)/C=N/NC(=S)Nc1ccccc1C(C)C] | 545.65 | 2.374 |
| C-23 | 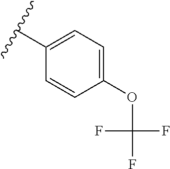 [SMILES: *c1ccc(OC(F)(F)F)cc1] | NCH₃ | N | CH | H | 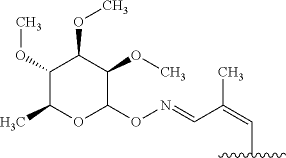 [SMILES: */C=C(C)\C=N\OC1O[C@@H](C)[C@H](OC)[C@@H](OC)[C@H]1OC] | 541 | 2.3 |
| C-24 | 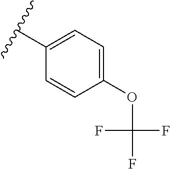 [SMILES: *c1ccc(OC(F)(F)F)cc1] | NCH₃ | N | CH | CH₃ | 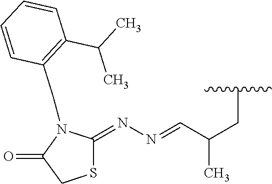 [SMILES: *CC(C)/C=N/N=C1\SCC(=O)N1Cc1ccccc1C(C)C] | 585.4 | 2.409 |
| C-25 | 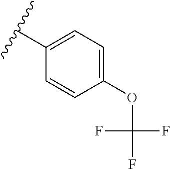 [SMILES: *c1ccc(OC(F)(F)F)cc1] | NCH₃ | N | CH | H | 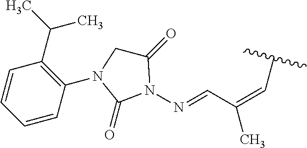 [SMILES: */C=C(C)\C=N\N1C(=O)CN(c2ccccc2C(C)C)C1=O] | 553.8 | 2.26 |
| C-26 | 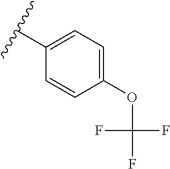 [SMILES: *c1ccc(OC(F)(F)F)cc1] | NCH₃ | CH | N | H | 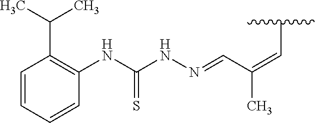 [SMILES: */C=C(C)\C=N\NC(=S)Nc1ccccc1C(C)C] | 529.3 | 2.256 |
| C-27 | 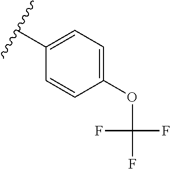 [SMILES: *c1ccc(OC(F)(F)F)cc1] | NCH₃ | CH | N | H | 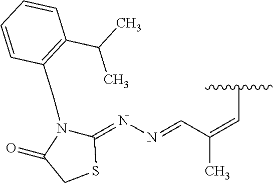 [SMILES: *\C=C(C)/C=N/N=C1\SCC(=O)N1Cc1ccccc1C(C)C] | 569.9 | 2.261 |
| C-28 | 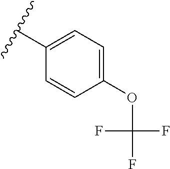 [SMILES: *c1ccc(OC(F)(F)F)cc1] | NCH₃ | CH | N | H | 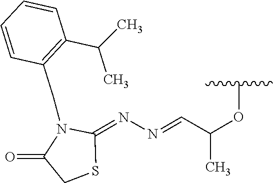 [SMILES: *OC(C)/C=N/N=C1\SCC(=O)N1Cc1ccccc1C(C)C] | 573.4 | 2.264 |
| C-29 | 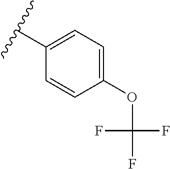 [SMILES: *c1ccc(OC(F)(F)F)cc1] | NCH₃ | CH | N | H | 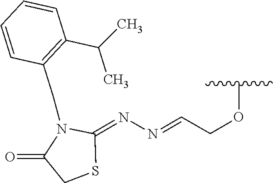 [SMILES: *OC/C=N/N=C1\SCC(=O)N1Cc1ccccc1C(C)C] | 559.55 | 2.189 |
| C-30 | 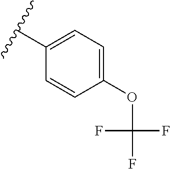 [SMILES: *c1ccc(OC(F)(F)F)cc1] | NCH₃ | CH | C—CH₃ | CH₃ | 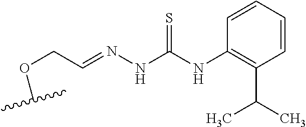 [SMILES: *OC/C=N/NC(=S)Nc1ccccc1C(C)C] | 546.3 | 2.37 |
| C-31 | 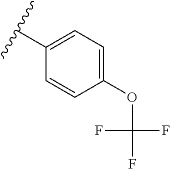 [SMILES: *c1ccc(OC(F)(F)F)cc1] | NCH₃ | CH | C—CH₃ | CH₃ | 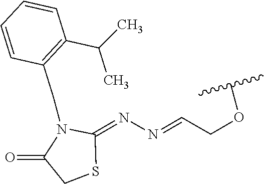 [SMILES: *OC/C=N/N=C1\SCC(=O)N1Cc1ccccc1C(C)C] | 586.4 | 2.419 |

### BIOLOGICAL EXAMPLES

### Example B1: Action on Yellow Fever Mosquito (Aedes aegypti)

For evaluating control of yellow fever mosquito (Aedes aegypti) the test unit consisted of 96-well-microtiter plates containing 200 μl of tap water per well and 5-15 freshly hatched A. aegypti larvae.

The active compounds were formulated using a solution containing 75% (v/v) water and 25% (v/v) DMSO. Different concentrations of formulated compounds or mixtures were sprayed onto the insect diet at 2.5 μl, using a custom built micro atomizer, at two replications.

After application, microtiter plates were incubated at 28±1° C., 80±5% RH for 2 days. Larval mortality was then visually assessed.

In this test, compounds C-1, C-2, C-3, C-4, C-5, C-6, C-7, C-9, C-12, C-16, C-17, C-23, C-27, and C-31 at 800 ppm showed at least 75% mortality in comparison with untreated controls.

### Example B2: Action on Orchid thrips (Dichromothrips corbetti)

Dichromothrips corbetti adults used for bioassay were obtained from a colony maintained continuously under laboratory conditions. For testing purposes, the test compound is diluted in a 1:1 mixture of acetone:water (vol:vol), plus Kinetic® HV at a rate of 0.01% v/v.

Thrips potency of each compound was evaluated by using a floral-immersion technique. All petals of individual, intact orchid flowers were dipped into treatment solution and allowed to dry in Petri dishes. Treated petals were placed into individual re-sealable plastic along with about 20 adult thrips. All test arenas were held under continuous light and a temperature of about 28° C. for duration of the assay. After 3 days, the numbers of live thrips were counted on each petal. The percent mortality was recorded 72 hours after treatment.

In this test, compounds C-1, C-3, C-5, C-6, C-7, C-9, C-12, C-14, C-15, C-16, C-17, C-23, C-26, C-27, and at 500 ppm showed at least 75% mortality in comparison with untreated controls.

### Example B3: Action on Boll Weevil (Anthonomus grandis)

For evaluating control of boll weevil (Anthonomus grandis) the test unit consisted of 96-well-microtiter plates containing an insect diet and 5-10 A. grandis eggs.

The compounds were formulated using a solution containing 75% v/v water and 25% v/v DMSO. Different concentrations of formulated compounds were sprayed onto the insect diet at 5 μl, using a custom built micro atomizer, at two replications.

After application, microtiter plates were incubated at about 25±1° C. and about 75±5% relative humidity for 5 days. Egg and larval mortality was then visually assessed.

In this test, compounds C-1, C-2, C-3, C-4, C-5, C-6, C-7, C-8, C-9, C-12, C-14, C-15, C-16, C-17, C-22, C-23, C-26, C-27, and C-31 at 800 ppm showed at least 75% mortality in comparison with untreated controls.

### Example B4: Action on Silverleaf Whitefly (Bemisia argentifoli) (Adults)

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 5 or 10 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and about 10 to 12 whitefly adults (approximately 3-5 days old) were introduced. The insects were collected using an aspirator and a nontoxic Tygon® tubing connected to a barrier pipette tip. The tip, containing the collected insects, was then gently inserted into the soil containing the treated plant, allowing insects to crawl out of the tip to reach the foliage for feeding. Cups were covered with a reusable screened lid. Test plants were maintained in a growth room at about 250C and about 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, compounds C-3, C-5, C-6, C-7, C-8, C-12, C-15, C-16, C-17, C-22, C-23, and C-26 at 300 ppm showed at least 75% mortality in comparison with untreated controls.

### Example B5: Action on Tobacco Budworm (Heliothis virescens)

For evaluating control of tobacco budworm (Helliothis virescens) the test unit consisted of 96-well-microtiter plates containing an insect diet and 15-25 H. virescens eggs.

The compounds were formulated using a solution containing 75% v/v water and 25% v/v DMSO. Different concentrations of formulated compounds were sprayed onto the insect diet at 10 μl, using a custom built micro atomizer, at two replications.

After application, microtiter plates were incubated at about 28±1° C. and about 80±5% relative humidity for 5 days. Egg and larval mortality was then visually assessed.

In this test, compounds C-1, C-2, C-3, C-4, C-5, C-6, C-7, C-8, C-9, C-10, C-11, C-12, C-13, C-14, C-15, C-16, C-17, C-22, C-23, C-24, C-26, C-27, C-30, and C-31 at 800 ppm showed at least 75% mortality in comparison with untreated controls.

### Example B6: Action on Diamond Back Moth (Plutella xylostella)

The active compound is dissolved at the desired concentration in a mixture of 1:1 (vol:vol) distilled water:acetone. Surfactant (Kinetic® HV) is added at a rate of 0.01% (vol/vol).The test solution is prepared at the day of use.

Leaves of cabbage were dipped in test solution and air-dried. Treated leaves were placed in petri dishes lined with moist filter paper and inoculated with ten 3^{rd} instar larvae. Mortality was recorded 72 hours after treatment. Feeding damages were also recorded using a scale of 0-100%.

In this test, compounds C-1, C-2, C-3, C-4, C-5, C-6, C-7, C-8, C-9, C-10, C-11, C-12, C-13, C-14, C-15, C-16, C-17, C-18, C-19, C-20, C-21, C-23, C-24, C-26, C-27, C-28, C-29, C-30, and C31 at 500 ppm showed at least 75% mortality in comparison with untreated controls.

### Example B7: Action on Southern Armyworm (Spodoptera eridania), 2nd Instar Larvae

The active compounds were formulated by a Tecan liquid handler in 100% cyclohexanone as a 10,000 ppm solution supplied in tubes. The 10,000 ppm solution was serially diluted in 100% cyclohexanone to make interim solutions. These served as stock solutions for which final dilutions were made by the Tecan in 50% acetone:50% water (v/v) into 10 or 20 ml glass vials. A nonionic surfactant (Kinetic®) was included in the solution at a volume of 0.01% (v/v). The vials were then inserted into an automated electrostatic sprayer equipped with an atomizing nozzle for application to plants/insects.

Lima bean plants (variety Sieva) were grown 2 plants to a pot and selected for treatment at the 1^{st} true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 250C and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds C-1, C-2, C-3, C-4, C-5, C-6, C-7, C-8, C-9, C-10, C-11, C-12, C-13, C-15, C-16, C-17, C-18, C-19, C-23, C-26, C-27, and C-31 at 300 ppm showed at least 75% mortality in comparison with untreated controls.

1-15. (canceled)

16. A compound of the formula I 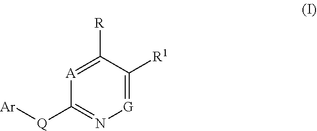
wherein
A is N or CR^{A};
G is N or CR^{B};
R, R^{A}, and R^{B} are H, halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₁-C₄-alkyl-C₃-C₆-cycloalkoxy, which are unsubstituted or substituted with halogen,
C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘR^{e}, one radical may also be phenyl, phenoxy, phenylcarbonyl, phenylthio, or benzyl, wherein the rings are unsubstituted or substituted with R^{f};
Q is NR², O, or S(═O)m, wherein
R² is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen,
C(O)—OR^{a}, C₁-C₆-alkylen-NR^{b}R^{c}, C₁-C₆-alkylen-CN,
C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
Ar is phenyl or 5- or 6-membered hetaryl, which are unsubstituted or substituted with R^{Ar}, wherein
R^{Ar} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which are unsubstituted or substituted with halogen,
C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘR^{e}, one radical may also be phenyl, phenoxy, phenylcarbonyl, phenylthio or benzyl, where the rings are unsubstituted or substituted with R^{f};
R¹ is a moiety of formula X—Y—Z-T-R″ or X—Y—Z-T-R¹²; wherein
X is —CR^{xa}R^{xb}—, —O—, —S—, —NR^{xc}—, —CR^{xa}═CR^{xb}—, —CR^{xa}R^{xb}—CR^{xa}R^{xb}—, —O—CR^{xa}R^{xb}—, —S—CR^{xa}R^{xb}—, —N═CR^{xa}—, —NR^{xc}—CR^{xa}R^{xb}—, —NR^{xc}—C(═S)—, —N═C(S—R^{e})—, or —NR^{xc}—C(═O)—;
Y is —CR^{ya}═N—, wherein the N is bound to Z;
—NR^{yc}—C(═O)—, wherein C(═O) is bound to Z; or
—NR^{yc}—C(═S)—, wherein C(═S) is bound to Z;
Z is a single bond;
—NR^{zc}—C(═S)—, wherein C(═S) is bound to T;
—NR^{zc}—C(═O)—, wherein C(═O) is bound to T;
—N═C(S—R^{za})—, wherein T is bound to the carbon atom;
—O—C(═O)—, wherein T is bound to the carbon atom;
—O—C(═S)—, wherein T is bound to the carbon atom; or
—NR^{zc}—C(S—R^{za})═, wherein T is bound to the carbon atom;
T is O, N or N—R^{T};
R¹¹ is C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₁-C₄-alkyl-C₃-C₆-cycloalkoxy, which are unsubstituted or substituted with halogen,
C₁-C₆-alkylen-NR^{b}R^{c}, C₁-C₆-alkylen-CN, C(O)—NR^{b}R^{c}, C(O)—R^{d}, aryl, arylcarbonyl, aryl-C₁-C₄-alkyl, aryloxy-C₁-C₄-alkyl, hetaryl, carbonyl-hetaryl, hetaryl-C₁-C₄-alkyl or hetaryloxy-C₁-C₄-alkyl, where the rings are unsubstituted or substituted with R^{g} and wherein the hetaryl is a 5- or 6-membered monocyclic hetaryl or a 8-, 9- or 10-membered bicyclic hetaryl;
R¹² is a radical of the formula A¹; 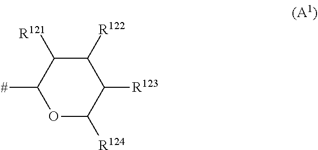
wherein # indicates the point of attachment to T;
R¹²¹, R¹²², R¹²³ are H, halogen, C₁-C₆-alkyl, C₁-C₆-haloalkyl, C₂-C₆-alkenyl, C₂-C₆-alkynyl, C₁-C₆-alkoxy-C₁-C₄-alkyl, C₁-C₆-alkoxy, C₁-C₆-haloalkoxy, C₂-C₆-alkenyloxy, C₂-C₆-alkynyloxy, C₁-C₆-alkoxy-C₁-C₄-alkoxy, C₁-C₆-alkylcarbonlyoxy, C₁-C₆-haloalkylcarbonlyoxy, C₁-C₆-alkenylcarbonlyoxy, C₃-C₆-cycloalkylcarbonlyoxy, or NR^{b}R^{c}, or one of R¹²¹, R¹²², R¹²³ may also be oxo;
R¹²⁴ is H, C₁-C₆-alkyl, C₁-C₆-haloalkyl, C₁-C₆-alkoxy-C₁-C₄-alkyl, C₁-C₆-alkoxy, C₁-C₆-haloalkoxy, or C₂-C₆-alkenyloxy;
and wherein
R^{xa}, R^{xb}, R^{ya} are H, halogen, C₁-C₆-alkyl, C₁-C₆-alkoxy, C₁-C₆-haloalkyl, C₁-C₆-haloalkoxy, C₂-C₆-alkenyl, C₂-C₆-alkynyl, C₁-C₆-alkoxy-C₁-C₄-alkyl, C₃-C₆-cycloalkyl, C₁-C₄-alkyl-C₃-C₆-cycloalkyl, C₁-C₄-alkyl-C₃-C₆-cycloalkoxy, which are unsubstituted or substituted with halogen, C(O)—OR^{a}, C₁-C₆-alkylen-NR^{b}R^{c}, C₁-C₆-alkylen-CN, C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
R^{xc}, R^{yc}, R^{zc} are H, C₁-C₆-alkyl, C₁-C₆-haloalkyl, C₂-C₆-alkenyl, C₂-C₆-alkynyl, C₁-C₄-alkyl-C₁-C₆-alkoxy, C₃-C₆-cycloalkyl, C₁-C₄-alkyl-C₃-C₆-cycloalkyl, or C₁-C₄-alkyl-C₃-C₆-cycloalkoxy, which are unsubstituted or substituted with halogen;
R^{T} is H, C₁-C₆-alkyl, C₁-C₆-haloalkyl, C₂-C₆-alkenyl, C₂-C₆-alkynyl, C₁-C₄-alkyl-C₁-C₆-alkoxy, C₃-C₆-cycloalkyl, C₃-C₆-cycloalkyl-C₁-C₄-alkyl, C₃-C₆-cycloalkoxy-C₁-C₄-alkyl, where the alkyl, which are unsubstituted or substituted with halogen,
C(O)—OR^{a}, C₁-C₆-alkylen-NR^{b}R^{c}, C₁-C₆-alkylen-CN, C(O)—NR^{b}R^{c}, C(O)—R^{d}, SO₂NR^{b}R^{c}, S(═O)ₘR^{e}, phenyl, or benzyl, wherein the rings are unsubstituted or substituted with R^{f};
R^{zc} together with R^{T} if present, may form C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene may be unsubstituted or substituted with R^{h};
R^{za} is H, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₄-alkyl-C₁-C₆-alkoxy, C₃-C₆-cycloalkyl, C₁-C₄-alkyl-C₃-C₆-cycloalkoxy, C₁-C₄-alkyl-C₃-C₆-cycloalkyl, which are unsubstituted or substituted with halogen,
C₁-C₆-alkylen-NR^{b}R^{c}, C₁-C₆-alkylen-CN, C(O)—NR^{b}R^{c}, C(O)—R^{d}, phenyl, phenylcarbonyl, or benzyl, wherein the rings are unsubstituted or substituted with R^{f};
R^{za} together with R^{T} if present, may form C₁-C₆-alkylene or a linear C₂-C₆-alkenylene group, where in the linear C₁-C₆-alkylene and the linear C₂-C₆-alkenylene a CH₂ moiety may be replaced by a carbonyl or a C═N—R′ and/or wherein 1 or 2 CH₂ moieties may be replaced by O or S and/or wherein the linear C₁-C₆-alkylene and the linear C₂-C₆-alkenylene may be unsubstituted or substituted with R^{h};
R^{a}, R^{b} and R^{c} independently of each other are H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen, C₁-C₆-alkylen-CN, phenyl, or benzyl, wherein the rings are unsubstituted or substituted with R^{f};
R^{d} is H, C₁-C₆-alkyl, C₁-C₆-haloalkyl, C₂-C₆-alkenyl, C₂-C₆-alkynyl, C₁-C₆-alkoxy-C₁-C₄-alkyl, C₃-C₆-cycloalkyl, C₃-C₆-cycloalkyl-C₁-C₄-alkyl, C₃-C₆-cycloalkoxy-C₁-C₄-alkyl, which are unsubstituted or substituted with halogen,
phenyl, or benzyl, wherein the rings are unsubstituted or substituted with R^{f};
R^{e} is C₁-C₆-alkyl, C₁-C₆-haloalkyl, C₃-C₆-cycloalkyl, C₃-C₆-cycloalkyl-C₁-C₄-alkyl, which are unsubstituted or substituted with halogen, phenyl and benzyl, wherein the rings are unsubstituted or substituted with R^{f};
R^{f} is halogen, N₃, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxyx-C₁-C₄-alkyl, which are unsubstituted or substituted with halogen,
C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘR^{e};
R^{g} is halogen, N3, OH, CN, NO₂, —SCN, —SF₅, C₁-C₆-alkyl, C₁-C₆-haloalkyl, C₁-C₆-alkoxy, C₁-C₆-haloalkoxy, C₂-C₆-alkenyl, tri-C₁-C₆-alkylsilyl, C₂-C₆-alkynyl, C₁-C₆-alkoxy-C₁-C₄-alkyl, C₁-C₆-alkoxy-C₁-C₄-alkoxy, C₃-C₆-cycloalkyl, C₃-C₆-cycloalkoxy, C₃-C₆-cycloalkyl-C₁-C₄-alkyl, C₃-C₆-cycloalkoxy-C₁-C₄-alkyl, which are unsubstituted or substituted with halogen,
C(O)—OR^{a}, NR^{b}R^{c}, C₁-C₆-alkylen-NR^{b}R^{c}, O—C₁-C₆-alkylen-NR^{b}R^{c}, C₁-C₆-alkylen-CN, NH—C₁-C₆-alkylen-NR^{b}R^{c}, C(O)—NR^{b}R^{c}, C(O)—R^{d}, SO₂NR^{b}R^{c}, or S(═O)ₘRe;
R^{h} is halogen, OH, C₁-C₆-alkyl, C₃-C₆-cycloalkyl, or CN;
m is 0, 1, or 2;
or an N-oxide, stereoisomer, tautomer or an agriculturally or veterinarily acceptable salt thereof.

17. The compound of claim 16, wherein A is CR^{A} and G is N.

18. The compound of claim 16, wherein A is N and G is CR^{B}.

19. The compound of claim 16, wherein A is N and G is N.

20. The compound of claim 16, wherein A is CR^{A} and G is CR^{B}.

21. The compound of claim 16, wherein Q is NR²

22. The compound of claim 16, wherein X—Y—Z-T are formulas XYZT-1 to XYZT-19 wherein 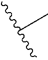
denotes attachment to the 6 membered hetaryl and # denotes attachment to R¹¹ or R¹²;

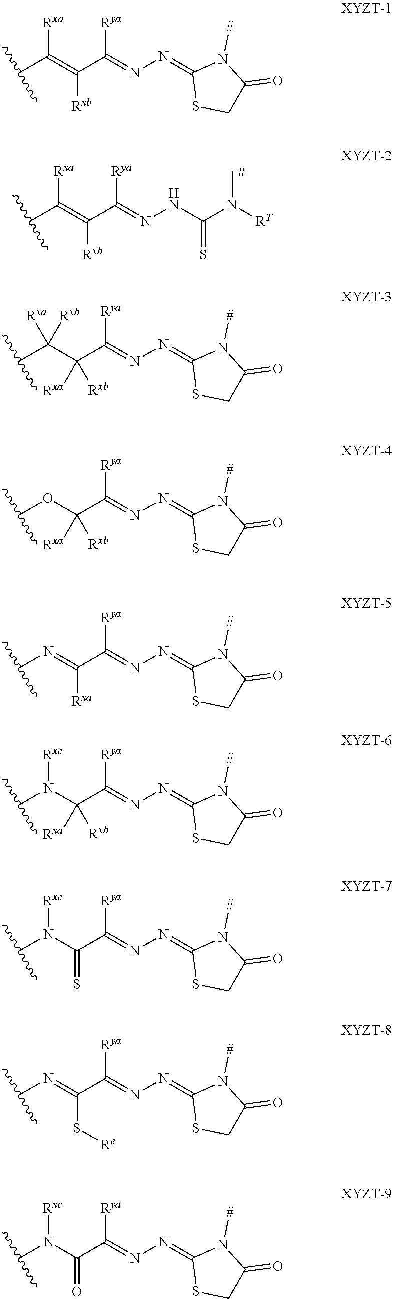

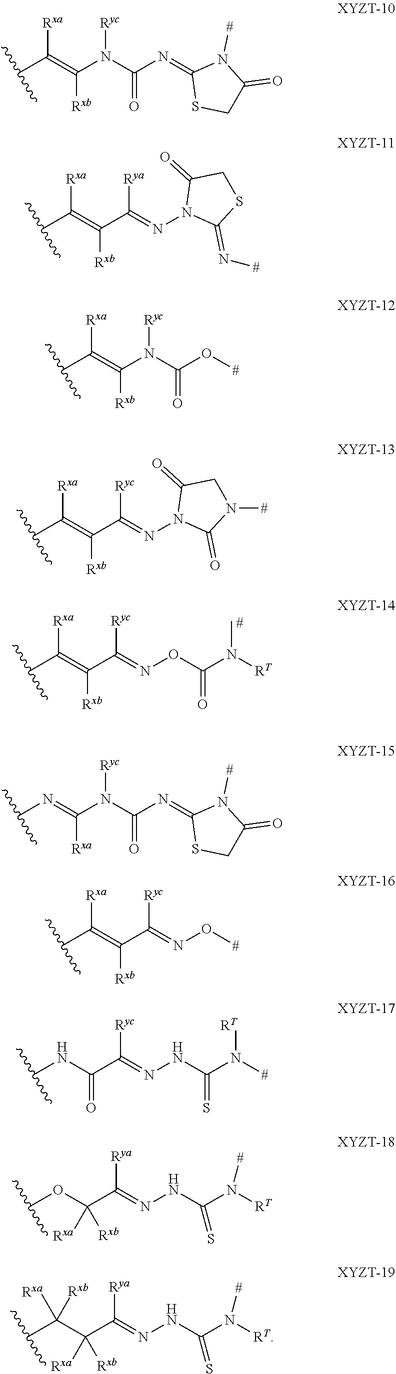

23. The compound of claim 16, wherein
A is N or CR^{A};
G is N or CR^{B};
Q is NH or NCH₃;
R is H or C₁-C₆-alkyl;
R^{A} is H or N(CH₃)₂;
R^{B} is H or CH₃;
Ar is Ar-2; 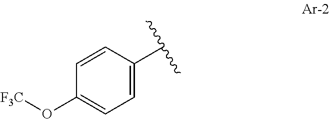 [SMILES: *c1ccc(OC(F)(F)F)cc1]
R¹ is a moiety of formula X—Y—Z-T-R″ or X—Y—Z-T-R¹²; wherein X—Y—Z-T is selected from X—Y—Z-T-1, X—Y—Z-T-2, X—Y—Z-T-3, X—Y—Z-T-4, X—Y—Z-T, X—Y—Z-T-9, X—Y—Z-T-13, X—Y—Z-T-16, X—Y—Z-T-17, X—Y—Z-T-18, and X—Y—Z-T-19;
R¹¹ is R¹¹-1 or R¹¹-10; 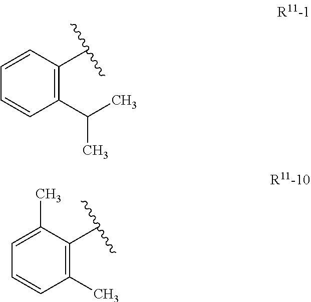
R¹² is formula A¹¹-1;

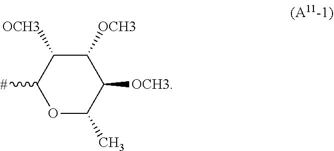

24. A composition comprising one compound of claim 16, an N-oxide or an agriculturally acceptable salt thereof.

25. The composition of claim 24, comprising additionally a further active substance.

26. A method for combating or controlling invertebrate pests, which method comprises contacting said pest or its food supply, habitat or breeding grounds with a pesticidally effective amount of at least one compound of claim 16.

27. A method for protecting growing plants from attack or infestation by invertebrate pests, which method comprises contacting a plant, or soil or water in which the plant is growing, with a pesticidally effective amount of at least one compound of claim 16.

28. Seed treated with the compound of claim 16, or an enantiomer, diastereomer or salt thereof, in an amount of from 0.1 g to 10 kg per 100 kg of seed.

29. A method for treating or protecting an animal from infestation or infection by invertebrate pests which comprises bringing the animal in contact with a pesticidally effective amount of at least one compound of the formula I of claim 16, a stereoisomer thereof and/or at least one veterinarily acceptable salt thereof.

30. A method for combating or controlling invertebrate pests, which method comprises contacting said pest or its food supply, habitat or breeding grounds with a pesticidally effective amount of the composition of claim 24.

31. A method for protecting growing plants from attack or infestation by invertebrate pests, which method comprises contacting a plant, or soil or water in which the plant is growing, with a pesticidally effective amount of the composition of claim 24.

32. The method of claim 26, wherein A is CR^{A} and G is N.

33. The method of claim 26, wherein A is N and G is CR^{B}.

34. The method of claim 26, wherein A is N and G is N.

35. The method of claim 26, wherein A is CR^{A} and G is CR^{B}.

36. The method of claim 26, wherein Q is NR²

37. The method of claim 26, wherein X—Y—Z-T are formulas XYZT-1 to XYZT-19 wherein 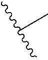
denotes attachment to the 6 membered hetaryl and # denotes attachment to R¹¹ or R¹²;

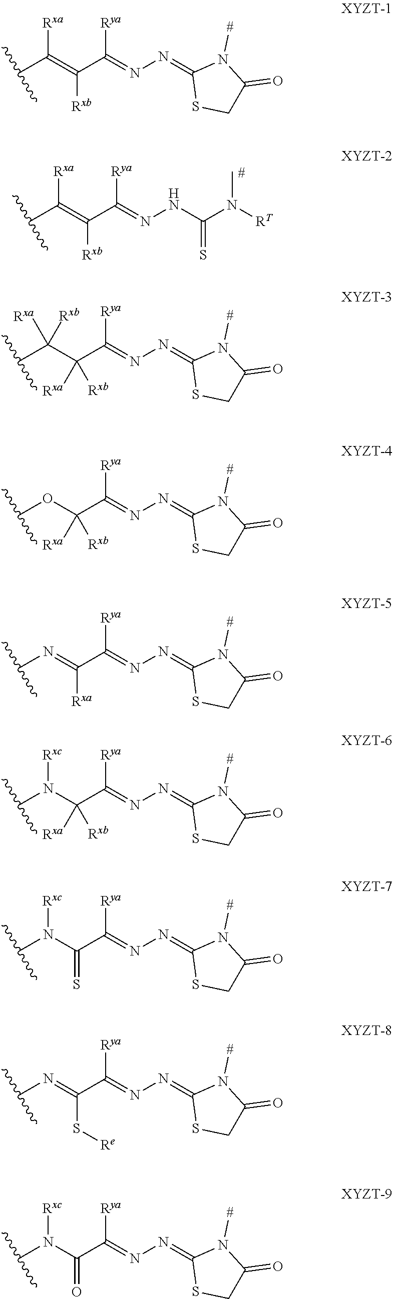

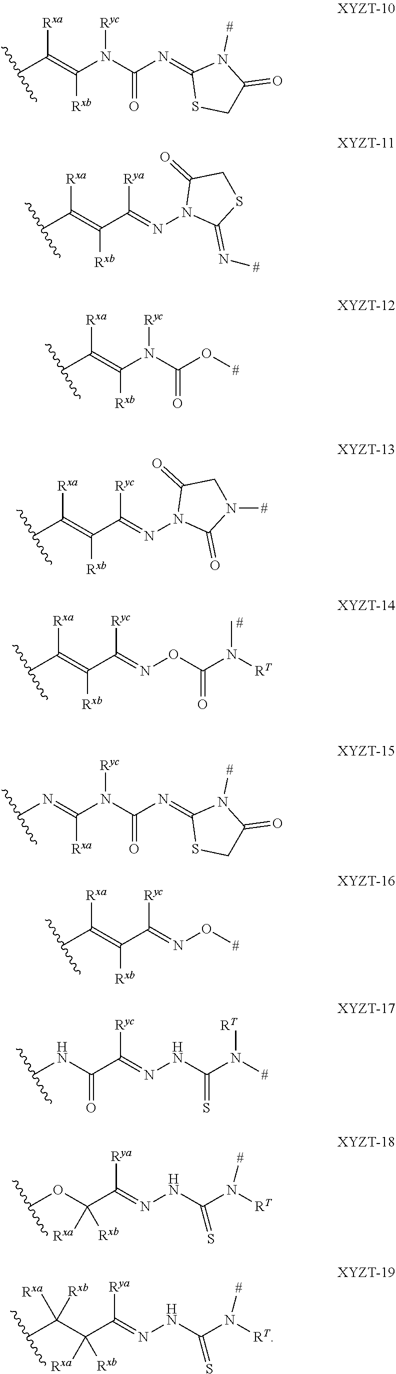

38. The method of claim 26, wherein
A is N or CR^{A};
G is N or CR^{B};
Q is NH or NCH₃;
R is H or C₁-C₆-alkyl;
R^{A} is H or N(CH₃)₂;
R^{B} is H or CH₃;
Ar is Ar-2; 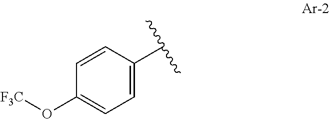 [SMILES: *c1ccc(OC(F)(F)F)cc1]
R¹ is a moiety of formula X—Y—Z-T-R″ or X—Y—Z-T-R¹²; wherein X—Y—Z-T is selected from X—Y—Z-T-1, X—Y—Z-T-2, X—Y—Z-T-3, X—Y—Z-T-4, X—Y—Z-T, X—Y—Z-T-9, X—Y—Z-T-13, X—Y—Z-T-16, X—Y—Z-T-17, X—Y—Z-T-18, and X—Y—Z-T-19;
R¹¹ is R¹¹-1 or R¹¹-10; 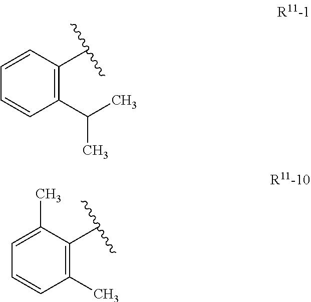
R¹² is formula A¹¹-1; [chemical structure]
